(19) United States
(12) Patent Application Publication
(10) Pub. No.: US 2023/0085358 A1
(43) Pub. Date: Mar. 16, 2023

(54) METHODS FOR CANCER TISSUE STRATIFICATION

(71) Applicant: GARVAN INSTITUTE OF MEDICAL RESEARCH, Darlinghurst (AU)

(72) Inventors: Ghamdan Abdulqawi Al-Eryani, Darlinghurst (AU); Daniel Lee Roden, Darlinghurst (AU); Sunny Ziyang Wu, Darlinghurst (AU); Alex Swarbrick, Darlinghurst (AU)

(21) Appl. No.: 17/849,470

(22) Filed: Jun. 24, 2022

(30) Foreign Application Priority Data
Jun. 25, 2021 (AU) 2021901939

(51) Int. Cl. C12Q 1/6886

(52) U.S. Cl. CPC C12Q 1/6886; C12Q 1/6869

(57) ABSTRACT

The present invention relates to methods for the classification and stratification of cells within tumour samples. In one aspect, the invention provides for methods for determining cell-type abundances in whole tumour samples and categorising these cell-type abundances into ecotypes.

### CROSS-REFERENCE TO RELATED APPLICATIONS

This application claims the benefit of and priority from Australian Provisional Application No. 2021901939, filed Jun. 25, 2021, the contents and disclosures of which are incorporated herein by reference in their entirety.

### TECHNICAL FIELD

The present invention relates to methods for the classification and stratification of cells within tumour samples. In one aspect, the invention provides for methods for determining cell-type abundances in whole tumour samples and categorising these cell-type abundances into ecotypes.

### BACKGROUND OF THE INVENTION

Cancer largely results from various molecular aberrations comprising somatic mutational events such as single nucleotide mutations, copy number changes and DNA methylations. In addition, cancer is viewed as a wildly heterogeneous disease, consisting of different subtypes with diverse molecular progression of oncogenesis and therapeutic responses. Many organ-specific cancers have established definitions of molecular subtypes on the basis of genomic, transcriptomic, and epigenomic characterizations, indicating diverse molecular oncogenic processes and clinical outcomes.

One such example is breast cancer (BrCa), which is stratified based on the expression of the estrogen receptor (ER), progesterone receptor (PR) and overexpression of HER2 or amplification of the HER2 gene ERBB2. This results in three broad clinical subtypes of BrCa: Luminal (ER+, PR+/−), HER2+(HER2+, ER+/−, PR+/−) and triple negative (TNBC; ER−, PR−, HER2−) that correlate with prognosis and define treatment strategies. Luminal cancers have an inherently less aggressive natural history than the Her2+ and TNBC subsets and are typically treated with systemic endocrine therapy targeting the Estrogen Receptor+/− cytotoxic chemotherapy. Her2+ cancers are treated with small molecule and antibody-based systemic drugs targeting the Her2 receptor plus cytotoxic chemotherapy. TNBC are typically only eligible for systemic cytotoxic chemotherapy and thus have the poorest outcomes of the 3 subtypes. BrCa are also stratified based on bulk transcriptomic profiling using the ‘PAM50’ gene signature into five ‘intrinsic’ molecular subtypes: luminal-like (LumA and LumB), HER2-enriched (HER2E), basal-like (BLBC) and normal-like. There is ˜70-80% concordance between molecular subtypes and clinical subtypes. For instance, the HER2E subtype is composed of clinically HER2+ and HER2− BrCa, as well as those that are ER+ and ER−3.

BrCa comprise diverse cellular microenvironments, whereby heterotypic interactions between neoplastic and non-neoplastic cells, such as stromal and immune cells, are important in defining disease etiology and response to treatment. So, while BrCa are generally considered to have a low mutational burden and immunogenicity, there is evidence that immune activation is pivotal in a subset of patients. It has followed that the presence of tumour infiltrating lymphocytes is a strong biomarker for good clinical outcome and complete pathological response to neoadjuvant chemotherapy. In contrast, tumour associated macrophages are often associated with poor prognosis and are recognised as important emerging targets for cancer immunotherapy. Moreover, mesenchymal cells have also emerged as important regulators of the malignant phenotype, chemotherapy response and anti-tumour immunity. Although these findings have elevated mesenchymal cells as critical mediators of tumour biology, progress has been impeded by a lack of a clear taxonomy of stromal subclasses.

Our understanding of the cellular heterogeneity and tissue architecture of human cancers has been largely derived from histology, bulk-sequencing, low dimensionality hypothesis-based studies and experimental model systems. As a consequence, information about the tumour microenvironment has not yet been integrated into clinical stratification and stromal-directed therapies are not yet in clinical practice.

A more detailed transcriptional atlas of various cancers at high molecular resolution, representative of all subtypes and cell types, is therefore required to further define the taxonomy of the disease and to determine how cells in the tumour microenvironment are organized as functional units in space. The identification of tumour heterogeneity is essential to the design of effective stratified treatments and for the discovery of treatments that can be extended to particular tumour subtypes.

In view of the above-described limitations, there is a need for improved methods for cancer stratification that overcome one or more of the above described limitations.

It will be clearly understood that, if a prior art publication is referred to herein, this reference does not constitute an admission that the publication forms part of the common general knowledge in the art in Australia or in any other country.

### SUMMARY OF THE INVENTION

In an aspect of the invention, there is provided a method for the identification of an ecotype within cancer samples, the method comprising:
i. performing or having performed single cell RNA sequencing on cancer sample training sets comprising different cell types and/or cell states;
ii. generating gene expression profiles from the cells of the cancer sample training sets based on the single cell RNA sequencing, wherein each gene expression profile correlates with a distinct cell type and/or cell state;
iii. generating cell abundance profiles, each cell abundance profile being based on the gene expression profile of a respective cancer sample training set; and
iv. using consensus-based clustering on the cancer samples with respect to the cell abundance profiles to identify an ecotype.

In an embodiment of the invention, the step of generating the gene expression profiles from the cells of the training set samples comprises annotating cells within each of the cancer sample training sets as a specific cell type and/or cell state.

In another aspect of the invention, there is provided a method for the identification of an ecotype within cancer samples, the method comprising:
i. generating cell abundance profiles, each cell abundance profile being based on a training set of a respective cancer sample; and
ii. using consensus-based clustering on the cancer samples with respect to the cell abundance profiles to identify an ecotype within the cancer samples.

In an embodiment, the step of generating a cell abundance profile based on the respective cancer sample training set comprises:
i. performing or having performed single cell RNA sequencing on the respective cancer sample training set comprising different cell types and/or cell states;

generating a cell gene expression profile for each cell of the respective cancer sample training set based on cell type or cell state, wherein the cell gene expression profile correlates with a distinct cell type and/or cell state within the respective In another aspect of the invention, there is provided a method for the identification of an ecotype within cancer samples, the method comprising:
i. performing or having performed single cell RNA sequencing on cancer sample training sets, each training set comprising different cell types and/or cell states;
ii. generating cell gene expression profiles from the cells of the cancer sample training sets based on the single cell RNA sequencing, each cell gene expression profile correlating with a distinct cell type and/or cell state;
iii. performing or having performed bulk gene expression RNA sequencing on cancer samples to generate a bulk gene expression profile of the cancer samples;
iv. processing the bulk gene expression profile based on the cell gene expression profiles to generate cell abundance profiles of the cancer samples, each cell abundance profile corresponding to a deconvolution of the bulk gene expression profile with respect to a respective cell gene expression profile; and
v. using consensus-based clustering on the cancer samples with respect to the cell abundance profiles to identify an ecotype within cancer samples.

In an embodiment of the invention, the method includes optionally applying the training set to a cancer sample from a subject by:
i. generating gene expression profiles of cancer samples;
ii. calculating cell-type abundances using a single-cell and/or bulk method; and
iii. assigning the cancer cells within the cancer sample to an ecotype, preferably using consensus-based clustering or machine learning.

In an embodiment, the step of generating a cell gene expression profiles comprises annotating cells within the cancer sample training sets as a specific cell type and/or cell state.

In another aspect of the invention, there is provided a method for the identification of an ecotype within cancer samples, the method comprising:
i. performing bulk gene expression RNA sequencing on cancer samples to generate a bulk gene expression profile of the cancer samples;
ii. processing the bulk gene expression profile based on cell gene expression profiles to generate cell type abundance profiles of the cancer samples, each cell abundance profile corresponding to a deconvolution of the bulk gene expression profile with respect to a respective cell gene expression profile; and
iii. using consensus-based clustering on the cancer samples with respect to the cell abundance profiles to identify an ecotype within cancer samples,
   wherein:
the cell gene expression profiles are generated from cells of the cancer sample training sets based on single cell RNA sequencing, each cell gene expression profile correlating with a distinct cell type and/or cell state.

In another aspect of the invention, there is provided a method for generating cell gene expression profiles based on which an ecotype within cancer samples can be determined, the method comprising:
i. performing single cell RNA sequencing on cancer sample training sets, each training set comprising different cell types and/or cell states; and
ii. generating cell gene expression profile from the cells of the cancer sample training sets based on the RNA sequencing, each cell gene expression profile correlating with a distinct cell type or cell state.

In an embodiment, from the cell gene expression profiles, an ecotype within cancer samples can be determined by:
i. performing bulk gene expression RNA sequencing on cancer samples to generate a bulk gene expression profile of the cancer samples;
ii. processing the bulk gene expression profile based on the cell gene expression profiles to generate cell abundance profiles of the cancer samples, each cell abundance profile corresponding to a deconvolution of the bulk gene expression profile with respect to a respective cell gene expression profile;
iii. using consensus-based clustering on the cancer samples with respect to the cell abundance profiles to identify an ecotype within cancer samples.

In an embodiment of the invention, the step of performing or having performed bulk gene expression RNA sequencing on cancer samples to generate a bulk gene expression profile of the cancer samples comprises the generation of bulk gene expression profiles from the same samples or the generation an independent dataset of bulk expression profiles, e.g., METABRIC.

In an embodiment of the invention, the ecotype may be selected from the group consisting of E1, E2, E3, E4, E5, E6, E7, E8 or E9.

In an embodiment of the invention, all steps of the methods described herein may be performed on a computer except for the initial generation of the single-cell or bulk gene expression profiles from the cancer sample.

In another aspect, there is provided a method for diagnosing or prognosing cancer in a subject, the method comprising:
i. performing or having performed single cell RNA sequencing on cancer sample training sets, each training set comprising different cell types and/or cell states;
ii. generating cell gene expression profiles from the cells of the cancer sample training sets based on the single cell RNA sequencing, each cell gene expression profile correlating with a distinct cell type and/or cell state;
iii. performing or having performed bulk gene expression RNA sequencing on cancer samples to generate a bulk gene expression profile of the cancer samples;
iv. processing the bulk gene expression profile based on the cell gene expression profiles to generate cell abundance profiles of the cancer samples, each cell abundance profile corresponding to a deconvolution of the bulk gene expression profile with respect to a respective cell gene expression profile;
v. using consensus-based clustering on the cancer samples with respect to the cell abundance profiles to identify an ecotype within cancer samples, and
vi. optionally administering a treatment to the subject based on the diagnosis or prognosis of cancer in the subject,
   wherein the ecotype is indicative of a diagnosis or prognosis of cancer in the subject.

In another aspect of the invention, there is provided a method for diagnosing or prognosing cancer in a subject, the method comprising:
i. performing or having performed single cell RNA sequencing on cancer sample training sets comprising different cell types and/or cell states;
ii. generating gene expression profiles from the cells of the cancer sample training sets based on the single cell RNA sequencing, wherein each gene expression profile correlates with a distinct cell type and/or cell state;
iii. generating cell abundance profiles, each cell abundance profile being based on the gene expression profile of a respective cancer sample training set; and
iv. using consensus-based clustering on the cancer samples with respect to the cell abundance profiles to identify an ecotype,
v. optionally administering a treatment to the subject based on the diagnosis or prognosis of cancer in the subject,
   wherein the ecotype is indicative of a diagnosis or prognosis of cancer in the subject.

In an embodiment of the invention, where an identification of ecotype, diagnosis, prognosis or prediction to drug treatment or survival is provided, the method may comprise:
i. training a predictor set of cancer samples from subjects with a known ecotype, diagnosis, prognosis, survival outcome or prediction to drug treatment; and
ii. applying the predictor to the cancer sample to determine ecotype, diagnosis, prognosis, survival or prediction to drug treatment of the subject.

Where the training of a predictor set of cancer samples from subjects with known diagnosis, prognosis or prediction to drug treatment or survival is required, the method may comprise:
i. performing cell deconvolution on bulk cancer cohorts (such as METABRIC);
ii. grouping those cancers into “ecotypes” based on the cell-type abundances, preferably by using a form of consensus clustering; and
iii. associating the ecotypes with diagnosis, prognosis, survival or prediction to drug treatment of the subject.

In another embodiment, where the training of a predictor set of cancer samples from subjects with known ecotype, diagnosis, prognosis or prediction to drug treatment or survival is required, the method may comprise applying the predictor set to test cancer sample from a subject by:
i. generating gene expression profiles of cancer samples;
ii. calculating cell-type abundances (using a single-cell and/or bulk method); and
iii. assigning the cancer cells within the cancer sample to an ecotype (e.g., using clustering or other classification methods such as machine learning).

In an embodiment of the invention, the method comprises identifying a treatment for the subject based on the identification of the ecotype the cancer sample. In this embodiment, the treatment may comprise chemotherapy, hormonal therapy, radiation therapy, biological therapy such as immunotherapy, small molecule therapy or antibody therapy, or a combination thereof. In another embodiment, the method comprises administering the identified treatment.

In an embodiment, the cancer may be any cancer known in the art or selected from the list consisting of include, but are not limited to, a basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intraepithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumours), and Meigs' syndrome.

In an embodiment, the subject from which the sample was obtained from a subject who has, or is suspected of having, breast cancer and exhibits one or more of the following symptoms:
presence of a lump in the breast or underarm;
thickening or swelling of part of the breast;
irritation or dimpling of breast skin;
redness or flaky skin in the nipple area or the breast;
pulling in of the nipple or pain in the nipple area;
nipple discharge including blood;
any change in the size or the shape of the breast; and
pain in an area of the breast.

In an embodiment, the cancer is diagnosed according to one or more clinical subtypes HR+/HER2− (“Luminal A”); HR−/HER2− (“Triple Negative”); HR+/HER2+(“Luminal B”) or HR−/HER2+(“HER2-enriched”). In another embodiment, the subject is diagnosed with a non-invasive or invasive carcinoma including ductal, lobular colloid (mucinous), medullary, micropapillary, papillary, and tubular invasive carcinoma.

In an embodiment, the method further comprises diagnosing the subject with any type of cancer defined herein or known in the art, preferably breast cancer. In another embodiment, the method further comprises a step of treating the subject for a period of time sufficient for a therapeutic response prior to obtaining the sample from the subject.

In an embodiment, the treatment comprises an adjuvant or neoadjuvant therapy. In another embodiment, the neoadjuvant or adjuvant therapy comprises or is selected from the group consisting of radiotherapy, chemotherapy, immunotherapy, biological response modifiers or hormone therapy.

In an embodiment, any gene expression profile or matrix described herein is generated using reverse transcription and real-time quantitative polymerase chain reaction (qPCR) with primers specific for each of the genes. In another embodiment, the gene expression profile is generated by microarray analysis with probes specific for each of the genes. In yet another embodiment, the gene expression profile or matrix is generated using RNA-Seq or other methods known in the art including Nanostring GeoMX DSP platform that uses hybridisation of probes, followed by elution and sequencing of probes to estimate GE; Spatial transcriptomics (commercialised as visium by 10× genomics) which uses spotted arrays of barcoded capture probes to perform something similar to a microarray; and methods that use sequencing in situ to perform targeted RNA-Seq in situ. In a preferred embodiment, the gene expression profile or matrix is generated using single-cell RNA sequencing.

In an embodiment, the gene expression profile is normalised to a control, preferably one or more housekeeping genes. In this embodiment, the housekeeping genes may be selected from RRN18S, ACTB, GAPDH, PGK1, PPIA, RPL13A, RPLPO, B2M, GUSB, HPRT1, TBP.

In another embodiment, the method comprises one or more of the following diagnostic tests:
ultrasound;
diagnostic x-ray;
magnetic resonance imaging (MRI); and
biopsy.

In another aspect, there is provided a method for predicting survival in a subject having or suspected of having cancer, the method comprising:
i. performing or having performed single cell RNA sequencing on cancer sample training sets, each training set comprising different cell types and/or cell states;
ii. generating cell gene expression profiles from the cells of the cancer sample training sets based on the single cell RNA sequencing, each cell gene expression profile correlating with a distinct cell type and/or cell state;
iii. performing or having performed bulk gene expression RNA sequencing on cancer samples to generate a bulk gene expression profile of the cancer samples;
iv. processing the bulk gene expression profile based on the cell gene expression profiles to generate cell abundance profiles of the cancer samples, each cell abundance profile corresponding to a deconvolution of the bulk gene expression profile with respect to a respective cell gene expression profile;
v. using consensus-based clustering on the cancer samples with respect to the cell abundance profiles to identify an ecotype within cancer samples,
wherein the ecotype is indicative of the survival of the subject having or suspected of having cancer.

In another aspect, there is provided a method for predicting survival in a subject having or suspected of having cancer, the method comprising:
i. performing or having performed single cell RNA sequencing on cancer sample training sets comprising different cell types and/or cell states;
ii. generating gene expression profiles from the cells of the cancer sample training sets based on the single cell RNA sequencing, wherein each gene expression profile correlates with a distinct cell type and/or cell state;
iii. generating cell abundance profiles, each cell abundance profile being based on the gene expression profile of a respective cancer sample training set; and
iv. using consensus-based clustering on the cancer samples with respect to the cell abundance profiles to identify an ecotype,
   wherein the ecotype is indicative of the survival of the subject having or suspected of having cancer.

In an embodiment, the prognosis or survival is selected from the group comprising or consisting of cancer specific survival, event-free survival, or response to therapy.

In an embodiment, samples with Basal-like and proliferative cells (or E3 as described herein) correlate with a poorer survival outcome or prognosis. In another embodiment, samples with HER2E and HER2E_SC cells (or E7 as described herein) correlate with a poorer survival outcome or prognosis. In another embodiment, samples with ecotypes comprising LumA and Normal-like cells (or E2 as described herein) correlate with a better survival outcome or prognosis. In another embodiment, samples with ecotypes comprising LumA, Normal-like cells as well as endothelial CXCL12+ and ACKR1+ cells, s1 MSC iCAFs and a depletion of cycling cells (or E2 as described herein) correlate with a better survival outcome or prognosis. Accordingly, ecotypes with a better survival outcome or prognosis have a better likelihood of cancer specific survival, event-free survival, or response to therapy.

In another aspect, there is provided a method for predicting a response to therapy in a subject having or suspected of having cancer, the method comprising:
i. performing or having performed single cell RNA sequencing on cancer sample training sets, each training set comprising different cell types and/or cell states;
ii. generating cell gene expression profiles from the cells of the cancer sample training sets based on the single cell RNA sequencing, each cell gene expression profile correlating with a distinct cell type and/or cell state;
iii. performing or having performed bulk gene expression RNA sequencing on cancer samples to generate a bulk gene expression profile of the cancer samples;
iv. processing the bulk gene expression profile based on the cell gene expression profiles to generate cell abundance profiles of the cancer samples, each cell abundance profile corresponding to a deconvolution of the bulk gene expression profile with respect to a respective cell gene expression profile;
v. using consensus-based clustering on the cancer samples with respect to the cell abundance profiles to identify an ecotype within cancer samples,
   wherein the ecotype is predictive of the response to therapy in the subject having or suspected of having cancer.

In another aspect, there is provided a method for predicting a response to therapy in a subject having or suspected of having cancer, the method comprising:
i. performing or having performed single cell RNA sequencing on cancer sample training sets comprising different cell types and/or cell states;
ii. generating gene expression profiles from the cells of the cancer sample training sets based on the single cell RNA sequencing, wherein each gene expression profile correlates with a distinct cell type and/or cell state;
iii. generating cell abundance profiles, each cell abundance profile being based on the gene expression profile of a respective cancer sample training set; and
iv. using consensus-based clustering on the cancer samples with respect to the cell abundance profiles to identify an ecotype,
   wherein the ecotype is indicative of the response to therapy in the subject having or suspected of having cancer.

In another aspect, there is provided a method for treating cancer in a subject having or suspected of having cancer, the method comprising:
i. performing or having performed single cell RNA sequencing on cancer sample training sets, each training set comprising different cell types and/or cell states;
ii. generating cell gene expression profiles from the cells of the cancer sample training sets based on the single cell RNA sequencing, each cell gene expression profile correlating with a distinct cell type and/or cell state;
iii. performing or having performed bulk gene expression RNA sequencing on cancer samples to generate a bulk gene expression profile of the cancer samples;
iv. processing the bulk gene expression profile based on the cell gene expression profiles to generate cell abundance profiles of the cancer samples, each cell abundance profile corresponding to a deconvolution of the bulk gene expression profile with respect to a respective cell gene expression profile;
v. using consensus-based clustering on the cancer samples with respect to the cell abundance profiles to identify an ecotype within cancer samples; and
ix. administering a treatment to the subject based on the ecotype in the cancer samples, thereby treating cancer in a subject having or suspected of having cancer.

In another aspect, there is provided a method for treating cancer in a subject having or suspected of having cancer, the method comprising:
i. performing or having performed single cell RNA sequencing on cancer sample training sets comprising different cell types and/or cell states;
ii. generating gene expression profiles from the cells of the cancer sample training sets based on the single cell RNA sequencing, wherein each gene expression profile correlates with a distinct cell type and/or cell state;
iii. generating cell abundance profiles, each cell abundance profile being based on the gene expression profile of a respective cancer sample training set;
iv. using consensus-based clustering on the cancer samples with respect to the cell abundance profiles to identify an ecotype; and
v. administering a treatment to the subject based on the ecotype of the cancer samples, thereby treating cancer in a subject having or suspected of having cancer.

In another aspect, there is provided use of a treatment in the preparation of a medicament for treating cancer in a subject having or suspected of having cancer, the use comprising:
i. performing or having performed single cell RNA sequencing on cancer sample training sets, each training set comprising different cell types and/or cell states;
ii. generating cell gene expression profiles from the cells of the cancer sample training sets based on the single cell RNA sequencing, each cell gene expression profile correlating with a distinct cell type and/or cell state;
iii. performing or having performed bulk gene expression RNA sequencing on cancer samples to generate a bulk gene expression profile of the cancer samples;
iv. processing the bulk gene expression profile based on the cell gene expression profiles to generate cell abundance profiles of the cancer samples, each cell abundance profile corresponding to a deconvolution of the bulk gene expression profile with respect to a respective cell gene expression profile,
v. using consensus-based clustering on the cancer samples with respect to the cell abundance profiles to identify an ecotype within cancer samples; and optionally
vi. administering a treatment to the subject based on the ecotype of the cancer samples.

In another aspect, there is provided use of a treatment in the preparation of a medicament for treating cancer in a subject having or suspected of having cancer, the use comprising:
i. performing or having performed single cell RNA sequencing on cancer sample training sets comprising different cell types and/or cell states;
ii. generating gene expression profiles from the cells of the cancer sample training sets based on the single cell RNA sequencing, wherein each gene expression profile correlates with a distinct cell type and/or cell state;
iii. generating cell abundance profiles, each cell abundance profile being based on the gene expression profile of a respective cancer sample training set; and
iv. using consensus-based clustering on the cancer samples with respect to the cell abundance profiles to identify an ecotype; and optionally
v. administering a treatment to the subject based on the ecotype of the cancer samples.

In an embodiment, the sample comprises ecotypes with cell type abundances selected from the group comprising or consisting of immune enriched cells; cycling cells; normal or healthy cells; PVLs; endothelial cells; myeloid cells; plasmablasts; B-cells; T-cells; innate lymphoid cells (ILCs); cancer associated fibroblasts; immune depleted; high cancer heterogenicity; and combinations thereof.

In an embodiment, the gene expression profile comprises a plurality of gene expression profiles, each of which correlates with a distinct cell type within a sample.

In an embodiment, the method comprises providing or having provided a cancer sample comprising different cell types.

In an embodiment, the sample comprises bulk tissue. In another embodiment, the sample comprises cells, blood or body fluid. In another embodiment, the sample comprises a formalin-fixed, paraffin-embedded (FFPE) tissue or a frozen tissue.

In a preferred embodiment, the cancer is breast cancer.

In an embodiment, the method comprises single cell RNA sequencing of least 1000, 2000, 3000, 4000 or 5000 cells.

In an embodiment, the deconvolution module comprises estimating cell type abundance using any known deconvolution method in the art, preferably the CIBERSORTx or DWLS method.

In another aspect, the invention provides a kit for identifying an ecotype in a cancer sample, the kit comprising reagents for the detection of the genes in the cancer sample. In an embodiment, the reagents comprise oligonucleotide primers and/or probes sufficient for the detection and/or quantitation of one or more of the genes in a cancer sample.

Any of the features described herein can be combined in any combination with any one or more of the other features described herein within the scope of the invention.

### BRIEF DESCRIPTION OF DRAWINGS

This patent application contains at least one drawing executed in color. Copies of this patent application with color drawing(s) will be provided by the Office upon request and payment of the necessary fee.

Various embodiments of the invention will be described with reference to the following drawings, in which:

FIGS. 1A-1C. H&E panel of all patients. Representative H&E images from all 26 breast tumours analysed by scRNA-Seq in this study. Scale bars represent 400 μm.

FIGS. 2A-2F. Single-cell RNA sequencing metrics and non-integrated data of stromal and immune cells. (FIGS. 2A-2B) Number of unique molecular identifiers (FIG. 2A) and genes (FIG. 2B) per tumour analyzed by scRNA-Seq in this study. Tumours are stratified by the clinical subtypes TNBC (red), HER2 (pink) and ER (blue). (FIGS. 2C-2D) Number of unique molecular identifiers (UMIs; C) and genes (FIG. 2D) per major lineage cell types identified in this study. These major lineage tiers are grouped by T-cells, B-cells, Plasmablasts, Myeloid, Epithelial, Cycling, Mesenchymal (cancer-associated fibroblasts and perivascular-like cells) and Endothelial. (FIGS. 2E-2F) UMAP visualization of all 71,220 stromal and immune cells without batch correction and data integration. UMAP dimensional reduction was performed using 100 principal components in the Seurat v3 package. Cells are grouped by tumour (FIG. 2E) and major lineage tiers (FIG. 2F) as identified using the Garnett cell classification method.

FIGS. 3A-3G. Cellular composition of primary breast cancers and the identification of malignant epithelial cells. (FIG. 3A) Integrated dataset overview of 130,246 cells analysed by scRNA-Seq. Clusters are annotated for their cell types as predicted using canonical markers and signature-based annotation using Garnett. (FIG. 3B) Log normalized expression of markers for epithelial cells (EPCAM), proliferating cells (MKI67), T-cells (CD3D), myeloid cells (CD68), B-cells (MS4A1), plasmablasts (JCHAIN), endothelial cells (PECAM1) and mesenchymal cells (fibroblasts/perivascular-like; PDGFRB). (FIG. 3C) Relative proportions of cell types highlighting a strong representation of the major lineages across tumours and clinical subtypes. (FIGS. 3D-3F) UMAP visualization of all epithelial cells, from tumours with at least 200 epithelial cells, colored by tumour (FIG. 3D), clinical subtype (FIG. 3E) and inferCNV classification (FIG. 3F). (FIG. 3G) InferCNV heatmaps of all malignant cells grouped by clinical subtypes. Common subtype-specific CNVs and a chr6 artefact reported by Tirosh et. al. are marked (Tirosh et al., (2016) Nature 539, 309-313).

FIGS. 4A and 4B. Identification of malignant epithelial cells using inferCNV. InferCNV heatmaps showing all epithelial cells and their associated inferCNV based classification for all tumours. For each cell, the normal cell call, copy number alteration (CNA) values, number of unique molecular identifiers (UMIs) and genes per cell are plotted on the right. Normal cell calls were classified as either Normal (green), Unassigned (grey) or Neoplastic (pink). These classifications are derived from a genomic instability score, which is estimated by the inferred changes at each genomic loci, as determined by inferCNV. High UMI and gene metrics in normal cells importantly show that they are not a product of coverage or low sequencing depth.

FIGS. 5A-5G. Data for scSubtype classifier. (FIG. 5A) Heirarchical Cluster of Allcells-Pseudobulk (Blue) and Ribozero mRNA-Seq (gold) profiles of the patient samples with TCGA patient mRNA-Seq data. (FIG. 5B) Zoomed in view of the basal cluster showing pairing of Allcells-Pseudobulk and Ribozero mRNA-Seq profiles of 2 representative tumours (dashed red boxes) in the present study. (FIG. 5C) Zoomed in view of the luminal cluster showing pairing of Allcells-Pseudobulk and Ribozero mRNA-Seq profiles of 4 representative tumours (dashed blue boxes) in the present study. (FIG. 5D) Heatmap of scSubtype gene sets across the training and test samples in each individual group. Colored outlined boxes highlighting the top expressed genes per group. (FIG. 5E) Barplot representing proportions of scSubtype calls in individual samples. Test dataset samples are highlighted within the golden colored outline. (FIG. 5F) Scatterplot of individual cancer cells plotted according to the Proliferation score (x-axis) and Differentiation—DScore (y-axis). Individual cells are colored based on the scSubtype calls. (FIG. 5G) Scatterplot of individual TCGA BrCa tumours plotted according to the Proliferation score (x-axis) and Differentiation—DScore (y-axis). Individual patients are colored based on the PAM50 subtype calls. Scatterplot of individual epithelial cells from 2 normal breast tissue samples showing the Proliferation score (x-axis) and Differentiation— DScore (y-axis). Individual cells are colored based on their classification into one of three human breast epithelial cell lineages (Mature luminal, Luminal Progenitor, and Basal/Myopeithelial).

FIGS. 6A-6H. Identifying drivers of neoplastic breast cancer cell heterogeneity. (FIG. 6A) Heatmap showing the average expression (scaled) of all cells assigned to each of the four scSubtypes. The top-5 most highly expressed genes in each subtype are shown, and selected others are highlighted. (FIG. 6B) Percentage of neoplastic cells in each tumour that are classified as each of the scSubtypes. Tumour samples are grouped according to their Allcells-pseudobulk classifications (NL=Normal-like). (FIG. 6C) CK5 and ER immunohistochemistry. Insert 1a/b represent CK5−/ER+ areas; Insert 2a/b represent CK5+/ER− areas. (FIG. 6D) Scatter plot of the proliferation scores and Differentiation Scores (DScores) of each neoplastic cell. Individual cancer cells are colored and grouped based on the scSubtype calls. All pairwise comparisons between cells from each scSubtype were significantly different (Wilcox test p<0.001) for both proliferation and DScores. (FIG. 6E) Gene-set enrichment, using ClusterProfiler, of the 200 genes in each of the gene-modules (GM1-7). Significantly enriched (adjusted p-value<0.05) gene-sets from the MSigDB HALLMARK collection are shown. (FIG. 6F) Proportion of cells assigned to each of the scSubtype subtypes grouped according to gene-module. (FIG. 6G) Scaled signature scores of each of the seven intra-tumour transcriptional heterogeneity gene-modules (rows) across all individual neoplastic cells (columns). Cells are ordered based on the strength of the gene-module signature score. (FIG. 6H) Percentage of neoplastic cells assigned to each of the seven gene-modules.

FIGS. 7A-7E. Data for breast cancer gene modules (FIG. 7A) The results from spherical k-means (skmeans) based consensus clustering of the Jaccard similarities between 574 signatures of neoplastic cell ITTH. This showed the probability (p1-p7) of each signature of ITTH being assigned to one of seven clusters/classes. Also shown is the Silhouette score for each signature. (FIG. 7B) Heatmap showing the scaled AUCell signature scores of each of the seven ITTH gene-modules (rows) across all individual neoplastic cells (columns) Hierarchical clustering was done using Pearson correlations and average linkage. (HER2_AMP=Clinical HER2 amplification status). (FIG. 7C) Boxplots showing the distributions of signature scores (z-score scaled) for each of the gene-module signatures. The cells are grouped according to the gene-module (GM1-7) cell-state that they are assigned. (FIG. 7D) Barchart showing the proportion of cells assigned to each of the gene-module cell-states (GM1-7) with cells grouped according to the scSubtypes that they are assigned. (FIG. 7E) Boxplots showing the distributions of scSubtype scores for each of the gene-module signatures. The cells are grouped according to the gene-module (GM1-7) cell-state that they are assigned. Kruskal-Wallis tests were performed to calculate the significance between the four scSubtype score groups in each of the gene-module groups, p-value shown. Wilcox tests were used to identify which scSubtype had significantly increased scSubtype scores in the cells assigned to each gene-module, the scores of each scSubtype were compared to the rest of the scSubtype scores (****: Holm adjusted p-value<0.0001, ns: Holm adjusted p-value>0.05).

FIGS. 8A-8I. Immune landscape of breast cancers reveals distinct T-cell and myeloid phenotypes across breast cancers. (FIG. 8A) Reclustering T-cells and innate lymphoid cells and their relative proportions across tumours and clinical subtypes. (FIG. 8B) Imputed CITE-Seq protein expression values for selected markers and checkpoint molecules. (FIG. 8C) Pairwise t-test comparisons revealing the significant enrichment of T-cells:IFIT1, T-cells:KI67, CD8+ T-cells:LAG3 in TNBC tumours, and significant depletion of LAM 1:FABP5 in HER2+ tumours. Statistical significance was determined using a student t-test in a pairwise comparison of means between groups. P-values denoted by asterisks: *p<0.05, p<0.01, *p<0.001 and ****p<0.0001. (FIG. 8D) Cluster averaged dysfunctional and cytotoxic effector gene signature scores in T-cells and innate lymphoid cells stratified by clinical subtypes. (FIG. 8E) Reclustered myeloid cells and their relative proportions across tumours and clinical subtypes. (FIG. 8F) Cluster averaged expression of various published gene signatures acquired from independent studies used for Myeloid cluster annotation. Selected genes of interest from each signature are listed. (FIG. 8G) Kaplain Meier plots showing associations between LAM 1:FABP5 and LAM 2: APOE with overall survival in METABRIC cohort. P-values were calculated using log-rank test. Time (x-axis) is represented in months. (FIG. 8H) Imputed CITE-Seq expression values for canonical markers and checkpoint molecules across Myeloid clusters. (FIG. 8I) Cluster averaged gene expression of clinically relevant immunotherapy targets. Clusters are grouped by breast cancer clinical subtype and immune cell type annotations. Genes are grouped as receptor (purple) or ligand (green), the inhibitory (red) or stimulatory status (blue) and the expected major lineage cell types known to express the gene (lymphocyte, green; myeloid, pink; both, light purple).

FIGS. 9A-9D. CITE-Seq vignette (FIG. 9A) UMAP Visualization of a TNBC sample with 157 DNA barcoded antibodies (data not shown). Cluster annotations were extracted from our final breast cancer atlas cell annotations. (FIG. 9B) Stacked violin plots of canonical gene expression markers for B-cells (MS4A1/CD20), fibroblasts/perivascular-like cells (COL1A1 and ACTA2), endothelial cells (PECAM1), monocyte and macrophages (LYZ), T-cell clusters (CD3D, CD4, CD8A) and NKT cells (NKG7). (FIG. 9C) Heatmap visualization of the cluster averaged antibody derived tag (ADT) values for the 157 CITE-seq antibody panel. Only immune cells are shown. (FIG. 9D) Expression featureplots of measured experimental ADT values (shown in top rows) against the CITE-Seq imputation ADT levels (shown in bottom rows), as determined using the seurat v3 method. Selected markers for immunophenotyping T-cells (CD4, CD8A, PD-1 and CD103) and myeloid cells (PD-L1, CD86, CD49f and CD14) are shown.

FIGS. 10A-10N. Data for T-cells, Myeloid, B-cells and Plasmablasts. (FIG. 10A) Dotplot visualizing averaged expression of canonical markers across T-cell and innate lymphoid clusters. (FIG. 10B) Cytotoxic and dysfunctional gene signature scores across T-cell and innate lymphoid clusters. A Kruskal-Wallis test was performed to compare multiple groups' significance. Additionally, a pairwise student t-test for each cluster to mean was used to determine significance. P-values denoted by asterisks: *p<0.05, **p<0.01, ***p<0.001 and ****p<0.0001. Red line marks median expression across clusters. (FIG. 10C) Dysfunctional gene signature scores of CD8: LAG3 and CD8+ T: IFNG clusters across BrCa subtypes. A pairwise student t-test for each cluster was performed to determine significance. P-values denoted by asterisks: *p<0.05, **p<0.01, ***p<0.001 and ****p<0.0001. (FIG. 10D) Differentially expressed immune modulator genes, stratified by T-cell and Myeloid clusters, found to be statistically significant when compared across breast cancer subtypes. A pairwise MAST comparison was performed to obtain bonferroni corrected p-values. P-values denoted by asterisks: *p<0.05, **p<0.01, ***p<0.001 and **** p<0.0001. (FIG. 10E) Pairwise t-test comparison of LAG3, CD27, PD-1 (PDCD1), CD70 and CD27 Log-normalised expression found in LAG3/c8 T-cells across breast cancer subtypes. (FIG. 10F) Enrichment of PDCD1, CD27, LAG3, CD70 expression in METABRIC cohort between BrCa subtypes. A pair-wise Wilcox test was performed to identify statistical significance. P-values denoted by asterisks: *p<0.05, **p<0.01, ***p<0.001 and ****p<0.0001. (FIG. 10G) UMAP visualization of all reclustered B-cells and Plasmablasts as annotated using canonical gene expression markers. (FIG. 10H) Featureplots of naïve B cells, memory B cells, and Plasmablasts. (FIG. 10I-10J) Tumour associated macrophage (TAM) signature score obtained from Cassetta et al., (2019) Cancer Cell, 35(4):588-602 and the expression of log-normalised levels of CCL8 across all myeloid clusters. A pairwise student t-test was performed to determine statistical significance for clusters of interest. P-values denoted by asterisks: *p<0.05, **p<0.01, ***p<0.001 and ****p<0.0001. Dashed red line marks median TAM gene score expression. A Kruskal-Wallis test was performed to compare multiple groups' significance. (FIG. 10K) LAM and DC:LAMP3 gene expression signatures acquired from Jaitin et al. (2019) Cell 178(3):686-698 and Zhang et al., (2019) Cell 179, 829-845 respectively, visualized on UMAP myeloid clusters. (FIG. 10L) Proportional change of myeloid subsets across different BrCa subtypes. Statistical significance was determined using a student t-test in a pairwise comparison of means between groups. P-values denoted by asterisks: *p<0.05, **p<0.01, ***p<0.001 and ****p<0.0001. Any comparison without asterisk means no significance was found. (FIG. 10M) Heatmap visualizing GO enrichment pathways across Myeloid clusters. (FIG. 10N) Violin plot of Imputed CITE-seq PD-L1 and PD-L2 expression values found on Myeloid cells.

FIG. 11. Gene expression of immune cell surface receptors across malignant, immune and mesenchymal clusters and breast cancer clinical subtypes. Averaged expression and clustering of 133 clinically targetable receptor or ligand immune modulator markers across all cell types grouped by clinical breast cancer subtypes (TNBC, HER2+ and ER+). Gene list was manually curated through systematic literature search of known immune modulating proteins expressed on the surface of cells. Default parameters for hierarchical clustering were used via the “pheatmap” package for the visualization of gene expression values.

FIGS. 12A-121. Supplementary data for mesenchymal cell states and subclusters. (FIG. 12A) UMAP visualization CAFs, PVL cells and endothelial cells using Seurat reclustered with default resolution parameters (0.8). (FIG. 12B) Genes driving Principal Component 1 for CAFs, PVL cells and endothelial cells, revealing an enrichment of mesenchymal cell activation and differentiation markers. (FIG. 12C) UMAP visualizations for CAFs, PVL cells and endothelial cells with monocle derived cell states overlaid (as determined in FIGS. 4C-4H). (FIG. 12D) Top 10 gene ontologies (GO) of each mesenchymal cell state, as determined using pathway enrichment with ClusterProfiler with all differentially expressed genes as input. (FIGS. 12E-12F) Signature scores for pancreatic ductal adenocarcinoma myofibroblast-like, inflammatory-like and antigen-presenting CAF sub-populations, as determined using AUCell. Signature scores are represented through single-cell violin plots (FIG. 12E) and cluster averaged heatmap (FIG. 12F). (FIG. 12G) Enrichment of antigen-presenting CAF markers PTGIS, CLU, CD74 and CAV1 in CAF sub-clusters c11, c12 and c5, determined using Seurat clustering rather than monocle derived cell states. (FIG. 12H) Subclusters of CAFs, PVL cells and endothelial cells determined using Seurat show a strong integration with three normal breast tissue datasets, highlighting similarities in subclusters across disease status and subtypes of breast cancer. (FIG. 12I) Cell states of CAFs, PVL cells and endothelial cells determined using monocle show a strong integration with three normal breast tissue datasets and breast cancer subtypes.

FIGS. 13A-13J. Transcriptional profiling and phenotyping of diverse mesenchymal differentiation states across clinical BrCa subtypes. (FIG. 13A) Reclustered mesenchymal cells, including CAFs (6,573 cells), perivascular-like (PVL) cells (5,423 cells), endothelial cells (7,899 cells; ECs), lymphatic ECs (203 cells) and cycling PVL (50 cells). Cell sub-states are defined using pseudotemporal ordering with the monocle 2 method (as in C-H below). (FIG. 13B) Featureplots of canonical markers for CAFs (PDGFRA, COL1A1, ACTA2, PDGFRB), PVL (ACTA2, PDGFRB and MCAM) and ECs (PECAM1, CD34 and VWF). (FIGS. 13C-13H) Pseudotemporal ordering and differentially expressed genes between states of CAFs (FIGS. 13C-13D), PVL cells (FIGS. 13E-13F) and ECs (FIGS. 13G-13H). Heatmaps for each cell type (right) show cell state averaged log normalised expression values for all differentially expressed genes determined using the MAST method, with select stromal markers highlighted. (FIGS. 13C-13D) CAFs fell into five cell states. CAF s1 and s2 both resemble mesenchymal stem cells (MSC; ALDH1A1 and KLF4) and inflammatory CAF-like states (MSC/iCAF; CXCL12 and C3). CAF s2 was distinct from s1 by DLK1. CAF s4 and s5 resemble myofibroblast-like CAF states (myCAF; ACTA2 and TAGLN) which were enriched for ECM genes (COL1A1). CAF s3 shared features of both MSC/iCAFs and myCAFs and resembled a transitioning state (s3). (FIGS. 13E-13F) PVL cells grouped into three states. PVL s1 and s2 resemble progenitor and immature states (imPVL; CD44). PVL s3 resembles a contractile and differentiated state (dPVL; MYH11). (FIGS. 13G-13H) ECs resemble a venular stalk-like state (s1; ACKR1) and two tip-like states (s2 and s3). s2 and s3 are distinguished by RGS5 and CXCL12, respectively. (FIG. 131) Featureplots of imputed CITE-Seq antibody-derived tag (ADT) protein levels for canonical markers of CAFs (Podoplanin), PVL cells (CD146/MCAM) and ECs (CD31 and CD34). UMAP coordinates correspond to those in A. (FIG. 13J) Heatmap of cluster averaged imputed CITE-Seq values for additional cell surface markers and functional molecules.

FIGS. 14A-14H. Deconvolution of breast cancer cohorts using single-cell signatures reveals robust ecotypes associated with patient survival and intrinsic subtypes. (FIG. 14A) Summary of the major epithelial, immune and stromal cell types identified in this study grouped by their major (inner), minor and subset (outer) level classification tiers. (FIG. 14B) Boxplot comparing the CIBERSORTx predicted scSubtype and Cycling cell-fractions in each METABRIC patient tumour, stratified by PAM50 subtypes. (FIG. 14C) Consensus clustering of all tumours (columns) in METABRIC showing nine robust tumour ecotypes and 4 groups of cell enrichments from 45 cell-types in the BrCa cell taxonomy. (FIG. 14D) Relative proportion of the PAM50 molecular subtypes of the tumours in each ecotype. (FIG. 14E) Relative average proportion of the major cell-types enriched in the tumours in each ecotype. (FIGS. 14F-14H) Kaplan-Meier (KM) plot of the patients with tumours in each of the nine ecotypes (FIG. 14F), patients with tumours in ecotypes E2 and E7 (FIG. 14G), patients with tumours in ecotypes E4 and E7 (FIG. 14H). p-values calculated using the log-rank test.

FIGS. 15A-15K. (FIG. 15A) Bar and boxplots (inset) of the Pearson correlation, for each of the 45 cell-types in the subset level of the BrCa cell taxonomy, between the actual cell-fractions captured by scRNA-Seq and the CIBERSORTx predicted fractions from pseudo-bulk expression profiles. * denotes a significant correlation p<0.05 between actual and predicted cell-type abundance. (FIG. 15B) Barplot comparing the Pearson correlation, for each of the cell-types in the subset level of the BrCa cell taxonomy, between the actual cell-fractions captured by scRNA-Seq and the CIBERSORTx and DWLS predicted fractions from pseudo-bulk expression profiles. * denotes a significant correlation p<0.05 between actual and predicted cell-type abundance. (FIG. 15C) Heatmap of ecotypes formed from the common METABRIC tumours (columns) identified from combining ecotypes generated using CIBERSORTx with all, or the 32 significantly correlated cell-types (rows), when using CIBERSORTx on pseudo-bulk samples. (FIG. 15D) Relative proportion of the PAM50 molecular subtypes of the common tumours in each ecotype, when combining CIBERSORTx consensus clustering results from using all or the 32 significant cell-types. (FIG. 15E) Relative average proportion of the major cell-types enriched in the common tumours in each ecotype, when combining CIBERSORTx consensus clustering results from using all or the 32 significant cell-types. (FIGS. 15F-15G) Kaplan-Meier (KM) plot of all patients with common tumours in each of the ecotypes (F), patients with tumours in ecotypes E4 and E7 (FIG. 15G), when combining CIBERSORTx consensus clustering results from using all or the 32 significant cell-types. p-values calculated using the log-rank test. (FIG. 15H) Relative proportion of the PAM50 molecular subtypes of the common tumours from combining CIBERSORT and DWLS generated ecotypes. (FIG. 15I) Relative average proportion of the major cell-types enriched in common tumours from combining CIBERSORT and DWLS generated ecotypes. (FIG. 15J) Kaplan-Meier (KM) plot of the patients with tumours in ecotypes E4 and E7, formed from combining CIBERSORT and DWLS generated ecotypes. p-value calculated using the log-rank test. (FIG. 15K) Relative proportion of the METABRIC integrative cluster annotations of the tumours in each ecotype (ecotypes generated using CIBERSORTx across all cell-types).

Preferred features, embodiments and variations of the invention may be discerned from the following Description which provides sufficient information for those skilled in the art to perform the invention. The following Description is not to be regarded as limiting the scope of the preceding Summary of the Invention in any way.

### DETAILED DESCRIPTION

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims. One skilled in the art will recognize many methods and materials similar or equivalent to those described herein, which could be used in the practice of the present invention. The present invention is in no way limited to the methods and materials described.

It will be understood that the invention disclosed and defined in this specification extends to all alternative combinations of two or more of the individual features mentioned or evident from the text or drawings. All of these different combinations constitute various alternative aspects of the invention. It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects, and vice versa, unless the context clearly dictates otherwise. For example, reference to “a” includes a single as well as two or more; reference to “an” includes a single as well as two or more; reference to “the” includes a single as well as two or more and so forth.

In the present specification and claims (if any), the word ‘comprising’ and its derivatives including ‘comprises’ and ‘comprise’ include each of the stated integers but does not exclude the inclusion of one or more further integers.

One skilled in the art will recognize many methods and materials similar or equivalent to those described herein, which could be used in the practice of the present invention. The present invention is in no way limited to the methods and materials described.

The present invention is not to be limited in scope by the specific examples described herein, which are intended for the purpose of exemplification only. Functionally-equivalent products, compositions and methods are clearly within the scope of the present invention.

Any example or embodiment of the present invention herein shall be taken to apply mutatis mutandis to any other example or embodiment of the invention unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Cancer largely results from various molecular aberrations comprising somatic mutational events such as single nucleotide mutations, copy number changes and DNA methylations. In addition, cancer is viewed as a wildly heterogeneous disease, consisting of different subtypes with diverse molecular progression of oncogenesis and therapeutic responses. Many organ-specific cancers have established definitions of molecular subtypes on the basis of genomic, transcriptomic, and epigenomic characterizations, indicating diverse molecular oncogenic processes and clinical outcomes.

The inventors show herein for the first time the development of a single cell method for the stratification of tumour samples into tumour ecotypes. In particular, by using single cell signatures, deconvolution of large breast cancer cohorts allows for the stratification of tumour samples into nine clusters, termed ‘ecotypes’, with unique cellular compositions and clinical outcomes.

This approach has advantages over previously described approaches including:
simultaneous inference of cell type-specific gene expression profiles (GEPs) and cell type abundance from bulk tissue transcriptomes;
accurate estimation of bulk tissue composition using scRNA-Seq-derived reference signatures;
cost-effective, high-throughput tissue characterization without antibodies, disaggregation, or viable cells;
stratification of cancers based on cell type abundance, rather than genomic or genetic features. As such it provides prognostic, diagnostic or predictive information orthogonal to those established methods;
by using deconvolution, cell type abundances can be estimated from bulk gene expression profiles, obviating the necessity for scRNA-Seq analysis of tumours, which is costly, complex and time-consuming.

Moreover, whilst WO 2019/018684 provides a computational framework for performing in silico tissue dissection to accurately infer cell type abundance and cell type (e.g., cell type-specific) gene expression from RNA profiles of intact tissues, the inventors work described herein provides for superior signatures that have been specifically extracted from breast cancers and provides for clustering of patients, optionally after deconvolution to stratify patients into groups with similar composition into ecotypes.

Tissue composition can be a major determinant of phenotypic variation and a key factor influencing disease outcomes. Although scRNA-Seq can be a powerful technique for characterizing cellular heterogeneity, it can be impractical for large sample cohorts and may not be applied to fixed specimens collected as part of routine clinical care. To overcome these challenges, the present disclosure provides a platform for in silico cytometry that can enable the simultaneous inference of cell type-specific gene expression profiles (GEPs) and cell type abundance from bulk tissue transcriptomes. Using the methods disclosed herein for in silico purification, bulk tissue composition can be accurately estimated using scRNA-Seq-derived reference signatures. The disclosed methods and systems may link unbiased cell type discovery with large-scale tissue dissection. Digital cytometry can augment single-cell profiling efforts, enabling cost-effective, high-throughput tissue characterization without antibodies, disaggregation, or viable cells.

Immunophenotyping approaches, such as flow cytometry and immunohistochemistry (IHC), can rely on small combinations of preselected marker genes, which can limit the number of cell types that can be simultaneously interrogated. By contrast, single-cell mRNA sequencing (scRNA-Seq) can be used for unbiased transcriptional profiling of hundreds to thousands of individual cells from a single-cell suspension (scRNA-Seq). Despite the power of this technology, analyses of large sample cohorts may not be practical, and many fixed clinical specimens (e.g., formalin-fixed, paraffin embedded (FFPE) samples) may not be dissociated into single-cell suspensions. Furthermore, the impact of tissue disaggregation on cell type representation may be poorly understood.

Computational techniques for dissecting cellular content directly from genomic profiles of mixture samples may rely on a specialized knowledgebase of cell type-specific “barcode” genes (e.g., a “signature matrix”), which is derived from FACS-purified or in vitro differentiated/stimulated cell subsets. Although useful when cell types of interest are well defined, such gene signatures may be suboptimal for the discovery of novel cell types and cell type gene expression profiles, and for capturing the full spectrum of major cell phenotypes in complex tissues.

The present disclosure provides a computational framework to accurately infer cell type abundance and cell type-specific gene expression from RNA profiles of intact tissues. By leveraging cell type expression signatures from single-cell experiments or sorted cell subsets, the methods of the present disclosure can provide comprehensive portraits of tissue composition without physical dissociation, antibodies, or living material. Such approaches may include, for example, a method for enumerating cell composition from tissue gene expression profiles with techniques for cross-platform data normalization and in silico cell purification. The latter can allow the transcriptomes of individual cell types of interest to be digitally “purified” from bulk RNA admixtures without physical isolation. As a result, changes in cell type-specific gene expression can be inferred without cell separation or prior knowledge. The results described herein illustrate that methods of the present disclosure are useful for deciphering complex tissues, with implications for high-resolution cell phenotyping in research and clinical settings.

The methods described herein can be used to decode cellular heterogeneity in complex tissues. This strategy can be used to “digitally gate” cell subsets of interest from single-cell transcriptomes, profile the identities and expression patterns of these cells in cohorts of bulk tissue gene expression profiles (e.g., fixed specimens from clinical trials), and systemically determine their associations with diverse metadata, including genomic features and clinical outcomes.

The term “scRNA-Seq,” as used herein, generally refers to a single-cell RNA sequencing method to obtain expression profiles of individual cells. For example, single-cell libraries can be prepared from single-cell suspensions of dissociated cancers (e.g., from cancer patients) using Chromium with v2 chemistry (10× Genomics). Such single-cell libraries can be sequenced (e.g., a NextSeq 500 (Illumina)). Sequencing reads may be processed, for example, by alignment, filtration, deduplication, and/or conversion into a digital count matrix using Cell Ranger 1.2 (10× Genomics).

Outlier cells may be identified and filtered based on (1) anomalously high/low mitochondrial gene expression (e.g., cells with >10 or <1 mitochondrial content may be removed) and/or (2) potential doublets/multiplets, as identified by comparing the number of expressed genes detected by per cell versus the number of unique molecular identifiers (UMIs) detected per cell (e.g., cells with greater than 3,500 and less than 500 expressed genes may be removed). Clusters may be identified (e.g., using Seurat v.1.4.0.16) by (1) regressing out the dependence of gene expression on the number of unique molecular identifiers (UMIs) and the percentage of mitochondrial content, and (2) by running “FindClusters” on a suitable number (e.g., 1, 2, 3, 4, 5, 6, 7, 8, 9, 10, or more than 10) of principal components of the data. Cell labels may be assigned according to the expression of canonical marker genes, for instance in leukocytes (e.g., MS4A1 high=B cells; CD8A high and GNLY low=CD8 T cells; CD3E high, CD8A low, and GNLY low=CD4 T cells; GNLY high and CD3E low=NK cells; GNLY high and CD3E high=NKT cells; CD14 high=monocytes). Publicly available PBMC datasets from healthy donors profiled by Chromium v2 (5′ and 3′ kits) may be downloaded (Table 1) and preprocessed as above, with the following minor modifications.

During quality control, cells with >5000 expressed genes for 5′ assays, >4000 expressed genes for 3′ assays, and <200 expressed genes may be excluded. Seurat “FindClusters” may be applied on the first 20 principal components, with the resolution parameter set to 0.6. Cell labels may be assigned as described above. In addition, myeloid cells may be defined by high CD68 expression, megakaryocytes may be defined by high PPBP expression, and dendritic cells may be defined by high FCER1A expression.

The term “bulk RNA-Seq,” as used herein, generally refers to a bulk RNA sequencing method to obtain expression profiles of bulk cell populations or tissues. For example, total RNA may be isolated from blood samples stored in, e.g., PAXgene tubes using, e.g., the PAXgene Blood RNA Kit (Qiagen) according to the manufacturer's recommendations. RNA may be quantitated and quality assessed using, e.g., a 2100 Bioanalyzer (Agilent). Library preparation may be performed using, e.g., an RNA exome kit (Illumina) per the manufacturer's recommendations. RNA-Seq libraries may be multiplexed together and sequenced using, e.g., a single HiSeq 4000 lane (Illumina) using 2×150 bp reads. For example, total RNA may be isolated from PBMC samples using TRIzol (Invitrogen) per the manufacturer's recommendations. RNA molecules may be quantitated and quality assessed, e.g., using a 2100 Bioanalyzer (Agilent) with a RNA 6000 Pico chip (Agilent). Library preparation of the RNA molecules may be performed, e.g., using the SMARTer Stranded Total RNA-Seq—Pico kit (Takara Biosciences) per the manufacturer's recommendations. Libraries may be quantified, e.g., with the dsDNA HS Assay kit (Thermo Fisher Scientific) using a Qubit 3.0 fluorometer (Thermo Fisher Scientific). Library quality may be assessed, e.g., using a 4200 TapeStation Instrument (Agilent) with D1000 ScreenTape. RNA-Seq libraries may be sequenced on a suitable sequencing instrument (e.g., a NextSeq 500 (Illumina) using 2×150 base-pair (bp) reads). As another example, total RNA may be extracted from bulk tumours (e.g., NSCLC) and sorted cell populations (e.g., in a range of about 100, about 200, about 300, about 400, about 500, about 1,000, about 5,000, about 10,000, about 15,000, about 20,000, about 25,000, or more than 25,000 cells), e.g., using an AllPrep DNA/RNA Micro kit (Qiagen).

An amount of total RNA (e.g., about 10 nanograms (ng), about 20 ng, about 30 ng, about 40 ng, about 50 ng, or more than 50 ng) may be amplified, e.g., using an Ovation RNA-Seq System V2 (NuGEN). The resulting complementary DNA (cDNA) may be sheared (e.g., by sonication (Covaris S2 System) to an average size of 150-200 bp) and used to construct DNA libraries (e.g., using the NEBNext DNA Library Prep Master Mix (New England Biolabs)). Libraries may be sequenced on a suitable sequencing instrument (e.g., a HiSeq 2000 (Illumina) to generate 100 bp paired end reads with an average of 100 million (M) reads per sample).

To maximize linearity in the context of deconvolution analyses, raw FASTQ reads may be processed (e.g., with Salmon v0.8.265) using GENCODE v23 reference transcripts, the—biasCorrect flag, and otherwise default parameters. RNA-Seq quantification results may be merged into a single gene-level TPM matrix using an R package, tximport.

Microarrays may be used to generate ground truth reference profiles using microarrays. Total RNA may be extracted from bulk FL specimens and sorted B cells and assessed for yield and quality. Complementary RNA (cRNA) may be prepared from 100 ng of total RNA following linear amplification (3′ IVT Express, Affymetrix), and then hybridized to HGU133 Plus 2.0 microarrays (Affymetrix) according to the manufacturer's protocol. Obtained CEL data files may be pooled with a publicly available Affymetrix dataset containing CD4 and CD8 tumorinfiltrating lymphocytes (TILs) which are FACS-sorted from FL lymph nodes (GSE2792840). Resulting datasets may be RMA normalized using the “affy” package in Bioconductor, mapped to NCBI Entrez gene identifiers using a custom chip definition file (e.g., Brainarray version 21.0; http://brainarray.mbni.med.umich.edu/Brainarray/), and converted to HUGO gene symbols. Replicates of sorted cell subsets may be combined to create ground truth reference profiles using the geometric mean of expression values.

External datasets may comprise next generation sequencing (NGS) datasets which are downloaded and analyzed using normalization settings. Such external datasets may comprise one or more of: transcripts per million (TPM), reads per kilobase of transcript per million (RPKM), or fragments per kilobase of transcript per million (FPKM) space. For analyses in log 2 space, values of 1 may be added to expression values prior to log 2 adjustment. Affymetrix microarray datasets may be summarized and normalized as described with microarrays, using RMA in cases where bulk tissues and ground truth cell subsets were profiled on the same Affymetrix platform, and otherwise using MASS normalization. NanoString nCounter data may be downloaded and analyzed with batch correction in non-log linear space, but without any additional preprocessing.

Single-cell expression values may be first normalized to transcript per million (TPM) and divided by 10 to better approximate the number of transcripts per cell. For each cell phenotype, genes with low average expression in log 2 space may be set to 0 as a quality control filter. Because of sparser gene coverage, filter may not be applied to data generated by 10× Chromium. For each cell type represented by at least 3 single cells, 50% of all available single cell GEPs may be selected using random sampling without replacement (fractional sample sizes may be rounded up such that 2 cells were sampled if only 3 were available). The profiles may be aggregated by summation in non-log linear space and each population-level GEP may be normalized into TPM. This process may be repeated in order to generate aggregated transcriptome replicates (e.g., 2, 3, 4, 5, or more than 5) per cell type. For example, scRNA-Seq and bulk RNA-Seq signature matrices may be generated as described previously with the following typical parameters: minimum number of genes per cell type=300, maximum number of genes per cell type=500, q-value of 0.01, and no quantile normalization.

### Genes for Cell Classification

In some embodiments, at least 20, at least 30, at least 40, at least 50, at least 60, at least 70, at least 80, at least 100, at least 120, at least 140, at least 160, at least 180, at least 200, at least 220, at least 240, at least 260, at least 280, at least 300 or more genes from a cancer sample are measured. In some embodiments, it is the combination of substantially all of the genes from a cancer sample that allows for the most accurate determination of abundance of the cell type in the sample and prognostication of outcome, diagnosis or therapeutic response to treatment. In a preferred embodiment, the methods described herein directly utilise single-cell RNA sequencing data rather than known gene-lists as input to generate a gene expression matrix or profile.

“Gene expression” as used herein refers to the relative levels of expression and/or pattern of expression of a gene. The expression of a gene may be measured at the level of DNA, cDNA, RNA, mRNA, or combinations thereof. “Gene expression profile” refers to the levels of expression of multiple different genes measured for the same sample. An expression profile can be derived from a biological sample collected from a subject at one or more time points prior to, during, or following diagnosis, treatment, or therapy for cancer (or any combination thereof), can be derived from a biological sample collected from a subject at one or more time points during which there is no treatment or therapy for cancer (e.g., to monitor progression of disease or to assess development of disease in a subject at risk for breast cancer), or can be collected from a healthy subject.

Gene expression profiles may be measured in a sample, such as samples comprising a variety of cell types, different tissues, different organs, or fluids (e.g., blood, urine, spinal fluid, sweat, saliva or serum) by various methods including but not limited to microarray technologies and quantitative and semi-quantitative RT-PCR techniques as well as single-cell transcriptome sequencing (sc-RNA-seq) and other methods known in the art.

### Deconvolution and Cell Subsets

The term “deconvolution,” as used herein, may refer to the process of identifying (e.g., estimating) the relative proportions or the abundance (e.g., an absolute or fractional abundance) of cell subsets or cell populations in a mixture of cell subsets or cell populations of a sample. Deconvolution methods generally work on the principle that the expression value of each gene, in a bulk, heterogenous sample, can be mathematically modelled as the gene expression contributions from each of the individual cell-types that constitute the sample (Cobos et al., (2018) Bioinformatics 34:11, 1969-1979), incorporated herein in its entirety).

Deconvolution methods are often broadly grouped into 3 common types of methods: ordinary least squares (OLS); linear least squares (LLS); or simply least squares (LS). A skilled person will understand suitable deconvolution that may be used in the methods described herein. The process of deconvolution may vary as understood by a skilled person in the art. Some processes of deconvolution use known gene-lists as input (e.g., the original CIBERSORT method). Others directly utilise single-cell RNA sequencing data (e.g., the newer CIBERSORTx method and DWLS methods). In a preferred embodiment, the methods described herein directly utilise single-cell RNA sequencing data rather than known gene-lists as input.

In an embodiment, the process of deconvolution includes:
Single-cell RNA-Seq is used to generate a summarised expression profile of cells in each sample/tumour;
Each individual cell is annotated as a specific cell-type and/or cell-state;
Cell-type specific signature expression profiles or matrices are generated;
A bulk gene expression profile or matrix of a tumour/sample is then generated;
The common genes are identified between the pre-determined cell-type signature matrices and the bulk gene expression profile of the bulk tumour/sample;
Cell-type deconvolution methods (such as DWLS and/or CIBERSORTx) are used to estimate the cell-type/state abundances present in the bulk tumour/sample.

According to the methods described herein, dampened weighted least squares (DWLS) or CIBERSORTx may be used to determine gene expression deconvolution, whereby cell-type composition of a bulk RNA-sequence data set is computationally inferred. However, a skilled person will understand that other known methods may be used to determine gene expression deconvolution and the methods described herein are not limited accordingly.

Batch correction techniques may be developed to minimize technical variation in expression profiling and may be applied to gene expression deconvolution. In an embodiment, a deconvolution method (e.g., to identify or quantify cell-type states from a mixture of different cell types) may comprise performing a batch correction procedure to reduce technical variation (e.g., between the cell signature profile and the bulk mixture profiles). For example, a bulk reference mode (e.g., B-mode) batch correction may be performed as follows. Generally, while a deconvolution method (e.g., CIBERSORT) may be applied to RNA-Seq, including to reference phenotypes derived from single-cell transcriptome profiling, such a method may not explicitly handle technical variation between the cell signature profile and bulk mixture profiles. Technical variation may include cross-platform technical variation or cross-sample technical variation. For example, technical variation may arise from obtaining feature profiles of the signature matrix and feature profiles of the bulk mixture across different platforms (e.g., RNA-Seq, scRNA-Seq, microarrays, 10× Chromium, SMART-Seq2, droplet-based techniques, UMI-based techniques, non-UMI-based techniques, 3 5′-biased techniques) and/or different sample types (e.g., fresh/frozen samples, FFPE samples, single-cell samples, bulk sorted cell populations or cell types, and samples containing mixtures of cell populations or cell types). For example, crossplatform technical variation may arise in cases where feature profiles with a same type of expression data (e.g., GEPs) are obtained using different platforms. Since technical variation can variably confound deconvolution results, a normalization workflow which may comprise at least two distinct strategies, can be applied to reliably apply gene expression deconvolution across platforms (e.g., RNA-Seq, microarrays) and tissue storage types (e.g., fresh/frozen versus FFPE). For example, a decision tree to guide users in selecting the most appropriate strategy may be used to assist in selecting a bulk-mode batch correction (e.g., B-mode) procedure and/or a single cell batch correction (e.g., S-mode) procedure to be performed.

The distinct cell subsets (e.g., cell types) of the biological sample according to the present disclosure may be any distinct cell types that contribute to the feature profile of the biological sample.

In an embodiment, the distinct cell types comprise any of:
immune enriched cells;
cycling cells;
normal or healthy cells;
Pervivascular-like cells (PVLs);
endothelial cells;
myeloid cells;
plasmablasts;
B-cells;
T-cells;
innate lymphoid cells (ILCs);
cancer associated fibroblasts;
immune depleted;
high cancer heterogenicity; and
combinations of these.

In an embodiment, the ecotypes may comprise the following qualitative parameters:
E1: Ecotype 1 comprises tumours of predominantly Luminal B subtype that are enriched for the LumB_SC cell-type;
E2: Ecotype 2 comprises tumours comprising of mostly Luminal A or Normal-like subtypes that are enriched with cell type abundances enriched for, among others, endothelial CXCL12+ and ACKR1+ cells, s1 MSC iCAFs and a depletion of cycling cells;
E3: Ecotype 3 comprises tumours of predominantly basal-like subtype that are enriched for the Basal_SC, Luminal Progenitor and cycling cell-types;
E4: Ecotype 4 comprises a similar mix of tumours of all subtypes (Luminal A, B, Her2E, basal-like and Normal-like) that are enriched for cell-types of the T-cell & ILCs and Myeloid lineage;
E5: Ecotype 5 comprises mostly luminal tumours, predominantly of the Luminal A subtype, are enriched for Mature Luminal, LumB_SC, and Plasmablast cell-types, have low cycling cell content;
E6: Ecotype 6 comprises mostly luminal tumours, predominantly of the Luminal A subtype, that are mostly enriched with LumA_SC cell-types, have low cycling cell content;
E7: Ecotype 7 comprises predominantly Her2-enriched tumours that are mostly enriched with Her2_SC cell-types;
E8: Ecotype 6 comprises mostly luminal tumours, predominantly of the Luminal B subtype, that are enriched with Myeloid_c1_LAM1_FABP5, CAF_myCAF_like_s4 and FOXP3+CD4 Treg cell-types;
E9: Ecotype 9 comprises mostly luminal A tumours, that are mostly enriched with myCAF-like (s4 and s5), Myeloid FCGR3A+, and Endothelial RGS5+ cell-types, have low cycling cell content.

A skilled person will understand that varying proportions of these subtypes can form a given ecotype. Within the cell types listed above, a skilled person will understand that each of the cell types can be further broken down into the five ‘intrinsic’ molecular subtypes: luminal-like (LumA and LumB), HER2-enriched (HER2E), basal-like (BLBC) and normal-like.

In some embodiments, the distinct subsets of cells comprise subsets of cells at different cell cycle stages. A subset of cells may include cells in any suitable cell cycle stage, including, but not limited to, interphase, mitotic phase or cytokinesis. In some embodiments, cells in a subset of cells are at prophase, metaphase, anaphase, or telophase. In some cases, the cells in a subset of cells is quiescent (Go phase), at the Gi checkpoint (Gi phase), replicated DNA but before mitosis (G2 phase), or undergoing DNA replication (S phase). A skilled person will understand that the term “cycling cell” refers to a cell at different cell cycle stages.

In some embodiments, the distinct cell subsets include different functional pathways within one or more cells. Functional pathways of interest include, without limitation, cellular signalling pathways, gene regulatory pathways, or metabolic pathways. Thus, in some embodiments, the method of the present disclosure may be a method estimating the relative activity of different signalling or metabolic pathways in a cell, a collection of cells, a tissue, etc., by measuring multiple features of the signalling or metabolic pathways (e.g., measuring activation state of proteins in a signalling pathway; measuring expression level of genes in a gene regulatory network; measuring the level of a metabolite in a metabolic pathway, etc.). The cellular signalling pathways of interest include any suitable signalling pathway, such as, without limitation, cytokine signalling, death factor signalling, growth factor signalling, survival factor signalling, hormone signalling, Wnt signalling, Hedgehog signalling, Notch signalling, extracellular matrix signalling, insulin signalling, calcium signalling, G-protein coupled receptor signalling, neurotransmitter signalling, and combinations thereof. The metabolic pathway may include any suitable metabolic pathway, such as, without limitation, glycolysis, gluconeogenesis, citric acid cycle, fermentation, urea cycle, fatty acid metabolism, pyrimidine biosynthesis, glutamate amino acid group synthesis, porphyrin metabolism, aspartate amino acid group synthesis, aromatic amino acid synthesis, histidine metabolism, branched amino acid synthesis, pentose phosphate pathway, purine biosynthesis, glucoronate metabolism, inositol metabolism, cellulose metabolism, sucrose metabolism, starch and glycogen metabolism, and combinations thereof.

In some embodiments, a cell subset may be any group of cells in a biological sample whose presence is characterized by one or more features (such as gene expression on the RNA level, protein expression, genomic mutations, biomarkers, and so forth). A cell subset may be, for example, a cell type or cell sub-type. In certain aspects, one or more cell subsets may be leukocytes (e.g., white blood cells or WBCs). Potential leukocyte cell subsets include monocytes, dendritic cells, neutrophils, eosinophils, basophils, and lymphocytes. These leukocyte subsets can be further subdivided, for example, lymphocyte cell subsets include natural killer cells (NK cells), T-cells (e.g., CD8 T cells, CD4 naive T cells, CD4 memory RO unactivated T cells, CD4 memory RO activated T cells, follicular helper T cells, regulatory T cells, and so forth) and B-cells (naive B cells, memory B cells, Plasma cells). Immune cells subsets may be further separated based on activation (or stimulation) state.

In certain embodiments, leukocytes may be from an individual with a leukocyte disorder, such as blood cancer, an autoimmune disease, myelodysplastic syndrome, and so forth. Examples of a blood disease include Acute lymphoblastic leukemia (ALL), Acute myelogenous leukemia (AML), Chronic lymphocytic leukemia (CLL), Chronic myelogenous leukemia (CML), Acute monocytic leukemia (AMoL), Hodgkin's lymphoma, Non-Hodgkin's lymphoma, and myeloma.

In certain embodiments, one or more cell subsets may include tumour infiltrating leukocytes (TILs). Tumour infiltrating leukocytes may be in mixture with cancer cells in the biological sample, or may be enriched by any methods described above or known in the art.

In certain aspects, one or more cell subsets may include cancer cells, such as blood cancer, breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, and brain cancer.

Cell subsets of interest may include brain cells, including neuronal cells, astrocytes, oligodendrocytes, and microglia, and progenitor cells thereof. Other cell subsets of interest include stem cells, pluripotent stem cells, and progenitor cells of any biological tissue, including blood, solid tissue from brain, lymph node, thymus, bone marrow, spleen, skeletal muscle, heart, colon, stomach, small intestine, kidney, liver, lung, and so forth.

### Cancer

Despite recent advances, the challenge of cancer treatment remains to target specific treatment regimens to distinct tumour types with different pathogenesis, and ultimately personalize tumour treatment in order to maximize outcome. In particular, once a patient is diagnosed with cancer, such as breast cancer, there is a need for methods that allow a practitioner to predict the expected course of disease, including the likelihood of cancer recurrence, long-term survival of the patient and the like, and select the most appropriate treatment options accordingly.

For the purposes of the present invention, “breast cancer” includes, for example, those conditions classified by biopsy or histology as malignant pathology. One of skill in the art will appreciate that breast cancer refers to any malignancy of the breast tissue, including, for example, carcinomas and sarcomas. Particular embodiments of breast cancer include ductal carcinoma in situ (DCIS), lobular carcinoma in situ (LCIS), or mucinous carcinoma. Breast cancer also refers to infiltrating ductal (IDC) or infiltrating lobular carcinoma (ILC). In most embodiments of the invention, the subject of interest is a human patient suspected of or having been diagnosed with breast cancer.

Breast cancer is a heterogeneous disease with respect to molecular alterations and cellular composition. This diversity creates a challenge for researchers trying to develop classifications that are clinically meaningful. Gene expression profiling by microarray has provided insight into the complexity of breast tumours and can be used to provide prognostic information beyond standard pathologic parameters.

Expression profiling of breast cancer identifies biologically and clinically distinct molecular subtypes which may require different treatment approaches. The major intrinsic subtypes of breast cancer referred to as Luminal A, Luminal B, HER2-enriched, Basal-like have distinct clinical features, relapse risk and response to treatment. The “intrinsic” subtypes known as Luminal A (LumA), Luminal B (LumB), HER2-enriched, Basal-like, and Normal-like were discovered using unsupervised hierarchical clustering of microarray data (Perou et al. (2000) Nature 406:747-752). Intrinsic genes, as described in Perou et al. (2000) Nature 406:747-752, are statistically selected to have low variation in expression between biological sample replicates from the same individual and high variation in expression across samples from different individuals. Thus, intrinsic genes are the classifier genes for breast cancer classification. Although clinical information was not used to derive the breast cancer intrinsic subtypes, this classification has proved to have prognostic significance (Sorlie et al. (2001) PNAS 98(19) 10869-10874).

Breast tumours of the “Luminal” subtype are ER positive and have a similar keratin expression profile as the epithelial cells lining the lumen of the breast ducts (Taylor Papadimitriou et al. (1989) J Cell Sci 94:403-413; Perou et al (2000) New Technologies for Life Sciences: A Trends Guide 67-7 6). Conversely, ER-negative tumours can be broken into two main subtypes, namely those that overexpress (and are DNA amplified for) HER-2 and GRB7 (HER-2-enriched) and “Basal-like” tumours that have an expression profile similar to basal epithelium and express Keratin 5, 6B, and 17. Both these tumour subtypes are aggressive and typically more deadly than Luminal tumours; however, there are subtypes of Luminal tumours with different outcomes. The Luminal tumours with poor outcomes consistently share the histopathological feature of being higher grade and the molecular feature of highly expressing proliferation genes.

The methods described herein may be further combined with information on clinical variables to generate a risk of relapse predictor or to aid diagnosis or prognosis or for use in any other method described herein.

As described herein, a number of clinical and prognostic breast cancer factors are known in the art and are used to predict treatment outcome and the likelihood of disease recurrence. Such factors include, for example, lymph node involvement, tumour size, histologic grade, estrogen and progesterone hormone receptor status, HER-2 levels, and tumour ploidy.

Methods of identifying breast cancer patients and staging the disease are well known and may include manual examination, biopsy, review of patient's and/or family history, and imaging techniques, such as mammography, magnetic resonance imaging (MRI), and positron emission tomography (PET). It will be understood that breast cancer stage is usually expressed as a number on a scale of 0 through IV with stage 0 describing non-invasive cancers that remain within their original location and stage IV describing invasive cancers that have spread outside the breast to other parts of the body.

Stage 0 is used to describe non-invasive breast cancers, such as DCIS (ductal carcinoma in situ). In stage 0, there is no evidence of cancer cells or non-cancerous abnormal cells breaking out of the part of the breast in which they started, or getting through to or invading neighbouring normal tissue. Stage I describes invasive breast cancer (cancer cells are breaking through to or invading normal surrounding breast tissue). Stage IA describes invasive breast cancer in which the tumour measures up to 2 centimeters (cm) and the cancer has not spread outside the breast; no lymph nodes are involved. Stage IB describes invasive breast cancer in which there is no tumour in the breast; instead, small groups of cancer cells—larger than 0.2 millimeter (mm) but not larger than 2 mm—are found in the lymph nodes or there is a tumour in the breast that is no larger than 2 cm, and there are small groups of cancer cells—larger than 0.2 mm but not larger than 2 mm—in the lymph nodes.

Stage II is divided into subcategories known as IIA and IIB. Stage IIA describes invasive breast cancer in which no tumour can be found in the breast, but cancer (larger than 2 millimeters [mm]) is found in 1 to 3 axillary lymph nodes (the lymph nodes under the arm) or in the lymph nodes near the breast bone (found during a sentinel node biopsy) or the tumour measures 2 centimeters (cm) or smaller and has spread to the axillary lymph nodes or the tumour is larger than 2 cm but not larger than 5 cm and has not spread to the axillary lymph nodes. Stage IIB describes invasive breast cancer in which the tumour is larger than 2 cm but no larger than 5 centimeters; small groups of breast cancer cells—larger than 0.2 mm but not larger than 2 mm—are found in the lymph nodes or the tumour is larger than 2 cm but no larger than 5 cm; cancer has spread to 1 to 3 axillary lymph nodes or to lymph nodes near the breastbone (found during a sentinel node biopsy) or the tumour is larger than 5 cm but has not spread to the axillary lymph nodes.

Stage III is divided into subcategories known as IIIA, HIB, and IHC. In general, stage IIIA describes invasive breast cancer in which either no tumour is found in the breast or the tumour may be any size; cancer is found in 4 to 9 axillary lymph nodes or in the lymph nodes near the breastbone (found during imaging tests or a physical exam) or the tumour is larger than 5 centimeters (cm); small groups of breast cancer cells (larger than 0.2 millimeter [mm] but not larger than 2 mm) are found in the lymph nodes or the tumour is larger than 5 cm; cancer has spread to 1 to 3 axillary lymph nodes or to the lymph nodes near the breastbone (found during a sentinel lymph node biopsy). Stage IIIB describes invasive breast cancer in which the tumour may be any size and has spread to the chest wall and/or skin of the breast and caused swelling or an ulcer and may have spread to up to 9 axillary lymph nodes or may have spread to lymph nodes near the breastbone.

Stage IIIC describes invasive breast cancer in which there may be no sign of cancer in the breast or, if there is a tumour, it may be any size and may have spread to the chest wall and/or the skin of the breast and the cancer has spread to 10 or more axillary lymph nodes or the cancer has spread to lymph nodes above or below the collarbone or the cancer has spread to axillary lymph nodes or to lymph nodes near the breastbone.

Stage IV describes invasive breast cancer that has spread beyond the breast and nearby lymph nodes to other organs of the body, such as the lungs, distant lymph nodes, skin, bones, liver, or brain.

Using the methods of the present invention, the diagnosis and/or prognosis of a breast cancer patient can be determined independent of, or in combination with assessment of these clinical factors. In some embodiments, combining the methods disclosed herein with evaluation of these clinical factors may permit a more accurate risk assessment.

The methods of the invention may be further coupled with analysis of, for example, estrogen receptor (ER) and progesterone receptor (PgR) status, and/or HER-2 expression levels. Other factors, such as patient clinical history, family history and menopausal status, may also be considered when evaluating breast cancer prognosis or diagnosis via the methods of the invention.

### Sample Source

In one embodiment of the present invention, abundance of cell type is assessed through the evaluation of gene expression profiles of the genes in one or more subject samples. For the purpose of discussion, the term subject, or subject sample, refers to an individual regardless of health and/or disease status. A subject can be a subject, a study participant, a control subject, a screening subject, or any other class of individual from whom sample is obtained and assessed in the context of the invention.

Accordingly, a subject can be diagnosed with breast cancer, can present with one or more symptoms of breast cancer, or a predisposing factor, such as a family (genetic) or medical history (medical) factor, for breast cancer, can be undergoing treatment or therapy for breast cancer, or the like. Alternatively, a subject can be healthy with respect to any of the aforementioned factors or criteria. It will be appreciated that the term “healthy” as used herein, is relative to breast cancer status. Thus, an individual defined as healthy with reference to any specified disease or disease criterion, can in fact be diagnosed with any other one or more diseases, or exhibit any other one or more disease criterion, including one or more cancers other than breast cancer. However, the healthy controls are preferably free of any cancer.

In particular embodiments, the methods for determining abundance of the cell type in the sample include collecting a sample comprising a cancer cell or tissue, such as a breast tissue sample or a primary breast tumour tissue sample.

A “sample” or “biological sample” is intended to mean any sampling of cells, tissues, or bodily fluids in which expression of one or more intrinsic genes can be determined. Examples of such biological samples include, but are not limited to, biopsies and smears. Bodily fluids useful in the present invention include blood, lymph, urine, saliva, nipple aspirates, gynecological fluids, or any other bodily secretion or derivative thereof. Blood can include whole blood, plasma, serum, or any derivative of blood. In some embodiments, the biological sample includes breast cells, particularly breast tissue from a biopsy, such as a breast tumour tissue sample. Biological samples may be obtained from a subject by a variety of techniques including, for example, by scraping or swabbing an area, by using a needle to aspirate cells or bodily fluids, or by removing a tissue sample (i.e., biopsy). Methods for collecting various biological samples are well known in the art. In some embodiments, a breast tissue sample is obtained by, for example, fine needle aspiration biopsy, core needle biopsy, or excisional biopsy. Fixative and staining solutions may be applied to the cells or tissues for preserving the specimen and for facilitating examination. Biological samples, particularly breast tissue samples, may be transferred to a glass slide for viewing under magnification. In one embodiment, the biological sample is a formalin-fixed, paraffin-embedded breast tissue sample, particularly a primary breast tumour sample.

### Detection of Gene Expression

Any methods available in the art for detecting expression of genes in a cancer sample are encompassed herein. By “detecting expression” is intended determining the quantity or presence of an RNA transcript or its expression product of an intrinsic gene.

Methods for detecting expression of the intrinsic genes of the invention, that is, gene expression profiling, include methods based on hybridization analysis of polynucleotides, methods based on sequencing of polynucleotides, immunohistochemistry methods, and proteomics based methods. The methods generally detect expression products (e.g., mRNA) of the genes in a cancer sample.

In embodiments, PCR-based methods, such as reverse transcription PCR (RT-PCR) (Weis et al., TIG 8:263-64, 1992), and array-based methods such as microarray (Schena et al., Science 270:467-70, 1995), preferably single-cell RNA sequencing, is used. By “microarray” is intended an ordered arrangement of hybridisable array elements, such as, for example, polynucleotide probes, on a substrate. The term “probe” refers to any molecule that is capable of selectively binding to a specifically intended target biomolecule, for example, a nucleotide transcript or a protein encoded by or corresponding to an intrinsic gene. Probes can be synthesized by one of skill in the art, or derived from appropriate biological preparations. Probes may be specifically designed to be labelled. Examples of molecules that can be utilized as probes include, but are not limited to, RNA, DNA, proteins, antibodies, and organic molecules.

Many expression detection methods use isolated RNA. The starting material is typically total RNA isolated from a biological sample, such as a tumour or tumour cell line, and corresponding normal tissue or cell line, respectively. If the source of RNA is a primary tumour, RNA (e.g., mRNA) can be extracted, for example, from frozen or archived paraffin embedded and fixed (e.g., formalin-fixed) tissue samples (e.g., pathologist-guided tissue core samples).

General methods for RNA extraction are well known in the art and are disclosed in standard textbooks of molecular biology, including Ausubel et al., ed., Current Protocols in Molecular Biology, John Wiley & Sons, New York 1987-1999. Methods for RNA extraction from paraffin embedded tissues are disclosed, for example, in Rupp and Locker (Lab Invest. 56:A67, 1987) and De Andres et al. (Biotechniques 18:42-44, 1995). In particular, RNA isolation can be performed using a purification kit, a buffer set and protease from commercial manufacturers, such as Qiagen (Valencia, Calif.), according to the manufacturer's instructions. For example, total RNA from cells in culture can be isolated using Qiagen RN easy mini-columns. Other commercially available RNA isolation kits include MASTERPURE™ Complete DNA and RNA Purification Kit (Epicentre, Madison, Wis.) and Paraffin Block RNA Isolation Kit (Ambion, Austin, Tex.). Total RNA from tissue samples can be isolated, for example, using RNA Stat-60 (Tel-Test, Friendswood, Tex.). RNA prepared from a tumour can be isolated, for example, by cesium chloride density gradient centrifugation. Additionally, large numbers of tissue samples can readily be processed using techniques well known to those of skill in the art, such as, for example, the single-step RNA isolation process of Chomczynski (U.S. Pat. No. 4,843,155).

Isolated RNA can be used in hybridization or amplification assays that include, but are not limited to, PCR analyses and probe arrays. One method for the detection of RNA levels involves contacting the isolated RNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 60, 10 0, 250, or 500 nucleotides in length and sufficient to specifically hybridize under stringent conditions to an intrinsic gene of the present invention, or any derivative DNA or RNA. Hybridization of an mRNA with the probe indicates that the intrinsic gene in question is being expressed.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probes are immobilized on a solid surface and the mRNA is contacted with the probes, for example, in an Agilent gene chip array. A skilled person can readily adapt known mRNA detection methods for use in detecting the level of expression of the intrinsic genes of the present invention.

An alternative method for determining the level of intrinsic gene expression product in a sample involves the process of nucleic acid amplification, for example, by RT-PCR (U.S. Pat. No. 4,683,202), ligase chain reaction (Barany, Proc. Natl. Acad. Sci. USA 88:189-93, 1991), self sustained sequence replication (Guatelli et al., Proc. Natl. Acad. Sci. USA 87: 187 4-78, 1990), transcriptional amplification system (Kwoh et al., Proc. Natl. Acad. Sci. USA 86:1173-77, 1989), Q-Beta Replicase (Lizardi et al., Bio/Technology 6:1197, 1988), rolling circle replication (U.S. Pat. No. 5,854,033), or any other nucleic acid amplification method, followed by the detection of the amplified molecules using techniques well known to those of skill in the art. These detection schemes are especially useful for the detection of nucleic acid molecules if such molecules are present in very low numbers.

In particular aspects of the invention, intrinsic gene expression is assessed by quantitative RT-PCR. Numerous different PCR or QPCR protocols are known in the art and exemplified herein below and can be directly applied or adapted for use using the presently described methods for the detection and/or quantification of the intrinsic genes listed in a cancer sample. Generally, in PCR, a target polynucleotide sequence is amplified by reaction with at least one oligonucleotide primer or pair of oligonucleotide primers. The primer(s) hybridize to a complementary region of the target nucleic acid and a DNA polymerase extends the primer(s) to amplify the target sequence. Under conditions sufficient to provide polymerase-based nucleic acid amplification products, a nucleic acid fragment of one size dominates the reaction products (the target polynucleotide sequence which is the amplification product). The amplification cycle is repeated to increase the concentration of the single target polynucleotide sequence. The reaction can be performed in any thermocycler commonly used for PCR. However, preferred are cyders with real-time fluorescence measurement capabilities, for example, SMARTCYCLER® (Cepheid, Sunnyvale, Calif.), ABI PRISM 7700® (Applied Biosystems, Foster City, Calif.), ROTOR-GENET™ (Corbett Research, Sydney, Australia), LIGHTCYCLER® (Roche Diagnostics Corp, Indianapolis, Ind.), !CYCLER® (Biorad Laboratories, Hercules, Calif.) and MX4000® (Stratagene, La Jolla, Calif.).

Quantitative PCR (QPCR) (also referred as realtime PCR) is preferred under some circumstances because it provides not only a quantitative measurement, but also reduced time and contamination. In some instances, the availability of full gene expression profiling techniques is limited due to requirements for fresh frozen tissue and specialized laboratory equipment, making the routine use of such technologies difficult in a clinical setting. However, QPCR gene measurement can be applied to standard formalin-fixed paraffin-embedded clinical tumour blocks, such as those used in archival tissue banks and routine surgical pathology specimens. As used herein, “quantitative PCR (or “real time QPCR”) refers to the direct monitoring of the progress of PCR amplification as it is occurring without the need for repeated sampling of the reaction products. In quantitative PCR, the reaction products may be monitored via a signalling mechanism (e.g., fluorescence) as they are generated and are tracked after the signal rises above a background level but before the reaction reaches a plateau. The number of cycles required to achieve a detectable or “threshold” level of fluorescence varies directly with the concentration of amplifiable targets at the beginning of the PCR process, enabling a measure of signal intensity to provide a measure of the amount of target nucleic acid in a sample in real time.

In another embodiment of the invention, microarrays are used for expression profiling. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labelled RNA or DNA is hybridized to complementary probes on the array and then detected by laser scanning. Hybridization intensities for each probe on the array are determined and converted to a quantitative value representing relative gene expression levels. See, for example, U.S. Pat. Nos. 6,040,138, 5,800,992 and 6,020,135, 6,033,860, and 6,344,316. High-density oligonucleotide arrays are particularly useful for determining the gene expression profile for a large number of RNAs in a sample. Techniques for the synthesis of these arrays using mechanical synthesis methods are described in, for example, U.S. Pat. No. 5,384,261. Although a planar array surface is generally used, the array can be fabricated on a surface of virtually any shape or even a multiplicity of surfaces. Arrays can be nucleic acids (or peptides) on beads, gels, polymeric surfaces, fibers (such as fiber optics), glass, or any other appropriate substrate. See, for example, U.S. Pat. Nos. 5,770,358, 5,789,162, 5,708,153, 6,040,193 and 5,800,992. Arrays can be packaged in such a manner as to allow for diagnostics or other manipulation of an all-inclusive device. See, for example, U.S. Pat. Nos. 5,856,174 and 5,922,591.

In a specific embodiment of the microarray technique, PCR amplified inserts of cDNA clones are applied to a substrate in a dense array. The microarrayed genes, immobilized on the microchip, are suitable for hybridization under stringent conditions. Fluorescently labelled cDNA probes can be generated through incorporation of fluorescent nucleotides by reverse transcription of RNA extracted from tissues of interest. Labelled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance.

With dual colour fluorescence, separately labelled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93:106-49, 1996). Microarray analysis can be performed by commercially available equipment, following manufacturer's protocols, such as by using the Affymetrix GenChip technology, or Agilent ink jet microarray technology. The development of microarray methods for large-scale analysis of gene expression makes it possible to search systematically for molecular markers of cancer classification and outcome prediction in a variety of tumour types.

### Data Processing

It is often useful to pre-process gene expression data, for example, by addressing missing data, translation, scaling, normalization, weighting, etc. Multivariate projection methods, such as principal component analysis (PCA) and partial least squares analysis (PLS), are so-called scaling sensitive methods. By using prior knowledge and experience about the type of data studied, the quality of the data prior to multivariate modelling can be enhanced by scaling and/or weighting. Adequate scaling and/or weighting can reveal important and interesting variation hidden within the data, and therefore make subsequent multivariate modelling more efficient. Scaling and weighting may be used to place the data in the correct metric, based on knowledge and experience of the studied system, and therefore reveal patterns already inherently present in the data.

If possible, missing data, for example gaps in column values, should be avoided. However, if necessary, such missing data may replaced or “filled” with, for example, the mean value of a column (“mean fill”); a random value (“random fill”); or a value based on a principal component analysis (“principal component fill”).

“Translation” of the descriptor coordinate axes can be useful. Examples of such translation include normalization and mean centering. “Normalization” may be used to remove sample-to-sample variation. For microarray data, the process of normalization aims to remove systematic errors by balancing the fluorescence intensities of the two labelling dyes. The dye bias can come from various sources including differences in dye labelling efficiencies, heat and light sensitivities, as well as scanner settings for scanning two channels. Some commonly used methods or calculating normalization factor include: (i) global normalization that uses all genes on the array; (ii) housekeeping genes normalization that uses constantly expressed housekeeping/invariant genes; and (iii) internal controls normalization that uses known amount of exogenous control genes added during hybridization (Quackenbush (2002) Nat. Genet. 32 (Suppl.), 496-501). In one embodiment, the intrinsic genes disclosed herein can be normalized to control housekeeping genes. For example, the housekeeping genes described in U.S. Patent Publication 2008/0032293, which is herein incorporated by reference in its entirety, can be used for normalization. Exemplary housekeeping genes include MRPL19, PSMC4, SF3A1, PUM1, ACTB, GAPD, GUSB, RPLP0, and TFRC. It will be understood by one of skill in the art that the methods disclosed herein are not bound by normalization to any particular housekeeping genes, and that any suitable housekeeping gene(s) known in the art can be used.

Many normalization approaches are possible, and they can often be applied at any of several points in the analysis. In one embodiment, microarray data is normalized using the LOWESS method, which is a global locally weighted scatterplot smoothing normalization function. In another embodiment, qPCR data is normalized to the geometric mean of set of multiple housekeeping genes.

“Mean centering” may also be used to simplify interpretation. Usually, for each descriptor, the average value of that descriptor for all samples is subtracted. In this way, the mean of a descriptor coincides with the origin, and all descriptors are “centered” at zero. In “unit variance scaling,” data can be scaled to equal variance. Usually, the value of each descriptor is scaled by 1/StDev, where StDev is the standard deviation for that descriptor for all samples. “Pareto scaling” is, in some sense, intermediate between mean centering and unit variance scaling. In pareto scaling, the value of each descriptor is scaled by 1/sqrt(StDev), where StDev is the standard deviation for that descriptor for all samples. In this way, each descriptor has a variance numerically equal to its initial standard deviation. The pareto scaling may be performed, for example, on raw data or mean centered data.

“Logarithmic scaling” may be used to assist interpretation when data have a positive skew and/or when data spans a large range, e.g., several orders of magnitude. Usually, for each descriptor, the value is replaced by the logarithm of that value. In “equal range scaling,” each descriptor is divided by the range of that descriptor for all samples. In this way, all descriptors have the same range, that is, 1. However, this method is sensitive to presence of outlier points. In “autoscaling,” each data vector is mean centered and unit variance scaled. This technique is a very useful because each descriptor is then weighted equally, and large and small values are treated with equal emphasis. This can be important for genes expressed at very low, but still detectable, levels.

In one embodiment, data is collected for one or more test samples and classified using the methods described herein. When comparing data from multiple analyses (e.g., comparing expression profiles for one or more test samples to the centroids constructed from samples collected and analyzed in an independent study), it will be necessary to normalize data across these data sets. In one embodiment, Distance Weighted Discrimination (DWD) is used to combine these data sets together (Benito et al. (2004) Bioinformatics 20(1):105-114, incorporated by reference herein in its entirety). DWD is a multivariate analysis tool that is able to identify systematic biases present in separate data sets and then make a global adjustment to compensate for these biases; in essence, each separate data set is a multidimensional cloud of data points, and DWD takes two points clouds and shifts one such that it more optimally overlaps the other.

The methods described herein may be implemented and/or the results recorded using any device capable of implementing the methods and/or recording the results. Examples of devices that may be used include but are not limited to electronic computational devices, including computers of all types. When the methods described herein are implemented and/or recorded in a computer, the computer program that may be used to configure the computer to carry out the steps of the methods may be contained in any computer readable medium capable of containing the computer program. Examples of computer readable medium that may be used include but are not limited to diskettes, CD-ROMs, DVDs, ROM, RAM, and other memory and computer storage devices. The computer program that may be used to configure the computer to carry out the steps of the methods and/or record the results may also be provided over an electronic network, for example, over the internet, an intranet, or other network.

In an embodiment, a processor of the computer is configured to perform the deconvolution method and the cell signature expression profile is stored in a computer readable medium.

### Prognosis, Diagnosis, Survival and Predicting Response to Therapy

Provided herein are methods for predicting cancer outcome. Outcome or prognosis may refer to overall or disease-specific survival, event-free survival, or outcome in response to a particular treatment or therapy. In particular, the methods may be used to predict the likelihood of long-term, disease-free survival. Predicting the likelihood of survival of a cancer patient is intended to assess the risk that a patient will die as a result of the underlying cancer. Long-term, disease-free survival is intended to mean that the patient does not die from or suffer a recurrence of the underlying cancer within a period of at least five years, or at least ten or more years, following initial diagnosis or treatment.

In one embodiment, outcome is predicted based on classification of a subject according to subtype. This classification is based on expression profiling using one more of the genes in a cancer sample. Generally, cell types abundance, when classified according to the methods described herein is indicative of not only prognosis but also response to treatment.

In an embodiment, the ecotypes may comprise the following qualitative parameters which correlate with the prognosis of a subject having or suspected of having cancer:
E1: Ecotype 1 comprises tumours of predominantly Luminal B subtype that are enriched for the LumB_SC cell-type and have an intermediate prognosis;
E2: Ecotype 2 comprises tumours comprising of mostly Luminal A or Normal-like subtypes that are enriched with cell type abundances enriched for, among others, endothelial CXCL12+ and ACKR1+ cells, s1 MSC iCAFs and a depletion of cycling cells and correlate with a better survival outcome or prognosis;
E3: Ecotype 3 comprises tumours of predominantly basal-like subtype that are enriched for the Basal_SC, Luminal Progenitor and cycling cell-types and have a poor prognosis;
E4: Ecotype 4 comprises a similar mix of tumours of all subtypes (Luminal A, B, Her2E, basal-like and Normal-like) that are enriched for cell-types of the T-cell & ILCs and Myeloid lineage and have an intermediate prognosis;
E5: Ecotype 5 comprises mostly luminal tumours, predominantly of the Luminal A subtype, are enriched for Mature Luminal, LumB_SC, and Plasmablast cell-types, have low cycling cell content and a reasonably good prognosis;
E6: Ecotype 6 comprises mostly luminal tumours, predominantly of the Luminal A subtype, that are mostly enriched with LumA_SC cell-types, have low cycling cell content, but a worse prognosis than E5;
E7: Ecotype 7 comprises predominantly Her2-enriched tumours that are mostly enriched with Her2_SC cell-types and have a poor prognosis;
E8: Ecotype 6 comprises mostly luminal tumours, predominantly of the Luminal B subtype, that are enriched with Myeloid_c1_LAM1_FABP5, CAF_myCAF_like_s4 and FOXP3+CD4 Treg cell-types, and the worse prognosis of the luminal related ecotypes;
E9: Ecotype 9 comprises mostly luminal A tumours, that are mostly enriched with myCAF-like (s4 and s5), Myeloid FCGR3A+, and Endothelial RGS5+ cell-types, have low cycling cell content, and a generally good prognosis.

In another embodiment, the methods described herein provide a determination of a Risk Of Relapse (ROR) score that can be used in any patient population regardless of disease status and treatment options. The ROR also have value in the prediction of pathological complete response in subjects treated with, for example, neoadjuvant taxane and anthracycline chemotherapy. Thus, in various embodiments of the present invention, a ROR method model is used to predict outcome. Using these risk models, subjects can be stratified into low, medium, and high risk of relapse groups. Calculation of ROR can provide prognostic information to guide treatment decisions and/or monitor response to therapy.

In some embodiments described herein, the prognostic performance of the defined ecotypes and/or other clinical parameters is assessed utilizing a Cox Proportional Hazards Model Analysis, which is a regression method for survival data that provides an estimate of the hazard ratio and its confidence interval. The Cox model is a well-recognized statistical technique for exploring the relationship between the survival of a patient and particular variables. This statistical method permits estimation of the hazard (i.e., risk) of individuals given their prognostic variables (e.g., intrinsic gene expression profile with or without additional clinical factors, as described herein). The “hazard ratio” is the risk of death at any given time point for patients displaying particular prognostic variables. See generally Spruance et al., Antimicrob. Agents & Chemo. 48:2787-92, 2004.

In an embodiment of the invention, where a diagnosis, prognosis or prediction to drug treatment is provided, it will be understood that the method will comprise:
training a predictor set of cancer samples from subjects with known diagnosis, prognosis or prediction to drug treatment; and
applying the predictor to the cancer sample to determine diagnosis, prognosis or prediction to drug treatment of the subject.

Where the training of a predictor set of cancer samples from subjects with known diagnosis, prognosis or prediction to drug treatment is required, the method may comprise:
performing cell-type deconvolution on bulk cancer cohorts (such as METABRIC);
grouping those patients/tumours into “tumour ecotypes” based on the cell-type abundances, preferably by using a form of consensus clustering; and
associating these tumour ecotypes with diagnosis, prognosis or prediction to drug treatment of the subject.

In another embodiment, where the training of a predictor set of cancer samples from subjects with known diagnosis, prognosis or prediction to drug treatment is required, the method may also comprise applying the predictor set to the cancer sample by:
generating gene expression profiles of tumour(s);
calculate cell-type abundances (using either single-cell and/or bulk methods); and
assigning the cancer cells within the cancer sample to an ecotype (e.g., using clustering or other classification methods such as machine learning).

Cancer is managed by several alternative strategies that may include, for example, surgery, radiation therapy, hormone therapy, chemotherapy, or some combination thereof. For example, as is known in the art, treatment decisions for individual breast cancer patients can be based on endocrine responsiveness of the tumour, menopausal status of the patient, the location and number of patient lymph nodes involved, estrogen and progesterone receptor status of the tumour, size of the primary tumour, patient age, and stage of the disease at diagnosis. Analysis of a variety of clinical factors and clinical trials has led to the development of recommendations and treatment guidelines for early-stage breast cancer by the International Consensus Panel of the St. Gallen Conference (2005). See, Goldhirsch et al., Annals Oneal. 16:1569-83, 2005. The guidelines recommend that patients be offered chemotherapy for endocrine non-responsive disease; endocrine therapy as the primary therapy for endocrine responsive disease, adding chemotherapy for some intermediate- and all high-risk groups in this category; and both chemotherapy and endocrine therapy for all patients in the uncertain endocrine response category except those in the low-risk group.

Stratification of patients according to risk of relapse and risk score disclosed herein provides an additional or alternative treatment decision-making factor. The methods comprise evaluating risk of relapse optionally in combination with one or more clinical variables, such as node status, tumour size, and ER status. The risk score can be used to guide treatment decisions. For example, a subject having a low risk score may not benefit from certain types of therapy, whereas a subject having a high risk score may be indicated for a more aggressive therapy.

The methods of the present invention find use in identifying high-risk, poor prognosis population of subjects and thereby determining which patients would benefit from continued and/or more aggressive therapy and close monitoring following treatment. For example, early-stage cancer patients assessed as having a high risk score by the methods disclosed herein may be selected for more aggressive adjuvant therapy, such as chemotherapy, following surgery and/or radiation treatment. In particular embodiments, the methods of the present invention may be used in conjunction with the treatment guidelines established by the St. Gallen Conference to permit practitioners to make more informed cancer treatment decisions.

The methods disclosed herein also find use in predicting the response of a cancer patient to a selected treatment. Predicting the response of a cancer patient to treatment is intended to mean assessing the likelihood that a patient will experience a positive or negative outcome with a particular treatment. As used herein, indicative of a positive treatment outcome refers to an increased likelihood that the patient will experience beneficial results from the selected treatment (e.g., complete or partial remission, reduced tumour size, etc.). Indicative of a negative treatment outcome is intended to mean an increased likelihood that the patient will not benefit from the selected treatment with respect to the progression of the underlying breast cancer.

In some embodiments, the relevant time for assessing prognosis or disease-free survival time begins with the surgical removal of the tumour or suppression, mitigation, or inhibition of tumour growth. In another embodiment, the risk score is calculated based on a sample obtained after initiation of neoadjuvant therapy such as endocrine therapy. The sample may be taken at any time following initiation of therapy, but is preferably obtained after about one month so that neoadjuvant therapy can be switched to chemotherapy in unresponsive patients. It has been shown that a subset of tumours indicated for endocrine treatment before surgery is non-responsive to this therapy. The model provided herein can be used to identify aggressive tumours that are likely to be refractory to endocrine therapy, even when tumours are positive for estrogen and/or progesterone receptors.

Survival analysis can be performed using any known method in the art, including the Kaplan-Meier method (as described in the Example herein). The Kaplan-Meier method estimates the survival function from life-time data. In medical research, it can be used to measure the fraction of patients living for a certain amount of time after treatment. A plot of the Kaplan-Meier method of the survival function is a series of horizontal steps of declining magnitude which, when a large enough sample is taken, approaches the true survival function for that population. The value of the survival function between successive distinct sampled observations (“clicks”) is assumed to be constant.

An important advantage of the Kaplan-Meier curve is that the method can take into account “censored” data-losses from the sample before the final outcome is observed (for instance, if a patient withdraws from a study). On the plot, small vertical tick-marks indicate losses, where patient data has been censored. When no truncation or censoring occurs, the Kaplan-Meier curve is equivalent to the empirical distribution.

In statistics, the log-rank test (also known as the Mantel-Cox test) is a hypothesis test to compare the survival distributions of two groups of patients. It is a nonparametric test and appropriate to use when the data are right censored. It is widely used in clinical trials to establish the efficacy of new drugs compared to a control group when the measurement is the time to event. The log-rank test statistic compares estimates of the hazard functions of the two groups at each observed event time. It is constructed by computing the observed and expected number of events in one of the groups at each observed event time and then adding these to obtain an overall summary across all time points where there is an event. The log-rank statistic can be derived as the score test for the Cox proportional hazards model comparing two groups. It is therefore asymptotically equivalent to the likelihood ratio test statistic based from that model.

The invention also provides for methods for diagnosing a breast cancer clinical subtype in a test sample from a subject. Diagnosis as used herein refers to the determination that a subject or patient has a type of breast cancer, or intrinsic subtype of breast cancer as described herein or known in the art. The type of breast cancer diagnosed according to the methods described herein may be any type known in the art or described herein.

In an embodiment, one or more of the following additional diagnostic tests may be used in addition to the methods for diagnosis described herein. These include:
breast ultrasound: to create sonograms of areas inside the breast;
diagnostic mammogram or a screening mammogram or x-ray;
magnetic resonance imaging (MRI) to analyse areas inside the breast;
biopsy which may include removal of tissue or fluid from the breast to be looked at under a microscope and/or do more testing. The biopsy may be a fine-needle aspiration, core biopsy or open biopsy.

In an embodiment, the subject may exhibit one or more of the following risk factors: age, preferably over 50 years of age; genetic mutations to certain genes, such as BRCA1 and BRCA2; early menstrual periods before age 12 and starting menopause after age 55; having dense breasts; personal history of breast cancer or certain non-cancerous breast diseases; family history of breast or ovarian cancer; previous treatment using radiation therapy; or history of taking the drug diethylstilbestrol (DES).

In some embodiments, the subject diagnosed with breast cancer exhibits one or more of the symptoms of breast cancer described herein or known in the art.

### Treatment

In an aspect of the invention, there is provided methods for diagnosing and treating breast cancer in a subject.

The terms “patient” and “subject” to be treated herein are used interchangeably and refer to patients and subjects of human or other mammal and includes any individual being examined or treated using the methods of the invention. Suitable mammals that fall within the scope of the invention include, but are not restricted to, primates, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., koalas, bears, wild cats, wild dogs, wolves, dingoes, foxes and the like).

In some embodiments, the treatment may include any of those described herein or known in the art including surgery; chemotherapy; hormonal therapy; biological therapy such as immunotherapy, small molecule therapy or antibody therapy; and radiation therapy. In a further embodiment, the chemotherapy may include the administration of one or more of:
anthracyclines such as epirubicin (Pharmorubicin®), doxorubicin (Adriamycin®);
mitotic inhibitors such as taxanes, eg paclitaxel (Taxol®), docetaxel (Taxotere®);
antimetabolites such as 5-fluorouracil (5FU), capecitabine, 5-fluorouracil (5-FU), gemcitabine (Gemzar®);
alkylating agents such as cyclophosphamide;
taxanes such as paclitaxel (Taxol®), docetaxel (Taxotere®);
vinorelbine (Navelbine®); and
targeted therapies such as trastuzumab (Herceptin®), lapatinib (Tykerb®), bevacizumab (Avastin®).

In yet another embodiment, the radiotherapy may include the administration of one or more of:
3D conformal radiation therapy;
Intensity-modulated radiation therapy (IMRT);
Volumetric modulated radiation therapy (VMAT);
Image-guided radiation therapy (IGRT);
Stereotactic radiosurgery (SRS);
Brachytherapy;
Superficial x-ray radiation therapy (SXRT); and
Intraoperative radiation therapy (IORT).

In an embodiment, the subject to be treated exhibits one or more symptoms of a disease associated with breast cancer described herein or known in the art. Non-limiting examples may include one or more of:
presence of a lump in the breast or underarm;
thickening or swelling of part of the breast;
irritation or dimpling of breast skin;
redness or flaky skin in the nipple area or the breast;
pulling in of the nipple or pain in the nipple area;
nipple discharge including blood;
any change in the size or the shape of the breast; and
pain in an area of the breast.

Thus, a positive response to treatment with a therapeutically effective amount of any drug or compound identified herein may include amelioration of one of more of the above described symptoms or other symptoms known in the art. For instance, an individual having a positive response to treatment with any drug or compound administered as a result of the methods described herein may have a reduced presence of a lump in the breast or underarm or alternatively this may be surgically excised. An individual having a positive response to treatment with any drug or compound administered as a result of the methods described herein may also have reduced thickening or swelling, reduced irritation of breast skin, reduced redness or flaky skin in the nipple area or the breast, reduced nipple discharge or lessened pain or the symptoms may have disappeared altogether.

“Therapeutically effective amount” is used herein to denote any amount of a drug identified by the methods defined herein which is capable of reducing one or more of the symptoms associated with breast cancer. A single administration of the therapeutically effective amount of the drug may be sufficient, or they may be applied repeatedly over a period of time, such as several times a day for a period of days or weeks. The amount of the active ingredient will vary with the conditions being treated, the stage of advancement of the condition, the age and type of host, and the type and concentration of the formulation being applied. Appropriate amounts in any given instance will be readily apparent to those skilled in the art or capable of determination by routine experimentation.

The terms “treatment” or “treating” a subject includes the application or administration of a drug or compound with the purpose of delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; making the final point of degeneration less debilitating; or improving a subject's physical or mental well-being.

### Pharmaceutical Compositions and Routes of Administration

The drugs or compounds that may be administered following the methods described herein may be provided in the form of a pharmaceutical composition comprising a therapeutically effective amount of any drug described herein or known in the art. In additional embodiments there is provided a pharmaceutical composition of any drug described herein or known in the art comprising a pharmaceutically acceptable salt.

The term “pharmaceutically acceptable salt” also refers to a salt of the compositions of the present invention having an acidic functional group, such as a carboxylic acid functional group, and a base. Pharmaceutically acceptable salts include, by way of non-limiting example, may include sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, phenylacetate, triftuoroacetate, acrylate, chlorobenzoate, dinitrobenzoate, hydroxybenzoate, methoxybenzoate, methylbenzoate, o-acetoxybenzoate, naphthalene-2-benzoate, isobutyrate, phenylbutyrate, a-hydroxybutyrate, butyne-1,4-dicarboxylate, hexyne-1,4-dicarboxylate, caprate, caprylate, cinnamate, glycolate, heptanoate, hippurate, malate, hydroxymaleate, malonate, mandelate, mesylate, nicotinate, phthalate, teraphthalate, propiolate, propionate, phenylpropionate, sebacate, suberate, p-brornobenzenesulfonate, chlorobenzenesulfonate, ethylsulfonate, 2-hydroxyethylsulfonate, methylsulfonate, naphthiene-1-sulfonate, naphthalene-2-sulfonate, naphthiene-1,5-sulfonate, xylenesulfonate, and tartarate salts.

Further, any drug described herein or known in the art can be administered to a subject as a component of a composition that comprises a pharmaceutically acceptable carrier or vehicle. Such compositions can optionally comprise a suitable amount of a pharmaceutically acceptable excipient so as to provide the form for proper administration.

Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and colouring agents can be used.

In one embodiment, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents.

In one embodiment, of any drug described herein or known in the art can take the form of solutions, suspensions, emulsion, drops, tablets, pills, pellets, capsules, capsules containing liquids, powders, sustained-release formulations, suppositories, emulsions, aerosols, sprays, suspensions, nanoparticles or microneedles or any other form suitable for use. In one embodiment, the composition is in the form of a capsule. Other examples of suitable pharmaceutical excipients are described in Remington's Pharmaceutical Sciences 1447-1676 (Alfonso R. Gennaro eds., 19th ed. 1995), incorporated herein by reference.

Where necessary, of any drug described herein or known in the art also includes a solubilizing agent. Also, the agents can be delivered with a suitable vehicle or delivery device as known in the art.

Any drug described herein or known in the art can be co-delivered in a single delivery vehicle or delivery device. Compositions for administration can optionally include a local anaesthetic such as, for example, lignocaine to lessen pain at the site of the injection.

Any drug described herein or known in the art may conveniently be presented in unit dosage forms and may be prepared by any of the methods well known in the art. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In one embodiment, of any drug described herein or known in the art is formulated in accordance with routine procedures as a composition adapted for a mode of administration described herein. In one aspect, the pharmaceutical composition is formulated for administration to the respiratory tract, the skin or the gastrointestinal tract. Accordingly, the pharmaceutical composition for administration to the respiratory tract may be formulated as an inhalable substance, such as common to the art and described herein. In another embodiment, the pharmaceutical composition for administration to the gastrointestinal tract may be formulated with an enteric coating, such as common to the art and described herein.

In an embodiment, the pharmaceutical composition may be administered in a single or as multiple doses. The pharmaceutical composition may be administered between one to three times in a 24 hour period, or daily over a 7 day period or longer. The frequency and timing of administration may be as known in the art.

Routes of administration include, for example: intradermal, intramuscular, intraperitoneal, intravenous, subcutaneous, intranasal, epidural, oral, sublingual, intracerebral, intra-lymph node, intratracheal, intravaginal, transdermal, rectally, by inhalation, or topically, particularly to the ears, nose, eyes, or skin. In some embodiments, the administering is effected orally or by parenteral injection. The mode of administration can be left to the discretion of the practitioner, and depends in-part upon the site of the medical condition. In most instances, administration results in the release of any agent described herein into the bloodstream.

In certain embodiments, the human suffering from or suspected of having breast cancer has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

### Kits

The present invention also provides kits useful for determining cell type abundance. These kits comprise a set of capture probes and/or primers specific for the intrinsic genes listed in a cancer sample, as well as reagents sufficient to facilitate detection and/or quantitation of the intrinsic gene expression product. The kit may further comprise a computer readable medium.

In one embodiment of the present invention, the capture probes are immobilized on an array. By “array” is intended a solid support or a substrate with peptide or nucleic acid probes attached to the support or substrate. Arrays typically comprise a plurality of different capture probes that are coupled to a surface of a substrate in different, known locations.

The arrays of the invention comprise a substrate having a plurality of capture probes that can specifically bind an intrinsic gene expression product. The number of capture probes on the substrate varies with the purpose for which the array is intended. The arrays may be low-density arrays or high-density arrays and may contain 4 or more, 8 or more, 12 or more, 16 or more, 3 2 or more addresses, but will minimally comprise capture probes for the intrinsic genes in a cancer sample.

Arrays may be packaged in such a manner as to allow for diagnostics or other manipulation on the device. See, for example, U.S. Pat. Nos. 5,856,174 and 5,922,591 herein incorporated by reference.

In another embodiment, the kit comprises a set of oligonucleotide primers sufficient for the detection and/or quantitation of each of the intrinsic genes in a cancer sample.

The oligonucleotide primers may be provided in a lyophilized or reconstituted form or may be provided as a set of nucleotide sequences. In one embodiment, the primers are provided in a microplate format, where each primer set occupies a well (or multiple wells, as in the case of replicates) in the microplate. The microplate may further comprise primers sufficient for the detection of one or more housekeeping genes as discussed infra. The kit may further comprise reagents and instructions sufficient for the amplification of expression products from the genes in a cancer sample.

In order that the invention may be readily understood and put into practical effect, particular preferred embodiments will now be described by way of the following non-limiting examples.

### EXAMPLE

The present example illustrates an embodiment of the invention. In particular, the example demonstrates, using single cell signatures, deconvolution of large breast cancer cohorts to stratify them into nine clusters, termed ‘ecotypes’, with unique cellular compositions and clinical outcomes.

### Experimental Procedures

### Patient Material, Ethics Approval and Consent for Publication

Primary untreated breast cancers used in this study were collected under protocols x13-0133, x19-0496, x16-018 and x17-155. Human research ethics committee approval was obtained through the Sydney Local Health District Ethics Committee, Royal Prince Alfred Hospital zone, and the St Vincent's hospital Ethics Committee. Site-specific approvals were obtained for all additional sites. Written consent was obtained from all patients prior to collection of tissue and clinical data stored in a de-identified manner, following pre-approved protocols. Consent into the study included the agreement to the use of all patient tissue and data for publication. Two TNBC samples used for Visium analysis (1142243F and 1160920F) were sourced from BioIVT Asterand®.

### Tissue Dissociation

Samples collected in this study (Table 1) were analysed from fresh surgical resections and cryopreserved tissue. Tumours were mechanically and enzymatically dissociated using Human Tumour Dissociation Kit (Miltenyi Biotec), following the manufacturer's protocol. For cryopreserved tissue, tumour tissues were thawed and washed twice with RPMI 1640 prior to dissociation, as previously described Wu et al., (2021) Genome Medicine, doi: 10.1186/s13073 00885-z. Following incubation at 37° C. for 30 to 60 min, the sample was resuspended in RPMI 1640 and filtered through MACS® SmartStrainers (70 μM; Miltenyi Biotec). The resulting single cell suspension was centrifuged at 300×g for 5 min. For fresh tissue processing, red blood cells were lysed with Lysing Buffer (Becton Dickinson) for 5 min and the resulting suspension was centrifuged at 300×g for 5 min. Where viability was <80%, viability enrichment was performed using the EasySep Dead Cell Removal (Annexin V) Kit (StemCell Technologies) as per manufacturer's protocol. Dissociated cells were resuspended in a final solution of PBS with 10% fetal calf serum (FCS) solution prior to loading on the 10× Chromium platform.

TABLE 1
Patient cohort details Clinical and pathology details for breast cancer patients
analysed by scRNA-Seq in this study.

| | | | | Cancer | | | HER2 | | | Subtype by | Treatment | Details of | | |
|---|---|---|---|---|---|---|---|---|---|---|---|---|---|---|
| Case ID | Gender | Age | Grade | Type | ER | PR | IHC | HER2 ISH (ratio) | Ki67 | IHC | status | treatment | Notable Pathological features | Stage |
| 3586 | Female | 43 | 3 | IDC | 100% 2-3 [symbol] | 100% 2-3 [symbol] | 3 [symbol] | Amplified (6.8) | 30-50% | HER2 [symbol] /ER [symbol] | Naïve | — | Multifocal tumour with associatied high | pT(m)2, N2a |
| | | | | | | | | | | | | | grade DCIS and extensive LVI | |
| 3838 | Female | 49 | 3 | IDC | 0 | 0 | 3 [symbol] | Amplified (8.91) | 60% | HER2 [symbol] | Naïve | — | Associated high grade DCIS. | pT2, N1a |
| 3921 | Female | 60 | 3 | IDC | 0 | 0 | 3 [symbol] | Amplified (10.46) | >50% | HER2 [symbol] | Naïve | — | Associated high grade DCIS and focal LVI | pT2, N2a |
| | | | | | | | | | | | | | | (Stage IIIA) |
| 3941 | Female | 50 | 2 | IDC | 90% 3 [symbol] | 90% 3 [symbol] | 2 [symbol] | Non-Amplified | 10% | ER [symbol] | Naïve | — | Multifocal tumour with associated high | pT1c, N1a, |
| | | | | | | | | | | | | | grade DCIS | Mx |
| 3946 | Female | 52 | 3 | IDC | 0 | 0 | 0 | Non-Amplified | 60% | TNBC | Naïve | — | Basal phenotype. Reactive lymphoid | pT2, N0, Mx |
| | | | | | | | | | | | | | inflitrate with germinal centres. | |
| 3948 | Female | 82 | 3 | IDC | 90% 2-3 [symbol] | 80% 2 [symbol] | 0 | Non-Amplified | ~10% | ER [symbol] | Naïve | — | Associated LCIS, with LVI and perineural | pT2, N2a |
| | | | | | | | | | | | | | invasion | |
| 3963 | Female | 61 | 3 | IDC | 30% 1 [symbol] | 0 | 0 | Non-Amplified | 43% | ER [symbol] | Treated | AC, Paclitaxel, | Probable recurrence from 3 years prior | pT2, pN0, |
| | | | | | | | | | | | | Herceptin | | Mx, Stage |
| | | | | | | | | | | | | (administered | | IIA |
| | | | | | | | | | | | | for Dx 3 years | | |
| | | | | | | | | | | | | prior) | | |
| 4040 | Female | 57 | 3 | IDC | 95% 3 [symbol] | 95% 2-3 [symbol] | 0 | Non-Amplified | >50% | ER [symbol] | Naïve | — | Associated high grade DCIS. | pT2, N0 |
| 4066 | Female | 41 | 2 | IDC | 70% 3 [symbol] | 0 | 3 [symbol] | Amplified (7.7) | 30% | HER2 [symbol] /ER [symbol] | Treated | Neoadjuvant | Associated high grade DCIS and extensive | pT2 N2a Mx |
| | | | | | | | | | | | | AC | LVI. RCB-III, minimal or no-response to | |
| | | | | | | | | | | | | | chemotherapy. | |
| 4067 | Female | 85 | 2 | IDC | 100% 3 [symbol] | 95% 3 [symbol] | 1 [symbol] | Non-Amplified | 3-4% | ER [symbol] | Naïve | — | Associated low grade DCIS and focal | pT2, N1(sn), |
| | | | | | | | | | | | | | perineural invasion. | Mx |
| 4290 | Female | 88 | 2 | IDC | 90% 3 [symbol] | 30% 2 [symbol] | 1 [symbol] | Non-Amplified | 10% | ER [symbol] | Naïve | — | Locally advanced, skin and chest wall | pT4b, Nx |
| | | | | | | | | | | | | | muscle involvement. | |
| 4398 | Female | 52 | 3 | IDC | 95% 2 [symbol] | 80% 2 [symbol] | 2 [symbol] | Non-Amplified | 75% | ER [symbol] | Treated | Neoadjuvant | Mixed morphology with associated high | pT3, pN2a, |
| | | | | | | | | | | | | FEC-D | grade DCIS, extensive LVI and perineural | pMx, Stage |
| | | | | | | | | | | | | | invasion. RCB-III, minimal or no-response | IIIA |
| | | | | | | | | | | | | | to chemotherapy. | |
| 4404-1 | Female | 35 | 3 | IDC | 0 | 0 | 0 | Non-Amplified | 70% | TNBC | Naïve | — | Associated high grade DCIS and focal | pT2, N1a, |
| | | | | | | | | | | | | | LVI. | Mx |
| 4461 | Female | 54 | 2 | IDC | 95% 3 [symbol] | ~5% 3 [symbol] | 2 [symbol] | Non-Amplified | 15% | ER [symbol] | Naive | — | Associated intermediate to high grade | pT3, N1a, |
| | | | | | | | | | | | | | DCIS, LVI and perineural invasion. | Mx |
| 4463 | Female | 58 | 2 | IDC | 100% 2-3 [symbol] | 80% 2-3 [symbol] | 0 | Non-Amplified | 50% | ER [symbol] | Naïve | — | IDC with areas of lobular-like growth | pT3, N1, Mx |
| | | | | | | | | | | | | | pattern, but is E-cadherin positive. | |
| | | | | | | | | | | | | | Associated low through high grade DCIS | |
| | | | | | | | | | | | | | and LVI. | |
| 4465 | Female | 54 | 3 | IDC | 0 | 0 | 0 | Non-Amplified | 70% | TNBC | Naïve | — | Basal phenotype—patchy CK5/6 and p63 | PT2, N0(sn) |
| | | | | | | | | | | | | | positivity. Associated high grade DCIS at | Mx |
| | | | | | | | | | | | | | periphery of tumour mass. | |
| 4471 | Female | 55 | 2 | ILC | 100% 3 [symbol] | 100% 3 [symbol] | 0 | Non-Amplified | 20% | ER [symbol] | Naïve | — | — | pT3, pN0 |
| | | | | | | | | | | | | | | (i [symbol] ) |
| 4495 | Female | 63 | 3 | IDC | 0 | 0 | 0 | Non-Amplified | 80% | TNBC | Naïve | — | Medullary features | pT1c, pN0 |
| 4497-1 | Female | 49 | 3 | IDC | 0 | 0 | 0 | Non-Amplified | 40% | TNBC | Naïve | — | Highly atypical cells with circumscribed | pT2, N1a, |
| | | | | | | | | | | | | | periphery, associated high grade DCIS and | Mx |
| | | | | | | | | | | | | | LVI. Accompanying lymphoid stroma. | |
| 4499-1 | Female | 47 | 3 | IDC | 0 | 0 | 0 | Non-Amplified | 60-70% | TNBC | Naïve | — | BRCA2 mutation | |
| 4513 | Female | 73 | 3 | MBC | 0 | 0 | 0 | Non-Amplified | 75% | TNBC | Treated | Neoadjuvant | Metaplastic, spindle cell carcinoma with | pT3, pN0, |
| | | | | | | | | | | | | AC (4x), | areas of sarcomatous appearance and | Mx, Stage |
| | | | | | | | | | | | | Paclitaxel (3x) | inflammatory infiltrate. LVI present. | IIB |
| | | | | | | | | | | | | | RCB-II, partial pathological response to | |
| | | | | | | | | | | | | | chemotherapy | |
| 4515 | Female | 67 | 3 | IDC | 0 | 0 | 0 | Non-Amplified | 60% | TNBC | Naïve | — | Basal phenotype: CK5/6 [symbol] focal 40%, | PpT1c, pN1, |
| | | | | | | | | | | | | | CK14 [symbol] focal 30%. Associated high grade | Mi, Stage |
| | | | | | | | | | | | | | DCIS and patchy lymphoid infiltrate. | IIA |
| 4517-1 | Female | 58 | 3 | IDC | 0 | 0 | 3 [symbol] | Amplified | 80% | HER2 [symbol] | Naïve | — | | |
| 4523 | Female | 52 | 3 | MBC | 0 | 0 | 1 [symbol] | Non-Amplified | 90% | TNBC | Treated | Neoadjuvant | Metaplastic carcinoma with sebaceous | pT2, pN0 |
| | | | | | | | | | | | | AC (4x), | differentiation. LVI present. RCB-II, | (i [symbol] ), pM0, |
| | | | | | | | | | | | | Paclitaxel (1x) | partial pathological response to | Stage IIA |
| | | | | | | | | | | | | | chemotherapy | |
| 4530 | Female | 42 | 2 | IDC | 95% 2 [symbol] | 95% 3 [symbol] | 1 [symbol] | Non-Amplified | 5% | ER [symbol] | Naive | — | Multifocal tumour with associated high | pT3, pN3, |
| | | | | | | | | | | | | | grade DCIS and LVI. | pMx, Stage |
| | | | | | | | | | | | | | | IIIA |
| 4535 | Female | 47 | 2 | ILC | 95% 3 [symbol] | 70% 2 [symbol] | 2 [symbol] | Non-Amplified | 10% | ER [symbol] | Naive | — | — | pT2, pN0 |
| | | | | | | | | | | | | | | (i [symbol] ), Stage |
| | | | | | | | | | | | | | | IIB |

### Single-Cell RNA Sequencing Using 10× Chromium

Single-cell sequencing was performed using the Chromium Single-Cell v2 3′ and 5′ Chemistry Library, Gel Bead, Multiplex and Chip Kits (10× Genomics) according to the manufacturer's protocol. A total of 5,000 to 7,000 cells were targeted per well. Libraries were sequenced on the NextSeq 500 platform (Illumina) with pair-ended sequencing and dual indexing. A total of 26, 8 and 98 cycles were run for Read 1, i7 index and Read 2, respectively.

### Data Processing, Cell Cluster Annotation and Data Integration

Raw bcl files were demultiplexed and mapped to the reference genome GRCh38 using the Cell Ranger Single Cell v2.0 software (10× Genomics). For individual samples, the EmptyDrops method66 was applied to filter the raw unique molecular identifiers (UMIs) count matrix for real barcodes from ambient background RNA cells. An additional cutoff was applied, filtering for cells with a gene and UMI count greater than 200 and 250, respectively. All cells with a mitochondrial UMI count percentage greater than 20% were removed. We used the Seurat v3 method (Stuart et al., (2019) Cell 177, 1888-1902 e21) in R for data normalisation, dimensionality reduction and clustering using default parameters. Cell clusters were annotated using the Garnett method (Pliner et al., Nature Methods (2019) 16, 983-986) using the default recommended parameters, with a classifier derived from an array of cell signatures for breast epithelial subsets from Lim et al. (2009) Nat Med 15, 907-13, and immune and stromal cell types from the XCell database (Aran et al., (2017) Genome Biol 18:220), including T-cells, B-cells, plasmablasts, monocyte/macrophages, endothelial, fibroblast and perivascular cell signatures.

Data integration was performed using Seurat v3 using default parameters (Stuart et al., (2019) Cell 177, 1888-1902 e21). A total of 2000 features for anchoring (FindIntegrationAnchors step) and 30 dimensions for alignment (IntegrateData step) were used. For reclustering immune and mesenchymal lineages, a total of 5000 features were used for anchoring (FindIntegrationAnchors step), with a total of 30, 20, and 10 Principal Components were used for clustering T-cells, Myeloid cells and B-cells, respectively. The default resolution of 0.8 was used (FindNeighbors and FindClusters step). For clustering without batch correction steps, we merged all individual dataset together (merge function) performed clustering steps (RunPCA, FindNeighbors and FindClusters steps) using the “RNA” assay with a total of 100 principal components.

Identifying Neoplastic from Normal Breast Cancer Epithelial Cells

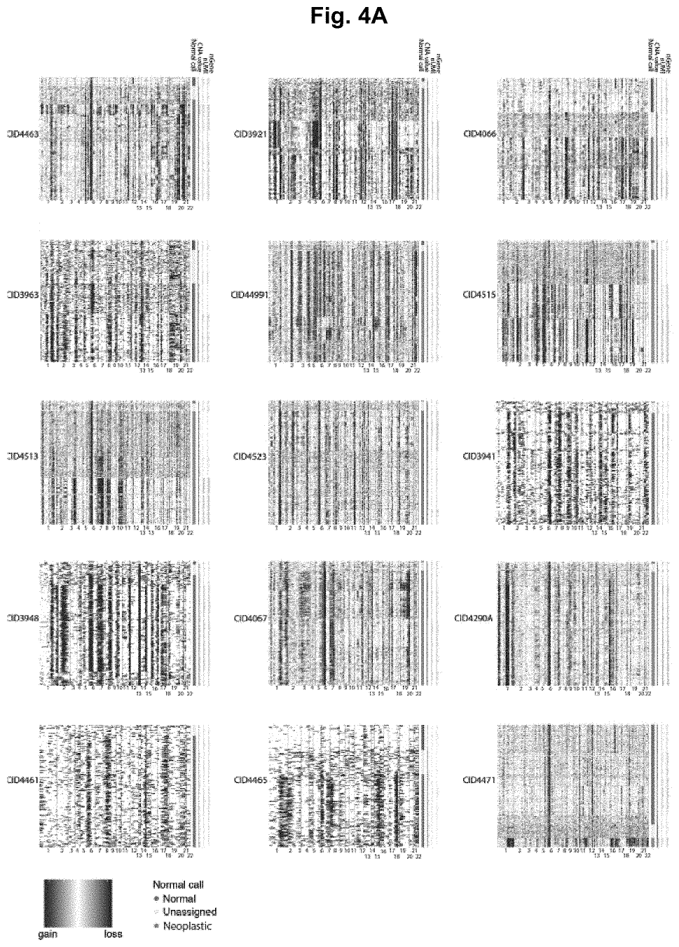

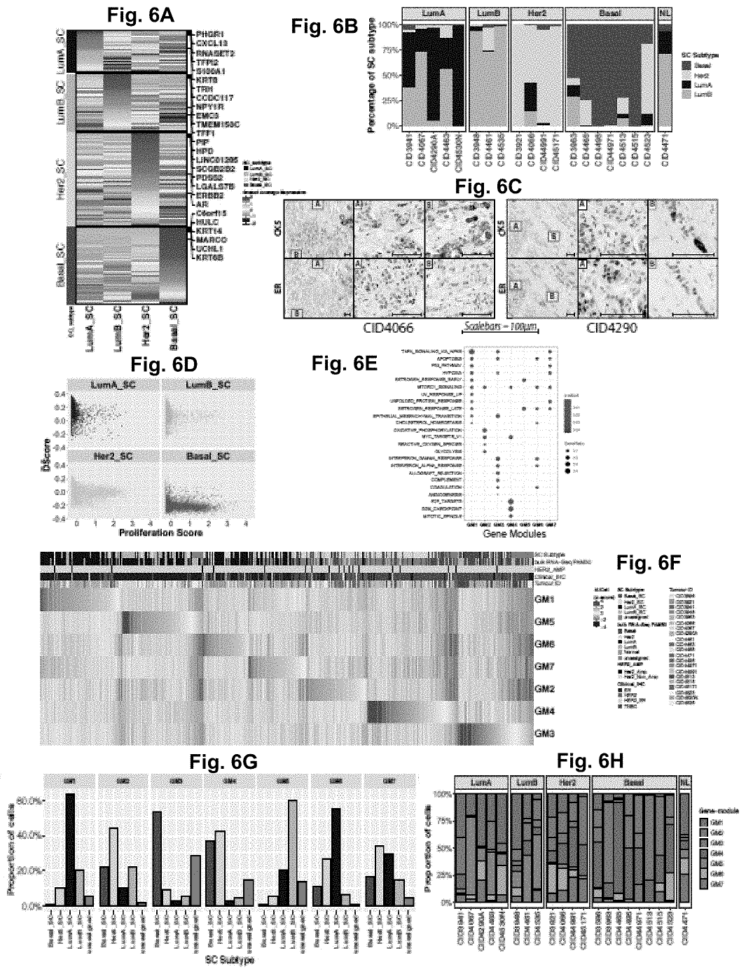

CNV signal for individual cells was estimated using the inferCNV method with a 100 gene sliding window. Genes with a mean count of less than 0.1 across all cells were filtered out prior to analysis, and signal was denoised using a dynamic threshold of 1.3 standard deviations from the mean Immune and endothelial cells were used to define the reference cell inferred copy-number profiles. Epithelial cells were used for the observations. Epithelial cells were classified into normal (non-neoplastic), neoplastic or unassigned using a similar method to that previously described by Neftel et al., (2019) Cell 178, 835-849 e21. Briefly, inferred changes at each genomic loci were scaled (between −1 and +1) and the mean of the squares of these values were used to define a genomic instability score for each cell. In each individual tumour, the top 5% of cells with the highest genomic instability scores were used to create an average CNV profile. Each cell was then correlated to this profile. Cells were plotted with respect to both their genomic instability and correlation scores. Partitioning around medoids (PAM) clustering was performed using the ‘pamk’ function in the R package ‘cluster’ to choose the optimum value for k (between 2-4) using silhouette scores, and the ‘pam’ function to apply the clustering. Thresholds defining normal and neoplastic cells were set at 2 cluster standard deviations to the left and 1.5 standard deviations below the first cancer cluster means. For tumours where PAM could not define more than 1 cluster, the thresholds were set at 1 standard deviation to the left and 1.25 standard deviations below the cluster means. This method was used to identify 27,506 neoplastic and 6084 normal cells in all tumours, the remaining 3208 cells were classed as unassigned (FIG. 6G and FIGS. 4A and 4B). Only tumours with at least 200 epithelial cells were used for this neoplastic cell classification step.

### Calling PAM50 on Pseudo-Bulks and Matching Bulk RNA-Seq

We constructed “pseudo-bulk” expression profiles for each tumour, where all the reads from all cells of a given tumour were added together, and then mapped as one sample. The resulting pseudo-bulk matrix thus constructed was named “Allcells-Pseudobulk” and was subsequently processed similarly to any bulk RNA-Seq sample (i.e. upper quartile normalized-log transformed) for calling molecular subtypes using the PAM50 method (Parker et al., (2009) J Clin Oncol 27, 1160-7). An important consideration made before PAM50 subtyping is to adjust a new sample set relative to the PAM50 training set according to their ER and HER2 status as detailed by Zhao et al., (2015) Breast Cancer Res 17, 29. Thus, after ER/HER2 group-based adjustments, and then applying the PAM50 centroid predictor to the pseudo-bulk data, the methodology identified 7 of 20 Basal-like (CID3963, CID4465, CID4495, CID44971, CID4513, CID4515, CID4523), 4 of 20 HER2E (CID3921, CID4066, CID44991, CID45171), 5 of 20 LumA (CID3941, CID4067, CID4290A, CID4463, CID4530N), 3 of 20 LumB (CID3948, CID4461, CID4535) and 1 of 20 as Normal-like (CID4471).

We performed whole-transcriptome RNA-Seq using Ribosomal Depletion on 18 matching tumour samples from our single-cell dataset. RNA was extracted from diagnostic FFPE blocks using the High Pure RNA Paraffin Kit (Roche #03 270 289 001). The Sequence alignment was done using Salmon (Patro et al., (2017) Nature Methods 14, 417-419). We then called PAM50 on each bulk tumour using Zhao et al., (2015) Breast Cancer Res 17, 29 normalization and then the PAM50 centroid predictor (Table 2).

Table 2: PAM50/scSubtype Comparative Table of all patient samples included in the scSubtype analysis showing their clinical Immunohistochemistry classification, PAM50 Subtype calls on pseudobulk RNA profiles from 10× scRNA-Seq and PAM50 Subtype calls on bulk RNA profiles using Ribozero mRNA-Seq data. Also, included are the number and percentage of individual neoplastic cells in each tumour assigned to each of the 4 scSubtype subtypes.

TABLE 2
PAM50/scSubtype Comparative Table of all patient samples

| | | | | | | Concordance | Concordance | |
|---|---|---|---|---|---|---|---|---|
| | | | | | | between | between | |
| | | scRNA-Seq | BulkRNA- | | | SCTyper | SCTyper | |
| | | Allcells- | Seq | | Majority | and Allcells- | and Bulk | Basal_SC |
| Tumour | Clinical | Pseudobulk | (Ribozero) | SCTyper | SCTyper | Pseudobulk | RNA-Seq | cells |
| ID | IHC | PAM50 | PAM50 | dataset | Subtype | subtypes | subtypes | (freq) |
| CID3948 | ER | LumB | LumA | Training | LumB | Discordant | Discordant | 0 |
| CID4290A | ER | LumA | LumA | Training | LumA | Concordant | Concordant | 35 |
| CID4530N | ER | LumA | LumA | Training | LumA | Concordant | Concordant | 2 |
| CID4535 | ER | LumB | LumB | Training | LumB | Concordant | Concordant | 3 |
| CID3921 | HER2 | Her2 | Her2 | Training | Her2 | Concordant | Concordant | 0 |
| CID45171 | HER2 | Her2 | Not | Training | Her2 | Not available | Not available | 17 |
| CID4495 | TNBC | Basal | Basal | Training | Basal | Concordant | Concordant | 1183 |
| CID44971 | TNBC | Basal | Basal | Training | Basal | Concordant | Concordant | 882 |
| CID44991 | TNBC | Her2 | Not | Training | Her2 | Not available | Not available | 167 |
| CID4515 | TNBC | Basal | Basal | Training | Basal | Concordant | Concordant | 2167 |
| CID3941 | ER | LumA | LumA | Testing | LumA | Concordant | Concordant | 9 |
| CID4067 | ER | LumA | LumA | Testing | LumB | Concordant | Discordant | 15 |
| CID4461 | ER | LumB | LumB | Testing | LumB | Concordant | Concordant | 5 |
| CID4463 | ER | LumA | LumB | Testing | LumB | Discordant | Concordant | 2 |
| CID4471 | ER | Normal | Normal | Testing | Normal | Concordant | Concordant | 11 |
| CID3963 | HER2 | Basal | Basal | Testing | Basal | Concordant | Concordant | 116 |
| CID4066 | HER2_ER | Her2 | Normal | Testing | Her2 | Discordant | Discordant | 4 |
| CID4465 | TNBC | Basal | Basal | Testing | Basal | Concordant | Concordant | 91 |
| CID4513 | TNBC | Basal | LumB | Testing | Basal | Discordant | Discordant | 756 |
| CID4523 | TNBC | Basal | Basal | Testing | Her2 | Concordant | Discordant | 218 |
| | | Her2e_SC | LumA_SC | LumB_SC | Basal_SC | Her2e_SC | LumA_SC | LumB_SC |
| | Tumour | cells | cells | cells | cells | cells | cells | cells |
| | ID | (freq) | (freq) | (freq) | (%) | (%) | (%) | (%) |
| | CID3948 | 3 | 13 | 245 | 0 | 1.15 | 4.98 | 93.87 |
| | CID4290A | 52 | 3748 | 218 | 0.86 | 1.28 | 92.47 | 5.38 |
| | CID4530N | 1 | 1706 | 6 | 0.12 | 0.06 | 99.48 | 0.35 |
| | CID4535 | 5 | 5 | 2210 | 0.13 | 0.22 | 0.22 | 99.42 |
| | CID3921 | 441 | 0 | 0 | 0 | 100 | 0 | 0 |
| | CID45171 | 792 | 1 | 3 | 2.09 | 97.42 | 0.12 | 0.37 |
| | CID4495 | 0 | 1 | 0 | 99.92 | 0 | 0.08 | 0 |
| | CID44971 | 6 | 4 | 2 | 98.66 | 0.67 | 0.45 | 0.22 |
| | CID44991 | 3712 | 78 | 61 | 4.16 | 92.38 | 1.94 | 1.52 |
| | CID4515 | 2 | 0 | 0 | 99.91 | 0.09 | 0 | 0 |
| | CID3941 | 5 | 105 | 77 | 4.59 | 2.55 | 53.57 | 39.29 |
| | CID4067 | 58 | 548 | 1731 | 0.64 | 2.47 | 23.3 | 73.6 |
| | CID4461 | 47 | 3 | 152 | 2.42 | 22.71 | 1.45 | 73.43 |
| | CID4463 | 81 | 198 | 378 | 0.3 | 12.29 | 30.05 | 57.36 |
| | CID4471 | 0 | 50 | 151 | 5.19 | 0 | 23.58 | 71.23 |
| | CID3963 | 15 | 24 | 67 | 52.25 | 6.76 | 10.81 | 30.18 |
| | CID4066 | 294 | 144 | 79 | 0.77 | 56.43 | 27.64 | 15.16 |
| | CID4465 | 32 | 1 | 0 | 73.39 | 25.81 | 0.81 | 0 |
| | CID4513 | 167 | 49 | 86 | 71.46 | 15.78 | 4.63 | 8.13 |
| | CID4523 | 795 | 134 | 20 | 18.68 | 68.12 | 11.48 | 1.71 |

Calling Intrinsic Subtype on scRNA-Seq Using scSubtype

To design and validate a new subtyping tool specific for scRNA-Seq data, we first divided our tumour samples into training and testing sets. The training dataset was defined by identifying tumours with unambiguous molecular subtypes. Here, we identified robust training set samples using two subtyping approaches: (i) PAM50 subtyping of the Allcells-Pseudobulk datasets (described above); and (ii) hierarchical clustering of the Allcells-Pseudobulk data with the 1,100 tumours in the TCGA BrCa RNA-Seq dataset using 2000 genes from an intrinsic breast cancer gene list (Parker, J. S. et al. (2009) J Clin Oncol 27, 1160-7). We first identified tumours that shared the same “concordant” subtype from both Allcells-Pseudobulk PAM50 calls and TCGA hierarchical clustering-based subtype classifications (Table 2). Next, since our methodology aimed to subtype cancer cells, we removed any tumours with <150 cancer cells. Finally, we did not include cells from the two metaplastic samples (CID4513 and CID4523) in the training data because this is a histological subtype not used in the original PAM50 training set. Using this approach, we identified 10 tumour samples in the training dataset: HER2E (CID3921, CID44991, CID45171), Basal-like (CID4495, CID44971, CID4515), LumA (CID4290, CID4530) and LumB (CID3948, CID4535). Only tumour cells with greater than 500 UMIs were used for training and test datasets in scSubtype (total of 24,889 cells).

Within each training set subtype, we utilized the cancer cells from each tumour sample and performed pairwise single cell integrations and differential gene expression calculations. The integration was carried out in a “within group” pairwise fashion using the FindIntegrationAnchors and IntegrateData functions in the Seurat v3 package (Stuart et al., (2019) Cell 177, 1888-1902 e21). Briefly, the first step identifies anchors between pairs of cells from each dataset using mutual nearest neighbors. The second step integrates the datasets together based on a distance based weights matrix constructed from the anchor pairs. Differentially expressed genes were calculated between each pair using a Wilcoxon Rank Sum test by the FindAllMarkers function within Seurat v3. As the number of cancer cells per tumour sample were highly variable, this strategy prevented a bias of identifying genes for a training group from a sample with the highest number of cells. The following pairs were analyzed: HER2E (CID3921-CID44991, CID44991-CID45171, CID45171-CID3921), Basal-like (CID4495-CID44971, CID44971-CID4515, CID4515-CID4495), LumA (CID4290-CID4530) and LumB (CID3948-CID4535). In this way we identified unique upregulated genes per sample, but also genes broadly highlighting cells within each respective training group or subtype. We removed any duplicate genes occurring between the 4 training groups, which yielded 4 sets of genes composed of 89 genes defining Basal_SC, 102 genes defining HER2E_SC, 46 genes defining LumA_SC and 65 genes defining LumB_SC, which we define as “scSubtype” gene signatures (Table 3). Table 3 represents the scSubtype gene table Gene lists used to define the single-cell scSubtype molecular subtype classifier, one for each scSubtype (Basal_SC, Her2E_SC, LumA_SC and LumB_SC).

TABLE 3
cSubtype gene table Gene lists used to define the
single-cell scSubtype molecular subtype classifier

| Basal_SC | Her2E_SC | LumA_SC | LumB_SC |
|---|---|---|---|
| EMP1 | PSMA2 | SH3BGRL | UGCG |
| TAGLN | PPP1R1B | HSPB1 | ARMT1 |
| TTYH1 | SYNGR2 | PHGR1 | ISOC1 |
| RTN4 | CNPY2 | SOX9 | GDF15 |
| TK1 | LGALS7B | CEBPD | ZFP36 |
| BUB3 | CYBA | CITED2 | PSMC5 |
| IGLV3.25 | FTH1 | TM4SF1 | DDX5 |
| FAM3C | MSL1 | S100P | TMEM150C |
| TMEM123 | IGKV3.15 | KCNK6 | NBEAL1 |
| KDM5B | STARD3 | AGR3 | CLEC3A |
| KRT14 | HPD | MPC2 | GADD45G |
| ALG3 | HMGCS2 | CXCL13 | MARCKS |
| KLK6 | ID3 | RNASET2 | FHL2 |
| EEF2 | NDUFB8 | DDIT4 | CCDC117 |
| NSMCE4A | COTL1 | SCUBE2 | LY6E |
| LYST | AIM1 | KRT8 | GJA1 |
| DEDD | MED24 | MZT2B | PSAP |
| HLA.DRA | CEACAM6 | IFI6 | TAF7 |
| PAPOLA | FABP7 | RPS26 | PIP |
| SOX4 | CRABP2 | TAGLN2 | HSPA2 |
| ACTR3B | NR4A2 | SPTSSA | DSCAM.AS1 |
| EIF3D | COX14 | ZFP36L1 | PSMB7 |
| CACYBP | ACADM | MGP | STARD10 |
| RARRES1 | PKM | KDELR2 | ATF3 |
| STRA13 | ECH1 | PPDPF | WBP11 |
| MFGE8 | C17orf89 | AZGP1 | MALAT1 |
| FRZB | NGRN | AP000769.1 | C6orf48 |
| SDHD | ATG5 | MYBPC1 | HLA.DRB1 |
| UCHL1 | SNHG25 | S100A1 | HIST1H2BD |
| TMEM176A | ETFB | TFPI2 | CCND1 |
| CAV2 | EGLN3 | JUN | STC2 |
| MARCO | CSNK2B | SLC25A6 | NR4A1 |
| P4HB | RHOC | HSP90AB1 | NPY1R |
| CHI3L2 | PSENEN | ARF5 | FOS |
| APOE | CDK12 | PMAIP1 | ZFAND2A |
| ATP1B1 | ATP5I | TNFRSF12A | CFL1 |
| C6orf15 | ENTHD2 | FXYD3 | RHOB |
| KRT6B | QRSL1 | RASD1 | LMNA |
| TAF1D | S100A7 | PYCARD | SLC40A1 |
| ACTA2 | TPM1 | PYDC1 | CYB5A |
| LY6D | ATP5C1 | PHLDA2 | SRSF5 |
| SAA2 | HIST1H1E | BZW2 | SEC61G |
| CYP27A1 | LGALS1 | HOXA9 | CTSD |
| DLK1 | GRB7 | XBP1 | DNAJC12 |
| IGKV1.5 | AQP3 | AGR2 | IFITM1 |
| CENPW | ALDH2 | HSP90AA1 | MAGED2 |
| RAB18 | EIF3E | | RBP1 |
| TNFRSF11B | ERBB2 | | TFF1 |
| VPS28 | LCN2 | | APLP2 |
| HULC | SLC38A10 | | TFF3 |
| KRT16 | TXN | | TRH |
| CDKN2A | DBI | | NUPR1 |
| AHNAK2 | RP11.206M11.7 | | EMC3 |
| SEC22B | TUBB | | TXNIP |
| CDC42EP1 | CRYAB | | ARPC4 |
| HMGA1 | CD9 | | KCNE4 |
| CAV1 | PDSS2 | | ANPEP |
| BAMBI | XIST | | MGST1 |
| TOMM22 | MED1 | | TOB1 |
| ATP6V0E2 | C6orf203 | | ADIRF |
| MTCH2 | PSMD3 | | TUBA1B |
| PRSS21 | TMC5 | | MYEOV2 |
| HDAC2 | UQCRQ | | MLLT4 |
| ZG16B | EFHD1 | | DHRS2 |
| GAL | BCAM | | IFITM2 |
| SCGB1D2 | GPX1 | | |
| S100A2 | EPHX1 | | |
| GSPT1 | AREG | | |
| ARPC1B | CDK2AP2 | | |
| NIT1 | SPINK8 | | |
| NEAT1 | PGAP3 | | |
| DSC2 | NFIC | | |
| RP1.60O19.1 | THRSP | | |
| MAL2 | LDHB | | |
| TMEM176B | MT1X | | |
| CYP1B1 | HIST1H4C | | |
| EIF3L | LRRC26 | | |
| FKBP4 | SLC16A3 | | |
| WFDC2 | BACE2 | | |
| SAA1 | MIEN1 | | |
| CXCL17 | AR | | |
| PFDN2 | CRIP2 | | |
| UCP2 | NME1 | | |
| RAB11B | DEGS2 | | |
| FDCSP | CASC3 | | |
| HLA.DPB1 | FOLR1 | | |
| PCSK1N | SIVA1 | | |
| C4orf48 | SLC25A39 | | |
| CTSC | IGHG1 | | |
| | ORMDL3 | | |
| | KRT81 | | |
| | SCGB2B2 | | |
| | LINC01285 | | |
| | CXCL8 | | |
| | KRT15 | | |
| | RSU1 | | |
| | ZFP36L2 | | |
| | DKK1 | | |
| | TMED10 | | |
| | IRX3 | | |
| | S100A9 | | |
| | YWHAZ | | |

To assign a subtype call to a cell we calculated the average (i.e. mean) read counts for each of the 4 signatures for each cell. The SC subtype with the highest signature score was then assigned to each cell. We utilized this method to subtype all 24,489 neoplastic cells, from both our training samples (n=10) and the remaining test (n=10) set samples.

### Calculating Proliferation and Differentiation Scores

As previously described, we calculated the degree of epithelial cell differentiation status (DScore), and proliferation signature status, on each and every tumour cell in our scRNA-Seq cohort, as well as the 1,100 tumours in TCGA dataset. The 11 genes used to compute the proliferation signature status are independent of the scSubtype gene lists, while the Dscore is computed using a centroid based predictor with information from ˜20 thousand genes.

### Histology and Immunohistochemical Staining of CK5 and ER

Tumour tissue was fixed in 10% neutral buffered formalin for 24 hrs and then processed for paraffin embedding. Diagnostic tumour blocks were accessed for samples that did not have a research block available. Blocks were sectioned at 4 uM. Sections were stained with Haematoxylin and Eosin for standard histological analysis Immunohistochemistry (IHC) was performed on serial sections with pre-diluted primary antibodies against ER (clone 6F11; leica PA0151) or CK5 (clone XM26; leica PA0468) using suggested protocols on the BOND RX Autostainer (Leica, Germany). Antigen retrieval was performed for 20 min using BOND Epitope Retrieval solution 1 for ER or solution 2 for CK5, followed by primary antibody incubation for 60 min and secondary staining with the Bond Refine detection system (Leica). Slides were imaged using the Aperio CS2 Digital Pathology Slide Scanner.

### Gene Module Analysis of Neoplastic Intra-Tumour Heterogeneity

For each individual tumour, with more than 50 neoplastic cells, the neoplastic cells were clustered using Seurat v337 at five resolutions (0.4, 0.8, 1.2, 1.6, 2.0). MAST69 was then used to identify the top-200 differentially regulated genes in each cluster. Only gene-signatures containing greater than 5 genes and originating from clusters of more than 5 cells were kept. In addition, redundancy was reduced by comparing all pairs of signatures within each sample and removing the pair with fewest genes from those pairs with a Jaccard index greater than 0.75. Across all tumours, a total of 574 gene-signatures of intra-tumour heterogeneity were identified.

Consensus clustering (using spherical k-means, skmeans, implemented in the cola R package: https://www.bioconductor.org/packages/release/bioc/html/cola.html) of the Jaccard similarities between these gene-signatures was used to identify 7 robust groups, or gene-modules. For each of these, a gene module was defined by taking the 200 genes that had the highest frequency of occurrence across clusters and individual tumours. These are defined as gene-modules GM1 to GM7. A gene-module signature was calculated for each cell using AUCell and each neoplastic cell was assigned to a module, using the maximum of the scaled AUCell gene-module signature scores. This resulted in 4,368, 3,288, 2,951, 4,326, 3,931, 2,500, 3,125 cells assigned to GM1 to GM7, respectively. These are defined as gene-module based neoplastic cell states.

### Differential Gene Expression, Module Scoring and Gene Ontology Enrichment

Differential gene expression was performed using the MAST method (Finak, G. et al., (2015). Genome Biol 16, 278) in Seurat (FindAllMarkers step) using default cutoff parameters. All DEGs from each cluster (data not shown) were used as input into the ClusterProfiler package for gene ontology functional enrichment. All ontologies within the enrichGO databases were used with the human org.Hs.eg.db database. Results were clustered, scaled and visualised using the pheatmap package in R. Cytotoxic, TAM and Dysfunctional T-cell gene expression signatures were assigned using the AddModuleScore function in Seurat v337. The list of genes used for dysfunctional T-cells were adopted from Li et al., (2019) Cell 176, 775-789 e18. The TAM gene list was adopted from Cassetta et al., (2019) Biomarkers, and Therapeutic Targets. Cancer Cell 35, 588-602 e10. The cytotoxic gene list consists of 12 genes which translate to effector cytotoxic proteins (GZMA, GZMB, GZMH, GZMK, GZMM, GNLY, PRF1 and FASLG) and well described cytotoxic T-cell activation markers (IFNG, TNF, IL2R and IL2).

### Pseudotemporal Ordering to Infer Cell Trajectories

Cell differentiation was inferred for mesenchymal cells (CAFs, PVL and Endothelial cells) using the Monocle 2 method with default parameters as recommended by developers. Integrated gene expression matrices from each cell type were first exported from Seurat v3 into Monocle to construct a CellDataSet. All variable genes defined by the differentialGeneTest function (q-val cutoff<0.001) were used for cell ordering with the setOrderingFilter function. Dimensionality reduction was performed with no normalisation methods and the DDRTree reduction method in the reduceDimension step.

### CITE-Seq Antibody Staining

Samples were stained with 10× Chromium 3′ mRNA capture compatible TotalSeq-A antibodies (Biolegend, USA). Staining was performed as previously described by Stoeckius et. al., (2017) Nat Methods 14, 865-868 with a few modifications listed below. A total of four cases from our scRNA-Seq cohort were analyzed, including one luminal (CID4040), one HER2 (CID383) and two TNBC (CID4515 and CID3956). A panel of 157 barcoded antibodies (data not shown) were used, which recognised a range of cell surface lineage and activation markers, in addition to a large collection of co-stimulatory and co-inhibitory receptors and ligands. Briefly, a maximum of 1 million cells per sample was resuspended in 120 ul of cell staining buffer (Biolegend, USA) with 5 ul of Fc receptor Block (TrueStain FcX, Bioelegend, USA) for 15 min. This was followed by a 30 min staining of the antibodies at 4° C. A concentration of 1 ug/100 ul was used for all antibody markers used in this study. The cells were then washed 3 times with PBS containing 10% FCS media followed by centrifugation (300×g for 5 min at 4° C.) and expungement of supernatant. The sample was then resuspended in PBS with 10% FCS for 10× Chromium capture.

### CITE-Seq Data Processing and Imputation

Demultiplexed reads were assigned to individual cells and antibodies with python package CITE-seq-count v.1.4.3 (https://github.com/Hoohm/CITE-seq-Count/tree/1.4.2). CITE counts were normalised and scaled with Seurat v.3.1.4. Imputation of CITE data was performed per individual cell type (B-cells, T-cells, myeloid cells, mesenchymal cells) for those antibodies that were differentially expressed between subclusters (FindAllMarkers step) for individual samples. We used anchoring based transfer learning to transfer protein expression levels from these four samples to the remaining BrCa cases.

Survival Analysis of scRNA-Seq Signatures

To assess impact of particular cell types described by scRNA-Seq (e.g. LAM1 and LAM2) on clinical outcome, we assessed the association between gene signatures (derived as described above) with patient overall survival in the METABRIC cohort. For each tumour from the bulk expression cohort, average gene signature expression was derived using the top 100 genes from the gene signature of interest. Patients were then stratified based on the top and bottom 30%, and survival curves were generated using the Kaplan Meier method with the ‘survival’ package in R (https://crans-projectorg/package—survival). We assessed the significance between two groups using the log-rank test statistics. Differences in survival between ecotypes were assessed using Kaplan-Meier analysis and log-rank test statistics, using the survival and survminer R packages.

### Tumour Ecotype Analysis Using Deconvolution of Bulk Sequencing Patient Cohorts

CIBERSORTx59 and DWLS60 were used to deconvolute predicted cell-fractions from a number of bulk transcript profiling datasets. To prevent confounding of cycling cell-types we first assigned all neoplastic epithelial cells with a proliferation score>0 as cycling and then combined these with “cycling” cell states from all other cell-types to generate a single “Cycling” cell-state. To generate cell-type signature matrices for each of the tiers of cell-type annotation described in this study, we randomly subsampled 15% of cells from each level of annotation type.

### CIBERSORTx

We then ran CIBERSORTx “cibersortx/fractions” to generate cell-type signature matrices using the following parameters: --single_cell TRUE --G.min 300 --G.max 500 --q.value 0.01 --filter FALSE --k.max 999 --replicates 5 --sampling 0.5 --fraction 0.75.

For cell-type deconvolution of bulk tumours we ran CIBERSORTx “cibersortx/fractions” to calculate the relative cell-type abundances in each tumour. S-mode batch correction was used for the METABRIC tumours.

### DWLS

For deconvolution analysis using DWLS we used the functions in the “Deconvolution_functions.R” script obtained from https://github.com/dtsoucas/DWLS. Cell-type signature matrices were generated using the buildSignatureMatrixMAST( ) function and then filtered to only contain genes that are present in both the bulk and single-cell derived signature matrices, using the trimData( ) function. Cell-type abundances were then calculated using the solveDampenedWLS( ) function.

### Bulk Expression Datasets

Pseudo-bulk expression matrices were generated from the scRNA-Seq datasets in this study by summing the unique molecular identifiers (UMIs) for each gene across all cells for each tumour. Normalised METABRIC expression matrices, clinical information and PAM50 subtype classifications were obtained from https://www.cbioportal.org/study/summary?id—brca_metabric.

### Tumour Ecotypes

Tumour ecotypes in the METABRIC cohort were identified using spherical k-means (skmeans) based consensus clustering (as implemented in the cola R package: https://www.bioconductor.org/packages/release/bioc/html/cola.html) of the predicted cell-fraction from either CIBERSORTx or DWLS, in each bulk METABRIC patient tumour. When comparing ecotypes between methods (i.e., consensus clustering results from using cell-abundances of all cell-types or just the 32 significantly correlated cell-types from CIBERSORTx deconvolution and consensus clustering results from CIBERSORTx or DWLS cell-abundances) the number of tumour ecotypes was fixed as 9 and the tumour overlaps between all ecotype pairs was calculated (Tables 4 and 5). Common ecotypes were then identified by identifying the ecotype pairs with the largest average METABRIC tumour overlap.

With reference to Table 4: The table columns are: ecotype_all: The ecotype ID when using all cell-types; ecotype_all_samples: number of tumours in ecotype from using all cell-types; ecotype_signif: The ecotype ID when using only the significantly correlated cell-types; ecotype_signif samples: number of tumours in ecotype from using only the significantly correlated cell-types; overlap: number of overlapping tumours between the ecotype pairs; ecotype_all_overlap: fraction of overlapping tumours from ecotypes generated using all cell-types; ecotype_signif overlap: fraction of overlapping tumours from ecotypes generated using only the significantly correlated cell-types; avg_overlap: the averaged fractional overlap (i.e., (ecotype_all_overlap+ecotype_signif overlap)/2)

TABLE 4
Tumour ecotype

| | ecotype_all_ | ecotype_ | ecotype_signif_ | | ecotype_all_ | ecotype_signif_ | |
|---|---|---|---|---|---|---|---|
| ecotype_all | samples | signif | samples | overlap | overlap | overlap | avg_overlap |
| 1 | 267 | 1 | 313 | 234 | 0.876404 | 0.747604 | 0.812004164 |
| 1 | 267 | 4 | 289 | 16 | 0.059925 | 0.055363 | 0.057644208 |
| 1 | 267 | 7 | 237 | 8 | 0.029963 | 0.033755 | 0.031858911 |
| 1 | 267 | 9 | 18 | 1 | 0.003745 | 0.055556 | 0.029650437 |
| 1 | 267 | 6 | 184 | 3 | 0.011236 | 0.016304 | 0.013770151 |
| 1 | 267 | 5 | 239 | 3 | 0.011236 | 0.012552 | 0.011894128 |
| 1 | 267 | 8 | 236 | 2 | 0.007491 | 0.008475 | 0.007982606 |
| 1 | 267 | 2 | 199 | 0 | 0 | 0 | 0 |
| 1 | 267 | 3 | 277 | 0 | 0 | 0 | 0 |
| 2 | 272 | 3 | 277 | 237 | 0.871324 | 0.855596 | 0.863459599 |
| 2 | 272 | 9 | 18 | 2 | 0.007353 | 0.111111 | 0.059232026 |
| 2 | 272 | 8 | 236 | 10 | 0.036765 | 0.042373 | 0.039568794 |
| 2 | 272 | 5 | 239 | 6 | 0.022059 | 0.025105 | 0.023581713 |
| 2 | 272 | 2 | 199 | 5 | 0.018382 | 0.025126 | 0.021753991 |
| 2 | 272 | 7 | 237 | 5 | 0.018382 | 0.021097 | 0.0197397 |
| 2 | 272 | 4 | 289 | 3 | 0.011029 | 0.010381 | 0.010705017 |
| 2 | 272 | 1 | 313 | 3 | 0.011029 | 0.009585 | 0.010307038 |
| 2 | 272 | 6 | 184 | 1 | 0.003676 | 0.005435 | 0.004555627 |
| 3 | 205 | 2 | 199 | 182 | 0.887805 | 0.914573 | 0.901188871 |
| 3 | 205 | 4 | 289 | 18 | 0.087805 | 0.062284 | 0.075044308 |
| 3 | 205 | 6 | 184 | 3 | 0.014634 | 0.016304 | 0.015469247 |
| 3 | 205 | 8 | 236 | 2 | 0.009756 | 0.008475 | 0.009115337 |
| 3 | 205 | 1 | 313 | 0 | 0 | 0 | 0 |
| 3 | 205 | 3 | 277 | 0 | 0 | 0 | 0 |
| 3 | 205 | 5 | 239 | 0 | 0 | 0 | 0 |
| 3 | 205 | 7 | 237 | 0 | 0 | 0 | 0 |
| 3 | 205 | 9 | 18 | 0 | 0 | 0 | 0 |
| 4 | 264 | 4 | 289 | 216 | 0.818182 | 0.747405 | 0.782793331 |
| 4 | 264 | 9 | 18 | 8 | 0.030303 | 0.444444 | 0.237373737 |
| 4 | 264 | 3 | 277 | 22 | 0.083333 | 0.079422 | 0.081377858 |
| 4 | 264 | 6 | 184 | 6 | 0.022727 | 0.032609 | 0.027667984 |
| 4 | 264 | 2 | 199 | 6 | 0.022727 | 0.030151 | 0.026439013 |
| 4 | 264 | 8 | 236 | 2 | 0.007576 | 0.008475 | 0.008025167 |
| 4 | 264 | 7 | 237 | 2 | 0.007576 | 0.008439 | 0.008007288 |
| 4 | 264 | 5 | 239 | 2 | 0.007576 | 0.008368 | 0.007971979 |
| 4 | 264 | 1 | 313 | 0 | 0 | 0 | 0 |
| 5 | 195 | 7 | 237 | 148 | 0.758974 | 0.624473 | 0.691723466 |
| 5 | 195 | 1 | 313 | 26 | 0.133333 | 0.083067 | 0.108200213 |
| 5 | 195 | 9 | 18 | 3 | 0.015385 | 0.166667 | 0.091025641 |
| 5 | 195 | 5 | 239 | 8 | 0.041026 | 0.033473 | 0.037249222 |
| 5 | 195 | 4 | 289 | 4 | 0.020513 | 0.013841 | 0.017176825 |
| 5 | 195 | 6 | 184 | 2 | 0.010256 | 0.01087 | 0.010562988 |
| 5 | 195 | 8 | 236 | 2 | 0.010256 | 0.008475 | 0.009365493 |
| 5 | 195 | 3 | 277 | 2 | 0.010256 | 0.00722 | 0.008738313 |
| 5 | 195 | 2 | 199 | 0 | 0 | 0 | 0 |
| 6 | 215 | 5 | 239 | 195 | 0.906977 | 0.8159 | 0.861438163 |
| 6 | 215 | 9 | 18 | 1 | 0.004651 | 0.055556 | 0.030103359 |
| 6 | 215 | 8 | 236 | 6 | 0.027907 | 0.025424 | 0.026665353 |
| 6 | 215 | 7 | 237 | 6 | 0.027907 | 0.025316 | 0.026611716 |
| 6 | 215 | 1 | 313 | 5 | 0.023256 | 0.015974 | 0.019615127 |
| 6 | 215 | 6 | 184 | 1 | 0.004651 | 0.005435 | 0.005042973 |
| 6 | 215 | 4 | 289 | 1 | 0.004651 | 0.00346 | 0.004055685 |
| 6 | 215 | 2 | 199 | 0 | 0 | 0 | 0 |
| 6 | 215 | 3 | 277 | 0 | 0 | 0 | 0 |
| 7 | 199 | 6 | 184 | 154 | 0.773869 | 0.836957 | 0.805412934 |
| 7 | 199 | 4 | 289 | 23 | 0.115578 | 0.079585 | 0.097581332 |
| 7 | 199 | 8 | 236 | 8 | 0.040201 | 0.033898 | 0.037049655 |
| 7 | 199 | 7 | 237 | 4 | 0.020101 | 0.016878 | 0.01848907 |
| 7 | 199 | 5 | 239 | 4 | 0.020101 | 0.016736 | 0.018418452 |
| 7 | 199 | 2 | 199 | 3 | 0.015075 | 0.015075 | 0.015075377 |
| 7 | 199 | 3 | 277 | 2 | 0.01005 | 0.00722 | 0.008635234 |
| 7 | 199 | 1 | 313 | 1 | 0.005025 | 0.003195 | 0.004110007 |
| 7 | 199 | 9 | 18 | 0 | 0 | 0 | 0 |
| 8 | 215 | 8 | 236 | 133 | 0.618605 | 0.563559 | 0.591081987 |
| 8 | 215 | 1 | 313 | 41 | 0.190698 | 0.13099 | 0.160844045 |
| 8 | 215 | 7 | 237 | 16 | 0.074419 | 0.067511 | 0.070964577 |
| 8 | 215 | 9 | 18 | 2 | 0.009302 | 0.111111 | 0.060206718 |
| 8 | 215 | 5 | 239 | 10 | 0.046512 | 0.041841 | 0.044176316 |
| 8 | 215 | 4 | 289 | 5 | 0.023256 | 0.017301 | 0.020278426 |
| 8 | 215 | 6 | 184 | 4 | 0.018605 | 0.021739 | 0.020171891 |
| 8 | 215 | 2 | 199 | 3 | 0.013953 | 0.015075 | 0.014514433 |
| 8 | 215 | 3 | 277 | 1 | 0.004651 | 0.00361 | 0.004130636 |
| 9 | 160 | 8 | 236 | 71 | 0.44375 | 0.300847 | 0.372298729 |
| 9 | 160 | 7 | 237 | 48 | 0.3 | 0.202532 | 0.251265823 |
| 9 | 160 | 3 | 277 | 13 | 0.08125 | 0.046931 | 0.064090704 |
| 9 | 160 | 6 | 184 | 10 | 0.0625 | 0.054348 | 0.058423913 |
| 9 | 160 | 5 | 239 | 11 | 0.06875 | 0.046025 | 0.057387552 |
| 9 | 160 | 9 | 18 | 1 | 0.00625 | 0.055556 | 0.030902778 |
| 9 | 160 | 4 | 289 | 3 | 0.01875 | 0.010381 | 0.014565311 |
| 9 | 160 | 1 | 313 | 3 | 0.01875 | 0.009585 | 0.014167332 |
| 9 | 160 | 2 | 199 | 0 | 0 | 0 | 0 |

With reference to Table 5: The table columns are: cibersortx_ecotype: The ecotype ID when using CIBERSORTx; cibersortx_ecotype_samples: number of tumours in ecotype from CIBERSORTx; dwls_ecotype: The ecotype ID when using DWLS; dwls_ecotype_samples: number of tumours in ecotype from using DWLS; overlap: number of overlapping tumours between the ecotype pairs; cibersortx_ecotype_overlap: fraction of overlapping tumours from ecotypes generated using CIBERSORTx; dwls_ecotype_overlap: fraction of overlapping tumours from ecotypes generated using DWLS; avg_overlap: the averaged fractional overlap (i.e., (cibersortx_ecotype_overlap+dwls_ecotype_overlap)/2)

TABLE 5
Tumour ecotype

| cibersortx_ | cibersortx_ | dwls_ | dwls_ | | cibersortx_ | dwls_ | |
|---|---|---|---|---|---|---|---|
| ecotype | ecotype_samples | ecotype | ecotype_samples | overlap | ecotype_overlap | ecotype_overlap | avg_overlap |
| 1 | 267 | 9 | 255 | 113 | 0.423220974 | 0.443137255 | 0.433179114 |
| 1 | 267 | 5 | 207 | 58 | 0.217228464 | 0.280193237 | 0.248710851 |
| 1 | 267 | 8 | 179 | 46 | 0.172284644 | 0.25698324 | 0.214633942 |
| 1 | 267 | 1 | 199 | 27 | 0.101123596 | 0.135678392 | 0.118400994 |
| 1 | 267 | 4 | 212 | 10 | 0.037453184 | 0.047169811 | 0.042311497 |
| 1 | 267 | 7 | 241 | 7 | 0.026217228 | 0.029045643 | 0.027631436 |
| 1 | 267 | 2 | 230 | 5 | 0.018726592 | 0.02173913 | 0.020232861 |
| 1 | 267 | 6 | 179 | 1 | 0.003745318 | 0.005586592 | 0.004665955 |
| 1 | 267 | 3 | 290 | 0 | 0 | 0 | 0 |
| 2 | 272 | 3 | 290 | 221 | 0.8125 | 0.762068966 | 0.787284483 |
| 2 | 272 | 7 | 241 | 14 | 0.051470588 | 0.058091286 | 0.054780937 |
| 2 | 272 | 8 | 179 | 11 | 0.040441176 | 0.061452514 | 0.050946845 |
| 2 | 272 | 5 | 207 | 11 | 0.040441176 | 0.053140097 | 0.046790637 |
| 2 | 272 | 1 | 199 | 8 | 0.029411765 | 0.040201005 | 0.034806385 |
| 2 | 272 | 6 | 179 | 4 | 0.014705882 | 0.022346369 | 0.018526126 |
| 2 | 272 | 9 | 255 | 2 | 0.007352941 | 0.007843137 | 0.007598039 |
| 2 | 272 | 2 | 230 | 1 | 0.003676471 | 0.004347826 | 0.004012148 |
| 2 | 272 | 4 | 212 | 0 | 0 | 0 | 0 |
| 3 | 205 | 6 | 179 | 157 | 0.765853659 | 0.877094972 | 0.821474315 |
| 3 | 205 | 2 | 230 | 22 | 0.107317073 | 0.095652174 | 0.101484624 |
| 3 | 205 | 4 | 212 | 11 | 0.053658537 | 0.051886792 | 0.052772665 |
| 3 | 205 | 9 | 255 | 8 | 0.03902439 | 0.031372549 | 0.03519847 |
| 3 | 205 | 3 | 290 | 3 | 0.014634146 | 0.010344828 | 0.012489487 |
| 3 | 205 | 7 | 241 | 2 | 0.009756098 | 0.008298755 | 0.009027426 |
| 3 | 205 | 8 | 179 | 1 | 0.004878049 | 0.005586592 | 0.00523232 |
| 3 | 205 | 5 | 207 | 1 | 0.004878049 | 0.004830918 | 0.004854483 |
| 3 | 205 | 1 | 199 | 0 | 0 | 0 | 0 |
| 4 | 264 | 2 | 230 | 185 | 0.700757576 | 0.804347826 | 0.752552701 |
| 4 | 264 | 3 | 290 | 30 | 0.113636364 | 0.103448276 | 0.10854232 |
| 4 | 264 | 4 | 212 | 16 | 0.060606061 | 0.075471698 | 0.068038879 |
| 4 | 264 | 9 | 255 | 11 | 0.041666667 | 0.043137255 | 0.042401961 |
| 4 | 264 | 6 | 179 | 8 | 0.03030303 | 0.044692737 | 0.037497884 |
| 4 | 264 | 5 | 207 | 7 | 0.026515152 | 0.033816425 | 0.030165788 |
| 4 | 264 | 7 | 241 | 5 | 0.018939394 | 0.020746888 | 0.019843141 |
| 4 | 264 | 8 | 179 | 2 | 0.007575758 | 0.011173184 | 0.009374471 |
| 4 | 264 | 1 | 199 | 0 | 0 | 0 | 0 |
| 5 | 195 | 8 | 179 | 64 | 0.328205128 | 0.357541899 | 0.342873514 |
| 5 | 195 | 1 | 199 | 54 | 0.276923077 | 0.271356784 | 0.27413993 |
| 5 | 195 | 9 | 255 | 35 | 0.179487179 | 0.137254902 | 0.158371041 |
| 5 | 195 | 5 | 207 | 17 | 0.087179487 | 0.082125604 | 0.084652546 |
| 5 | 195 | 4 | 212 | 9 | 0.046153846 | 0.04245283 | 0.044303338 |
| 5 | 195 | 7 | 241 | 9 | 0.046153846 | 0.037344398 | 0.041749122 |
| 5 | 195 | 3 | 290 | 6 | 0.030769231 | 0.020689655 | 0.025729443 |
| 5 | 195 | 6 | 179 | 1 | 0.005128205 | 0.005586592 | 0.005357399 |
| 5 | 195 | 2 | 230 | 0 | 0 | 0 | 0 |
| 6 | 215 | 1 | 199 | 58 | 0.269767442 | 0.291457286 | 0.280612364 |
| 6 | 215 | 8 | 179 | 38 | 0.176744186 | 0.212290503 | 0.194517344 |
| 6 | 215 | 9 | 255 | 36 | 0.16744186 | 0.141176471 | 0.154309166 |
| 6 | 215 | 5 | 207 | 32 | 0.148837209 | 0.154589372 | 0.151713291 |
| 6 | 215 | 7 | 241 | 34 | 0.158139535 | 0.141078838 | 0.149609187 |
| 6 | 215 | 4 | 212 | 11 | 0.051162791 | 0.051886792 | 0.051524792 |
| 6 | 215 | 3 | 290 | 6 | 0.027906977 | 0.020689655 | 0.024298316 |
| 6 | 215 | 2 | 230 | 0 | 0 | 0 | 0 |
| 6 | 215 | 6 | 179 | 0 | 0 | 0 | 0 |
| 7 | 199 | 4 | 212 | 145 | 0.728643216 | 0.683962264 | 0.70630274 |
| 7 | 199 | 9 | 255 | 15 | 0.075376884 | 0.058823529 | 0.067100207 |
| 7 | 199 | 2 | 230 | 11 | 0.055276382 | 0.047826087 | 0.051551234 |
| 7 | 199 | 1 | 199 | 9 | 0.045226131 | 0.045226131 | 0.045226131 |
| 7 | 199 | 3 | 290 | 6 | 0.030150754 | 0.020689655 | 0.025420204 |
| 7 | 199 | 6 | 179 | 4 | 0.020100503 | 0.022346369 | 0.021223436 |
| 7 | 199 | 5 | 207 | 4 | 0.020100503 | 0.019323671 | 0.019712087 |
| 7 | 199 | 7 | 241 | 3 | 0.015075377 | 0.012448133 | 0.013761755 |
| 7 | 199 | 8 | 179 | 2 | 0.010050251 | 0.011173184 | 0.010611718 |
| 8 | 215 | 7 | 241 | 104 | 0.48372093 | 0.43153527 | 0.4576281 |
| 8 | 215 | 5 | 207 | 46 | 0.213953488 | 0.222222222 | 0.218087855 |
| 8 | 215 | 9 | 255 | 26 | 0.120930233 | 0.101960784 | 0.111445508 |
| 8 | 215 | 1 | 199 | 11 | 0.051162791 | 0.055276382 | 0.053219586 |
| 8 | 215 | 8 | 179 | 9 | 0.041860465 | 0.05027933 | 0.046069897 |
| 8 | 215 | 3 | 290 | 6 | 0.027906977 | 0.020689655 | 0.024298316 |
| 8 | 215 | 4 | 212 | 5 | 0.023255814 | 0.023584906 | 0.02342036 |
| 8 | 215 | 2 | 230 | 5 | 0.023255814 | 0.02173913 | 0.022497472 |
| 8 | 215 | 6 | 179 | 3 | 0.013953488 | 0.016759777 | 0.015356632 |
| 9 | 160 | 7 | 241 | 63 | 0.39375 | 0.261410788 | 0.327580394 |
| 9 | 160 | 1 | 199 | 32 | 0.2 | 0.16080402 | 0.18040201 |
| 9 | 160 | 5 | 207 | 31 | 0.19375 | 0.149758454 | 0.171754227 |
| 9 | 160 | 3 | 290 | 12 | 0.075 | 0.04137931 | 0.058189655 |
| 9 | 160 | 9 | 255 | 9 | 0.05625 | 0.035294118 | 0.045772059 |
| 9 | 160 | 8 | 179 | 6 | 0.0375 | 0.033519553 | 0.035509777 |
| 9 | 160 | 4 | 212 | 5 | 0.03125 | 0.023584906 | 0.027417453 |
| 9 | 160 | 6 | 179 | 1 | 0.00625 | 0.005586592 | 0.005918296 |
| 9 | 160 | 2 | 230 | 1 | 0.00625 | 0.004347826 | 0.005298913 |

### Results

### A High-Resolution Cellular Landscape of Human Breast Cancers

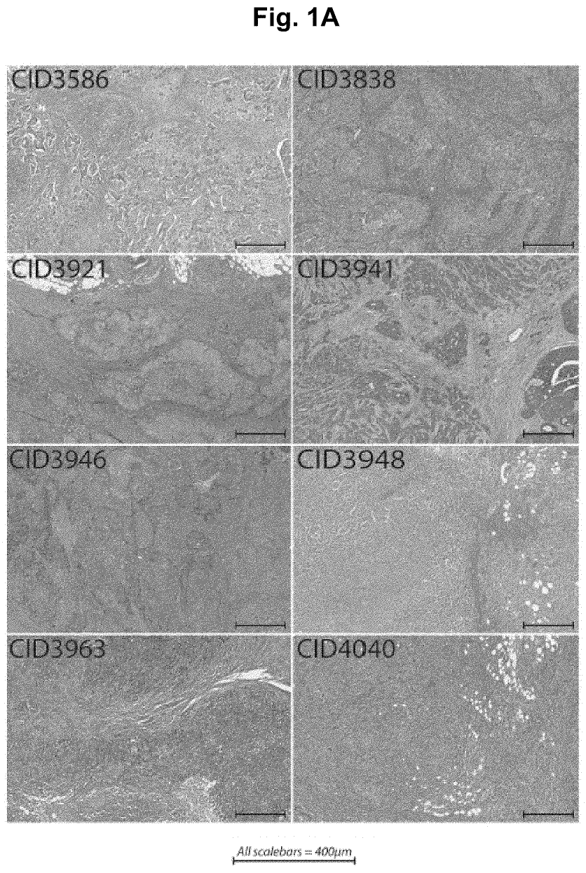

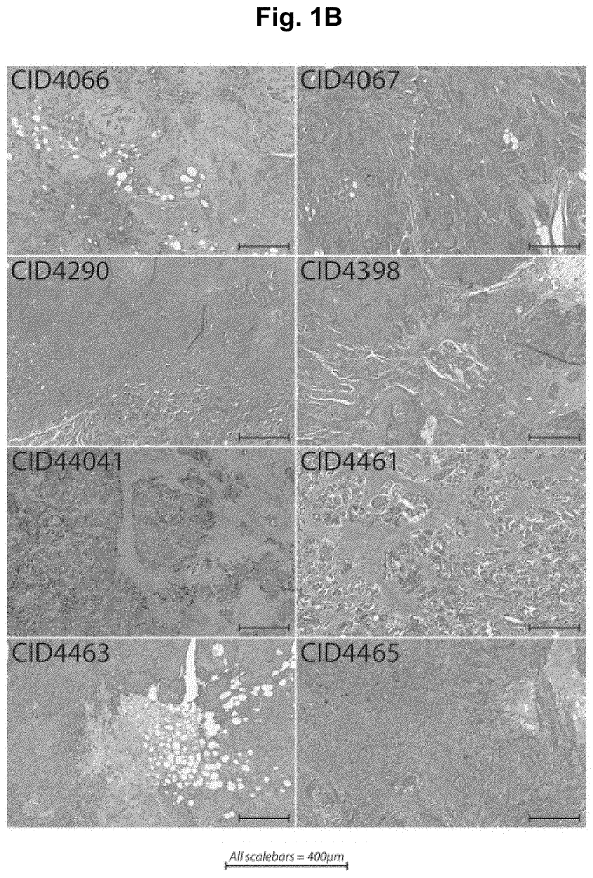

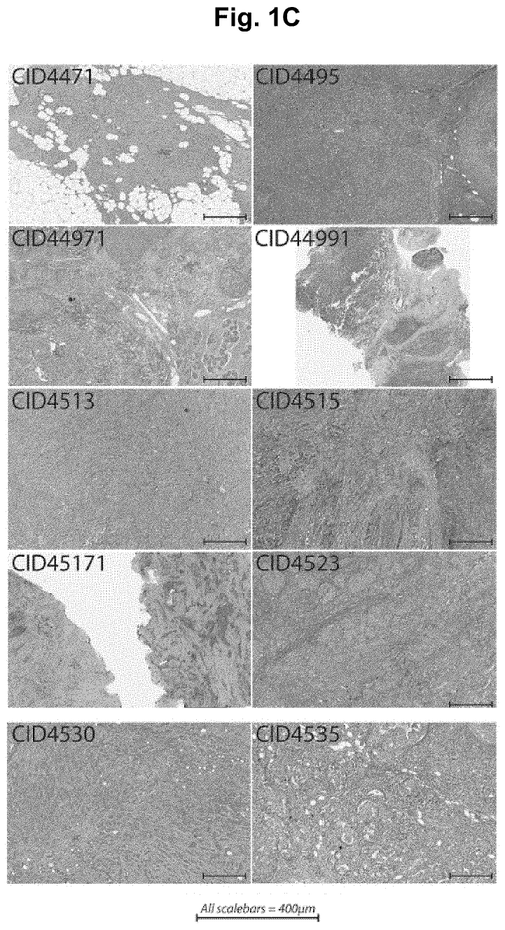

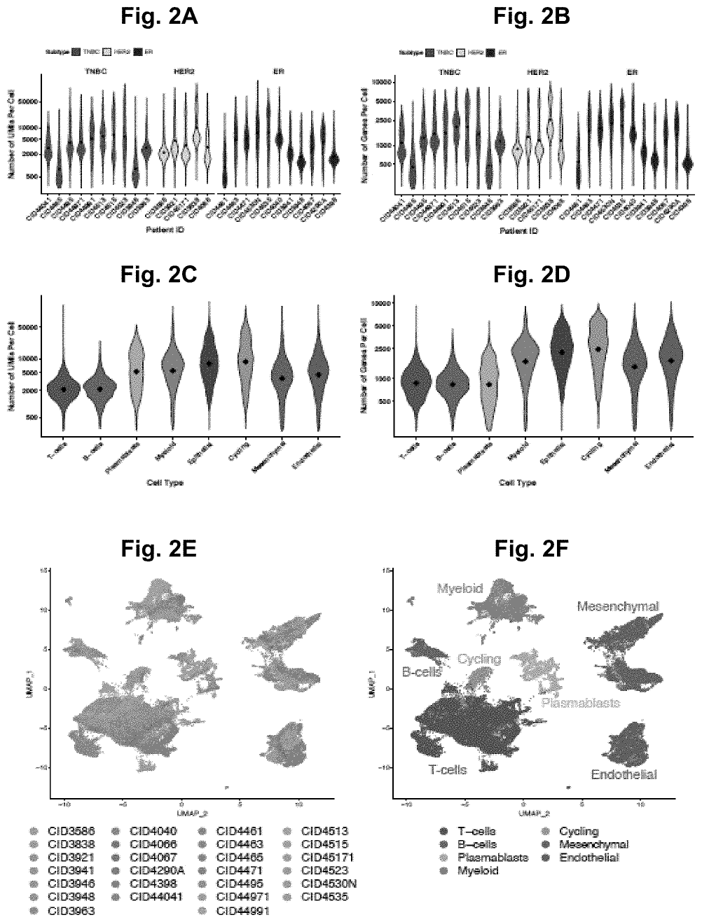

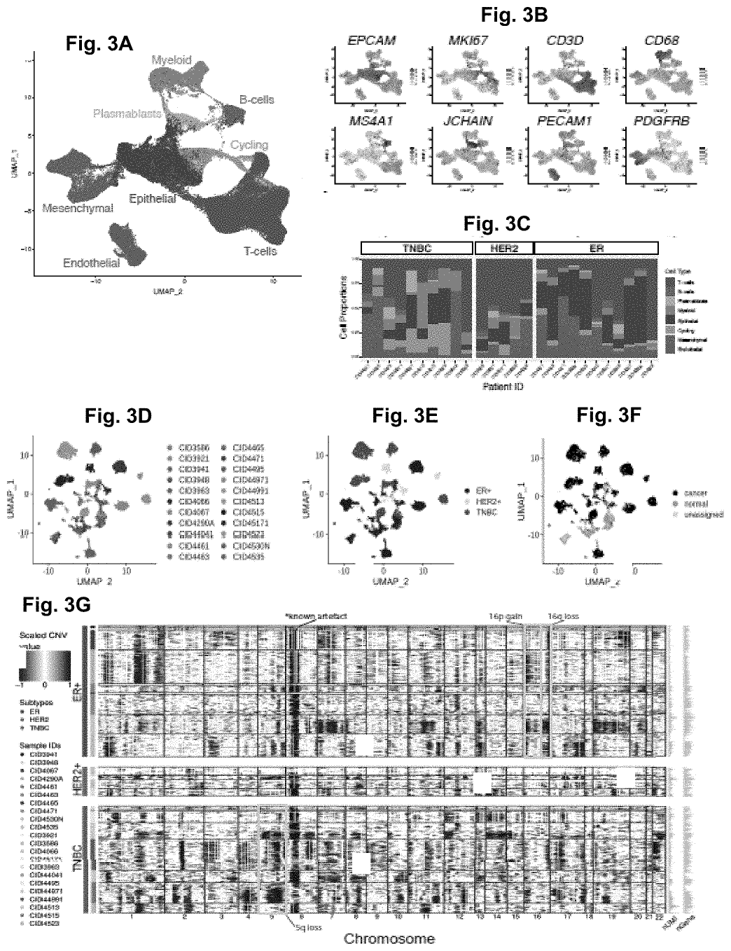

To elucidate the cellular architecture of BrCa, we analysed 26 primary pre-treatment human BrCa, including 11 ER+, 5 HER2+ and 10 TNBCs, by scRNA-Seq (Table 1; FIGS. 1A-1C). In total, 130,246 single-cells passed quality control (FIGS. 2A-2D) and were annotated using canonical lineage markers (FIG. 3A-3B). These high-level annotations were further confirmed using published gene signatures. All major cell types were represented across all tumours and clinical subtypes of BrCa (FIG. 3C; FIG. 2E).

As previously reported in other cancer types, UMAP visualization showed a clear separation of epithelial cells by tumour, although three clusters contained cells from multiple patients and subtypes (FIG. 3D-3E). We hypothesised that these were normal breast epithelial cells. In contrast, UMAP visualization of stromal and immune cells across tumours clustered together without batch correction (FIG. 2F). Since BrCa is largely driven by DNA copy number changes, we estimated single-cell copy number variant (CNV) profiles using InferCNV to distinguish neoplastic from normal epithelial cells (FIGS. 3F-3G). Cells confidently assigned as normal were re-clustered and annotated as one of the three main lineages of breast epithelia: myoepithelial, luminal progenitor and mature luminal. Within the neoplastic populations, we observed substantial levels of large-scale genomic rearrangement across a majority of cells (FIG. 3G; FIGS. 4A-4B; Table 6). This revealed patient-unique copy number changes as well as those commonly seen in BrCa, such as chr1q and chr16p gain and chr16q loss in luminal cancers; and chr5q loss in ER− basal-like breast cancers.

TABLE 6
inferCNV classifications Number (n) of neoplastic,
normal and unassigned epithelial cells per
tumour, as determined using inferCNV

| sample_id | normal_cell_call | n |
|---|---|---|
| CID3586 | neoplastic | 50 |
| CID3586 | normal | 1017 |
| CID3586 | unassigned | 90 |
| CID3921 | neoplastic | 522 |
| CID3921 | normal | 16 |
| CID3921 | unassigned | 31 |
| CID3941 | neoplastic | 259 |
| CID3941 | normal | 2 |
| CID3941 | unassigned | 24 |
| CID3948 | neoplastic | 289 |
| CID3948 | normal | 7 |
| CID3948 | unassigned | 27 |
| CID3963 | neoplastic | 300 |
| CID3963 | normal | 36 |
| CID3963 | unassigned | 134 |
| CID4066 | neoplastic | 629 |
| CID4066 | normal | 343 |
| CID4066 | unassigned | 250 |
| CID4067 | neoplastic | 2476 |
| CID4067 | normal | 22 |
| CID4067 | unassigned | 179 |
| CID4290A | neoplastic | 4292 |
| CID4290A | normal | 72 |
| CID4290A | unassigned | 303 |
| CID44041 | neoplastic | 6 |
| CID44041 | normal | 211 |
| CID44041 | unassigned | 18 |
| CID4461 | neoplastic | 224 |
| CID4461 | normal | 0 |
| CID4461 | unassigned | 22 |
| CID4463 | neoplastic | 675 |
| CID4463 | normal | 56 |
| CID4463 | unassigned | 92 |
| CID4465 | neoplastic | 154 |
| CID4465 | normal | 54 |
| CID4465 | unassigned | 51 |
| CID4471 | neoplastic | 212 |
| CID4471 | normal | 2330 |
| CID4471 | unassigned | 318 |
| CID4495 | neoplastic | 1423 |
| CID4495 | normal | 15 |
| CID4495 | unassigned | 146 |
| CID44971 | neoplastic | 921 |
| CID44971 | normal | 1059 |
| CID44971 | unassigned | 259 |
| CID44991 | neoplastic | 4035 |
| CID44991 | normal | 137 |
| CID44991 | unassigned | 229 |
| CID4513 | neoplastic | 1519 |
| CID4513 | normal | 28 |
| CID4513 | unassigned | 115 |
| CID4515 | neoplastic | 2659 |
| CID4515 | normal | 50 |
| CID4515 | unassigned | 168 |
| CID45171 | neoplastic | 952 |
| CID45171 | normal | 8 |
| CID45171 | unassigned | 89 |
| CID4523 | neoplastic | 1241 |
| CID4523 | normal | 7 |
| CID4523 | unassigned | 103 |
| CID4530N | neoplastic | 1718 |
| CID4530N | normal | 565 |
| CID4530N | unassigned | 270 |
| CID4535 | neoplastic | 2950 |
| CID4535 | normal | 49 |
| CID4535 | unassigned | 290 |

scSubtype: Intrinsic Subtyping for Single Cell RNA-Seq Data

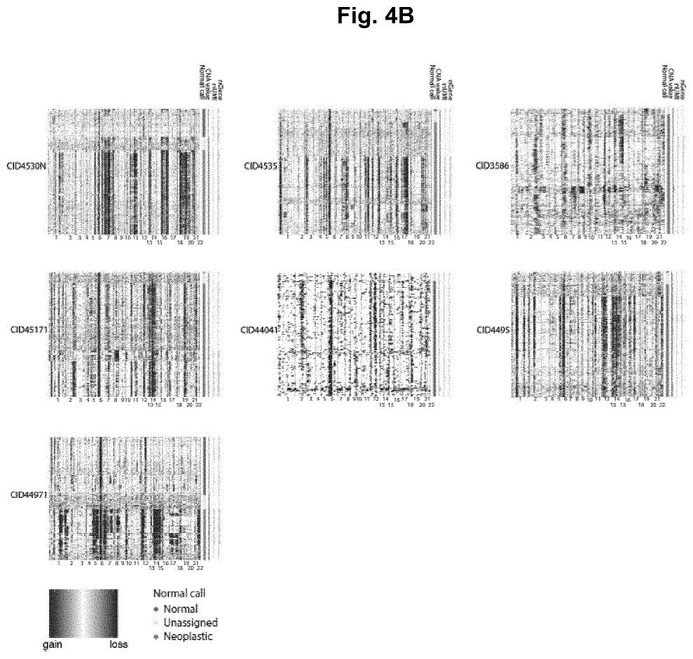

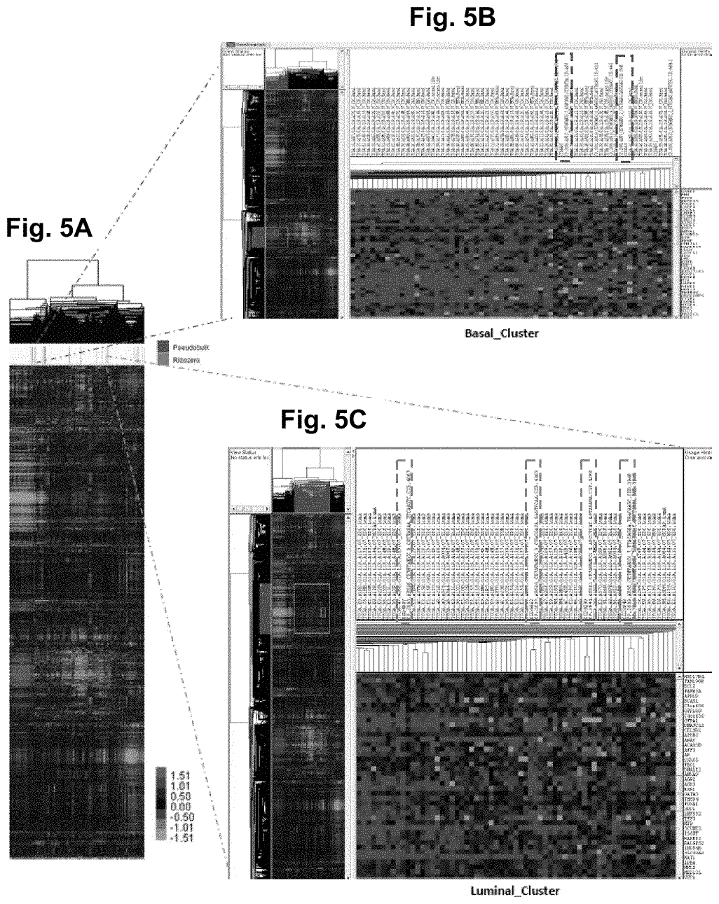

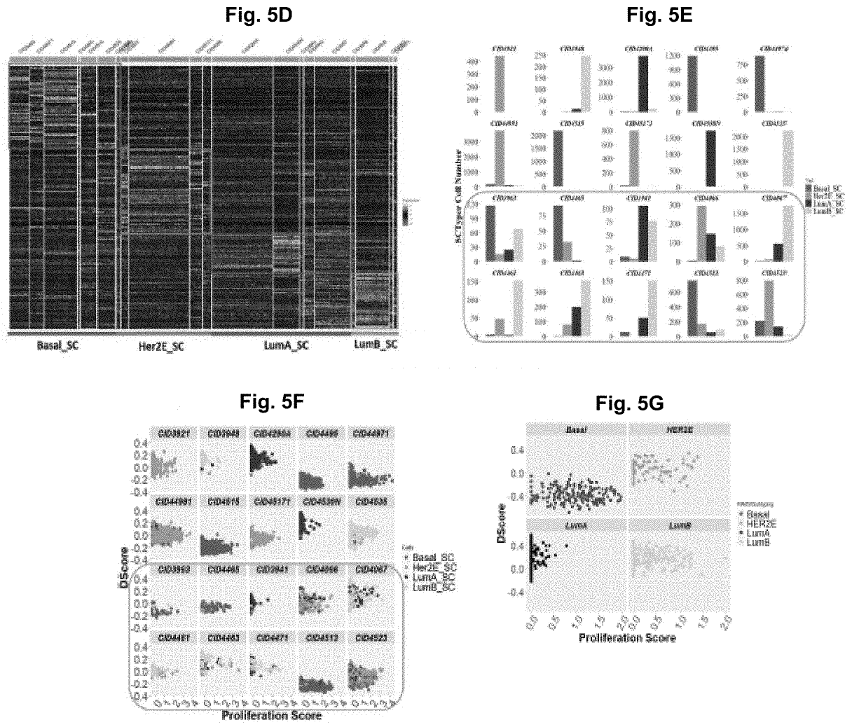

As unsupervised clustering could not be used to find recurring neoplastic cell gene expression features between tumours, we asked whether we could classify cells using the established PAM50 method. Due to the inherent sparsity of single-cell data, we took the opportunity to develop a scRNA-Seq compatible method for intrinsic molecular subtyping. We constructed “pseudo-bulk” profiles from scRNA-Seq for each tumour, with at least 150 neoplastic cells, and applied the PAM50 centroid predictor. This identified 7 Basal-like, 4 HER2E, 5 LumA, 3 LumB and 1 Normal-like BrCa. To identify a robust training set, we used hierarchical clustering of the pseudo-bulk samples with the TCGA dataset of 1,100 BrCa using an 2,000 gene intrinsic BrCa genelist4 (FIGS. 5A-5C). Training samples were selected from those with concordance between pseudo-bulk PAM50 subtype calls and TCGA hierarchical clustering subtype classifications.

For each PAM50 subtype within the training dataset, we performed pairwise single cell integrations and differential gene expression to identify 4 sets of genes that would define our single-cell derived molecular subtypes (89 genes Basal_SC; 102 genes HER2E_SC; 46 genes LumA_SC; 65 genes LumB_SC; methods). We defined these genes as the “scSubtype” gene signatures (FIG. 6A; FIG. 5D). Only four of these genes showed overlap with the original PAM50 gene list, including two from the Basal_SC set (ACTR3B and KRT14) and two from the Her2E_SC set (ERBB2 and GRB7). A subtype call for a given cell was based on the maximum scSubtype score. An overall tumour subtype was then assigned based on the largest population of cell subtypes. This majority scSubtype approach showed 100% agreement with the PAM50 pseudo-bulk calls in the 10 training set samples and 66% agreement on the test set samples (FIG. 5E). Of the 3 test set disagreements, two were LumA vs LumB, which are related profiles that may be hard to distinguish with a limited sample size, and the third was a metaplastic TNBC sample, which is a histological subtype not included in the original PAM50 training or testing datasets.

As another means of assessing the accuracy of scSubtype, we performed “true bulk” whole transcriptome RNA-Seq on 18 matching tumours in our scRNA-Seq cohort. As scSubtype does not include a Normal-like subtype, the two tumours called as Normal-like by RNA-Seq were not included in the comparison. We observed concordance between the majority scSubtype cell calls and the overall bulk tumour FFPE RNA-Seq profile in 12 of the remaining 16 BrCa, including 7 of the 8 matching training set tumours. We also clustered the true bulk RNA-Seq data with TCGA and confirmed that the true bulk clustered with the pseudo-bulk profiles for 14 of 18 samples (FIGS. 5A-5C). These results highlight the strong concordance between our three methods of subtyping when applied across both bulk and scRNA-Seq datasets.

scSubtype revealed that 13 of 20 samples had less than 90% of neoplastic cells falling under one molecular subtype, while only one tumour (CID3921; HER2E) composed of neoplastic cells with a completely homogenous molecular subtype (FIG. 6B). For instance, in some luminal and HER2E tumours, scSubtype predicted small numbers of basal-like cells, which was validated by IHC in 2 cases. These two cases, which were clinically ER+, showed small pockets of morphologically malignant cells that were negative for ER and positive for cytokeratin-5 (CK5), a basal cell marker, among otherwise ER-positive tumour cells (FIG. 6C). The utility of scSubtype is further demonstrated by its ability to correctly assign a low cellularity lobular carcinoma (10% neoplastic cells; CID4471), evident both by histology (FIGS. 1A-1C) and inferCNV (FIGS. 4A-4B; Table 2), as a mixture of mostly LumB and LumA cells, which is consistent with the clinical IHC result. Bulk and pseudo-bulk RNA-Seq analyses incorrectly assigned CID4471 as a Normal-like tumour, emphasizing the power of dissecting tumour biology at cellular resolution.

To further support the validity of scSubtype, we calculated the degree of epithelial cell differentiation (DScore) and proliferation, both of which are independently associated with the molecular intrinsic subtype of each tumour cell (FIG. 6D; FIG. 5F). We also plotted the same for the 1,100 tumours of the TCGA dataset (FIG. 5G). Basal_SC cells tended to have low DScores and high proliferation scores whereas LumA_SC cells showed high DScores and low proliferation scores, as observed for whole tumours in TCGA.

### Integrative Analysis Identifies Recurrent Gene Modules Driving Neoplastic Cell Heterogeneity

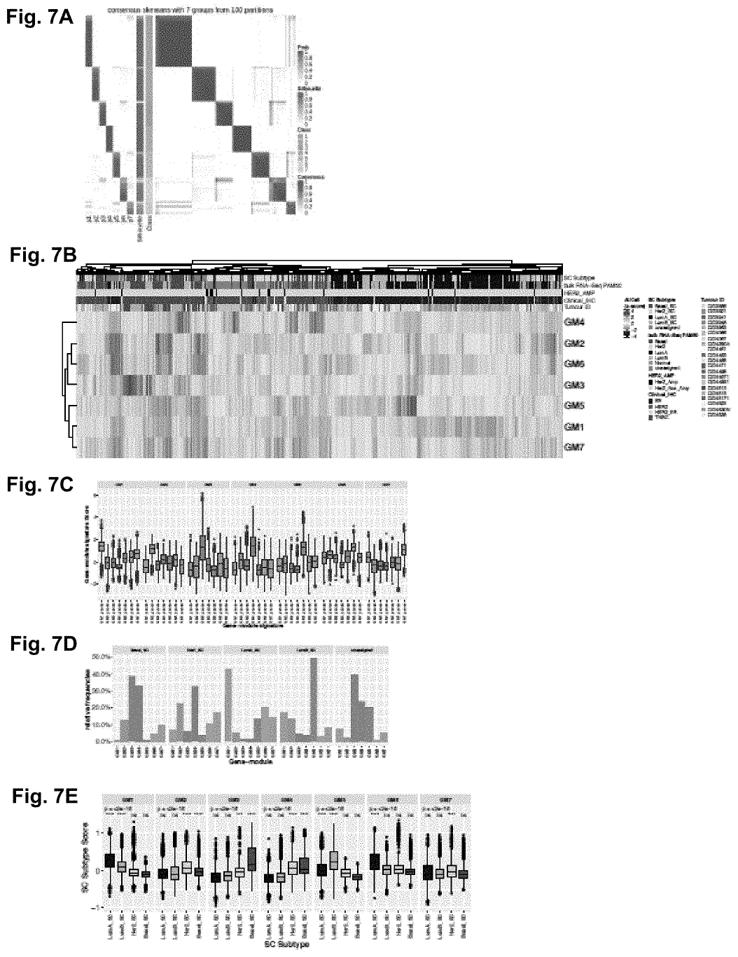

We investigated the biological pathways driving intra-tumour transcriptional heterogeneity (ITTH) in an unsupervised manner using integrative clustering, of tumours with at least 50 neoplastic cells, to generate 574 gene-signatures of ITTH. Across all tumours, we used these gene-signatures to identify 7 robust groups, “gene-modules”, based on their Jaccard similarity (FIG. 7A). Each gene module (GM) was defined with 200 genes that had the highest frequency of occurrence across the ITTH gene-signatures as well as individual tumours (Table 7), thus minimizing the contribution of a single tumour to any particular module.

TABLE 7
BrCa gene module list Gene lists for each
of the 7 ITTH gene-modules (GM1-7)

| gene | gene_module |
|---|---|
| ATF3 | 1 |
| JUN | 1 |
| NR4A1 | 1 |
| IER2 | 1 |
| DUSP1 | 1 |
| ZFP36 | 1 |
| JUNB | 1 |
| FOS | 1 |
| FOSB | 1 |
| PPP1R15A | 1 |
| KLF6 | 1 |
| DNAJB1 | 1 |
| EGR1 | 1 |
| BTG2 | 1 |
| HSPA1B | 1 |
| HSPA1A | 1 |
| RHOB | 1 |
| CLDN4 | 1 |
| MAFF | 1 |
| GADD45B | 1 |
| IRF1 | 1 |
| EFNA1 | 1 |
| SERTAD1 | 1 |
| TSC22D1 | 1 |
| CEBPD | 1 |
| CCNL1 | 1 |
| TRIB1 | 1 |
| MYC | 1 |
| ELF3 | 1 |
| LMNA | 1 |
| NFKBIA | 1 |
| TOB1 | 1 |
| HSPB1 | 1 |
| BRD2 | 1 |
| MCL1 | 1 |
| PNRC1 | 1 |
| IER3 | 1 |
| KLF4 | 1 |
| ZFP36L2 | 1 |
| SAT1 | 1 |
| ZFP36L1 | 1 |
| DNAJB4 | 1 |
| PHLDA2 | 1 |
| NEAT1 | 1 |
| MAP3K8 | 1 |
| GPRC5A | 1 |
| RASD1 | 1 |
| NFKBIZ | 1 |
| CTD-3252C9.4 | 1 |
| BAMBI | 1 |
| RND1 | 1 |
| HES1 | 1 |
| PIM3 | 1 |
| SQSTM1 | 1 |
| HSPH1 | 1 |
| ZFAND5 | 1 |
| AREG | 1 |
| CD55 | 1 |
| CDKN1A | 1 |
| UBC | 1 |
| CLDN3 | 1 |
| DDIT3 | 1 |
| BHLHE40 | 1 |
| BTG1 | 1 |
| ANKRD37 | 1 |
| SOCS3 | 1 |
| NAMPT | 1 |
| SOX4 | 1 |
| LDLR | 1 |
| TIPARP | 1 |
| TM4SF1 | 1 |
| CSRNP1 | 1 |
| GDF15 | 1 |
| ZFAND2A | 1 |
| NR4A2 | 1 |
| ERRFI1 | 1 |
| RAB11FIP1 | 1 |
| TRAF4 | 1 |
| MYADM | 1 |
| ZC3H12A | 1 |
| HERPUD1 | 1 |
| CKS2 | 1 |
| BAG3 | 1 |
| TGIF1 | 1 |
| ID3 | 1 |
| JUND | 1 |
| PMAIP1 | 1 |
| TACSTD2 | 1 |
| ETS2 | 1 |
| DNAJA1 | 1 |
| PDLIM3 | 1 |
| KLF10 | 1 |
| CYR61 | 1 |
| MXD1 | 1 |
| TNFAIP3 | 1 |
| NCOA7 | 1 |
| OVOL1 | 1 |
| TSC22D3 | 1 |
| HSP90AA1 | 1 |
| HSPA6 | 1 |
| C15orf48 | 1 |
| RHOV | 1 |
| DUSP4 | 1 |
| B4GALT1 | 1 |
| SDC4 | 1 |
| C8orf4 | 1 |
| DNAJB6 | 1 |
| ICAM1 | 1 |
| DNAJA4 | 1 |
| MRPL18 | 1 |
| GRB7 | 1 |
| HNRNPA0 | 1 |
| BCL3 | 1 |
| DUSP10 | 1 |
| EDN1 | 1 |
| FHL2 | 1 |
| CXCL2 | 1 |
| TNFRSF12A | 1 |
| S100P | 1 |
| HSPB8 | 1 |
| INSIG1 | 1 |
| PLK3 | 1 |
| EZR | 1 |
| IGFBP5 | 1 |
| SLC38A2 | 1 |
| DNAJB9 | 1 |
| H3F3B | 1 |
| TPM4 | 1 |
| TNFSF10 | 1 |
| RSRP1 | 1 |
| ARL5B | 1 |
| ATP1B1 | 1 |
| HSPA8 | 1 |
| IER5 | 1 |
| SCGB2A1 | 1 |
| YPEL2 | 1 |
| TMC5 | 1 |
| FBXO32 | 1 |
| MAP1LC3B | 1 |
| MIDN | 1 |
| GADD45G | 1 |
| VMP1 | 1 |
| HSPA5 | 1 |
| SCGB2A2 | 1 |
| TUBA1A | 1 |
| WEE1 | 1 |
| PDK4 | 1 |
| STAT3 | 1 |
| PERP | 1 |
| RBBP6 | 1 |
| KCNQ1OT1 | 1 |
| OSER1 | 1 |
| SERP1 | 1 |
| UBE2B | 1 |
| HSPE1 | 1 |
| SOX9 | 1 |
| MLF1 | 1 |
| UBB | 1 |
| MDK | 1 |
| YPEL5 | 1 |
| HMGCS1 | 1 |
| PTP4A1 | 1 |
| WSB1 | 1 |
| CEBPB | 1 |
| EIF4A2 | 1 |
| S100A10 | 1 |
| ELMSAN1 | 1 |
| ISG15 | 1 |
| CCNI | 1 |
| CLU | 1 |
| TIMP3 | 1 |
| ARL4A | 1 |
| SERPINH1 | 1 |
| SCGB1D2 | 1 |
| UGDH | 1 |
| FUS | 1 |
| BAG1 | 1 |
| IFRD1 | 1 |
| TFF1 | 1 |
| SERTAD3 | 1 |
| IGFBP4 | 1 |
| TPM1 | 1 |
| PKIB | 1 |
| MALAT1 | 1 |
| XBP1 | 1 |
| HEBP2 | 1 |
| GEM | 1 |
| EGR2 | 1 |
| ID2 | 1 |
| EGR3 | 1 |
| HSPD1 | 1 |
| GLUL | 1 |
| DDIT4 | 1 |
| CDC42EP1 | 1 |
| RBM39 | 1 |
| MT-ND5 | 1 |
| CSNK1A1 | 1 |
| SLC25A25 | 1 |
| PEG10 | 1 |
| DEDD2 | 1 |
| AZGP1 | 2 |
| ATP5C1 | 2 |
| ATP5F1 | 2 |
| NHP2 | 2 |
| MGP | 2 |
| RPN2 | 2 |
| C14orf2 | 2 |
| NQO1 | 2 |
| REEP5 | 2 |
| SSR2 | 2 |
| NDUFA8 | 2 |
| ATP5E | 2 |
| SH3BGRL | 2 |
| PIP | 2 |
| PRDX2 | 2 |
| RAB25 | 2 |
| EIF3L | 2 |
| PRDX1 | 2 |
| USMG5 | 2 |
| DAD1 | 2 |
| SEC61G | 2 |
| CCT3 | 2 |
| NDUFA4 | 2 |
| APOD | 2 |
| CHCHD10 | 2 |
| DDIT4 | 2 |
| MRPL24 | 2 |
| NME1 | 2 |
| DCXR | 2 |
| NDUFAB1 | 2 |
| ATP5A1 | 2 |
| ATP5B | 2 |
| ATOX1 | 2 |
| SLC50A1 | 2 |
| POLR2I | 2 |
| TIMM8B | 2 |
| VPS29 | 2 |
| TIMP1 | 2 |
| AHCY | 2 |
| PRDX3 | 2 |
| RBM3 | 2 |
| GSTM3 | 2 |
| ABRACL | 2 |
| RBX1 | 2 |
| PAFAH1B3 | 2 |
| AP1S1 | 2 |
| RPL34 | 2 |
| ATPIF1 | 2 |
| PGD | 2 |
| CANX | 2 |
| SELENBP1 | 2 |
| ATP5J | 2 |
| PSME2 | 2 |
| PSME1 | 2 |
| SDHC | 2 |
| AKR1A1 | 2 |
| GSTP1 | 2 |
| RARRES3 | 2 |
| ISCU | 2 |
| NPM1 | 2 |
| SPDEF | 2 |
| BLVRB | 2 |
| NDUFB3 | 2 |
| RPL36A | 2 |
| MDH1 | 2 |
| MYEOV2 | 2 |
| MAGED2 | 2 |
| CRIP2 | 2 |
| SEC11C | 2 |
| CD151 | 2 |
| COPE | 2 |
| PFN2 | 2 |
| ALDH2 | 2 |
| SNRPD2 | 2 |
| TSTD1 | 2 |
| RPL13A | 2 |
| HIGD2A | 2 |
| NDUFC1 | 2 |
| PYCARD | 2 |
| FIS1 | 2 |
| ITM2B | 2 |
| PSMB3 | 2 |
| G6PD | 2 |
| CST3 | 2 |
| SH3BGRL3 | 2 |
| TAGLN2 | 2 |
| NDUFA1 | 2 |
| TMEM183A | 2 |
| S100A10 | 2 |
| NGFRAP1 | 2 |
| DEGS2 | 2 |
| ARPC5 | 2 |
| TM7SF2 | 2 |
| RPS10 | 2 |
| LAMTOR5 | 2 |
| TMEM256 | 2 |
| UQCRB | 2 |
| TMEM141 | 2 |
| KRTCAP2 | 2 |
| HM13 | 2 |
| NDUFS6 | 2 |
| PARK7 | 2 |
| PSMD4 | 2 |
| NDUFB11 | 2 |
| TOMM7 | 2 |
| EIF6 | 2 |
| UQCRHL | 2 |
| ADI1 | 2 |
| VDAC1 | 2 |
| C9orf16 | 2 |
| ETFA | 2 |
| LSM3 | 2 |
| UQCRH | 2 |
| CYB5A | 2 |
| SNRPE | 2 |
| BSG | 2 |
| SSR3 | 2 |
| DPM3 | 2 |
| LAMTOR4 | 2 |
| RPS11 | 2 |
| FAM195A | 2 |
| TMEM261 | 2 |
| ATP5I | 2 |
| EIF5A | 2 |
| PIN4 | 2 |
| ATXN10 | 2 |
| ATP5G3 | 2 |
| ARPC3 | 2 |
| UBA52 | 2 |
| BEX4 | 2 |
| ROMO1 | 2 |
| SLC25A6 | 2 |
| SDCBP | 2 |
| EIF4EBP1 | 2 |
| PFDN6 | 2 |
| PSMA3 | 2 |
| RNF7 | 2 |
| SPCS2 | 2 |
| CYSTM1 | 2 |
| CAPG | 2 |
| CD9 | 2 |
| GRHPR | 2 |
| SEPP1 | 2 |
| ESF1 | 2 |
| TFF3 | 2 |
| ARPC1B | 2 |
| ANXA5 | 2 |
| WDR83OS | 2 |
| LYPLA1 | 2 |
| COMT | 2 |
| MDH2 | 2 |
| DNPH1 | 2 |
| RAB13 | 2 |
| EIF3K | 2 |
| PTGR1 | 2 |
| LGALS3 | 2 |
| TPI1 | 2 |
| COPZ1 | 2 |
| LDHA | 2 |
| PSMD8 | 2 |
| EIF2S3 | 2 |
| NME3 | 2 |
| EIF3E | 2 |
| MRPL13 | 2 |
| ZFAND6 | 2 |
| FAM162A | 2 |
| ATP6V0E1 | 2 |
| TMED10 | 2 |
| HNRNPA3 | 2 |
| PPA1 | 2 |
| SNX17 | 2 |
| APOA1BP | 2 |
| TUFM | 2 |
| ECHS1 | 2 |
| GLTSCR2 | 2 |
| RPS27L | 2 |
| NDUFB1 | 2 |
| SSBP1 | 2 |
| PRDX6 | 2 |
| ENO1 | 2 |
| PPP4C | 2 |
| COA3 | 2 |
| TCEAL4 | 2 |
| MRPL54 | 2 |
| LAMTOR2 | 2 |
| PAIP2 | 2 |
| DAP | 2 |
| RPL22L1 | 2 |
| C6orf203 | 2 |
| TECR | 2 |
| PEBP1 | 2 |
| TMED9 | 2 |
| ATP6V1F | 2 |
| ESD | 2 |
| EIF3I | 2 |
| SCO2 | 2 |
| ATP5D | 2 |
| UAP1 | 2 |
| TMEM258 | 2 |
| COX17 | 2 |
| HLA-B | 3 |
| HLA-A | 3 |
| VIM | 3 |
| CD74 | 3 |
| SRGN | 3 |
| HLA-C | 3 |
| IFI27 | 3 |
| HLA-E | 3 |
| IFITM1 | 3 |
| PSMB9 | 3 |
| RGCC | 3 |
| S100A4 | 3 |
| HLA-DRA | 3 |
| ISG15 | 3 |
| IL32 | 3 |
| SPARC | 3 |
| TAGLN | 3 |
| IFITM3 | 3 |
| IFITM2 | 3 |
| IGFBP7 | 3 |
| CALD1 | 3 |
| HLA-DPB1 | 3 |
| HLA-DPA1 | 3 |
| B2M | 3 |
| TIMP1 | 3 |
| RGS1 | 3 |
| FN1 | 3 |
| ACTA2 | 3 |
| HLA-DRB1 | 3 |
| SERPING1 | 3 |
| ANXA1 | 3 |
| TPM2 | 3 |
| TMSB4X | 3 |
| CD69 | 3 |
| CCL4 | 3 |
| LAPTM5 | 3 |
| GSN | 3 |
| APOE | 3 |
| STAT1 | 3 |
| SPARCL1 | 3 |
| IFI6 | 3 |
| DUSP1 | 3 |
| CXCR4 | 3 |
| CCL5 | 3 |
| UBE2L6 | 3 |
| MYL9 | 3 |
| SLC2A3 | 3 |
| BST2 | 3 |
| CAV1 | 3 |
| CD52 | 3 |
| ZFP36L2 | 3 |
| HLA-DQB1 | 3 |
| PDLIM1 | 3 |
| TNFAIP3 | 3 |
| CORO1A | 3 |
| RARRES3 | 3 |
| TYMP | 3 |
| C1S | 3 |
| PTRF | 3 |
| PSME2 | 3 |
| CYTIP | 3 |
| COL1A1 | 3 |
| PSMB8 | 3 |
| NNMT | 3 |
| HLA-DQA1 | 3 |
| DUSP2 | 3 |
| COL1A2 | 3 |
| ARHGDIB | 3 |
| COL6A2 | 3 |
| FOS | 3 |
| CCL2 | 3 |
| BGN | 3 |
| ID3 | 3 |
| TUBA1A | 3 |
| RAC2 | 3 |
| LBH | 3 |
| HLA-DRB5 | 3 |
| FCER1G | 3 |
| GBP1 | 3 |
| C1QA | 3 |
| COTL1 | 3 |
| LUM | 3 |
| MYL6 | 3 |
| GBP2 | 3 |
| BTG1 | 3 |
| CD37 | 3 |
| HCST | 3 |
| LIMD2 | 3 |
| IFIT3 | 3 |
| IL7R | 3 |
| PTPRC | 3 |
| NKG7 | 3 |
| FYB | 3 |
| TAP1 | 3 |
| LTB | 3 |
| S100A6 | 3 |
| COL3A1 | 3 |
| EMP3 | 3 |
| A2M | 3 |
| JUNB | 3 |
| TPM1 | 3 |
| FABP4 | 3 |
| TXNIP | 3 |
| SAT1 | 3 |
| FXYD5 | 3 |
| CD3E | 3 |
| HLA-DMA | 3 |
| CTSC | 3 |
| TSC22D3 | 3 |
| MYL12A | 3 |
| CST3 | 3 |
| CNN2 | 3 |
| PHLDA1 | 3 |
| LYZ | 3 |
| IFI44L | 3 |
| MARCKS | 3 |
| ID1 | 3 |
| DCN | 3 |
| TGFBI | 3 |
| BIRC3 | 3 |
| THY1 | 3 |
| LGALS1 | 3 |
| GPX1 | 3 |
| C1QB | 3 |
| CD2 | 3 |
| CST7 | 3 |
| COL6A3 | 3 |
| ACAP1 | 3 |
| IFI16 | 3 |
| ITM2B | 3 |
| POSTN | 3 |
| LDHB | 3 |
| FLNA | 3 |
| FILIP1L | 3 |
| CDKN1A | 3 |
| IRF1 | 3 |
| LGALS3 | 3 |
| SERPINH1 | 3 |
| EFEMP1 | 3 |
| PSME1 | 3 |
| SH3BGRL3 | 3 |
| IL2RG | 3 |
| CD3D | 3 |
| SFRP2 | 3 |
| TIMP3 | 3 |
| ALOX5AP | 3 |
| GMFG | 3 |
| CYBA | 3 |
| TAGLN2 | 3 |
| LAP3 | 3 |
| RGS2 | 3 |
| CLEC2B | 3 |
| TRBC2 | 3 |
| NR4A2 | 3 |
| S100A8 | 3 |
| PSMB10 | 3 |
| OPTN | 3 |
| CTSB | 3 |
| FTL | 3 |
| KRT17 | 3 |
| AREG | 3 |
| MYH9 | 3 |
| MMP7 | 3 |
| COL6A1 | 3 |
| GZMA | 3 |
| RNASE1 | 3 |
| PCOLCE | 3 |
| PTN | 3 |
| PYCARD | 3 |
| ARPC2 | 3 |
| SGK1 | 3 |
| COL18A1 | 3 |
| GSTP1 | 3 |
| NPC2 | 3 |
| SOD3 | 3 |
| MFGE8 | 3 |
| COL4A1 | 3 |
| ADIRF | 3 |
| HLA-F | 3 |
| CD7 | 3 |
| APOC1 | 3 |
| TYROBP | 3 |
| C1QC | 3 |
| TAPBP | 3 |
| STK4 | 3 |
| RHOH | 3 |
| RNF213 | 3 |
| SOD2 | 3 |
| TPM4 | 3 |
| CALM1 | 3 |
| CTGF | 3 |
| PNRC1 | 3 |
| CD27 | 3 |
| CD3G | 3 |
| PRKCDBP | 3 |
| PARP14 | 3 |
| IGKC | 3 |
| IGFBP5 | 3 |
| IFIT1 | 3 |
| LY6E | 3 |
| STMN1 | 4 |
| H2AFZ | 4 |
| UBE2C | 4 |
| TUBA1B | 4 |
| BIRC5 | 4 |
| HMGB2 | 4 |
| ZWINT | 4 |
| TUBB | 4 |
| HMGB1 | 4 |
| DEK | 4 |
| CDK1 | 4 |
| HMGN2 | 4 |
| UBE2T | 4 |
| TK1 | 4 |
| RRM2 | 4 |
| RANBP1 | 4 |
| TYMS | 4 |
| CENPW | 4 |
| MAD2L1 | 4 |
| CKS2 | 4 |
| CKS1B | 4 |
| NUSAP1 | 4 |
| TUBA1C | 4 |
| PTTG1 | 4 |
| KPNA2 | 4 |
| PCNA | 4 |
| CENPF | 4 |
| HIST1H4C | 4 |
| CDKN3 | 4 |
| UBE2S | 4 |
| CCNB1 | 4 |
| HMGA1 | 4 |
| DTYMK | 4 |
| SNRPB | 4 |
| CDC20 | 4 |
| NASP | 4 |
| MCM7 | 4 |
| PLP2 | 4 |
| TUBB4B | 4 |
| PLK1 | 4 |
| CCNB2 | 4 |
| MKI67 | 4 |
| TOP2A | 4 |
| TPX2 | 4 |
| PKMYT1 | 4 |
| PRC1 | 4 |
| SMC4 | 4 |
| CENPU | 4 |
| RAN | 4 |
| DUT | 4 |
| PA2G4 | 4 |
| BUB3 | 4 |
| RAD21 | 4 |
| SPC25 | 4 |
| HN1 | 4 |
| CDCA3 | 4 |
| H2AFV | 4 |
| HNRNPA2B1 | 4 |
| CCNA2 | 4 |
| PBK | 4 |
| LSM5 | 4 |
| DNAJC9 | 4 |
| RPA3 | 4 |
| TMPO | 4 |
| SNRPD1 | 4 |
| CENPA | 4 |
| KIF20B | 4 |
| USP1 | 4 |
| H2AFX | 4 |
| PPM1G | 4 |
| NUF2 | 4 |
| SNRPG | 4 |
| KIF22 | 4 |
| KIAA0101 | 4 |
| DEPDC1 | 4 |
| RNASEH2A | 4 |
| MT2A | 4 |
| STRA13 | 4 |
| ANLN | 4 |
| CACYBP | 4 |
| NCL | 4 |
| NUDT1 | 4 |
| ECT2 | 4 |
| LSM4 | 4 |
| ASF1B | 4 |
| CENPN | 4 |
| TMEM106C | 4 |
| CCT5 | 4 |
| HSPA8 | 4 |
| HMMR | 4 |
| SRSF3 | 4 |
| AURKB | 4 |
| GGH | 4 |
| AURKA | 4 |
| TRIP13 | 4 |
| CDCA8 | 4 |
| HMGB3 | 4 |
| HNRNPAB | 4 |
| FAM83D | 4 |
| CDC25B | 4 |
| GGCT | 4 |
| KNSTRN | 4 |
| CCT6A | 4 |
| PTGES3 | 4 |
| ANP32E | 4 |
| CENPK | 4 |
| MCM3 | 4 |
| DDX21 | 4 |
| HSPD1 | 4 |
| SKA2 | 4 |
| CALM2 | 4 |
| UHRF1 | 4 |
| HINT1 | 4 |
| ORC6 | 4 |
| MZT1 | 4 |
| MIS18BP1 | 4 |
| WDR34 | 4 |
| NAP1L1 | 4 |
| TEX30 | 4 |
| SFN | 4 |
| HSPE1 | 4 |
| CENPM | 4 |
| TROAP | 4 |
| CDCA5 | 4 |
| RACGAP1 | 4 |
| SLC25A5 | 4 |
| ATAD2 | 4 |
| DBF4 | 4 |
| KIF23 | 4 |
| CEP55 | 4 |
| SIVA1 | 4 |
| SAC3D1 | 4 |
| PSIP1 | 4 |
| CLSPN | 4 |
| CCT2 | 4 |
| DLGAP5 | 4 |
| PSMA4 | 4 |
| SMC2 | 4 |
| AP2S1 | 4 |
| RAD51AP1 | 4 |
| MND1 | 4 |
| ILF2 | 4 |
| DNMT1 | 4 |
| NUCKS1 | 4 |
| LMNB1 | 4 |
| RFC4 | 4 |
| EIF5A | 4 |
| NPM3 | 4 |
| ARL6IP1 | 4 |
| ASPM | 4 |
| GTSE1 | 4 |
| TOMM40 | 4 |
| HNRNPA1 | 4 |
| GMNN | 4 |
| FEN1 | 4 |
| CDCA7 | 4 |
| SLBP | 4 |
| TNFRSF12A | 4 |
| TM4SF1 | 4 |
| CKAP2 | 4 |
| CENPE | 4 |
| SRP9 | 4 |
| DDX39A | 4 |
| COMMD4 | 4 |
| RBM8A | 4 |
| CALM3 | 4 |
| RRM1 | 4 |
| ENO1 | 4 |
| ANP32B | 4 |
| SRSF7 | 4 |
| FAM96A | 4 |
| TPRKB | 4 |
| FABP5 | 4 |
| PPIF | 4 |
| SERPINE1 | 4 |
| TACC3 | 4 |
| RBBP7 | 4 |
| NEK2 | 4 |
| CALM1 | 4 |
| GMPS | 4 |
| EMP2 | 4 |
| HMG20B | 4 |
| SMC3 | 4 |
| HSPA9 | 4 |
| NAA20 | 4 |
| NUDC | 4 |
| RPL39L | 4 |
| PRKDC | 4 |
| CDCA4 | 4 |
| HIST1H1A | 4 |
| HES6 | 4 |
| SUPT16H | 4 |
| PTMS | 4 |
| VDAC3 | 4 |
| PSMC3 | 4 |
| ATP5G1 | 4 |
| PSMA3 | 4 |
| PGP | 4 |
| KIF2C | 4 |
| CARHSP1 | 4 |
| GJA1 | 5 |
| SCGB2A2 | 5 |
| ARMT1 | 5 |
| MAGED2 | 5 |
| PIP | 5 |
| SCGB1D2 | 5 |
| CLTC | 5 |
| MYBPC1 | 5 |
| PDZK1 | 5 |
| MGP | 5 |
| SLC39A6 | 5 |
| CCND1 | 5 |
| SLC9A3R1 | 5 |
| NAT1 | 5 |
| SUB1 | 5 |
| CYP4X1 | 5 |
| STC2 | 5 |
| CROT | 5 |
| CTSD | 5 |
| FASN | 5 |
| PBX1 | 5 |
| SLC4A7 | 5 |
| FOXA1 | 5 |
| MCCC2 | 5 |
| IDH1 | 5 |
| H2AFJ | 5 |
| CYP4Z1 | 5 |
| IFI27 | 5 |
| TBC1D9 | 5 |
| ANPEP | 5 |
| DHRS2 | 5 |
| TFF3 | 5 |
| LGALS3BP | 5 |
| GATA3 | 5 |
| LTF | 5 |
| IFITM2 | 5 |
| IFITM1 | 5 |
| AHNAK | 5 |
| SEPPI | 5 |
| ACADSB | 5 |
| PDCD4 | 5 |
| MUCL1 | 5 |
| CERS6 | 5 |
| LRRC26 | 5 |
| ASS1 | 5 |
| SEMA3C | 5 |
| APLP2 | 5 |
| AMFR | 5 |
| CDV3 | 5 |
| VTCN1 | 5 |
| PREX1 | 5 |
| TP53INP1 | 5 |
| LRIG1 | 5 |
| ANK3 | 5 |
| ACLY | 5 |
| CLSTN1 | 5 |
| GNB1 | 5 |
| C1orf64 | 5 |
| STARD10 | 5 |
| CA12 | 5 |
| SCGB2A1 | 5 |
| MGST1 | 5 |
| PSAP | 5 |
| GNAS | 5 |
| MRPS30 | 5 |
| MSMB | 5 |
| DDIT4 | 5 |
| TTC36 | 5 |
| S100A1 | 5 |
| FAM208B | 5 |
| STT3B | 5 |
| SLC38A1 | 5 |
| DMKN | 5 |
| SEC14L2 | 5 |
| FMO5 | 5 |
| DCAF10 | 5 |
| WFDC2 | 5 |
| GFRA1 | 5 |
| LDLRAD4 | 5 |
| TXNIP | 5 |
| SCGB3A1 | 5 |
| APOD | 5 |
| N4BP2L2 | 5 |
| TNC | 5 |
| ADIRF | 5 |
| NPY1R | 5 |
| NBPF1 | 5 |
| TMEM176A | 5 |
| GLUL | 5 |
| BMP2K | 5 |
| SLC44A1 | 5 |
| GFPT1 | 5 |
| PSD3 | 5 |
| CCNG2 | 5 |
| CGNL1 | 5 |
| TMED7 | 5 |
| NOVA1 | 5 |
| ARCN1 | 5 |
| NEK10 | 5 |
| GPC6 | 5 |
| SCGB1B2P | 5 |
| IGHG4 | 5 |
| SYT1 | 5 |
| SYNGR2 | 5 |
| HSPA1A | 5 |
| ATP6AP1 | 5 |
| TSPAN13 | 5 |
| MT-ND2 | 5 |
| NIFK | 5 |
| MT-ATP8 | 5 |
| MT-ATP6 | 5 |
| MT-CO3 | 5 |
| EVL | 5 |
| GRN | 5 |
| ERH | 5 |
| CD81 | 5 |
| NUPR1 | 5 |
| SELENBP1 | 5 |
| C1orf56 | 5 |
| LMO3 | 5 |
| PLK2 | 5 |
| HACD3 | 5 |
| RBBP8 | 5 |
| CANX | 5 |
| ENAH | 5 |
| SCD | 5 |
| CREB3L2 | 5 |
| SYNCRIP | 5 |
| TBL1XR1 | 5 |
| DDR1 | 5 |
| ERBB3 | 5 |
| CHPT1 | 5 |
| BANF1 | 5 |
| UGDH | 5 |
| SCUBE2 | 5 |
| UQCR10 | 5 |
| COX6C | 5 |
| ATP5G1 | 5 |
| PRSS23 | 5 |
| MYEOV2 | 5 |
| PITX1 | 5 |
| MT-ND4L | 5 |
| TPM1 | 5 |
| HMGCS2 | 5 |
| ADIPOR2 | 5 |
| UGCG | 5 |
| FAM129B | 5 |
| TNIP1 | 5 |
| IFI6 | 5 |
| CA2 | 5 |
| ESR1 | 5 |
| TMBIM4 | 5 |
| NFIX | 5 |
| PDCD6IP | 5 |
| CRIM1 | 5 |
| ARHGEF12 | 5 |
| ENTPD5 | 5 |
| PATZ1 | 5 |
| ZBTB41 | 5 |
| UCP1 | 5 |
| ANO1 | 5 |
| RP11-356O9.1 | 5 |
| MYB | 5 |
| ZBTB44 | 5 |
| SCPEP1 | 5 |
| HIPK2 | 5 |
| CDK2AP1 | 5 |
| CYHR1 | 5 |
| SPINK8 | 5 |
| FKBP10 | 5 |
| ISOC1 | 5 |
| CD59 | 5 |
| RAMP1 | 5 |
| AFF3 | 5 |
| MT-CYB | 5 |
| PPP1CB | 5 |
| PKM | 5 |
| ALDH2 | 5 |
| PRSS8 | 5 |
| NPW | 5 |
| SPR | 5 |
| PRDX3 | 5 |
| SCOC | 5 |
| TMED10 | 5 |
| KIAA0196 | 5 |
| NDP | 5 |
| ZSWIM7 | 5 |
| AP2A1 | 5 |
| PLAT | 5 |
| SUSD3 | 5 |
| CRABP2 | 5 |
| DNAJC12 | 5 |
| DHCR24 | 5 |
| PPT1 | 5 |
| FAM234B | 5 |
| DDX17 | 5 |
| LRP2 | 5 |
| ABCD3 | 5 |
| CDH1 | 5 |
| NFIA | 5 |
| AGR2 | 6 |
| TFF3 | 6 |
| SELM | 6 |
| CD63 | 6 |
| CTSD | 6 |
| MDK | 6 |
| CD74 | 6 |
| S100A13 | 6 |
| IFITM3 | 6 |
| HLA-B | 6 |
| AZGP1 | 6 |
| FXYD3 | 6 |
| IFITM2 | 6 |
| RABAC1 | 6 |
| S100A14 | 6 |
| CRABP2 | 6 |
| LTF | 6 |
| RARRES1 | 6 |
| HLA-A | 6 |
| PPIB | 6 |
| HLA-C | 6 |
| S100A10 | 6 |
| S100A9 | 6 |
| TIMP1 | 6 |
| DDIT4 | 6 |
| S100A16 | 6 |
| LGALS1 | 6 |
| LAPTM4A | 6 |
| SSR4 | 6 |
| S100A6 | 6 |
| CD59 | 6 |
| BST2 | 6 |
| PDIA3 | 6 |
| KRT19 | 6 |
| CD9 | 6 |
| FXYD5 | 6 |
| SCGB2A2 | 6 |
| NUCB2 | 6 |
| TMED3 | 6 |
| LY6E | 6 |
| CFD | 6 |
| ITM2B | 6 |
| PDZK1IP1 | 6 |
| LGALS3 | 6 |
| NUPR1 | 6 |
| SLPI | 6 |
| CLU | 6 |
| TMED9 | 6 |
| HLA-DRA | 6 |
| SPTSSB | 6 |
| TMEM59 | 6 |
| KRT8 | 6 |
| CALR | 6 |
| HLA-DRB1 | 6 |
| IFI6 | 6 |
| NNMT | 6 |
| CALML5 | 6 |
| S100P | 6 |
| TFF1 | 6 |
| ATP1B1 | 6 |
| SPINT2 | 6 |
| PDIA6 | 6 |
| S100A8 | 6 |
| HSP90B1 | 6 |
| LMAN1 | 6 |
| RARRES3 | 6 |
| SELENBP1 | 6 |
| CEACAM6 | 6 |
| TMEM176A | 6 |
| EPCAM | 6 |
| MAGED2 | 6 |
| SNCG | 6 |
| DUSP4 | 6 |
| CD24 | 6 |
| PERP | 6 |
| WFDC2 | 6 |
| HM13 | 6 |
| TMBIM6 | 6 |
| C12orf57 | 6 |
| DKK1 | 6 |
| MAGED1 | 6 |
| PYCARD | 6 |
| RAMP1 | 6 |
| C11orf31 | 6 |
| STOM | 6 |
| TNFSF10 | 6 |
| BSG | 6 |
| TMED10 | 6 |
| ASS1 | 6 |
| PDLIM1 | 6 |
| CST3 | 6 |
| PDIA4 | 6 |
| NDUFA4 | 6 |
| GSTP1 | 6 |
| TYMP | 6 |
| SH3BGRL3 | 6 |
| PRSS23 | 6 |
| P4HA1 | 6 |
| MUC5B | 6 |
| S100A1 | 6 |
| PSAP | 6 |
| TAGLN2 | 6 |
| MGST3 | 6 |
| PRDX5 | 6 |
| SMIM22 | 6 |
| NPC2 | 6 |
| MESP1 | 6 |
| MYDGF | 6 |
| ASAH1 | 6 |
| APP | 6 |
| NGFRAP1 | 6 |
| TMEM176B | 6 |
| C8orf4 | 6 |
| KRT81 | 6 |
| VIMP | 6 |
| CXCL17 | 6 |
| MUC1 | 6 |
| COMMD6 | 6 |
| TSPAN13 | 6 |
| TFPI | 6 |
| C15orf48 | 6 |
| CD151 | 6 |
| TACSTD2 | 6 |
| PSME2 | 6 |
| CLDN7 | 6 |
| ATP6AP2 | 6 |
| CUTA | 6 |
| MT2A | 6 |
| CYB5A | 6 |
| CD164 | 6 |
| TM4SF1 | 6 |
| SCGB1D2 | 6 |
| GSTM3 | 6 |
| EGLN3 | 6 |
| LMAN2 | 6 |
| IFI27 | 6 |
| PPP1R1B | 6 |
| B2M | 6 |
| ANXA2 | 6 |
| SARAF | 6 |
| MUCL1 | 6 |
| CSRP1 | 6 |
| NPW | 6 |
| SLC3A2 | 6 |
| PYDC1 | 6 |
| QSOX1 | 6 |
| TSPAN1 | 6 |
| GPX1 | 6 |
| TMSB4X | 6 |
| FGG | 6 |
| GUK1 | 6 |
| IL32 | 6 |
| ATP6V0E1 | 6 |
| BCAP31 | 6 |
| CHCHD10 | 6 |
| TSPO | 6 |
| TNFRSF12A | 6 |
| MT1X | 6 |
| PDE4B | 6 |
| HSPA5 | 6 |
| SCD | 6 |
| SERINC2 | 6 |
| PSCA | 6 |
| VAMP8 | 6 |
| ELF3 | 6 |
| TSC22D3 | 6 |
| S100A7 | 6 |
| GLUL | 6 |
| ZG16B | 6 |
| TMEM45A | 6 |
| APMAP | 6 |
| RPS26 | 6 |
| CALU | 6 |
| OSTC | 6 |
| NCCRP1 | 6 |
| SQLE | 6 |
| RPS28 | 6 |
| SSR2 | 6 |
| SOX4 | 6 |
| CLEC3A | 6 |
| TMEM9 | 6 |
| RPL10 | 6 |
| MUC5AC | 6 |
| HLA-DPA1 | 6 |
| ZNHIT1 | 6 |
| AQP5 | 6 |
| CAPG | 6 |
| SPINT1 | 6 |
| NDFIP1 | 6 |
| FKBP2 | 6 |
| C1S | 6 |
| LDHA | 6 |
| NEAT1 | 6 |
| RPL36A | 6 |
| S100A11 | 6 |
| LCN2 | 6 |
| TUBA1A | 6 |
| GSTK1 | 6 |
| SEPW1 | 6 |
| P4HB | 6 |
| KCNQ1OT1 | 7 |
| AKAP9 | 7 |
| RHOB | 7 |
| SOX4 | 7 |
| VEGFA | 7 |
| CCNL1 | 7 |
| RSRP1 | 7 |
| RRBP1 | 7 |
| ELF3 | 7 |
| H1FX | 7 |
| FUS | 7 |
| NEAT1 | 7 |
| N4BP2L2 | 7 |
| SLC38A2 | 7 |
| BRD2 | 7 |
| PNISR | 7 |
| CLDN4 | 7 |
| MALAT1 | 7 |
| SOX9 | 7 |
| DDIT3 | 7 |
| TAF1D | 7 |
| FOSB | 7 |
| ZNF83 | 7 |
| ARGLU1 | 7 |
| DSC2 | 7 |
| MACF1 | 7 |
| GTF2I | 7 |
| SEPP1 | 7 |
| ANKRD30A | 7 |
| PRLR | 7 |
| MAFB | 7 |
| NFIA | 7 |
| ZFAS1 | 7 |
| MTRNR2L12 | 7 |
| RNMT | 7 |
| NUPR1 | 7 |
| MT-ND6 | 7 |
| RBM39 | 7 |
| HSPA1A | 7 |
| HSPA1B | 7 |
| RGS16 | 7 |
| SUCO | 7 |
| XIST | 7 |
| PDIA6 | 7 |
| VMP1 | 7 |
| SUGP2 | 7 |
| LPIN1 | 7 |
| NDRG1 | 7 |
| PRRC2C | 7 |
| CELF1 | 7 |
| HSP90B1 | 7 |
| JUND | 7 |
| ACADVL | 7 |
| PTPRF | 7 |
| LMAN1 | 7 |
| HEBP2 | 7 |
| ATF3 | 7 |
| BTG1 | 7 |
| GNAS | 7 |
| TSPYL2 | 7 |
| ZFP36L2 | 7 |
| RHOBTB3 | 7 |
| TFAP2A | 7 |
| RAB6A | 7 |
| KMT2C | 7 |
| POLR2J3 | 7 |
| CTNND1 | 7 |
| PRRC2B | 7 |
| RNF43 | 7 |
| CAV1 | 7 |
| RSPO3 | 7 |
| IMPA2 | 7 |
| FAM84A | 7 |
| FOS | 7 |
| IGFBP5 | 7 |
| NCOA3 | 7 |
| WSB1 | 7 |
| MBNL2 | 7 |
| MMP24-AS1 | 7 |
| DDX5 | 7 |
| AP000769.1 | 7 |
| MIA3 | 7 |
| ID2 | 7 |
| HNRNPH1 | 7 |
| FKBP2 | 7 |
| SEL1L | 7 |
| PSAT1 | 7 |
| ASNS | 7 |
| SLC3A2 | 7 |
| EIF4EBP1 | 7 |
| HSPH1 | 7 |
| SNHG19 | 7 |
| RNF19A | 7 |
| GRHL1 | 7 |
| WBP1 | 7 |
| SRRM2 | 7 |
| RUNX1 | 7 |
| ASH1L | 7 |
| HIST1H4C | 7 |
| RBM25 | 7 |
| ZNF292 | 7 |
| RNF213 | 7 |
| PRPF38B | 7 |
| DSP | 7 |
| EPC1 | 7 |
| FNBP4 | 7 |
| ETV6 | 7 |
| SPAG9 | 7 |
| SIAH2 | 7 |
| RBM33 | 7 |
| CAND1 | 7 |
| CEBPB | 7 |
| CD44 | 7 |
| NOC2L | 7 |
| LY6E | 7 |
| ANGPTL4 | 7 |
| GABPB1-AS1 | 7 |
| MTSS1 | 7 |
| DDX42 | 7 |
| PIK3C2G | 7 |
| IAH1 | 7 |
| ATL2 | 7 |
| ADAM17 | 7 |
| PHIP | 7 |
| MPZ | 7 |
| CYP27A1 | 7 |
| IER2 | 7 |
| ACTR3B | 7 |
| PDCD4 | 7 |
| COLCA1 | 7 |
| KIAA1324 | 7 |
| TFAP2C | 7 |
| CTSC | 7 |
| MYC | 7 |
| MT1X | 7 |
| VIMP | 7 |
| SERHL2 | 7 |
| YPEL3 | 7 |
| MKNK2 | 7 |
| ZNF552 | 7 |
| CDH1 | 7 |
| LUC7L3 | 7 |
| DDIT4 | 7 |
| HNRNPR | 7 |
| IFRD1 | 7 |
| RASSF7 | 7 |
| SNHG8 | 7 |
| EPB41L4A-AS1 | 7 |
| ZC3H11A | 7 |
| SNHG15 | 7 |
| CREB3L2 | 7 |
| ERBB3 | 7 |
| THUMPD3-AS1 | 7 |
| RBBP6 | 7 |
| GPBP1 | 7 |
| NARF | 7 |
| SNRNP70 | 7 |
| RP11-290D2.6 | 7 |
| SAT1 | 7 |
| GRB7 | 7 |
| H1F0 | 7 |
| EDEM3 | 7 |
| KIAA0907 | 7 |
| ATF4 | 7 |
| DNAJC3 | 7 |
| DKK1 | 7 |
| SF1 | 7 |
| NAMPT | 7 |
| SETD5 | 7 |
| DYNC1H1 | 7 |
| GOLGB1 | 7 |
| C4orf48 | 7 |
| CLIC3 | 7 |
| TECR | 7 |
| HOOK3 | 7 |
| WDR60 | 7 |
| TMEM101 | 7 |
| SYCP2 | 7 |
| C6orf62 | 7 |
| METTL12 | 7 |
| HIST1H2BG | 7 |
| PCMTD1 | 7 |
| PWWP2A | 7 |
| HIST1H3H | 7 |
| NCK1 | 7 |
| CRACR2B | 7 |
| NPW | 7 |
| RAB3GAP1 | 7 |
| TMEM63A | 7 |
| MGP | 7 |
| ANKRD17 | 7 |
| CALD1 | 7 |
| PRKAR1A | 7 |
| PBX1 | 7 |
| ATXN2L | 7 |
| FAM120A | 7 |
| SAT2 | 7 |
| TAF10 | 7 |
| SFRP1 | 7 |
| CITED2 | 7 |

Gene-set enrichment identified a number of shared and distinct functional features of these GMs (FIG. 6E). For instance, GM4 was uniquely enriched for hallmarks of cell-cycle and proliferation (e.g., E2F_TARGETS), driven by genes including MKI67, PCNA and CDK1. GM3 was predominately enriched for hallmarks of interferon response (IFITM1/2/3, IRF1), antigen presentation (B2M; HLA-A/B) and Epithelial-Mesenchymal-Transition (EMT; VIM, ACTA2). GM1 and GM5 showed characteristics of estrogen response pathways, while GM1 was also enriched for hypoxia, TNFa and p53 signalling and apoptosis. Similar functional associations were also seen when correlating signature scores across all neoplastic cells (FIG. 7B).

For each neoplastic cell, we calculated signature scores for each of the 7 GMs and used hierarchical clustering to identify correlations between cells (FIGS. 7A-7B). This unsupervised approach clearly separated neoplastic cells into groups, reducing the large inter-tumour variability seen in FIGS. 3D-3F. We assigned each neoplastic cell to a module using the maximum of the scaled scores (FIG. 7C). Some modules significantly associated with scSubtype calls, whereas others displayed a more hybrid/diverse subtype association (FIG. 6F-6G; FIGS. 7D-7E). Cells assigned to GM1 and GM5 were predominantly enriched for the luminal subtype. Interestingly, GM1 was almost exclusively composed of cells from LumA cases whereas GM5 was mostly composed of LumB cells. As proliferative cells were classified separately as GM4, this suggests that there were subsets of cells within LumA BrCa with unique properties not found in LumB BrCa. Finally, we used the gene module-based cell state assignments to get a view into the heterogeneity of the neoplastic cells in each tumour. Similar to the scSubtype approach (FIG. 6B), we saw evidence for cellular heterogeneity that broadly aligns with, but was not constrained by, the subtype of the tumour (FIG. 6H). scSubtype and gene module analysis provide complementary new approaches to classifying neoplastic ITTH and further evidence that cancer cells manifest diverse phenotypes within most tumours.

### The Immune Milieu of Breast Cancer

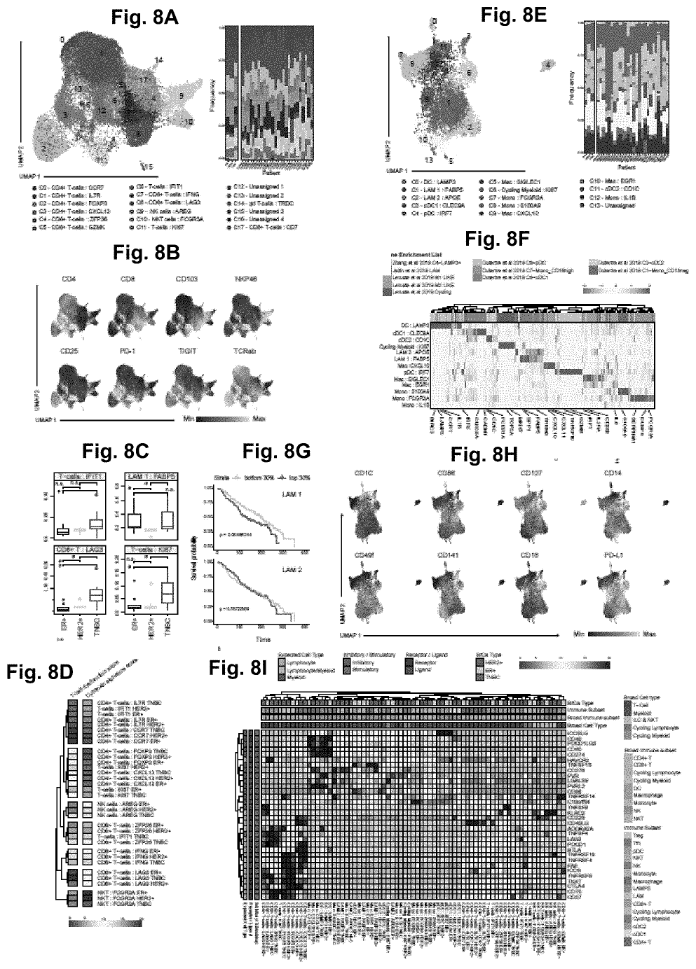

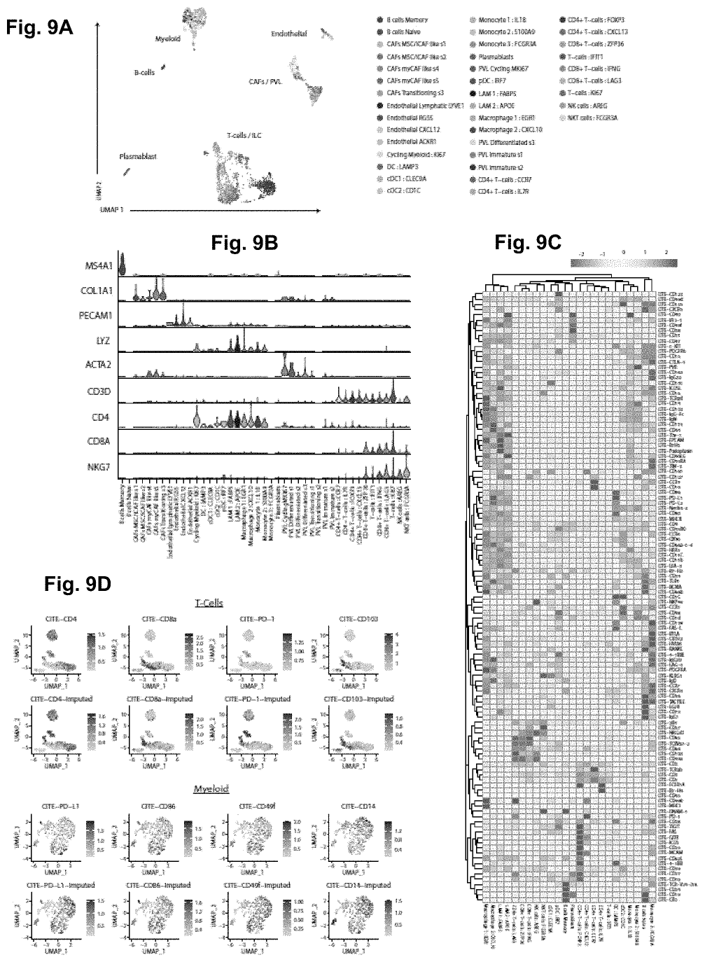

Immune checkpoint inhibitors have revolutionized cancer therapy but have shown minimal efficacy for the treatment of BrCa, mostly restricted to TNBC. To examine the BrCa immune milieu at high resolution, we reclustered immune cells to identify T cells and innate lymphoid cells (FIGS. 8A-8D; 35,233 cells), myeloid cells (FIGS. 8E-8H; 9,678 cells), B cells (3,202 cells), and plasmablasts (3,525 cells) (Table 8). To aid in the annotation of cell phenotypes, we applied CITE-Seq to four samples, which generates simultaneous scRNA-Seq and high dimensional cell surface protein expression data, using barcoded antibodies. We used anchoring based transfer learning to transfer protein expression levels from those four samples to the remaining BrCa cases, which revealed a high correlation to experimentally measured values (FIGS. 9A-9D).

TABLE 8
Cell numbers and proportions per patient. Number and proportion of cells for each
of the three classification tiers (major, minor and subset level) by patient.

| patient ID | cell type | cell number | cell proportion | clinical subtype |
|---|---|---|---|---|
| CID3586 | B-cells | 321 | 0.051958563 | HER2+ |
| CID3921 | B-cells | 162 | 0.053571429 | HER2+ |
| CID45171 | B-cells | 56 | 0.022885166 | HER2+ |
| CID3838 | B-cells | 47 | 0.019974501 | HER2+ |
| CID4066 | B-cells | 38 | 0.007157657 | HER2+ |
| CID44041 | B-cells | 176 | 0.082590333 | TNBC |
| CID4465 | B-cells | 33 | 0.021099744 | TNBC |
| CID4495 | B-cells | 773 | 0.096806512 | TNBC |
| CID44971 | B-cells | 369 | 0.04620586 | TNBC |
| CID44991 | B-cells | 88 | 0.012530258 | TNBC |
| CID4513 | B-cells | 43 | 0.007652607 | TNBC |
| CID4515 | B-cells | 494 | 0.119064835 | TNBC |
| CID4523 | B-cells | 0 | 0 | TNBC |
| CID3946 | B-cells | 0 | 0 | TNBC |
| CID3963 | B-cells | 0 | 0 | TNBC |
| CID4461 | B-cells | 0 | 0 | ER+ |
| CID4463 | B-cells | 0 | 0 | ER+ |
| CID4471 | B-cells | 99 | 0.011499593 | ER+ |
| CID4530N | B-cells | 0 | 0 | ER+ |
| CID4535 | B-cells | 56 | 0.014137844 | ER+ |
| CID4040 | B-cells | 105 | 0.041485579 | ER+ |
| CID3941 | B-cells | 55 | 0.087163233 | ER+ |
| CID3948 | B-cells | 85 | 0.036527718 | ER+ |
| CID4067 | B-cells | 53 | 0.014080765 | ER+ |
| CID4290A | B-cells | 117 | 0.020210745 | ER+ |
| CID4398 | B-cells | 36 | 0.00808807 | ER+ |
| CID3586 | CAFs | 185 | 0.029944966 | HER2+ |
| CID3921 | CAFs | 106 | 0.03505291 | HER2+ |
| CID45171 | CAFs | 32 | 0.013077237 | HER2+ |
| CID3838 | CAFs | 203 | 0.086272843 | HER2+ |
| CID4066 | CAFs | 923 | 0.173855717 | HER2+ |
| CID44041 | CAFs | 681 | 0.319568278 | TNBC |
| CID4465 | CAFs | 379 | 0.242327366 | TNBC |
| CID4495 | CAFs | 232 | 0.029054477 | TNBC |
| CID44971 | CAFs | 582 | 0.072877536 | TNBC |
| CID44991 | CAFs | 245 | 0.034885377 | TNBC |
| CID4513 | CAFs | 13 | 0.002313579 | TNBC |
| CID4515 | CAFs | 187 | 0.045071101 | TNBC |
| CID4523 | CAFs | 42 | 0.023945268 | TNBC |
| CID3946 | CAFs | 167 | 0.215762274 | TNBC |
| CID3963 | CAFs | 23 | 0.006521123 | TNBC |
| CID4461 | CAFs | 41 | 0.064976228 | ER+ |
| CID4463 | CAFs | 25 | 0.021968366 | ER+ |
| CID4471 | CAFs | 1292 | 0.150075502 | ER+ |
| CID4530N | CAFs | 368 | 0.083465638 | ER+ |
| CID4535 | CAFs | 102 | 0.025751073 | ER+ |
| CID4040 | CAFs | 129 | 0.050967997 | ER+ |
| CID3941 | CAFs | 8 | 0.012678288 | ER+ |
| CID3948 | CAFs | 15 | 0.006446068 | ER+ |
| CID4067 | CAFs | 135 | 0.0358661 | ER+ |
| CID4290A | CAFs | 280 | 0.048367594 | ER+ |
| CID4398 | CAFs | 178 | 0.039991013 | ER+ |
| CID3586 | Cancer Epithelial | 0 | 0 | HER2+ |
| CID3921 | Cancer Epithelial | 441 | 0.145833333 | HER2+ |
| CID45171 | Cancer Epithelial | 813 | 0.332243564 | HER2+ |
| CID3838 | Cancer Epithelial | 0 | 0 | HER2+ |
| CID4066 | Cancer Epithelial | 521 | 0.098135242 | HER2+ |
| CID44041 | Cancer Epithelial | 0 | 0 | TNBC |
| CID4465 | Cancer Epithelial | 124 | 0.079283887 | TNBC |
| CID4495 | Cancer Epithelial | 1184 | 0.148278021 | TNBC |
| CID44971 | Cancer Epithelial | 894 | 0.111945905 | TNBC |
| CID44991 | Cancer Epithelial | 4018 | 0.572120177 | TNBC |
| CID4513 | Cancer Epithelial | 1058 | 0.188289731 | TNBC |
| CID4515 | Cancer Epithelial | 2169 | 0.522776573 | TNBC |
| CID4523 | Cancer Epithelial | 1167 | 0.665336374 | TNBC |
| CID3946 | Cancer Epithelial | 0 | 0 | TNBC |
| CID3963 | Cancer Epithelial | 222 | 0.062943011 | TNBC |
| CID4461 | Cancer Epithelial | 207 | 0.328050713 | ER+ |
| CID4463 | Cancer Epithelial | 659 | 0.579086116 | ER+ |
| CID4471 | Cancer Epithelial | 212 | 0.024625392 | ER+ |
| CID4530N | Cancer Epithelial | 1715 | 0.388977092 | ER+ |
| CID4535 | Cancer Epithelial | 2223 | 0.561221914 | ER+ |
| CID4040 | Cancer Epithelial | 0 | 0 | ER+ |
| CID3941 | Cancer Epithelial | 196 | 0.310618067 | ER+ |
| CID3948 | Cancer Epithelial | 261 | 0.112161581 | ER+ |
| CID4067 | Cancer Epithelial | 2352 | 0.624867163 | ER+ |
| CID4290A | Cancer Epithelial | 4053 | 0.700120919 | ER+ |
| CID4398 | Cancer Epithelial | 0 | 0 | ER+ |
| CID3586 | Endothelial | 157 | 0.025412755 | HER2+ |
| CID3921 | Endothelial | 210 | 0.069444444 | HER2+ |
| CID45171 | Endothelial | 15 | 0.006129955 | HER2+ |
| CID3838 | Endothelial | 99 | 0.042073948 | HER2+ |
| CID4066 | Endothelial | 535 | 0.100772273 | HER2+ |
| CID44041 | Endothelial | 148 | 0.069450962 | TNBC |
| CID4465 | Endothelial | 294 | 0.18797954 | TNBC |
| CID4495 | Endothelial | 184 | 0.023043206 | TNBC |
| CID44971 | Endothelial | 217 | 0.027172552 | TNBC |
| CID44991 | Endothelial | 41 | 0.005837961 | TNBC |
| CID4513 | Endothelial | 162 | 0.028830753 | TNBC |
| CID4515 | Endothelial | 122 | 0.029404676 | TNBC |
| CID4523 | Endothelial | 3 | 0.001710376 | TNBC |
| CID3946 | Endothelial | 110 | 0.142118863 | TNBC |
| CID3963 | Endothelial | 102 | 0.028919762 | TNBC |
| CID4461 | Endothelial | 182 | 0.288431062 | ER+ |
| CID4463 | Endothelial | 79 | 0.069420035 | ER+ |
| CID4471 | Endothelial | 2778 | 0.322685562 | ER+ |
| CID4530N | Endothelial | 1016 | 0.230437741 | ER+ |
| CID4535 | Endothelial | 219 | 0.055289068 | ER+ |
| CID4040 | Endothelial | 218 | 0.086131964 | ER+ |
| CID3941 | Endothelial | 44 | 0.069730586 | ER+ |
| CID3948 | Endothelial | 85 | 0.036527718 | ER+ |
| CID4067 | Endothelial | 186 | 0.049415515 | ER+ |
| CID4290A | Endothelial | 298 | 0.051476939 | ER+ |
| CID4398 | Endothelial | 101 | 0.02269153 | ER+ |
| CID3586 | Myeloid | 200 | 0.032372936 | HER2+ |
| CID3921 | Myeloid | 385 | 0.127314815 | HER2+ |
| CID45171 | Myeloid | 172 | 0.070290151 | HER2+ |
| CID3838 | Myeloid | 444 | 0.188695283 | HER2+ |
| CID4066 | Myeloid | 221 | 0.041627425 | HER2+ |
| CID44041 | Myeloid | 105 | 0.049272642 | TNBC |
| CID4465 | Myeloid | 181 | 0.1157289 | TNBC |
| CID4495 | Myeloid | 897 | 0.112335629 | TNBC |
| CID44971 | Myeloid | 684 | 0.085649887 | TNBC |
| CID44991 | Myeloid | 206 | 0.029332194 | TNBC |
| CID4513 | Myeloid | 2795 | 0.49741947 | TNBC |
| CID4515 | Myeloid | 563 | 0.135695348 | TNBC |
| CID4523 | Myeloid | 355 | 0.202394527 | TNBC |
| CID3946 | Myeloid | 157 | 0.202842377 | TNBC |
| CID3963 | Myeloid | 479 | 0.13580947 | TNBC |
| CID4461 | Myeloid | 53 | 0.083993661 | ER+ |
| CID4463 | Myeloid | 101 | 0.088752197 | ER+ |
| CID4471 | Myeloid | 285 | 0.03310489 | ER+ |
| CID4530N | Myeloid | 96 | 0.021773645 | ER+ |
| CID4535 | Myeloid | 255 | 0.064377682 | ER+ |
| CID4040 | Myeloid | 50 | 0.019755038 | ER+ |
| CID3941 | Myeloid | 37 | 0.058637084 | ER+ |
| CID3948 | Myeloid | 122 | 0.052428019 | ER+ |
| CID4067 | Myeloid | 266 | 0.070669501 | ER+ |
| CID4290A | Myeloid | 341 | 0.058904819 | ER+ |
| CID4398 | Myeloid | 225 | 0.050550438 | ER+ |
| CID3586 | Normal Epithelial | 698 | 0.112981547 | HER2+ |
| CID3921 | Normal Epithelial | 0 | 0 | HER2+ |
| CID45171 | Normal Epithelial | 0 | 0 | HER2+ |
| CID3838 | Normal Epithelial | 0 | 0 | HER2+ |
| CID4066 | Normal Epithelial | 270 | 0.050857035 | HER2+ |
| CID44041 | Normal Epithelial | 151 | 0.070858752 | TNBC |
| CID4465 | Normal Epithelial | 10 | 0.006393862 | TNBC |
| CID4495 | Normal Epithelial | 0 | 0 | TNBC |
| CID44971 | Normal Epithelial | 735 | 0.092036063 | TNBC |
| CID44991 | Normal Epithelial | 24 | 0.003417343 | TNBC |
| CID4513 | Normal Epithelial | 0 | 0 | TNBC |
| CID4515 | Normal Epithelial | 36 | 0.00867679 | TNBC |
| CID4523 | Normal Epithelial | 0 | 0 | TNBC |
| CID3946 | Normal Epithelial | 0 | 0 | TNBC |
| CID3963 | Normal Epithelial | 1 | 0.000283527 | TNBC |
| CID4461 | Normal Epithelial | 0 | 0 | ER+ |
| CID4463 | Normal Epithelial | 26 | 0.0228471 | ER+ |
| CID4471 | Normal Epithelial | 1966 | 0.228365664 | ER+ |
| CID4530N | Normal Epithelial | 398 | 0.090269902 | ER+ |
| CID4535 | Normal Epithelial | 22 | 0.005554153 | ER+ |
| CID4040 | Normal Epithelial | 0 | 0 | ER+ |
| CID3941 | Normal Epithelial | 0 | 0 | ER+ |
| CID3948 | Normal Epithelial | 0 | 0 | ER+ |
| CID4067 | Normal Epithelial | 0 | 0 | ER+ |
| CID4290A | Normal Epithelial | 18 | 0.003109345 | ER+ |
| CID4398 | Normal Epithelial | 0 | 0 | ER+ |
| CID3586 | Plasmablasts | 0 | 0 | HER2+ |
| CID3921 | Plasmablasts | 175 | 0.05787037 | HER2+ |
| CID45171 | Plasmablasts | 0 | 0 | HER2+ |
| CID3838 | Plasmablasts | 51 | 0.021674458 | HER2+ |
| CID4066 | Plasmablasts | 0 | 0 | HER2+ |
| CID44041 | Plasmablasts | 0 | 0 | TNBC |
| CID4465 | Plasmablasts | 110 | 0.070332481 | TNBC |
| CID4495 | Plasmablasts | 1020 | 0.127739512 | TNBC |
| CID44971 | Plasmablasts | 48 | 0.006010518 | TNBC |
| CID44991 | Plasmablasts | 1453 | 0.206891642 | TNBC |
| CID4513 | Plasmablasts | 0 | 0 | TNBC |
| CID4515 | Plasmablasts | 36 | 0.00867679 | TNBC |
| CID4523 | Plasmablasts | 0 | 0 | TNBC |
| CID3946 | Plasmablasts | 0 | 0 | TNBC |
| CID3963 | Plasmablasts | 0 | 0 | TNBC |
| CID4461 | Plasmablasts | 32 | 0.050713154 | ER+ |
| CID4463 | Plasmablasts | 0 | 0 | ER+ |
| CID4471 | Plasmablasts | 51 | 0.005924033 | ER+ |
| CID4530N | Plasmablasts | 55 | 0.012474484 | ER+ |
| CID4535 | Plasmablasts | 96 | 0.024236304 | ER+ |
| CID4040 | Plasmablasts | 74 | 0.029237456 | ER+ |
| CID3941 | Plasmablasts | 0 | 0 | ER+ |
| CID3948 | Plasmablasts | 232 | 0.099699183 | ER+ |
| CID4067 | Plasmablasts | 0 | 0 | ER+ |
| CID4290A | Plasmablasts | 0 | 0 | ER+ |
| CID4398 | Plasmablasts | 91 | 0.020444844 | ER+ |
| CID3586 | PVL | 21 | 0.003399158 | HER2+ |
| CID3921 | PVL | 72 | 0.023809524 | HER2+ |
| CID45171 | PVL | 13 | 0.005312628 | HER2+ |
| CID3838 | PVL | 158 | 0.067148321 | HER2+ |
| CID4066 | PVL | 630 | 0.118666416 | HER2+ |
| CID44041 | PVL | 128 | 0.060065697 | TNBC |
| CID4465 | PVL | 317 | 0.202685422 | TNBC |
| CID4495 | PVL | 191 | 0.02391985 | TNBC |
| CID44971 | PVL | 91 | 0.011394941 | TNBC |
| CID44991 | PVL | 46 | 0.006549907 | TNBC |
| CID4513 | PVL | 82 | 0.014593344 | TNBC |
| CID4515 | PVL | 123 | 0.029645698 | TNBC |
| CID4523 | PVL | 10 | 0.005701254 | TNBC |
| CID3946 | PVL | 248 | 0.320413437 | TNBC |
| CID3963 | PVL | 28 | 0.007938758 | TNBC |
| CID4461 | PVL | 48 | 0.076069731 | ER+ |
| CID4463 | PVL | 31 | 0.027240773 | ER+ |
| CID4471 | PVL | 1285 | 0.1492624 | ER+ |
| CID4530N | PVL | 469 | 0.106373327 | ER+ |
| CID4535 | PVL | 592 | 0.149457208 | ER+ |
| CID4040 | PVL | 443 | 0.175029633 | ER+ |
| CID3941 | PVL | 25 | 0.039619651 | ER+ |
| CID3948 | PVL | 62 | 0.026643747 | ER+ |
| CID4067 | PVL | 83 | 0.02205101 | ER+ |
| CID4290A | PVL | 140 | 0.024183797 | ER+ |
| CID4398 | PVL | 87 | 0.019546169 | ER+ |
| CID3586 | T-cells | 4596 | 0.743930074 | HER2+ |
| CID3921 | T-cells | 1473 | 0.487103175 | HER2+ |
| CID45171 | T-cells | 1346 | 0.5500613 | HER2+ |
| CID3838 | T-cells | 1351 | 0.574160646 | HER2+ |
| CID4066 | T-cells | 2171 | 0.408928235 | HER2+ |
| CID44041 | T-cells | 742 | 0.348193336 | TNBC |
| CID4465 | T-cells | 116 | 0.074168798 | TNBC |
| CID4495 | T-cells | 3504 | 0.438822793 | TNBC |
| CID44971 | T-cells | 4366 | 0.546706737 | TNBC |
| CID44991 | T-cells | 902 | 0.128435142 | TNBC |
| CID4513 | T-cells | 1466 | 0.260900516 | TNBC |
| CID4515 | T-cells | 419 | 0.10098819 | TNBC |
| CID4523 | T-cells | 177 | 0.100912201 | TNBC |
| CID3946 | T-cells | 92 | 0.118863049 | TNBC |
| CID3963 | T-cells | 2672 | 0.757584349 | TNBC |
| CID4461 | T-cells | 68 | 0.107765452 | ER+ |
| CID4463 | T-cells | 217 | 0.190685413 | ER+ |
| CID4471 | T-cells | 641 | 0.074456964 | ER+ |
| CID4530N | T-cells | 292 | 0.06622817 | ER+ |
| CID4535 | T-cells | 396 | 0.099974754 | ER+ |
| CID4040 | T-cells | 1512 | 0.597392335 | ER+ |
| CID3941 | T-cells | 266 | 0.42155309 | ER+ |
| CID3948 | T-cells | 1465 | 0.629565965 | ER+ |
| CID4067 | T-cells | 689 | 0.183049947 | ER+ |
| CID4290A | T-cells | 542 | 0.093625842 | ER+ |
| CID4398 | T-cells | 3733 | 0.838687935 | ER+ |
| CID3586 | B cells Memory | 289 | 0.046778893 | HER2+ |
| CID3921 | B cells Memory | 159 | 0.052579365 | HER2+ |
| CID45171 | B cells Memory | 56 | 0.022885166 | HER2+ |
| CID3838 | B cells Memory | 45 | 0.019124522 | HER2+ |
| CID4066 | B cells Memory | 38 | 0.007157657 | HER2+ |
| CID44041 | B cells Memory | 176 | 0.082590333 | TNBC |
| CID4465 | B cells Memory | 33 | 0.021099744 | TNBC |
| CID4495 | B cells Memory | 526 | 0.065873513 | TNBC |
| CID44971 | B cells Memory | 273 | 0.034184823 | TNBC |
| CID44991 | B cells Memory | 83 | 0.011818311 | TNBC |
| CID4513 | B cells Memory | 43 | 0.007652607 | TNBC |
| CID4515 | B cells Memory | 258 | 0.062183659 | TNBC |
| CID4523 | B cells Memory | 0 | 0 | TNBC |
| CID3946 | B cells Memory | 0 | 0 | TNBC |
| CID3963 | B cells Memory | 0 | 0 | TNBC |
| CID4461 | B cells Memory | 0 | 0 | ER+ |
| CID4463 | B cells Memory | 0 | 0 | ER+ |
| CID4471 | B cells Memory | 99 | 0.011499593 | ER+ |
| CID4530N | B cells Memory | 0 | 0 | ER+ |
| CID4535 | B cells Memory | 56 | 0.014137844 | ER+ |
| CID4040 | B cells Memory | 102 | 0.040300277 | ER+ |
| CID3941 | B cells Memory | 55 | 0.087163233 | ER+ |
| CID3948 | B cells Memory | 84 | 0.03609798 | ER+ |
| CID4067 | B cells Memory | 53 | 0.014080765 | ER+ |
| CID4290A | B cells Memory | 117 | 0.020210745 | ER+ |
| CID4398 | B cells Memory | 36 | 0.00808807 | ER+ |
| CID3586 | B cells Naive | 32 | 0.00517967 | HER2+ |
| CID3921 | B cells Naive | 3 | 0.000992063 | HER2+ |
| CID45171 | B cells Naive | 0 | 0 | HER2+ |
| CID3838 | B cells Naive | 2 | 0.000849979 | HER2+ |
| CID4066 | B cells Naive | 0 | 0 | HER2+ |
| CID44041 | B cells Naive | 0 | 0 | TNBC |
| CID4465 | B cells Naive | 0 | 0 | TNBC |
| CID4495 | B cells Naive | 247 | 0.030932999 | TNBC |
| CID44971 | B cells Naive | 96 | 0.012021037 | TNBC |
| CID44991 | B cells Naive | 5 | 0.000711946 | TNBC |
| CID4513 | B cells Naive | 0 | 0 | TNBC |
| CID4515 | B cells Naive | 236 | 0.056881176 | TNBC |
| CID4523 | B cells Naive | 0 | 0 | TNBC |
| CID3946 | B cells Naive | 0 | 0 | TNBC |
| CID3963 | B cells Naive | 0 | 0 | TNBC |
| CID4461 | B cells Naive | 0 | 0 | ER+ |
| CID4463 | B cells Naive | 0 | 0 | ER+ |
| CID4471 | B cells Naive | 0 | 0 | ER+ |
| CID4530N | B cells Naive | 0 | 0 | ER+ |
| CID4535 | B cells Naive | 0 | 0 | ER+ |
| CID4040 | B cells Naive | 3 | 0.001185302 | ER+ |
| CID3941 | B cells Naive | 0 | 0 | ER+ |
| CID3948 | B cells Naive | 1 | 0.000429738 | ER+ |
| CID4067 | B cells Naive | 0 | 0 | ER+ |
| CID4290A | B cells Naive | 0 | 0 | ER+ |
| CID4398 | B cells Naive | 0 | 0 | ER+ |
| CID3586 | CAFs MSC iCAF-like | 146 | 0.023632243 | HER2+ |
| CID3921 | CAFs MSC iCAF-like | 44 | 0.014550265 | HER2+ |
| CID45171 | CAFs MSC iCAF-like | 17 | 0.006947282 | HER2+ |
| CID3838 | CAFs MSC iCAF-like | 49 | 0.020824479 | HER2+ |
| CID4066 | CAFs MSC iCAF-like | 323 | 0.060840083 | HER2+ |
| CID44041 | CAFs MSC iCAF-like | 376 | 0.176442985 | TNBC |
| CID4465 | CAFs MSC iCAF-like | 130 | 0.083120205 | TNBC |
| CID4495 | CAFs MSC iCAF-like | 120 | 0.015028178 | TNBC |
| CID44971 | CAFs MSC iCAF-like | 421 | 0.052717255 | TNBC |
| CID44991 | CAFs MSC iCAF-like | 66 | 0.009397693 | TNBC |
| CID4513 | CAFs MSC iCAF-like | 6 | 0.001067806 | TNBC |
| CID4515 | CAFs MSC iCAF-like | 91 | 0.021932996 | TNBC |
| CID4523 | CAFs MSC iCAF-like | 26 | 0.014823261 | TNBC |
| CID3946 | CAFs MSC iCAF-like | 24 | 0.031007752 | TNBC |
| CID3963 | CAFs MSC iCAF-like | 8 | 0.002268217 | TNBC |
| CID4461 | CAFs MSC iCAF-like | 17 | 0.026941363 | ER+ |
| CID4463 | CAFs MSC iCAF-like | 6 | 0.005272408 | ER+ |
| CID4471 | CAFs MSC iCAF-like | 761 | 0.088395865 | ER+ |
| CID4530N | CAFs MSC iCAF-like | 179 | 0.040598775 | ER+ |
| CID4535 | CAFs MSC iCAF-like | 58 | 0.014642767 | ER+ |
| CID4040 | CAFs MSC iCAF-like | 47 | 0.018569735 | ER+ |
| CID3941 | CAFs MSC iCAF-like | 5 | 0.00792393 | ER+ |
| CID3948 | CAFs MSC iCAF-like | 4 | 0.001718951 | ER+ |
| CID4067 | CAFs MSC iCAF-like | 37 | 0.009829968 | ER+ |
| CID4290A | CAFs MSC iCAF-like | 87 | 0.015028502 | ER+ |
| CID4398 | CAFs MSC iCAF-like | 105 | 0.023590204 | ER+ |
| CID3586 | CAFs myCAF-like | 39 | 0.006312723 | HER2+ |
| CID3921 | CAFs myCAF-like | 62 | 0.020502646 | HER2+ |
| CID45171 | CAFs myCAF-like | 15 | 0.006129955 | HER2+ |
| CID3838 | CAFs myCAF-like | 154 | 0.065448364 | HER2+ |
| CID4066 | CAFs myCAF-like | 600 | 0.113015634 | HER2+ |
| CID44041 | CAFs myCAF-like | 305 | 0.143125293 | TNBC |
| CID4465 | CAFs myCAF-like | 249 | 0.159207161 | TNBC |
| CID4495 | CAFs myCAF-like | 112 | 0.014026299 | TNBC |
| CID44971 | CAFs myCAF-like | 161 | 0.02016028 | TNBC |
| CID44991 | CAFs myCAF-like | 179 | 0.025487683 | TNBC |
| CID4513 | CAFs myCAF-like | 7 | 0.001245773 | TNBC |
| CID4515 | CAFs myCAF-like | 96 | 0.023138106 | TNBC |
| CID4523 | CAFs myCAF-like | 16 | 0.009122007 | TNBC |
| CID3946 | CAFs myCAF-like | 143 | 0.184754522 | TNBC |
| CID3963 | CAFs myCAF-like | 15 | 0.004252906 | TNBC |
| CID4461 | CAFs myCAF-like | 24 | 0.038034865 | ER+ |
| CID4463 | CAFs myCAF-like | 19 | 0.016695958 | ER+ |
| CID4471 | CAFs myCAF-like | 531 | 0.061679638 | ER+ |
| CID4530N | CAFs myCAF-like | 189 | 0.042866863 | ER+ |
| CID4535 | CAFs myCAF-like | 44 | 0.011108306 | ER+ |
| CID4040 | CAFs myCAF-like | 82 | 0.032398262 | ER+ |
| CID3941 | CAFs myCAF-like | 3 | 0.004754358 | ER+ |
| CID3948 | CAFs myCAF-like | 11 | 0.004727116 | ER+ |
| CID4067 | CAFs myCAF-like | 98 | 0.026036132 | ER+ |
| CID4290A | CAFs myCAF-like | 193 | 0.033339091 | ER+ |
| CID4398 | CAFs myCAF-like | 73 | 0.016400809 | ER+ |
| CID3586 | Cancer Basal SC | 0 | 0 | HER2+ |
| CID3921 | Cancer Basal SC | 0 | 0 | HER2+ |
| CID45171 | Cancer Basal SC | 1 | 0.000408664 | HER2+ |
| CID3838 | Cancer Basal SC | 0 | 0 | HER2+ |
| CID4066 | Cancer Basal SC | 2 | 0.000376719 | HER2+ |
| CID44041 | Cancer Basal SC | 0 | 0 | TNBC |
| CID4465 | Cancer Basal SC | 22 | 0.014066496 | TNBC |
| CID4495 | Cancer Basal SC | 711 | 0.089041954 | TNBC |
| CID44971 | Cancer Basal SC | 646 | 0.08089156 | TNBC |
| CID44991 | Cancer Basal SC | 369 | 0.052541649 | TNBC |
| CID4513 | Cancer Basal SC | 502 | 0.08933974 | TNBC |
| CID4515 | Cancer Basal SC | 1200 | 0.28922632 | TNBC |
| CID4523 | Cancer Basal SC | 545 | 0.310718358 | TNBC |
| CID3946 | Cancer Basal SC | 0 | 0 | TNBC |
| CID3963 | Cancer Basal SC | 182 | 0.051601928 | TNBC |
| CID4461 | Cancer Basal SC | 0 | 0 | ER+ |
| CID4463 | Cancer Basal SC | 3 | 0.002636204 | ER+ |
| CID4471 | Cancer Basal SC | 10 | 0.001161575 | ER+ |
| CID4530N | Cancer Basal SC | 71 | 0.016103425 | ER+ |
| CID4535 | Cancer Basal SC | 1 | 0.000252461 | ER+ |
| CID4040 | Cancer Basal SC | 0 | 0 | ER+ |
| CID3941 | Cancer Basal SC | 0 | 0 | ER+ |
| CID3948 | Cancer Basal SC | 0 | 0 | ER+ |
| CID4067 | Cancer Basal SC | 1 | 0.000265675 | ER+ |
| CID4290A | Cancer Basal SC | 46 | 0.007946105 | ER+ |
| CID4398 | Cancer Basal SC | 0 | 0 | ER+ |
| CID3586 | Cancer Cycling | 0 | 0 | HER2+ |
| CID3921 | Cancer Cycling | 64 | 0.021164021 | HER2+ |
| CID45171 | Cancer Cycling | 236 | 0.096444626 | HER2+ |
| CID3838 | Cancer Cycling | 0 | 0 | HER2+ |
| CID4066 | Cancer Cycling | 112 | 0.021096252 | HER2+ |
| CID44041 | Cancer Cycling | 0 | 0 | TNBC |
| CID4465 | Cancer Cycling | 97 | 0.06202046 | TNBC |
| CID4495 | Cancer Cycling | 459 | 0.05748278 | TNBC |
| CID44971 | Cancer Cycling | 246 | 0.030803907 | TNBC |
| CID44991 | Cancer Cycling | 1583 | 0.22540225 | TNBC |
| CID4513 | Cancer Cycling | 500 | 0.088983805 | TNBC |
| CID4515 | Cancer Cycling | 927 | 0.223427332 | TNBC |
| CID4523 | Cancer Cycling | 531 | 0.302736602 | TNBC |
| CID3946 | Cancer Cycling | 0 | 0 | TNBC |
| CID3963 | Cancer Cycling | 29 | 0.008222285 | TNBC |
| CID4461 | Cancer Cycling | 33 | 0.05229794 | ER+ |
| CID4463 | Cancer Cycling | 47 | 0.041300527 | ER+ |
| CID4471 | Cancer Cycling | 28 | 0.00325241 | ER+ |
| CID4530N | Cancer Cycling | 15 | 0.003402132 | ER+ |
| CID4535 | Cancer Cycling | 195 | 0.049229992 | ER+ |
| CID4040 | Cancer Cycling | 0 | 0 | ER+ |
| CID3941 | Cancer Cycling | 7 | 0.011093502 | ER+ |
| CID3948 | Cancer Cycling | 13 | 0.005586592 | ER+ |
| CID4067 | Cancer Cycling | 117 | 0.031083953 | ER+ |
| CID4290A | Cancer Cycling | 120 | 0.020728969 | ER+ |
| CID4398 | Cancer Cycling | 0 | 0 | ER+ |
| CID3586 | Cancer Her2 SC | 0 | 0 | HER2+ |
| CID3921 | Cancer Her2 SC | 377 | 0.124669312 | HER2+ |
| CID45171 | Cancer Her2 SC | 567 | 0.231712301 | HER2+ |
| CID3838 | Cancer Her2 SC | 0 | 0 | HER2+ |
| CID4066 | Cancer Her2 SC | 393 | 0.07402524 | HER2+ |
| CID44041 | Cancer Her2 SC | 0 | 0 | TNBC |
| CID4465 | Cancer Her2 SC | 0 | 0 | TNBC |
| CID4495 | Cancer Her2 SC | 0 | 0 | TNBC |
| CID44971 | Cancer Her2 SC | 1 | 0.000125219 | TNBC |
| CID44991 | Cancer Her2 SC | 1912 | 0.272248327 | TNBC |
| CID4513 | Cancer Her2 SC | 31 | 0.005516996 | TNBC |
| CID4515 | Cancer Her2 SC | 30 | 0.007230658 | TNBC |
| CID4523 | Cancer Her2 SC | 67 | 0.038198404 | TNBC |
| CID3946 | Cancer Her2 SC | 0 | 0 | TNBC |
| CID3963 | Cancer Her2 SC | 2 | 0.000567054 | TNBC |
| CID4461 | Cancer Her2 SC | 0 | 0 | ER+ |
| CID4463 | Cancer Her2 SC | 2 | 0.001757469 | ER+ |
| CID4471 | Cancer Her2 SC | 5 | 0.000580788 | ER+ |
| CID4530N | Cancer Her2 SC | 33 | 0.00748469 | ER+ |
| CID4535 | Cancer Her2 SC | 2 | 0.000504923 | ER+ |
| CID4040 | Cancer Her2 SC | 0 | 0 | ER+ |
| CID3941 | Cancer Her2 SC | 0 | 0 | ER+ |
| CID3948 | Cancer Her2 SC | 0 | 0 | ER+ |
| CID4067 | Cancer Her2 SC | 6 | 0.001594049 | ER+ |
| CID4290A | Cancer Her2 SC | 280 | 0.048367594 | ER+ |
| CID4398 | Cancer Her2 SC | 0 | 0 | ER+ |
| CID3586 | Cancer LumA SC | 0 | 0 | HER2+ |
| CID3921 | Cancer LumA SC | 0 | 0 | HER2+ |
| CID45171 | Cancer LumA SC | 0 | 0 | HER2+ |
| CID3838 | Cancer LumA SC | 0 | 0 | HER2+ |
| CID4066 | Cancer LumA SC | 8 | 0.001506875 | HER2+ |
| CID44041 | Cancer LumA SC | 0 | 0 | TNBC |
| CID4465 | Cancer LumA SC | 0 | 0 | TNBC |
| CID4495 | Cancer LumA SC | 2 | 0.00025047 | TNBC |
| CID44971 | Cancer LumA SC | 0 | 0 | TNBC |
| CID44991 | Cancer LumA SC | 51 | 0.007261854 | TNBC |
| CID4513 | Cancer LumA SC | 14 | 0.002491547 | TNBC |
| CID4515 | Cancer LumA SC | 0 | 0 | TNBC |
| CID4523 | Cancer LumA SC | 2 | 0.001140251 | TNBC |
| CID3946 | Cancer LumA SC | 0 | 0 | TNBC |
| CID3963 | Cancer LumA SC | 8 | 0.002268217 | TNBC |
| CID4461 | Cancer LumA SC | 0 | 0 | ER+ |
| CID4463 | Cancer LumA SC | 582 | 0.51142355 | ER+ |
| CID4471 | Cancer LumA SC | 169 | 0.019630619 | ER+ |
| CID4530N | Cancer LumA SC | 1145 | 0.259696076 | ER+ |
| CID4535 | Cancer LumA SC | 0 | 0 | ER+ |
| CID4040 | Cancer LumA SC | 0 | 0 | ER+ |
| CID3941 | Cancer LumA SC | 187 | 0.296354992 | ER+ |
| CID3948 | Cancer LumA SC | 194 | 0.083369145 | ER+ |
| CID4067 | Cancer LumA SC | 1827 | 0.485387885 | ER+ |
| CID4290A | Cancer LumA SC | 3553 | 0.613750216 | ER+ |
| CID4398 | Cancer LumA SC | 0 | 0 | ER+ |
| CID3586 | Cancer LumB SC | 0 | 0 | HER2+ |
| CID3921 | Cancer LumB SC | 0 | 0 | HER2+ |
| CID45171 | Cancer LumB SC | 9 | 0.003677973 | HER2+ |
| CID3838 | Cancer LumB SC | 0 | 0 | HER2+ |
| CID4066 | Cancer LumB SC | 6 | 0.001130156 | HER2+ |
| CID44041 | Cancer LumB SC | 0 | 0 | TNBC |
| CID4465 | Cancer LumB SC | 5 | 0.003196931 | TNBC |
| CID4495 | Cancer LumB SC | 12 | 0.001502818 | TNBC |
| CID44971 | Cancer LumB SC | 1 | 0.000125219 | TNBC |
| CID44991 | Cancer LumB SC | 103 | 0.014666097 | TNBC |
| CID4513 | Cancer LumB SC | 11 | 0.001957644 | TNBC |
| CID4515 | Cancer LumB SC | 12 | 0.002892263 | TNBC |
| CID4523 | Cancer LumB SC | 22 | 0.012542759 | TNBC |
| CID3946 | Cancer LumB SC | 0 | 0 | TNBC |
| CID3963 | Cancer LumB SC | 1 | 0.000283527 | TNBC |
| CID4461 | Cancer LumB SC | 174 | 0.275752773 | ER+ |
| CID4463 | Cancer LumB SC | 25 | 0.021968366 | ER+ |
| CID4471 | Cancer LumB SC | 0 | 0 | ER+ |
| CID4530N | Cancer LumB SC | 451 | 0.102290769 | ER+ |
| CID4535 | Cancer LumB SC | 2025 | 0.511234537 | ER+ |
| CID4040 | Cancer LumB SC | 0 | 0 | ER+ |
| CID3941 | Cancer LumB SC | 2 | 0.003169572 | ER+ |
| CID3948 | Cancer LumB SC | 54 | 0.023205844 | ER+ |
| CID4067 | Cancer LumB SC | 401 | 0.1065356 | ER+ |
| CID4290A | Cancer LumB SC | 54 | 0.009328036 | ER+ |
| CID4398 | Cancer LumB SC | 0 | 0 | ER+ |
| CID3586 | Cycling PVL | 0 | 0 | HER2+ |
| CID3921 | Cycling PVL | 0 | 0 | HER2+ |
| CID45171 | Cycling PVL | 0 | 0 | HER2+ |
| CID3838 | Cycling PVL | 4 | 0.001699958 | HER2+ |
| CID4066 | Cycling PVL | 6 | 0.001130156 | HER2+ |
| CID44041 | Cycling PVL | 0 | 0 | TNBC |
| CID4465 | Cycling PVL | 7 | 0.004475703 | TNBC |
| CID4495 | Cycling PVL | 2 | 0.00025047 | TNBC |
| CID44971 | Cycling PVL | 0 | 0 | TNBC |
| CID44991 | Cycling PVL | 6 | 0.000854336 | TNBC |
| CID4513 | Cycling PVL | 2 | 0.000355935 | TNBC |
| CID4515 | Cycling PVL | 2 | 0.000482044 | TNBC |
| CID4523 | Cycling PVL | 0 | 0 | TNBC |
| CID3946 | Cycling PVL | 0 | 0 | TNBC |
| CID3963 | Cycling PVL | 1 | 0.000283527 | TNBC |
| CID4461 | Cycling PVL | 0 | 0 | ER+ |
| CID4463 | Cycling PVL | 0 | 0 | ER+ |
| CID4471 | Cycling PVL | 0 | 0 | ER+ |
| CID4530N | Cycling PVL | 0 | 0 | ER+ |
| CID4535 | Cycling PVL | 10 | 0.002524615 | ER+ |
| CID4040 | Cycling PVL | 4 | 0.001580403 | ER+ |
| CID3941 | Cycling PVL | 1 | 0.001584786 | ER+ |
| CID3948 | Cycling PVL | 0 | 0 | ER+ |
| CID4067 | Cycling PVL | 2 | 0.00053135 | ER+ |
| CID4290A | Cycling PVL | 1 | 0.000172741 | ER+ |
| CID4398 | Cycling PVL | 2 | 0.000449337 | ER+ |
| CID3586 | Cycling T-cells | 56 | 0.009064422 | HER2+ |
| CID3921 | Cycling T-cells | 34 | 0.011243386 | HER2+ |
| CID45171 | Cycling T-cells | 18 | 0.007355946 | HER2+ |
| CID3838 | Cycling T-cells | 42 | 0.017849554 | HER2+ |
| CID4066 | Cycling T-cells | 38 | 0.007157657 | HER2+ |
| CID44041 | Cycling T-cells | 5 | 0.002346316 | TNBC |
| CID4465 | Cycling T-cells | 10 | 0.006393862 | TNBC |
| CID4495 | Cycling T-cells | 430 | 0.053850971 | TNBC |
| CID44971 | Cycling T-cells | 271 | 0.033934385 | TNBC |
| CID44991 | Cycling T-cells | 149 | 0.021216005 | TNBC |
| CID4513 | Cycling T-cells | 181 | 0.032212137 | TNBC |
| CID4515 | Cycling T-cells | 20 | 0.004820439 | TNBC |
| CID4523 | Cycling T-cells | 14 | 0.007981756 | TNBC |
| CID3946 | Cycling T-cells | 1 | 0.00129199 | TNBC |
| CID3963 | Cycling T-cells | 73 | 0.020697477 | TNBC |
| CID4461 | Cycling T-cells | 8 | 0.012678288 | ER+ |
| CID4463 | Cycling T-cells | 5 | 0.004393673 | ER+ |
| CID4471 | Cycling T-cells | 8 | 0.00092926 | ER+ |
| CID4530N | Cycling T-cells | 1 | 0.000226809 | ER+ |
| CID4535 | Cycling T-cells | 19 | 0.004796768 | ER+ |
| CID4040 | Cycling T-cells | 25 | 0.009877519 | ER+ |
| CID3941 | Cycling T-cells | 5 | 0.00792393 | ER+ |
| CID3948 | Cycling T-cells | 24 | 0.010313709 | ER+ |
| CID4067 | Cycling T-cells | 6 | 0.001594049 | ER+ |
| CID4290A | Cycling T-cells | 8 | 0.001381931 | ER+ |
| CID4398 | Cycling T-cells | 77 | 0.017299483 | ER+ |
| CID3586 | Cycling_Myeloid | 11 | 0.001780511 | HER2+ |
| CID3921 | Cycling_Myeloid | 18 | 0.005952381 | HER2+ |
| CID45171 | Cycling_Myeloid | 2 | 0.000817327 | HER2+ |
| CID3838 | Cycling_Myeloid | 21 | 0.008924777 | HER2+ |
| CID4066 | Cycling_Myeloid | 10 | 0.001883594 | HER2+ |
| CID44041 | Cycling_Myeloid | 2 | 0.000938527 | TNBC |
| CID4465 | Cycling_Myeloid | 21 | 0.01342711 | TNBC |
| CID4495 | Cycling_Myeloid | 42 | 0.005259862 | TNBC |
| CID44971 | Cycling_Myeloid | 46 | 0.00576008 | TNBC |
| CID44991 | Cycling_Myeloid | 3 | 0.000427168 | TNBC |
| CID4513 | Cycling_Myeloid | 147 | 0.026161239 | TNBC |
| CID4515 | Cycling_Myeloid | 30 | 0.007230658 | TNBC |
| CID4523 | Cycling_Myeloid | 11 | 0.00627138 | TNBC |
| CID3946 | Cycling_Myeloid | 3 | 0.003875969 | TNBC |
| CID3963 | Cycling_Myeloid | 24 | 0.00680465 | TNBC |
| CID4461 | Cycling_Myeloid | 5 | 0.00792393 | ER+ |
| CID4463 | Cycling_Myeloid | 10 | 0.008787346 | ER+ |
| CID4471 | Cycling_Myeloid | 12 | 0.00139389 | ER+ |
| CID4530N | Cycling_Myeloid | 3 | 0.000680426 | ER+ |
| CID4535 | Cycling_Myeloid | 8 | 0.002019692 | ER+ |
| CID4040 | Cycling_Myeloid | 3 | 0.001185302 | ER+ |
| CID3941 | Cycling_Myeloid | 0 | 0 | ER+ |
| CID3948 | Cycling_Myeloid | 4 | 0.001718951 | ER+ |
| CID4067 | Cycling_Myeloid | 3 | 0.000797024 | ER+ |
| CID4290A | Cycling_Myeloid | 13 | 0.002245638 | ER+ |
| CID4398 | Cycling_Myeloid | 11 | 0.002471355 | ER+ |
| CID3586 | DCs | 56 | 0.009064422 | HER2+ |
| CID3921 | DCs | 52 | 0.017195767 | HER2+ |
| CID45171 | DCs | 21 | 0.008581937 | HER2+ |
| CID3838 | DCs | 32 | 0.01359966 | HER2+ |
| CID4066 | DCs | 31 | 0.005839141 | HER2+ |
| CID44041 | DCs | 19 | 0.008916002 | TNBC |
| CID4465 | DCs | 12 | 0.007672634 | TNBC |
| CID4495 | DCs | 99 | 0.012398247 | TNBC |
| CID44971 | DCs | 167 | 0.020911595 | TNBC |
| CID44991 | DCs | 23 | 0.003274954 | TNBC |
| CID4513 | DCs | 62 | 0.011033992 | TNBC |
| CID4515 | DCs | 63 | 0.015184382 | TNBC |
| CID4523 | DCs | 7 | 0.003990878 | TNBC |
| CID3946 | DCs | 2 | 0.002583979 | TNBC |
| CID3963 | DCs | 28 | 0.007938758 | TNBC |
| CID4461 | DCs | 6 | 0.009508716 | ER+ |
| CID4463 | DCs | 6 | 0.005272408 | ER+ |
| CID4471 | DCs | 35 | 0.004065513 | ER+ |
| CID4530N | DCs | 17 | 0.00385575 | ER+ |
| CID4535 | DCs | 56 | 0.014137844 | ER+ |
| CID4040 | DCs | 5 | 0.001975504 | ER+ |
| CID3941 | DCs | 1 | 0.001584786 | ER+ |
| CID3948 | DCs | 15 | 0.006446068 | ER+ |
| CID4067 | DCs | 32 | 0.008501594 | ER+ |
| CID4290A | DCs | 28 | 0.004836759 | ER+ |
| CID4398 | DCs | 80 | 0.017973489 | ER+ |
| CID3586 | Endothelial ACKR1 | 80 | 0.012949174 | HER2+ |
| CID3921 | Endothelial ACKR1 | 121 | 0.040013228 | HER2+ |
| CID45171 | Endothelial ACKR1 | 10 | 0.004086637 | HER2+ |
| CID3838 | Endothelial ACKR1 | 48 | 0.02039949 | HER2+ |
| CID4066 | Endothelial ACKR1 | 299 | 0.056319458 | HER2+ |
| CID44041 | Endothelial ACKR1 | 84 | 0.039418114 | TNBC |
| CID4465 | Endothelial ACKR1 | 192 | 0.122762148 | TNBC |
| CID4495 | Endothelial ACKR1 | 58 | 0.007263619 | TNBC |
| CID44971 | Endothelial ACKR1 | 106 | 0.013273228 | TNBC |
| CID44991 | Endothelial ACKR1 | 15 | 0.002135839 | TNBC |
| CID4513 | Endothelial ACKR1 | 74 | 0.013169603 | TNBC |
| CID4515 | Endothelial ACKR1 | 77 | 0.018558689 | TNBC |
| CID4523 | Endothelial ACKR1 | 1 | 0.000570125 | TNBC |
| CID3946 | Endothelial ACKR1 | 65 | 0.083979328 | TNBC |
| CID3963 | Endothelial ACKR1 | 59 | 0.016728098 | TNBC |
| CID4461 | Endothelial ACKR1 | 106 | 0.167987322 | ER+ |
| CID4463 | Endothelial ACKR1 | 43 | 0.037785589 | ER+ |
| CID4471 | Endothelial ACKR1 | 2065 | 0.239865257 | ER+ |
| CID4530N | Endothelial ACKR1 | 573 | 0.129961443 | ER+ |
| CID4535 | Endothelial ACKR1 | 44 | 0.011108306 | ER+ |
| CID4040 | Endothelial ACKR1 | 98 | 0.038719874 | ER+ |
| CID3941 | Endothelial ACKR1 | 24 | 0.038034865 | ER+ |
| CID3948 | Endothelial ACKR1 | 43 | 0.018478728 | ER+ |
| CID4067 | Endothelial ACKR1 | 111 | 0.029489904 | ER+ |
| CID4290A | Endothelial ACKR1 | 158 | 0.027293142 | ER+ |
| CID4398 | Endothelial ACKR1 | 57 | 0.012806111 | ER+ |
| CID3586 | Endothelial CXCL12 | 38 | 0.006150858 | HER2+ |
| CID3921 | Endothelial CXCL12 | 44 | 0.014550265 | HER2+ |
| CID45171 | Endothelial CXCL12 | 3 | 0.001225991 | HER2+ |
| CID3838 | Endothelial CXCL12 | 24 | 0.010199745 | HER2+ |
| CID4066 | Endothelial CXCL12 | 142 | 0.026747033 | HER2+ |
| CID44041 | Endothelial CXCL12 | 32 | 0.015016424 | TNBC |
| CID4465 | Endothelial CXCL12 | 52 | 0.033248082 | TNBC |
| CID4495 | Endothelial CXCL12 | 64 | 0.008015028 | TNBC |
| CID44971 | Endothelial CXCL12 | 47 | 0.005885299 | TNBC |
| CID44991 | Endothelial CXCL12 | 13 | 0.001851061 | TNBC |
| CID4513 | Endothelial CXCL12 | 44 | 0.007830575 | TNBC |
| CID4515 | Endothelial CXCL12 | 27 | 0.006507592 | TNBC |
| CID4523 | Endothelial CXCL12 | 1 | 0.000570125 | TNBC |
| CID3946 | Endothelial CXCL12 | 28 | 0.036175711 | TNBC |
| CID3963 | Endothelial CXCL12 | 25 | 0.007088177 | TNBC |
| CID4461 | Endothelial CXCL12 | 42 | 0.066561014 | ER+ |
| CID4463 | Endothelial CXCL12 | 23 | 0.020210896 | ER+ |
| CID4471 | Endothelial CXCL12 | 359 | 0.041700546 | ER+ |
| CID4530N | Endothelial CXCL12 | 268 | 0.060784758 | ER+ |
| CID4535 | Endothelial CXCL12 | 128 | 0.032315072 | ER+ |
| CID4040 | Endothelial CXCL12 | 67 | 0.02647175 | ER+ |
| CID3941 | Endothelial CXCL12 | 11 | 0.017432647 | ER+ |
| CID3948 | Endothelial CXCL12 | 22 | 0.009454233 | ER+ |
| CID4067 | Endothelial CXCL12 | 34 | 0.009032944 | ER+ |
| CID4290A | Endothelial CXCL12 | 72 | 0.012437381 | ER+ |
| CID4398 | Endothelial CXCL12 | 34 | 0.007638733 | ER+ |
| CID3586 | Endothelial Lymphatic LYVE1 | 10 | 0.001618647 | HER2+ |
| CID3921 | Endothelial Lymphatic LYVE1 | 10 | 0.003306878 | HER2+ |
| CID45171 | Endothelial Lymphatic LYVE1 | 0 | 0 | HER2+ |
| CID3838 | Endothelial Lymphatic LYVE1 | 4 | 0.001699958 | HER2+ |
| CID4066 | Endothelial Lymphatic LYVE1 | 7 | 0.001318516 | HER2+ |
| CID44041 | Endothelial Lymphatic LYVE1 | 6 | 0.00281558 | TNBC |
| CID4465 | Endothelial Lymphatic LYVE1 | 14 | 0.008951407 | TNBC |
| CID4495 | Endothelial Lymphatic LYVE1 | 28 | 0.003506575 | TNBC |
| CID44971 | Endothelial Lymphatic LYVE1 | 12 | 0.00150263 | TNBC |
| CID44991 | Endothelial Lymphatic LYVE1 | 1 | 0.000142389 | TNBC |
| CID4513 | Endothelial Lymphatic LYVE1 | 0 | 0 | TNBC |
| CID4515 | Endothelial Lymphatic LYVE1 | 3 | 0.000723066 | TNBC |
| CID4523 | Endothelial Lymphatic LYVE1 | 0 | 0 | TNBC |
| CID3946 | Endothelial Lymphatic LYVE1 | 0 | 0 | TNBC |
| CID3963 | Endothelial Lymphatic LYVE1 | 0 | 0 | TNBC |
| CID4461 | Endothelial Lymphatic LYVE1 | 3 | 0.004754358 | ER+ |
| CID4463 | Endothelial Lymphatic LYVE1 | 2 | 0.001757469 | ER+ |
| CID4471 | Endothelial Lymphatic LYVE1 | 46 | 0.005343245 | ER+ |
| CID4530N | Endothelial Lymphatic LYVE1 | 20 | 0.004536176 | ER+ |
| CID4535 | Endothelial Lymphatic LYVE1 | 5 | 0.001262307 | ER+ |
| CID4040 | Endothelial Lymphatic LYVE1 | 13 | 0.00513631 | ER+ |
| CID3941 | Endothelial Lymphatic LYVE1 | 1 | 0.001584786 | ER+ |
| CID3948 | Endothelial Lymphatic LYVE1 | 6 | 0.002578427 | ER+ |
| CID4067 | Endothelial Lymphatic LYVE1 | 2 | 0.00053135 | ER+ |
| CID4290A | Endothelial Lymphatic LYVE1 | 5 | 0.000863707 | ER+ |
| CID4398 | Endothelial Lymphatic LYVE1 | 5 | 0.001123343 | ER+ |
| CID3586 | Endothelial RGS5 | 29 | 0.004694076 | HER2+ |
| CID3921 | Endothelial RGS5 | 35 | 0.011574074 | HER2+ |
| CID45171 | Endothelial RGS5 | 2 | 0.000817327 | HER2+ |
| CID3838 | Endothelial RGS5 | 23 | 0.009774756 | HER2+ |
| CID4066 | Endothelial RGS5 | 87 | 0.016387267 | HER2+ |
| CID44041 | Endothelial RGS5 | 26 | 0.012200845 | TNBC |
| CID4465 | Endothelial RGS5 | 36 | 0.023017903 | TNBC |
| CID4495 | Endothelial RGS5 | 34 | 0.004257984 | TNBC |
| CID44971 | Endothelial RGS5 | 52 | 0.006511395 | TNBC |
| CID44991 | Endothelial RGS5 | 12 | 0.001708672 | TNBC |
| CID4513 | Endothelial RGS5 | 44 | 0.007830575 | TNBC |
| CID4515 | Endothelial RGS5 | 15 | 0.003615329 | TNBC |
| CID4523 | Endothelial RGS5 | 1 | 0.000570125 | TNBC |
| CID3946 | Endothelial RGS5 | 17 | 0.021963824 | TNBC |
| CID3963 | Endothelial RGS5 | 18 | 0.005103487 | TNBC |
| CID4461 | Endothelial RGS5 | 31 | 0.049128368 | ER+ |
| CID4463 | Endothelial RGS5 | 11 | 0.009666081 | ER+ |
| CID4471 | Endothelial RGS5 | 308 | 0.035776513 | ER+ |
| CID4530N | Endothelial RGS5 | 155 | 0.035155364 | ER+ |
| CID4535 | Endothelial RGS5 | 42 | 0.010603383 | ER+ |
| CID4040 | Endothelial RGS5 | 40 | 0.01580403 | ER+ |
| CID3941 | Endothelial RGS5 | 8 | 0.012678288 | ER+ |
| CID3948 | Endothelial RGS5 | 14 | 0.00601633 | ER+ |
| CID4067 | Endothelial RGS5 | 39 | 0.010361318 | ER+ |
| CID4290A | Endothelial RGS5 | 63 | 0.010882709 | ER+ |
| CID4398 | Endothelial RGS5 | 5 | 0.001123343 | ER+ |
| CID3586 | Luminal Progenitors | 471 | 0.076238265 | HER2+ |
| CID3921 | Luminal Progenitors | 0 | 0 | HER2+ |
| CID45171 | Luminal Progenitors | 0 | 0 | HER2+ |
| CID3838 | Luminal Progenitors | 0 | 0 | HER2+ |
| CID4066 | Luminal Progenitors | 106 | 0.019966095 | HER2+ |
| CID44041 | Luminal Progenitors | 57 | 0.026748006 | TNBC |
| CID4465 | Luminal Progenitors | 4 | 0.002557545 | TNBC |
| CID4495 | Luminal Progenitors | 0 | 0 | TNBC |
| CID44971 | Luminal Progenitors | 442 | 0.055346857 | TNBC |
| CID44991 | Luminal Progenitors | 11 | 0.001566282 | TNBC |
| CID4513 | Luminal Progenitors | 0 | 0 | TNBC |
| CID4515 | Luminal Progenitors | 9 | 0.002169197 | TNBC |
| CID4523 | Luminal Progenitors | 0 | 0 | TNBC |
| CID3946 | Luminal Progenitors | 0 | 0 | TNBC |
| CID3963 | Luminal Progenitors | 1 | 0.000283527 | TNBC |
| CID4461 | Luminal Progenitors | 0 | 0 | ER+ |
| CID4463 | Luminal Progenitors | 12 | 0.010544815 | ER+ |
| CID4471 | Luminal Progenitors | 655 | 0.076083169 | ER+ |
| CID4530N | Luminal Progenitors | 207 | 0.046949422 | ER+ |
| CID4535 | Luminal Progenitors | 7 | 0.00176723 | ER+ |
| CID4040 | Luminal Progenitors | 0 | 0 | ER+ |
| CID3941 | Luminal Progenitors | 0 | 0 | ER+ |
| CID3948 | Luminal Progenitors | 0 | 0 | ER+ |
| CID4067 | Luminal Progenitors | 0 | 0 | ER+ |
| CID4290A | Luminal Progenitors | 10 | 0.001727414 | ER+ |
| CID4398 | Luminal Progenitors | 0 | 0 | ER+ |
| CID3586 | Macrophage | 95 | 0.015377145 | HER2+ |
| CID3921 | Macrophage | 232 | 0.076719577 | HER2+ |
| CID45171 | Macrophage | 17 | 0.006947282 | HER2+ |
| CID3838 | Macrophage | 319 | 0.135571611 | HER2+ |
| CID4066 | Macrophage | 133 | 0.025051799 | HER2+ |
| CID44041 | Macrophage | 66 | 0.030971375 | TNBC |
| CID4465 | Macrophage | 112 | 0.071611253 | TNBC |
| CID4495 | Macrophage | 547 | 0.068503444 | TNBC |
| CID44971 | Macrophage | 316 | 0.039569246 | TNBC |
| CID44991 | Macrophage | 145 | 0.020646447 | TNBC |
| CID4513 | Macrophage | 1894 | 0.337070653 | TNBC |
| CID4515 | Macrophage | 276 | 0.066522054 | TNBC |
| CID4523 | Macrophage | 207 | 0.118015964 | TNBC |
| CID3946 | Macrophage | 108 | 0.139534884 | TNBC |
| CID3963 | Macrophage | 356 | 0.100935639 | TNBC |
| CID4461 | Macrophage | 36 | 0.057052298 | ER+ |
| CID4463 | Macrophage | 67 | 0.05887522 | ER+ |
| CID4471 | Macrophage | 186 | 0.021605297 | ER+ |
| CID4530N | Macrophage | 47 | 0.010660014 | ER+ |
| CID4535 | Macrophage | 119 | 0.030042918 | ER+ |
| CID4040 | Macrophage | 31 | 0.012248123 | ER+ |
| CID3941 | Macrophage | 28 | 0.04437401 | ER+ |
| CID3948 | Macrophage | 76 | 0.032660077 | ER+ |
| CID4067 | Macrophage | 189 | 0.05021254 | ER+ |
| CID4290A | Macrophage | 249 | 0.04301261 | ER+ |
| CID4398 | Macrophage | 78 | 0.017524152 | ER+ |
| CID3586 | Mature Luminal | 91 | 0.014729686 | HER2+ |
| CID3921 | Mature Luminal | 0 | 0 | HER2+ |
| CID45171 | Mature Luminal | 0 | 0 | HER2+ |
| CID3838 | Mature Luminal | 0 | 0 | HER2+ |
| CID4066 | Mature Luminal | 61 | 0.011489923 | HER2+ |
| CID44041 | Mature Luminal | 85 | 0.039887377 | TNBC |
| CID4465 | Mature Luminal | 6 | 0.003836317 | TNBC |
| CID4495 | Mature Luminal | 0 | 0 | TNBC |
| CID44971 | Mature Luminal | 169 | 0.021162034 | TNBC |
| CID44991 | Mature Luminal | 9 | 0.001281504 | TNBC |
| CID4513 | Mature Luminal | 0 | 0 | TNBC |
| CID4515 | Mature Luminal | 18 | 0.004338395 | TNBC |
| CID4523 | Mature Luminal | 0 | 0 | TNBC |
| CID3946 | Mature Luminal | 0 | 0 | TNBC |
| CID3963 | Mature Luminal | 0 | 0 | TNBC |
| CID4461 | Mature Luminal | 0 | 0 | ER+ |
| CID4463 | Mature Luminal | 10 | 0.008787346 | ER+ |
| CID4471 | Mature Luminal | 654 | 0.075967011 | ER+ |
| CID4530N | Mature Luminal | 145 | 0.032887276 | ER+ |
| CID4535 | Mature Luminal | 13 | 0.003281999 | ER+ |
| CID4040 | Mature Luminal | 0 | 0 | ER+ |
| CID3941 | Mature Luminal | 0 | 0 | ER+ |
| CID3948 | Mature Luminal | 0 | 0 | ER+ |
| CID4067 | Mature Luminal | 0 | 0 | ER+ |
| CID4290A | Mature Luminal | 4 | 0.000690966 | ER+ |
| CID4398 | Mature Luminal | 0 | 0 | ER+ |
| CID3586 | Monocyte | 38 | 0.006150858 | HER2+ |
| CID3921 | Monocyte | 83 | 0.02744709 | HER2+ |
| CID45171 | Monocyte | 132 | 0.053943604 | HER2+ |
| CID3838 | Monocyte | 72 | 0.030599235 | HER2+ |
| CID4066 | Monocyte | 47 | 0.008852891 | HER2+ |
| CID44041 | Monocyte | 18 | 0.008446739 | TNBC |
| CID4465 | Monocyte | 36 | 0.023017903 | TNBC |
| CID4495 | Monocyte | 209 | 0.026174076 | TNBC |
| CID44971 | Monocyte | 155 | 0.019408966 | TNBC |
| CID44991 | Monocyte | 35 | 0.004983625 | TNBC |
| CID4513 | Monocyte | 692 | 0.123153586 | TNBC |
| CID4515 | Monocyte | 194 | 0.046758255 | TNBC |
| CID4523 | Monocyte | 130 | 0.074116306 | TNBC |
| CID3946 | Monocyte | 44 | 0.056847545 | TNBC |
| CID3963 | Monocyte | 71 | 0.020130422 | TNBC |
| CID4461 | Monocyte | 6 | 0.009508716 | ER+ |
| CID4463 | Monocyte | 18 | 0.015817223 | ER+ |
| CID4471 | Monocyte | 52 | 0.00604019 | ER+ |
| CID4530N | Monocyte | 29 | 0.006577455 | ER+ |
| CID4535 | Monocyte | 72 | 0.018177228 | ER+ |
| CID4040 | Monocyte | 11 | 0.004346108 | ER+ |
| CID3941 | Monocyte | 8 | 0.012678288 | ER+ |
| CID3948 | Monocyte | 27 | 0.011602922 | ER+ |
| CID4067 | Monocyte | 42 | 0.011158342 | ER+ |
| CID4290A | Monocyte | 51 | 0.008809812 | ER+ |
| CID4398 | Monocyte | 56 | 0.012581442 | ER+ |
| CID3586 | Myoepithelial | 136 | 0.022013597 | HER2+ |
| CID3921 | Myoepithelial | 0 | 0 | HER2+ |
| CID45171 | Myoepithelial | 0 | 0 | HER2+ |
| CID3838 | Myoepithelial | 0 | 0 | HER2+ |
| CID4066 | Myoepithelial | 103 | 0.019401017 | HER2+ |
| CID44041 | Myoepithelial | 9 | 0.004223369 | TNBC |
| CID4465 | Myoepithelial | 0 | 0 | TNBC |
| CID4495 | Myoepithelial | 0 | 0 | TNBC |
| CID44971 | Myoepithelial | 124 | 0.015527173 | TNBC |
| CID44991 | Myoepithelial | 4 | 0.000569557 | TNBC |
| CID4513 | Myoepithelial | 0 | 0 | TNBC |
| CID4515 | Myoepithelial | 9 | 0.002169197 | TNBC |
| CID4523 | Myoepithelial | 0 | 0 | TNBC |
| CID3946 | Myoepithelial | 0 | 0 | TNBC |
| CID3963 | Myoepithelial | 0 | 0 | TNBC |
| CID4461 | Myoepithelial | 0 | 0 | ER+ |
| CID4463 | Myoepithelial | 4 | 0.003514938 | ER+ |
| CID4471 | Myoepithelial | 657 | 0.076315484 | ER+ |
| CID4530N | Myoepithelial | 46 | 0.010433205 | ER+ |
| CID4535 | Myoepithelial | 2 | 0.000504923 | ER+ |
| CID4040 | Myoepithelial | 0 | 0 | ER+ |
| CID3941 | Myoepithelial | 0 | 0 | ER+ |
| CID3948 | Myoepithelial | 0 | 0 | ER+ |
| CID4067 | Myoepithelial | 0 | 0 | ER+ |
| CID4290A | Myoepithelial | 4 | 0.000690966 | ER+ |
| CID4398 | Myoepithelial | 0 | 0 | ER+ |
| CID3586 | NK cells | 130 | 0.021042409 | HER2+ |
| CID3921 | NK cells | 60 | 0.01984127 | HER2+ |
| CID45171 | NK cells | 87 | 0.035553739 | HER2+ |
| CID3838 | NK cells | 75 | 0.031874203 | HER2+ |
| CID4066 | NK cells | 101 | 0.019024298 | HER2+ |
| CID44041 | NK cells | 21 | 0.009854528 | TNBC |
| CID4465 | NK cells | 2 | 0.001278772 | TNBC |
| CID4495 | NK cells | 52 | 0.00651221 | TNBC |
| CID44971 | NK cells | 94 | 0.011770599 | TNBC |
| CID44991 | NK cells | 20 | 0.002847786 | TNBC |
| CID4513 | NK cells | 205 | 0.03648336 | TNBC |
| CID4515 | NK cells | 41 | 0.009881899 | TNBC |
| CID4523 | NK cells | 44 | 0.025085519 | TNBC |
| CID3946 | NK cells | 1 | 0.00129199 | TNBC |
| CID3963 | NK cells | 273 | 0.077402892 | TNBC |
| CID4461 | NK cells | 2 | 0.003169572 | ER+ |
| CID4463 | NK cells | 3 | 0.002636204 | ER+ |
| CID4471 | NK cells | 30 | 0.003484725 | ER+ |
| CID4530N | NK cells | 11 | 0.002494897 | ER+ |
| CID4535 | NK cells | 25 | 0.006311537 | ER+ |
| CID4040 | NK cells | 107 | 0.04227578 | ER+ |
| CID3941 | NK cells | 18 | 0.028526149 | ER+ |
| CID3948 | NK cells | 58 | 0.024924796 | ER+ |
| CID4067 | NK cells | 48 | 0.012752391 | ER+ |
| CID4290A | NK cells | 50 | 0.00863707 | ER+ |
| CID4398 | NK cells | 288 | 0.064704561 | ER+ |
| CID3586 | NKT cells | 95 | 0.015377145 | HER2+ |
| CID3921 | NKT cells | 17 | 0.005621693 | HER2+ |
| CID45171 | NKT cells | 206 | 0.084184716 | HER2+ |
| CID3838 | NKT cells | 28 | 0.011899703 | HER2+ |
| CID4066 | NKT cells | 39 | 0.007346016 | HER2+ |
| CID44041 | NKT cells | 6 | 0.00281558 | TNBC |
| CID4465 | NKT cells | 5 | 0.003196931 | TNBC |
| CID4495 | NKT cells | 31 | 0.003882279 | TNBC |
| CID44971 | NKT cells | 43 | 0.005384423 | TNBC |
| CID44991 | NKT cells | 47 | 0.006692297 | TNBC |
| CID4513 | NKT cells | 45 | 0.008008542 | TNBC |
| CID4515 | NKT cells | 73 | 0.017594601 | TNBC |
| CID4523 | NKT cells | 12 | 0.006841505 | TNBC |
| CID3946 | NKT cells | 4 | 0.005167959 | TNBC |
| CID3963 | NKT cells | 94 | 0.026651545 | TNBC |
| CID4461 | NKT cells | 3 | 0.004754358 | ER+ |
| CID4463 | NKT cells | 19 | 0.016695958 | ER+ |
| CID4471 | NKT cells | 32 | 0.00371704 | ER+ |
| CID4530N | NKT cells | 45 | 0.010206396 | ER+ |
| CID4535 | NKT cells | 15 | 0.003786922 | ER+ |
| CID4040 | NKT cells | 22 | 0.008692217 | ER+ |
| CID3941 | NKT cells | 9 | 0.014263074 | ER+ |
| CID3948 | NKT cells | 40 | 0.017189514 | ER+ |
| CID4067 | NKT cells | 39 | 0.010361318 | ER+ |
| CID4290A | NKT cells | 24 | 0.004145794 | ER+ |
| CID4398 | NKT cells | 129 | 0.028982251 | ER+ |
| CID3586 | Plasmablasts | 0 | 0 | HER2+ |
| CID3921 | Plasmablasts | 175 | 0.05787037 | HER2+ |
| CID45171 | Plasmablasts | 0 | 0 | HER2+ |
| CID3838 | Plasmablasts | 51 | 0.021674458 | HER2+ |
| CID4066 | Plasmablasts | 0 | 0 | HER2+ |
| CID44041 | Plasmablasts | 0 | 0 | TNBC |
| CID4465 | Plasmablasts | 110 | 0.070332481 | TNBC |
| CID4495 | Plasmablasts | 1020 | 0.127739512 | TNBC |
| CID44971 | Plasmablasts | 48 | 0.006010518 | TNBC |
| CID44991 | Plasmablasts | 1453 | 0.206891642 | TNBC |
| CID4513 | Plasmablasts | 0 | 0 | TNBC |
| CID4515 | Plasmablasts | 36 | 0.00867679 | TNBC |
| CID4523 | Plasmablasts | 0 | 0 | TNBC |
| CID3946 | Plasmablasts | 0 | 0 | TNBC |
| CID3963 | Plasmablasts | 0 | 0 | TNBC |
| CID4461 | Plasmablasts | 32 | 0.050713154 | ER+ |
| CID4463 | Plasmablasts | 0 | 0 | ER+ |
| CID4471 | Plasmablasts | 51 | 0.005924033 | ER+ |
| CID4530N | Plasmablasts | 55 | 0.012474484 | ER+ |
| CID4535 | Plasmablasts | 96 | 0.024236304 | ER+ |
| CID4040 | Plasmablasts | 74 | 0.029237456 | ER+ |
| CID3941 | Plasmablasts | 0 | 0 | ER+ |
| CID3948 | Plasmablasts | 232 | 0.099699183 | ER+ |
| CID4067 | Plasmablasts | 0 | 0 | ER+ |
| CID4290A | Plasmablasts | 0 | 0 | ER+ |
| CID4398 | Plasmablasts | 91 | 0.020444844 | ER+ |
| CID3586 | PVL Differentiated | 10 | 0.001618647 | HER2+ |
| CID3921 | PVL Differentiated | 46 | 0.01521164 | HER2+ |
| CID45171 | PVL Differentiated | 10 | 0.004086637 | HER2+ |
| CID3838 | PVL Differentiated | 82 | 0.034849129 | HER2+ |
| CID4066 | PVL Differentiated | 402 | 0.075720475 | HER2+ |
| CID44041 | PVL Differentiated | 66 | 0.030971375 | TNBC |
| CID4465 | PVL Differentiated | 187 | 0.119565217 | TNBC |
| CID4495 | PVL Differentiated | 112 | 0.014026299 | TNBC |
| CID44971 | PVL Differentiated | 29 | 0.003631355 | TNBC |
| CID44991 | PVL Differentiated | 20 | 0.002847786 | TNBC |
| CID4513 | PVL Differentiated | 49 | 0.008720413 | TNBC |
| CID4515 | PVL Differentiated | 86 | 0.020727886 | TNBC |
| CID4523 | PVL Differentiated | 5 | 0.002850627 | TNBC |
| CID3946 | PVL Differentiated | 175 | 0.226098191 | TNBC |
| CID3963 | PVL Differentiated | 12 | 0.003402325 | TNBC |
| CID4461 | PVL Differentiated | 38 | 0.06022187 | ER+ |
| CID4463 | PVL Differentiated | 26 | 0.0228471 | ER+ |
| CID4471 | PVL Differentiated | 868 | 0.100824718 | ER+ |
| CID4530N | PVL Differentiated | 340 | 0.077114992 | ER+ |
| CID4535 | PVL Differentiated | 430 | 0.108558445 | ER+ |
| CID4040 | PVL Differentiated | 271 | 0.107072303 | ER+ |
| CID3941 | PVL Differentiated | 12 | 0.019017433 | ER+ |
| CID3948 | PVL Differentiated | 28 | 0.01203266 | ER+ |
| CID4067 | PVL Differentiated | 43 | 0.011424017 | ER+ |
| CID4290A | PVL Differentiated | 79 | 0.013646571 | ER+ |
| CID4398 | PVL Differentiated | 61 | 0.013704785 | ER+ |
| CID3586 | PVL Immature | 11 | 0.001780511 | HER2+ |
| CID3921 | PVL Immature | 26 | 0.008597884 | HER2+ |
| CID45171 | PVL Immature | 3 | 0.001225991 | HER2+ |
| CID3838 | PVL Immature | 72 | 0.030599235 | HER2+ |
| CID4066 | PVL Immature | 222 | 0.041815785 | HER2+ |
| CID44041 | PVL Immature | 62 | 0.029094322 | TNBC |
| CID4465 | PVL Immature | 123 | 0.078644501 | TNBC |
| CID4495 | PVL Immature | 77 | 0.009643081 | TNBC |
| CID44971 | PVL Immature | 62 | 0.007763586 | TNBC |
| CID44991 | PVL Immature | 20 | 0.002847786 | TNBC |
| CID4513 | PVL Immature | 31 | 0.005516996 | TNBC |
| CID4515 | PVL Immature | 35 | 0.008435768 | TNBC |
| CID4523 | PVL Immature | 5 | 0.002850627 | TNBC |
| CID3946 | PVL Immature | 73 | 0.094315245 | TNBC |
| CID3963 | PVL Immature | 15 | 0.004252906 | TNBC |
| CID4461 | PVL Immature | 10 | 0.015847861 | ER+ |
| CID4463 | PVL Immature | 5 | 0.004393673 | ER+ |
| CID4471 | PVL Immature | 417 | 0.048437681 | ER+ |
| CID4530N | PVL Immature | 129 | 0.029258335 | ER+ |
| CID4535 | PVL Immature | 152 | 0.038374148 | ER+ |
| CID4040 | PVL Immature | 168 | 0.066376926 | ER+ |
| CID3941 | PVL Immature | 12 | 0.019017433 | ER+ |
| CID3948 | PVL Immature | 34 | 0.014611087 | ER+ |
| CID4067 | PVL Immature | 38 | 0.010095643 | ER+ |
| CID4290A | PVL Immature | 60 | 0.010364484 | ER+ |
| CID4398 | PVL Immature | 24 | 0.005392047 | ER+ |
| CID3586 | T cells CD4+ | 2908 | 0.470702493 | HER2+ |
| CID3921 | T cells CD4+ | 975 | 0.322420635 | HER2+ |
| CID45171 | T cells CD4+ | 704 | 0.287699224 | HER2+ |
| CID3838 | T cells CD4+ | 915 | 0.388865278 | HER2+ |
| CID4066 | T cells CD4+ | 1108 | 0.208702204 | HER2+ |
| CID44041 | T cells CD4+ | 432 | 0.202721727 | TNBC |
| CID4465 | T cells CD4+ | 64 | 0.040920716 | TNBC |
| CID4495 | T cells CD4+ | 1741 | 0.218033813 | TNBC |
| CID44971 | T cells CD4+ | 2247 | 0.281367393 | TNBC |
| CID44991 | T cells CD4+ | 470 | 0.066922967 | TNBC |
| CID4513 | T cells CD4+ | 597 | 0.106246663 | TNBC |
| CID4515 | T cells CD4+ | 164 | 0.039527597 | TNBC |
| CID4523 | T cells CD4+ | 60 | 0.034207526 | TNBC |
| CID3946 | T cells CD4+ | 50 | 0.064599483 | TNBC |
| CID3963 | T cells CD4+ | 1065 | 0.301956337 | TNBC |
| CID4461 | T cells CD4+ | 44 | 0.069730586 | ER+ |
| CID4463 | T cells CD4+ | 115 | 0.101054482 | ER+ |
| CID4471 | T cells CD4+ | 412 | 0.047856894 | ER+ |
| CID4530N | T cells CD4+ | 127 | 0.028804718 | ER+ |
| CID4535 | T cells CD4+ | 239 | 0.060338298 | ER+ |
| CID4040 | T cells CD4+ | 830 | 0.327933623 | ER+ |
| CID3941 | T cells CD4+ | 108 | 0.171156894 | ER+ |
| CID3948 | T cells CD4+ | 845 | 0.363128492 | ER+ |
| CID4067 | T cells CD4+ | 353 | 0.093783209 | ER+ |
| CID4290A | T cells CD4+ | 345 | 0.059595785 | ER+ |
| CID4398 | T cells CD4+ | 2313 | 0.519658504 | ER+ |
| CID3586 | T cells CD8+ | 1407 | 0.227743606 | HER2+ |
| CID3921 | T cells CD8+ | 387 | 0.12797619 | HER2+ |
| CID45171 | T cells CD8+ | 331 | 0.135267675 | HER2+ |
| CID3838 | T cells CD8+ | 291 | 0.123671908 | HER2+ |
| CID4066 | T cells CD8+ | 885 | 0.16669806 | HER2+ |
| CID44041 | T cells CD8+ | 278 | 0.130455185 | TNBC |
| CID4465 | T cells CD8+ | 35 | 0.022378517 | TNBC |
| CID4495 | T cells CD8+ | 1250 | 0.156543519 | TNBC |
| CID44971 | T cells CD8+ | 1711 | 0.214249937 | TNBC |
| CID44991 | T cells CD8+ | 216 | 0.030756087 | TNBC |
| CID4513 | T cells CD8+ | 438 | 0.077949813 | TNBC |
| CID4515 | T cells CD8+ | 121 | 0.029163654 | TNBC |
| CID4523 | T cells CD8+ | 47 | 0.026795895 | TNBC |
| CID3946 | T cells CD8+ | 36 | 0.046511628 | TNBC |
| CID3963 | T cells CD8+ | 1167 | 0.330876099 | TNBC |
| CID4461 | T cells CD8+ | 11 | 0.017432647 | ER+ |
| CID4463 | T cells CD8+ | 75 | 0.065905097 | ER+ |
| CID4471 | T cells CD8+ | 159 | 0.018469044 | ER+ |
| CID4530N | T cells CD8+ | 108 | 0.02449535 | ER+ |
| CID4535 | T cells CD8+ | 98 | 0.024741227 | ER+ |
| CID4040 | T cells CD8+ | 528 | 0.208613196 | ER+ |
| CID3941 | T cells CD8+ | 126 | 0.199683043 | ER+ |
| CID3948 | T cells CD8+ | 498 | 0.214009454 | ER+ |
| CID4067 | T cells CD8+ | 243 | 0.06455898 | ER+ |
| CID4290A | T cells CD8+ | 115 | 0.019865262 | ER+ |
| CID4398 | T cells CD8+ | 926 | 0.208043136 | ER+ |
| CID3586 | B cells Memory | 289 | 0.046778893 | HER2+ |
| CID3921 | B cells Memory | 159 | 0.052579365 | HER2+ |
| CID45171 | B cells Memory | 56 | 0.022885166 | HER2+ |
| CID3838 | B cells Memory | 45 | 0.019124522 | HER2+ |
| CID4066 | B cells Memory | 38 | 0.007157657 | HER2+ |
| CID44041 | B cells Memory | 176 | 0.082590333 | TNBC |
| CID4465 | B cells Memory | 33 | 0.021099744 | TNBC |
| CID4495 | B cells Memory | 526 | 0.065873513 | TNBC |
| CID44971 | B cells Memory | 273 | 0.034184823 | TNBC |
| CID44991 | B cells Memory | 83 | 0.011818311 | TNBC |
| CID4513 | B cells Memory | 43 | 0.007652607 | TNBC |
| CID4515 | B cells Memory | 258 | 0.062183659 | TNBC |
| CID4523 | B cells Memory | 0 | 0 | TNBC |
| CID3946 | B cells Memory | 0 | 0 | TNBC |
| CID3963 | B cells Memory | 0 | 0 | TNBC |
| CID4461 | B cells Memory | 0 | 0 | ER+ |
| CID4463 | B cells Memory | 0 | 0 | ER+ |
| CID4471 | B cells Memory | 99 | 0.011499593 | ER+ |
| CID4530N | B cells Memory | 0 | 0 | ER+ |
| CID4535 | B cells Memory | 56 | 0.014137844 | ER+ |
| CID4040 | B cells Memory | 102 | 0.040300277 | ER+ |
| CID3941 | B cells Memory | 55 | 0.087163233 | ER+ |
| CID3948 | B cells Memory | 84 | 0.03609798 | ER+ |
| CID4067 | B cells Memory | 53 | 0.014080765 | ER+ |
| CID4290A | B cells Memory | 117 | 0.020210745 | ER+ |
| CID4398 | B cells Memory | 36 | 0.00808807 | ER+ |
| CID3586 | B cells Naive | 32 | 0.00517967 | HER2+ |
| CID3921 | B cells Naive | 3 | 0.000992063 | HER2+ |
| CID45171 | B cells Naive | 0 | 0 | HER2+ |
| CID3838 | B cells Naive | 2 | 0.000849979 | HER2+ |
| CID4066 | B cells Naive | 0 | 0 | HER2+ |
| CID44041 | B cells Naive | 0 | 0 | TNBC |
| CID4465 | B cells Naive | 0 | 0 | TNBC |
| CID4495 | B cells Naive | 247 | 0.030932999 | TNBC |
| CID44971 | B cells Naive | 96 | 0.012021037 | TNBC |
| CID44991 | B cells Naive | 5 | 0.000711946 | TNBC |
| CID4513 | B cells Naive | 0 | 0 | TNBC |
| CID4515 | B cells Naive | 236 | 0.056881176 | TNBC |
| CID4523 | B cells Naive | 0 | 0 | TNBC |
| CID3946 | B cells Naive | 0 | 0 | TNBC |
| CID3963 | B cells Naive | 0 | 0 | TNBC |
| CID4461 | B cells Naive | 0 | 0 | ER+ |
| CID4463 | B cells Naive | 0 | 0 | ER+ |
| CID4471 | B cells Naive | 0 | 0 | ER+ |
| CID4530N | B cells Naive | 0 | 0 | ER+ |
| CID4535 | B cells Naive | 0 | 0 | ER+ |
| CID4040 | B cells Naive | 3 | 0.001185302 | ER+ |
| CID3941 | B cells Naive | 0 | 0 | ER+ |
| CID3948 | B cells Naive | 1 | 0.000429738 | ER+ |
| CID4067 | B cells Naive | 0 | 0 | ER+ |
| CID4290A | B cells Naive | 0 | 0 | ER+ |
| CID4398 | B cells Naive | 0 | 0 | ER+ |
| CID3586 | CAFs MSC iCAF-like s1 | 59 | 0.009550016 | HER2+ |
| CID3921 | CAFs MSC iCAF-like s1 | 38 | 0.012566138 | HER2+ |
| CID45171 | CAFs MSC iCAF-like s1 | 7 | 0.002860646 | HER2+ |
| CID3838 | CAFs MSC iCAF-like s1 | 42 | 0.017849554 | HER2+ |
| CID4066 | CAFs MSC iCAF-like s1 | 272 | 0.051233754 | HER2+ |
| CID44041 | CAFs MSC iCAF-like s1 | 227 | 0.106522759 | TNBC |
| CID4465 | CAFs MSC iCAF-like s1 | 99 | 0.063299233 | TNBC |
| CID4495 | CAFs MSC iCAF-like s1 | 57 | 0.007138384 | TNBC |
| CID44971 | CAFs MSC iCAF-like s1 | 246 | 0.030803907 | TNBC |
| CID44991 | CAFs MSC iCAF-like s1 | 16 | 0.002278229 | TNBC |
| CID4513 | CAFs MSC iCAF-like s1 | 1 | 0.000177968 | TNBC |
| CID4515 | CAFs MSC iCAF-like s1 | 78 | 0.018799711 | TNBC |
| CID4523 | CAFs MSC iCAF-like s1 | 3 | 0.001710376 | TNBC |
| CID3946 | CAFs MSC iCAF-like s1 | 1 | 0.00129199 | TNBC |
| CID3963 | CAFs MSC iCAF-like s1 | 1 | 0.000283527 | TNBC |
| CID4461 | CAFs MSC iCAF-like s1 | 16 | 0.025356577 | ER+ |
| CID4463 | CAFs MSC iCAF-like s1 | 5 | 0.004393673 | ER+ |
| CID4471 | CAFs MSC iCAF-like s1 | 718 | 0.083401092 | ER+ |
| CID4530N | CAFs MSC iCAF-like s1 | 176 | 0.039918349 | ER+ |
| CID4535 | CAFs MSC iCAF-like s1 | 12 | 0.003029538 | ER+ |
| CID4040 | CAFs MSC iCAF-like s1 | 28 | 0.011062821 | ER+ |
| CID3941 | CAFs MSC iCAF-like s1 | 2 | 0.003169572 | ER+ |
| CID3948 | CAFs MSC iCAF-like s1 | 1 | 0.000429738 | ER+ |
| CID4067 | CAFs MSC iCAF-like s1 | 27 | 0.00717322 | ER+ |
| CID4290A | CAFs MSC iCAF-like s1 | 74 | 0.012782864 | ER+ |
| CID4398 | CAFs MSC iCAF-like s1 | 103 | 0.023140867 | ER+ |
| CID3586 | CAFs MSC iCAF-like s2 | 87 | 0.014082227 | HER2+ |
| CID3921 | CAFs MSC iCAF-like s2 | 6 | 0.001984127 | HER2+ |
| CID45171 | CAFs MSC iCAF-like s2 | 10 | 0.004086637 | HER2+ |
| CID3838 | CAFs MSC iCAF-like s2 | 7 | 0.002974926 | HER2+ |
| CID4066 | CAFs MSC iCAF-like s2 | 51 | 0.009606329 | HER2+ |
| CID44041 | CAFs MSC iCAF-like s2 | 149 | 0.069920225 | TNBC |
| CID4465 | CAFs MSC iCAF-like s2 | 31 | 0.019820972 | TNBC |
| CID4495 | CAFs MSC iCAF-like s2 | 63 | 0.007889793 | TNBC |
| CID44971 | CAFs MSC iCAF-like s2 | 175 | 0.021913348 | TNBC |
| CID44991 | CAFs MSC iCAF-like s2 | 50 | 0.007119465 | TNBC |
| CID4513 | CAFs MSC iCAF-like s2 | 5 | 0.000889838 | TNBC |
| CID4515 | CAFs MSC iCAF-like s2 | 13 | 0.003133285 | TNBC |
| CID4523 | CAFs MSC iCAF-like s2 | 23 | 0.013112885 | TNBC |
| CID3946 | CAFs MSC iCAF-like s2 | 23 | 0.029715762 | TNBC |
| CID3963 | CAFs MSC iCAF-like s2 | 7 | 0.00198469 | TNBC |
| CID4461 | CAFs MSC iCAF-like s2 | 1 | 0.001584786 | ER+ |
| CID4463 | CAFs MSC iCAF-like s2 | 1 | 0.000878735 | ER+ |
| CID4471 | CAFs MSC iCAF-like s2 | 43 | 0.004994773 | ER+ |
| CID4530N | CAFs MSC iCAF-like s2 | 3 | 0.000680426 | ER+ |
| CID4535 | CAFs MSC iCAF-like s2 | 46 | 0.011613229 | ER+ |
| CID4040 | CAFs MSC iCAF-like s2 | 19 | 0.007506914 | ER+ |
| CID3941 | CAFs MSC iCAF-like s2 | 3 | 0.004754358 | ER+ |
| CID3948 | CAFs MSC iCAF-like s2 | 3 | 0.001289214 | ER+ |
| CID4067 | CAFs MSC iCAF-like s2 | 10 | 0.002656748 | ER+ |
| CID4290A | CAFs MSC iCAF-like s2 | 13 | 0.002245638 | ER+ |
| CID4398 | CAFs MSC iCAF-like s2 | 2 | 0.000449337 | ER+ |
| CID3586 | CAFs myCAF like s4 | 11 | 0.001780511 | HER2+ |
| CID3921 | CAFs myCAF like s4 | 7 | 0.002314815 | HER2+ |
| CID45171 | CAFs myCAF like s4 | 5 | 0.002043318 | HER2+ |
| CID3838 | CAFs myCAF like s4 | 5 | 0.002124947 | HER2+ |
| CID4066 | CAFs myCAF like s4 | 69 | 0.012996798 | HER2+ |
| CID44041 | CAFs myCAF like s4 | 123 | 0.057719381 | TNBC |
| CID4465 | CAFs myCAF like s4 | 34 | 0.02173913 | TNBC |
| CID4495 | CAFs myCAF like s4 | 27 | 0.00338134 | TNBC |
| CID44971 | CAFs myCAF like s4 | 37 | 0.004633108 | TNBC |
| CID44991 | CAFs myCAF like s4 | 62 | 0.008828136 | TNBC |
| CID4513 | CAFs myCAF like s4 | 3 | 0.000533903 | TNBC |
| CID4515 | CAFs myCAF like s4 | 8 | 0.001928175 | TNBC |
| CID4523 | CAFs myCAF like s4 | 8 | 0.004561003 | TNBC |
| CID3946 | CAFs myCAF like s4 | 103 | 0.133074935 | TNBC |
| CID3963 | CAFs myCAF like s4 | 4 | 0.001134108 | TNBC |
| CID4461 | CAFs myCAF like s4 | 2 | 0.003169572 | ER+ |
| CID4463 | CAFs myCAF like s4 | 1 | 0.000878735 | ER+ |
| CID4471 | CAFs myCAF like s4 | 17 | 0.001974678 | ER+ |
| CID4530N | CAFs myCAF like s4 | 3 | 0.000680426 | ER+ |
| CID4535 | CAFs myCAF like s4 | 18 | 0.004544307 | ER+ |
| CID4040 | CAFs myCAF like s4 | 12 | 0.004741209 | ER+ |
| CID3941 | CAFs myCAF like s4 | 2 | 0.003169572 | ER+ |
| CID3948 | CAFs myCAF like s4 | 1 | 0.000429738 | ER+ |
| CID4067 | CAFs myCAF like s4 | 18 | 0.004782147 | ER+ |
| CID4290A | CAFs myCAF like s4 | 17 | 0.002936604 | ER+ |
| CID4398 | CAFs myCAF like s4 | 5 | 0.001123343 | ER+ |
| CID3586 | CAFs myCAF like s5 | 22 | 0.003561023 | HER2+ |
| CID3921 | CAFs myCAF like s5 | 51 | 0.016865079 | HER2+ |
| CID45171 | CAFs myCAF like s5 | 9 | 0.003677973 | HER2+ |
| CID3838 | CAFs myCAF like s5 | 119 | 0.050573736 | HER2+ |
| CID4066 | CAFs myCAF like s5 | 428 | 0.080617819 | HER2+ |
| CID44041 | CAFs myCAF like s5 | 124 | 0.058188644 | TNBC |
| CID4465 | CAFs myCAF like s5 | 182 | 0.116368286 | TNBC |
| CID4495 | CAFs myCAF like s5 | 72 | 0.009016907 | TNBC |
| CID44971 | CAFs myCAF like s5 | 94 | 0.011770599 | TNBC |
| CID44991 | CAFs myCAF like s5 | 103 | 0.014666097 | TNBC |
| CID4513 | CAFs myCAF like s5 | 3 | 0.000533903 | TNBC |
| CID4515 | CAFs myCAF like s5 | 77 | 0.018558689 | TNBC |
| CID4523 | CAFs myCAF like s5 | 7 | 0.003990878 | TNBC |
| CID3946 | CAFs myCAF like s5 | 30 | 0.03875969 | TNBC |
| CID3963 | CAFs myCAF like s5 | 11 | 0.003118798 | TNBC |
| CID4461 | CAFs myCAF like s5 | 17 | 0.026941363 | ER+ |
| CID4463 | CAFs myCAF like s5 | 13 | 0.01142355 | ER+ |
| CID4471 | CAFs myCAF like s5 | 412 | 0.047856894 | ER+ |
| CID4530N | CAFs myCAF like s5 | 149 | 0.033794511 | ER+ |
| CID4535 | CAFs myCAF like s5 | 22 | 0.005554153 | ER+ |
| CID4040 | CAFs myCAF like s5 | 56 | 0.022125642 | ER+ |
| CID3941 | CAFs myCAF like s5 | 1 | 0.001584786 | ER+ |
| CID3948 | CAFs myCAF like s5 | 8 | 0.003437903 | ER+ |
| CID4067 | CAFs myCAF like s5 | 64 | 0.017003188 | ER+ |
| CID4290A | CAFs myCAF like s5 | 141 | 0.024356538 | ER+ |
| CID4398 | CAFs myCAF like s5 | 50 | 0.011233431 | ER+ |
| CID3586 | CAFs Transitioning s3 | 6 | 0.000971188 | HER2+ |
| CID3921 | CAFs Transitioning s3 | 4 | 0.001322751 | HER2+ |
| CID45171 | CAFs Transitioning s3 | 1 | 0.000408664 | HER2+ |
| CID3838 | CAFs Transitioning s3 | 30 | 0.012749681 | HER2+ |
| CID4066 | CAFs Transitioning s3 | 103 | 0.019401017 | HER2+ |
| CID44041 | CAFs Transitioning s3 | 58 | 0.027217269 | TNBC |
| CID4465 | CAFs Transitioning s3 | 33 | 0.021099744 | TNBC |
| CID4495 | CAFs Transitioning s3 | 13 | 0.001628053 | TNBC |
| CID44971 | CAFs Transitioning s3 | 30 | 0.003756574 | TNBC |
| CID44991 | CAFs Transitioning s3 | 14 | 0.00199345 | TNBC |
| CID4513 | CAFs Transitioning s3 | 1 | 0.000177968 | TNBC |
| CID4515 | CAFs Transitioning s3 | 11 | 0.002651241 | TNBC |
| CID4523 | CAFs Transitioning s3 | 1 | 0.000570125 | TNBC |
| CID3946 | CAFs Transitioning s3 | 10 | 0.012919897 | TNBC |
| CID3963 | CAFs Transitioning s3 | 0 | 0 | TNBC |
| CID4461 | CAFs Transitioning s3 | 5 | 0.00792393 | ER+ |
| CID4463 | CAFs Transitioning s3 | 5 | 0.004393673 | ER+ |
| CID4471 | CAFs Transitioning s3 | 102 | 0.011848066 | ER+ |
| CID4530N | CAFs Transitioning s3 | 37 | 0.008391926 | ER+ |
| CID4535 | CAFs Transitioning s3 | 4 | 0.001009846 | ER+ |
| CID4040 | CAFs Transitioning s3 | 14 | 0.005531411 | ER+ |
| CID3941 | CAFs Transitioning s3 | 0 | 0 | ER+ |
| CID3948 | CAFs Transitioning s3 | 2 | 0.000859476 | ER+ |
| CID4067 | CAFs Transitioning s3 | 16 | 0.004250797 | ER+ |
| CID4290A | CAFs Transitioning s3 | 35 | 0.006045949 | ER+ |
| CID4398 | CAFs Transitioning s3 | 18 | 0.004044035 | ER+ |
| CID3586 | Cancer Basal SC | 0 | 0 | HER2+ |
| CID3921 | Cancer Basal SC | 0 | 0 | HER2+ |
| CID45171 | Cancer Basal SC | 1 | 0.000408664 | HER2+ |
| CID3838 | Cancer Basal SC | 0 | 0 | HER2+ |
| CID4066 | Cancer Basal SC | 2 | 0.000376719 | HER2+ |
| CID44041 | Cancer Basal SC | 0 | 0 | TNBC |
| CID4465 | Cancer Basal SC | 22 | 0.014066496 | TNBC |
| CID4495 | Cancer Basal SC | 711 | 0.089041954 | TNBC |
| CID44971 | Cancer Basal SC | 646 | 0.08089156 | TNBC |
| CID44991 | Cancer Basal SC | 369 | 0.052541649 | TNBC |
| CID4513 | Cancer Basal SC | 502 | 0.08933974 | TNBC |
| CID4515 | Cancer Basal SC | 1200 | 0.28922632 | TNBC |
| CID4523 | Cancer Basal SC | 545 | 0.310718358 | TNBC |
| CID3946 | Cancer Basal SC | 0 | 0 | TNBC |
| CID3963 | Cancer Basal SC | 182 | 0.051601928 | TNBC |
| CID4461 | Cancer Basal SC | 0 | 0 | ER+ |
| CID4463 | Cancer Basal SC | 3 | 0.002636204 | ER+ |
| CID4471 | Cancer Basal SC | 10 | 0.001161575 | ER+ |
| CID4530N | Cancer Basal SC | 71 | 0.016103425 | ER+ |
| CID4535 | Cancer Basal SC | 1 | 0.000252461 | ER+ |
| CID4040 | Cancer Basal SC | 0 | 0 | ER+ |
| CID3941 | Cancer Basal SC | 0 | 0 | ER+ |
| CID3948 | Cancer Basal SC | 0 | 0 | ER+ |
| CID4067 | Cancer Basal SC | 1 | 0.000265675 | ER+ |
| CID4290A | Cancer Basal SC | 46 | 0.007946105 | ER+ |
| CID4398 | Cancer Basal SC | 0 | 0 | ER+ |
| CID3586 | Cancer Cycling | 0 | 0 | HER2+ |
| CID3921 | Cancer Cycling | 64 | 0.021164021 | HER2+ |
| CID45171 | Cancer Cycling | 236 | 0.096444626 | HER2+ |
| CID3838 | Cancer Cycling | 0 | 0 | HER2+ |
| CID4066 | Cancer Cycling | 112 | 0.021096252 | HER2+ |
| CID44041 | Cancer Cycling | 0 | 0 | TNBC |
| CID4465 | Cancer Cycling | 97 | 0.06202046 | TNBC |
| CID4495 | Cancer Cycling | 459 | 0.05748278 | TNBC |
| CID44971 | Cancer Cycling | 246 | 0.030803907 | TNBC |
| CID44991 | Cancer Cycling | 1583 | 0.22540225 | TNBC |
| CID4513 | Cancer Cycling | 500 | 0.088983805 | TNBC |
| CID4515 | Cancer Cycling | 927 | 0.223427332 | TNBC |
| CID4523 | Cancer Cycling | 531 | 0.302736602 | TNBC |
| CID3946 | Cancer Cycling | 0 | 0 | TNBC |
| CID3963 | Cancer Cycling | 29 | 0.008222285 | TNBC |
| CID4461 | Cancer Cycling | 33 | 0.05229794 | ER+ |
| CID4463 | Cancer Cycling | 47 | 0.041300527 | ER+ |
| CID4471 | Cancer Cycling | 28 | 0.00325241 | ER+ |
| CID4530N | Cancer Cycling | 15 | 0.003402132 | ER+ |
| CID4535 | Cancer Cycling | 195 | 0.049229992 | ER+ |
| CID4040 | Cancer Cycling | 0 | 0 | ER+ |
| CID3941 | Cancer Cycling | 7 | 0.011093502 | ER+ |
| CID3948 | Cancer Cycling | 13 | 0.005586592 | ER+ |
| CID4067 | Cancer Cycling | 117 | 0.031083953 | ER+ |
| CID4290A | Cancer Cycling | 120 | 0.020728969 | ER+ |
| CID4398 | Cancer Cycling | 0 | 0 | ER+ |
| CID3586 | Cancer Her2 SC | 0 | 0 | HER2+ |
| CID3921 | Cancer Her2 SC | 377 | 0.124669312 | HER2+ |
| CID45171 | Cancer Her2 SC | 567 | 0.231712301 | HER2+ |
| CID3838 | Cancer Her2 SC | 0 | 0 | HER2+ |
| CID4066 | Cancer Her2 SC | 393 | 0.07402524 | HER2+ |
| CID44041 | Cancer Her2 SC | 0 | 0 | TNBC |
| CID4465 | Cancer Her2 SC | 0 | 0 | TNBC |
| CID4495 | Cancer Her2 SC | 0 | 0 | TNBC |
| CID44971 | Cancer Her2 SC | 1 | 0.000125219 | TNBC |
| CID44991 | Cancer Her2 SC | 1912 | 0.272248327 | TNBC |
| CID4513 | Cancer Her2 SC | 31 | 0.005516996 | TNBC |
| CID4515 | Cancer Her2 SC | 30 | 0.007230658 | TNBC |
| CID4523 | Cancer Her2 SC | 67 | 0.038198404 | TNBC |
| CID3946 | Cancer Her2 SC | 0 | 0 | TNBC |
| CID3963 | Cancer Her2 SC | 2 | 0.000567054 | TNBC |
| CID4461 | Cancer Her2 SC | 0 | 0 | ER+ |
| CID4463 | Cancer Her2 SC | 2 | 0.001757469 | ER+ |
| CID4471 | Cancer Her2 SC | 5 | 0.000580788 | ER+ |
| CID4530N | Cancer Her2 SC | 33 | 0.00748469 | ER+ |
| CID4535 | Cancer Her2 SC | 2 | 0.000504923 | ER+ |
| CID4040 | Cancer Her2 SC | 0 | 0 | ER+ |
| CID3941 | Cancer Her2 SC | 0 | 0 | ER+ |
| CID3948 | Cancer Her2 SC | 0 | 0 | ER+ |
| CID4067 | Cancer Her2 SC | 6 | 0.001594049 | ER+ |
| CID4290A | Cancer Her2 SC | 280 | 0.048367594 | ER+ |
| CID4398 | Cancer Her2 SC | 0 | 0 | ER+ |
| CID3586 | Cancer LumA SC | 0 | 0 | HER2+ |
| CID3921 | Cancer LumA SC | 0 | 0 | HER2+ |
| CID45171 | Cancer LumA SC | 0 | 0 | HER2+ |
| CID3838 | Cancer LumA SC | 0 | 0 | HER2+ |
| CID4066 | Cancer LumA SC | 8 | 0.001506875 | HER2+ |
| CID44041 | Cancer LumA SC | 0 | 0 | TNBC |
| CID4465 | Cancer LumA SC | 0 | 0 | TNBC |
| CID4495 | Cancer LumA SC | 2 | 0.00025047 | TNBC |
| CID44971 | Cancer LumA SC | 0 | 0 | TNBC |
| CID44991 | Cancer LumA SC | 51 | 0.007261854 | TNBC |
| CID4513 | Cancer LumA SC | 14 | 0.002491547 | TNBC |
| CID4515 | Cancer LumA SC | 0 | 0 | TNBC |
| CID4523 | Cancer LumA SC | 2 | 0.001140251 | TNBC |
| CID3946 | Cancer LumA SC | 0 | 0 | TNBC |
| CID3963 | Cancer LumA SC | 8 | 0.002268217 | TNBC |
| CID4461 | Cancer LumA SC | 0 | 0 | ER+ |
| CID4463 | Cancer LumA SC | 582 | 0.51142355 | ER+ |
| CID4471 | Cancer LumA SC | 169 | 0.019630619 | ER+ |
| CID4530N | Cancer LumA SC | 1145 | 0.259696076 | ER+ |
| CID4535 | Cancer LumA SC | 0 | 0 | ER+ |
| CID4040 | Cancer LumA SC | 0 | 0 | ER+ |
| CID3941 | Cancer LumA SC | 187 | 0.296354992 | ER+ |
| CID3948 | Cancer LumA SC | 194 | 0.083369145 | ER+ |
| CID4067 | Cancer LumA SC | 1827 | 0.485387885 | ER+ |
| CID4290A | Cancer LumA SC | 3553 | 0.613750216 | ER+ |
| CID4398 | Cancer LumA SC | 0 | 0 | ER+ |
| CID3586 | Cancer LumB SC | 0 | 0 | HER2+ |
| CID3921 | Cancer LumB SC | 0 | 0 | HER2+ |
| CID45171 | Cancer LumB SC | 9 | 0.003677973 | HER2+ |
| CID3838 | Cancer LumB SC | 0 | 0 | HER2+ |
| CID4066 | Cancer LumB SC | 6 | 0.001130156 | HER2+ |
| CID44041 | Cancer LumB SC | 0 | 0 | TNBC |
| CID4465 | Cancer LumB SC | 5 | 0.003196931 | TNBC |
| CID4495 | Cancer LumB SC | 12 | 0.001502818 | TNBC |
| CID44971 | Cancer LumB SC | 1 | 0.000125219 | TNBC |
| CID44991 | Cancer LumB SC | 103 | 0.014666097 | TNBC |
| CID4513 | Cancer LumB SC | 11 | 0.001957644 | TNBC |
| CID4515 | Cancer LumB SC | 12 | 0.002892263 | TNBC |
| CID4523 | Cancer LumB SC | 22 | 0.012542759 | TNBC |
| CID3946 | Cancer LumB SC | 0 | 0 | TNBC |
| CID3963 | Cancer LumB SC | 1 | 0.000283527 | TNBC |
| CID4461 | Cancer LumB SC | 174 | 0.275752773 | ER+ |
| CID4463 | Cancer LumB SC | 25 | 0.021968366 | ER+ |
| CID4471 | Cancer LumB SC | 0 | 0 | ER+ |
| CID4530N | Cancer LumB SC | 451 | 0.102290769 | ER+ |
| CID4535 | Cancer LumB SC | 2025 | 0.511234537 | ER+ |
| CID4040 | Cancer LumB SC | 0 | 0 | ER+ |
| CID3941 | Cancer LumB SC | 2 | 0.003169572 | ER+ |
| CID3948 | Cancer LumB SC | 54 | 0.023205844 | ER+ |
| CID4067 | Cancer LumB SC | 401 | 0.1065356 | ER+ |
| CID4290A | Cancer LumB SC | 54 | 0.009328036 | ER+ |
| CID4398 | Cancer LumB SC | 0 | 0 | ER+ |
| CID3586 | Cycling PVL | 0 | 0 | HER2+ |
| CID3921 | Cycling PVL | 0 | 0 | HER2+ |
| CID45171 | Cycling PVL | 0 | 0 | HER2+ |
| CID3838 | Cycling PVL | 4 | 0.001699958 | HER2+ |
| CID4066 | Cycling PVL | 6 | 0.001130156 | HER2+ |
| CID44041 | Cycling PVL | 0 | 0 | TNBC |
| CID4465 | Cycling PVL | 7 | 0.004475703 | TNBC |
| CID4495 | Cycling PVL | 2 | 0.00025047 | TNBC |
| CID44971 | Cycling PVL | 0 | 0 | TNBC |
| CID44991 | Cycling PVL | 6 | 0.000854336 | TNBC |
| CID4513 | Cycling PVL | 2 | 0.000355935 | TNBC |
| CID4515 | Cycling PVL | 2 | 0.000482044 | TNBC |
| CID4523 | Cycling PVL | 0 | 0 | TNBC |
| CID3946 | Cycling PVL | 0 | 0 | TNBC |
| CID3963 | Cycling PVL | 1 | 0.000283527 | TNBC |
| CID4461 | Cycling PVL | 0 | 0 | ER+ |
| CID4463 | Cycling PVL | 0 | 0 | ER+ |
| CID4471 | Cycling PVL | 0 | 0 | ER+ |
| CID4530N | Cycling PVL | 0 | 0 | ER+ |
| CID4535 | Cycling PVL | 10 | 0.002524615 | ER+ |
| CID4040 | Cycling PVL | 4 | 0.001580403 | ER+ |
| CID3941 | Cycling PVL | 1 | 0.001584786 | ER+ |
| CID3948 | Cycling PVL | 0 | 0 | ER+ |
| CID4067 | Cycling PVL | 2 | 0.00053135 | ER+ |
| CID4290A | Cycling PVL | 1 | 0.000172741 | ER+ |
| CID4398 | Cycling PVL | 2 | 0.000449337 | ER+ |
| CID3586 | Cycling_Myeloid | 11 | 0.001780511 | HER2+ |
| CID3921 | Cycling_Myeloid | 18 | 0.005952381 | HER2+ |
| CID45171 | Cycling_Myeloid | 2 | 0.000817327 | HER2+ |
| CID3838 | Cycling_Myeloid | 21 | 0.008924777 | HER2+ |
| CID4066 | Cycling_Myeloid | 10 | 0.001883594 | HER2+ |
| CID44041 | Cycling_Myeloid | 2 | 0.000938527 | TNBC |
| CID4465 | Cycling_Myeloid | 21 | 0.01342711 | TNBC |
| CID4495 | Cycling_Myeloid | 42 | 0.005259862 | TNBC |
| CID44971 | Cycling_Myeloid | 46 | 0.00576008 | TNBC |
| CID44991 | Cycling_Myeloid | 3 | 0.000427168 | TNBC |
| CID4513 | Cycling_Myeloid | 147 | 0.026161239 | TNBC |
| CID4515 | Cycling_Myeloid | 30 | 0.007230658 | TNBC |
| CID4523 | Cycling_Myeloid | 11 | 0.00627138 | TNBC |
| CID3946 | Cycling_Myeloid | 3 | 0.003875969 | TNBC |
| CID3963 | Cycling_Myeloid | 24 | 0.00680465 | TNBC |
| CID4461 | Cycling_Myeloid | 5 | 0.00792393 | ER+ |
| CID4463 | Cycling_Myeloid | 10 | 0.008787346 | ER+ |
| CID4471 | Cycling_Myeloid | 12 | 0.00139389 | ER+ |
| CID4530N | Cycling_Myeloid | 3 | 0.000680426 | ER+ |
| CID4535 | Cycling_Myeloid | 8 | 0.002019692 | ER+ |
| CID4040 | Cycling_Myeloid | 3 | 0.001185302 | ER+ |
| CID3941 | Cycling_Myeloid | 0 | 0 | ER+ |
| CID3948 | Cycling_Myeloid | 4 | 0.001718951 | ER+ |
| CID4067 | Cycling_Myeloid | 3 | 0.000797024 | ER+ |
| CID4290A | Cycling_Myeloid | 13 | 0.002245638 | ER+ |
| CID4398 | Cycling_Myeloid | 11 | 0.002471355 | ER+ |
| CID3586 | Endothelial ACKR1 | 80 | 0.012949174 | HER2+ |
| CID3921 | Endothelial ACKR1 | 121 | 0.040013228 | HER2+ |
| CID45171 | Endothelial ACKR1 | 10 | 0.004086637 | HER2+ |
| CID3838 | Endothelial ACKR1 | 48 | 0.02039949 | HER2+ |
| CID4066 | Endothelial ACKR1 | 299 | 0.056319458 | HER2+ |
| CID44041 | Endothelial ACKR1 | 84 | 0.039418114 | TNBC |
| CID4465 | Endothelial ACKR1 | 192 | 0.122762148 | TNBC |
| CID4495 | Endothelial ACKR1 | 58 | 0.007263619 | TNBC |
| CID44971 | Endothelial ACKR1 | 106 | 0.013273228 | TNBC |
| CID44991 | Endothelial ACKR1 | 15 | 0.002135839 | TNBC |
| CID4513 | Endothelial ACKR1 | 74 | 0.013169603 | TNBC |
| CID4515 | Endothelial ACKR1 | 77 | 0.018558689 | TNBC |
| CID4523 | Endothelial ACKR1 | 1 | 0.000570125 | TNBC |
| CID3946 | Endothelial ACKR1 | 65 | 0.083979328 | TNBC |
| CID3963 | Endothelial ACKR1 | 59 | 0.016728098 | TNBC |
| CID4461 | Endothelial ACKR1 | 106 | 0.167987322 | ER+ |
| CID4463 | Endothelial ACKR1 | 43 | 0.037785589 | ER+ |
| CID4471 | Endothelial ACKR1 | 2065 | 0.239865257 | ER+ |
| CID4530N | Endothelial ACKR1 | 573 | 0.129961443 | ER+ |
| CID4535 | Endothelial ACKR1 | 44 | 0.011108306 | ER+ |
| CID4040 | Endothelial ACKR1 | 98 | 0.038719874 | ER+ |
| CID3941 | Endothelial ACKR1 | 24 | 0.038034865 | ER+ |
| CID3948 | Endothelial ACKR1 | 43 | 0.018478728 | ER+ |
| CID4067 | Endothelial ACKR1 | 111 | 0.029489904 | ER+ |
| CID4290A | Endothelial ACKR1 | 158 | 0.027293142 | ER+ |
| CID4398 | Endothelial ACKR1 | 57 | 0.012806111 | ER+ |
| CID3586 | Endothelial CXCL12 | 38 | 0.006150858 | HER2+ |
| CID3921 | Endothelial CXCL12 | 44 | 0.014550265 | HER2+ |
| CID45171 | Endothelial CXCL12 | 3 | 0.001225991 | HER2+ |
| CID3838 | Endothelial CXCL12 | 24 | 0.010199745 | HER2+ |
| CID4066 | Endothelial CXCL12 | 142 | 0.026747033 | HER2+ |
| CID44041 | Endothelial CXCL12 | 32 | 0.015016424 | TNBC |
| CID4465 | Endothelial CXCL12 | 52 | 0.033248082 | TNBC |
| CID4495 | Endothelial CXCL12 | 64 | 0.008015028 | TNBC |
| CID44971 | Endothelial CXCL12 | 47 | 0.005885299 | TNBC |
| CID44991 | Endothelial CXCL12 | 13 | 0.001851061 | TNBC |
| CID4513 | Endothelial CXCL12 | 44 | 0.007830575 | TNBC |
| CID4515 | Endothelial CXCL12 | 27 | 0.006507592 | TNBC |
| CID4523 | Endothelial CXCL12 | 1 | 0.000570125 | TNBC |
| CID3946 | Endothelial CXCL12 | 28 | 0.036175711 | TNBC |
| CID3963 | Endothelial CXCL12 | 25 | 0.007088177 | TNBC |
| CID4461 | Endothelial CXCL12 | 42 | 0.066561014 | ER+ |
| CID4463 | Endothelial CXCL12 | 23 | 0.020210896 | ER+ |
| CID4471 | Endothelial CXCL12 | 359 | 0.041700546 | ER+ |
| CID4530N | Endothelial CXCL12 | 268 | 0.060784758 | ER+ |
| CID4535 | Endothelial CXCL12 | 128 | 0.032315072 | ER+ |
| CID4040 | Endothelial CXCL12 | 67 | 0.02647175 | ER+ |
| CID3941 | Endothelial CXCL12 | 11 | 0.017432647 | ER+ |
| CID3948 | Endothelial CXCL12 | 22 | 0.009454233 | ER+ |
| CID4067 | Endothelial CXCL12 | 34 | 0.009032944 | ER+ |
| CID4290A | Endothelial CXCL12 | 72 | 0.012437381 | ER+ |
| CID4398 | Endothelial CXCL12 | 34 | 0.007638733 | ER+ |
| CID3586 | Endothelial Lymphatic LYVE1 | 10 | 0.001618647 | HER2+ |
| CID3921 | Endothelial Lymphatic LYVE1 | 10 | 0.003306878 | HER2+ |
| CID45171 | Endothelial Lymphatic LYVE1 | 0 | 0 | HER2+ |
| CID3838 | Endothelial Lymphatic LYVE1 | 4 | 0.001699958 | HER2+ |
| CID4066 | Endothelial Lymphatic LYVE1 | 7 | 0.001318516 | HER2+ |
| CID44041 | Endothelial Lymphatic LYVE1 | 6 | 0.00281558 | TNBC |
| CID4465 | Endothelial Lymphatic LYVE1 | 14 | 0.008951407 | TNBC |
| CID4495 | Endothelial Lymphatic LYVE1 | 28 | 0.003506575 | TNBC |
| CID44971 | Endothelial Lymphatic LYVE1 | 12 | 0.00150263 | TNBC |
| CID44991 | Endothelial Lymphatic LYVE1 | 1 | 0.000142389 | TNBC |
| CID4513 | Endothelial Lymphatic LYVE1 | 0 | 0 | TNBC |
| CID4515 | Endothelial Lymphatic LYVE1 | 3 | 0.000723066 | TNBC |
| CID4523 | Endothelial Lymphatic LYVE1 | 0 | 0 | TNBC |
| CID3946 | Endothelial Lymphatic LYVE1 | 0 | 0 | TNBC |
| CID3963 | Endothelial Lymphatic LYVE1 | 0 | 0 | TNBC |
| CID4461 | Endothelial Lymphatic LYVE1 | 3 | 0.004754358 | ER+ |
| CID4463 | Endothelial Lymphatic LYVE1 | 2 | 0.001757469 | ER+ |
| CID4471 | Endothelial Lymphatic LYVE1 | 46 | 0.005343245 | ER+ |
| CID4530N | Endothelial Lymphatic LYVE1 | 20 | 0.004536176 | ER+ |
| CID4535 | Endothelial Lymphatic LYVE1 | 5 | 0.001262307 | ER+ |
| CID4040 | Endothelial Lymphatic LYVE1 | 13 | 0.00513631 | ER+ |
| CID3941 | Endothelial Lymphatic LYVE1 | 1 | 0.001584786 | ER+ |
| CID3948 | Endothelial Lymphatic LYVE1 | 6 | 0.002578427 | ER+ |
| CID4067 | Endothelial Lymphatic LYVE1 | 2 | 0.00053135 | ER+ |
| CID4290A | Endothelial Lymphatic LYVE1 | 5 | 0.000863707 | ER+ |
| CID4398 | Endothelial Lymphatic LYVE1 | 5 | 0.001123343 | ER+ |
| CID3586 | Endothelial RGS5 | 29 | 0.004694076 | HER2+ |
| CID3921 | Endothelial RGS5 | 35 | 0.011574074 | HER2+ |
| CID45171 | Endothelial RGS5 | 2 | 0.000817327 | HER2+ |
| CID3838 | Endothelial RGS5 | 23 | 0.009774756 | HER2+ |
| CID4066 | Endothelial RGS5 | 87 | 0.016387267 | HER2+ |
| CID44041 | Endothelial RGS5 | 26 | 0.012200845 | TNBC |
| CID4465 | Endothelial RGS5 | 36 | 0.023017903 | TNBC |
| CID4495 | Endothelial RGS5 | 34 | 0.004257984 | TNBC |
| CID44971 | Endothelial RGS5 | 52 | 0.006511395 | TNBC |
| CID44991 | Endothelial RGS5 | 12 | 0.001708672 | TNBC |
| CID4513 | Endothelial RGS5 | 44 | 0.007830575 | TNBC |
| CID4515 | Endothelial RGS5 | 15 | 0.003615329 | TNBC |
| CID4523 | Endothelial RGS5 | 1 | 0.000570125 | TNBC |
| CID3946 | Endothelial RGS5 | 17 | 0.021963824 | TNBC |
| CID3963 | Endothelial RGS5 | 18 | 0.005103487 | TNBC |
| CID4461 | Endothelial RGS5 | 31 | 0.049128368 | ER+ |
| CID4463 | Endothelial RGS5 | 11 | 0.009666081 | ER+ |
| CID4471 | Endothelial RGS5 | 308 | 0.035776513 | ER+ |
| CID4530N | Endothelial RGS5 | 155 | 0.035155364 | ER+ |
| CID4535 | Endothelial RGS5 | 42 | 0.010603383 | ER+ |
| CID4040 | Endothelial RGS5 | 40 | 0.01580403 | ER+ |
| CID3941 | Endothelial RGS5 | 8 | 0.012678288 | ER+ |
| CID3948 | Endothelial RGS5 | 14 | 0.00601633 | ER+ |
| CID4067 | Endothelial RGS5 | 39 | 0.010361318 | ER+ |
| CID4290A | Endothelial RGS5 | 63 | 0.010882709 | ER+ |
| CID4398 | Endothelial RGS5 | 5 | 0.001123343 | ER+ |
| CID3586 | Luminal Progenitors | 471 | 0.076238265 | HER2+ |
| CID3921 | Luminal Progenitors | 0 | 0 | HER2+ |
| CID45171 | Luminal Progenitors | 0 | 0 | HER2+ |
| CID3838 | Luminal Progenitors | 0 | 0 | HER2+ |
| CID4066 | Luminal Progenitors | 106 | 0.019966095 | HER2+ |
| CID44041 | Luminal Progenitors | 57 | 0.026748006 | TNBC |
| CID4465 | Luminal Progenitors | 4 | 0.002557545 | TNBC |
| CID4495 | Luminal Progenitors | 0 | 0 | TNBC |
| CID44971 | Luminal Progenitors | 442 | 0.055346857 | TNBC |
| CID44991 | Luminal Progenitors | 11 | 0.001566282 | TNBC |
| CID4513 | Luminal Progenitors | 0 | 0 | TNBC |
| CID4515 | Luminal Progenitors | 9 | 0.002169197 | TNBC |
| CID4523 | Luminal Progenitors | 0 | 0 | TNBC |
| CID3946 | Luminal Progenitors | 0 | 0 | TNBC |
| CID3963 | Luminal Progenitors | 1 | 0.000283527 | TNBC |
| CID4461 | Luminal Progenitors | 0 | 0 | ER+ |
| CID4463 | Luminal Progenitors | 12 | 0.010544815 | ER+ |
| CID4471 | Luminal Progenitors | 655 | 0.076083169 | ER+ |
| CID4530N | Luminal Progenitors | 207 | 0.046949422 | ER+ |
| CID4535 | Luminal Progenitors | 7 | 0.00176723 | ER+ |
| CID4040 | Luminal Progenitors | 0 | 0 | ER+ |
| CID3941 | Luminal Progenitors | 0 | 0 | ER+ |
| CID3948 | Luminal Progenitors | 0 | 0 | ER+ |
| CID4067 | Luminal Progenitors | 0 | 0 | ER+ |
| CID4290A | Luminal Progenitors | 10 | 0.001727414 | ER+ |
| CID4398 | Luminal Progenitors | 0 | 0 | ER+ |
| CID3586 | Mature Luminal | 91 | 0.014729686 | HER2+ |
| CID3921 | Mature Luminal | 0 | 0 | HER2+ |
| CID45171 | Mature Luminal | 0 | 0 | HER2+ |
| CID3838 | Mature Luminal | 0 | 0 | HER2+ |
| CID4066 | Mature Luminal | 61 | 0.011489923 | HER2+ |
| CID44041 | Mature Luminal | 85 | 0.039887377 | TNBC |
| CID4465 | Mature Luminal | 6 | 0.003836317 | TNBC |
| CID4495 | Mature Luminal | 0 | 0 | TNBC |
| CID44971 | Mature Luminal | 169 | 0.021162034 | TNBC |
| CID44991 | Mature Luminal | 9 | 0.001281504 | TNBC |
| CID4513 | Mature Luminal | 0 | 0 | TNBC |
| CID4515 | Mature Luminal | 18 | 0.004338395 | TNBC |
| CID4523 | Mature Luminal | 0 | 0 | TNBC |
| CID3946 | Mature Luminal | 0 | 0 | TNBC |
| CID3963 | Mature Luminal | 0 | 0 | TNBC |
| CID4461 | Mature Luminal | 0 | 0 | ER+ |
| CID4463 | Mature Luminal | 10 | 0.008787346 | ER+ |
| CID4471 | Mature Luminal | 654 | 0.075967011 | ER+ |
| CID4530N | Mature Luminal | 145 | 0.032887276 | ER+ |
| CID4535 | Mature Luminal | 13 | 0.003281999 | ER+ |
| CID4040 | Mature Luminal | 0 | 0 | ER+ |
| CID3941 | Mature Luminal | 0 | 0 | ER+ |
| CID3948 | Mature Luminal | 0 | 0 | ER+ |
| CID4067 | Mature Luminal | 0 | 0 | ER+ |
| CID4290A | Mature Luminal | 4 | 0.000690966 | ER+ |
| CID4398 | Mature Luminal | 0 | 0 | ER+ |
| CID3586 | Myeloid_c0_DC_LAMP3 | 3 | 0.000485594 | HER2+ |
| CID3921 | Myeloid_c0_DC_LAMP3 | 5 | 0.001653439 | HER2+ |
| CID45171 | Myeloid_c0_DC_LAMP3 | 3 | 0.001225991 | HER2+ |
| CID3838 | Myeloid_c0_DC_LAMP3 | 7 | 0.002974926 | HER2+ |
| CID4066 | Myeloid_c0_DC_LAMP3 | 5 | 0.000941797 | HER2+ |
| CID44041 | Myeloid_c0_DC_LAMP3 | 4 | 0.001877053 | TNBC |
| CID4465 | Myeloid_c0_DC_LAMP3 | 2 | 0.001278772 | TNBC |
| CID4495 | Myeloid_c0_DC_LAMP3 | 5 | 0.000626174 | TNBC |
| CID44971 | Myeloid_c0_DC_LAMP3 | 25 | 0.003130478 | TNBC |
| CID44991 | Myeloid_c0_DC_LAMP3 | 4 | 0.000569557 | TNBC |
| CID4513 | Myeloid_c0_DC_LAMP3 | 8 | 0.001423741 | TNBC |
| CID4515 | Myeloid_c0_DC_LAMP3 | 7 | 0.001687154 | TNBC |
| CID4523 | Myeloid_c0_DC_LAMP3 | 2 | 0.001140251 | TNBC |
| CID3946 | Myeloid_c0_DC_LAMP3 | 0 | 0 | TNBC |
| CID3963 | Myeloid_c0_DC_LAMP3 | 4 | 0.001134108 | TNBC |
| CID4461 | Myeloid_c0_DC_LAMP3 | 2 | 0.003169572 | ER+ |
| CID4463 | Myeloid_c0_DC_LAMP3 | 2 | 0.001757469 | ER+ |
| CID4471 | Myeloid_c0_DC_LAMP3 | 4 | 0.00046463 | ER+ |
| CID4530N | Myeloid_c0_DC_LAMP3 | 2 | 0.000453618 | ER+ |
| CID4535 | Myeloid_c0_DC_LAMP3 | 5 | 0.001262307 | ER+ |
| CID4040 | Myeloid_c0_DC_LAMP3 | 0 | 0 | ER+ |
| CID3941 | Myeloid_c0_DC_LAMP3 | 0 | 0 | ER+ |
| CID3948 | Myeloid_c0_DC_LAMP3 | 4 | 0.001718951 | ER+ |
| CID4067 | Myeloid_c0_DC_LAMP3 | 1 | 0.000265675 | ER+ |
| CID4290A | Myeloid_c0_DC_LAMP3 | 3 | 0.000518224 | ER+ |
| CID4398 | Myeloid_c0_DC_LAMP3 | 24 | 0.005392047 | ER+ |
| CID3586 | Myeloid_c1_LAM1_FABP5 | 36 | 0.005827129 | HER2+ |
| CID3921 | Myeloid_c1_LAM1_FABP5 | 71 | 0.023478836 | HER2+ |
| CID45171 | Myeloid_c1_LAM1_FABP5 | 10 | 0.004086637 | HER2+ |
| CID3838 | Myeloid_c1_LAM1_FABP5 | 70 | 0.029749256 | HER2+ |
| CID4066 | Myeloid_c1_LAM1_FABP5 | 38 | 0.007157657 | HER2+ |
| CID44041 | Myeloid_c1_LAM1_FABP5 | 22 | 0.010323792 | TNBC |
| CID4465 | Myeloid_c1_LAM1_FABP5 | 105 | 0.06713555 | TNBC |
| CID4495 | Myeloid_c1_LAM1_FABP5 | 158 | 0.019787101 | TNBC |
| CID44971 | Myeloid_c1_LAM1_FABP5 | 126 | 0.015777611 | TNBC |
| CID44991 | Myeloid_c1_LAM1_FABP5 | 48 | 0.006834686 | TNBC |
| CID4513 | Myeloid_c1_LAM1_FABP5 | 434 | 0.077237943 | TNBC |
| CID4515 | Myeloid_c1_LAM1_FABP5 | 129 | 0.031091829 | TNBC |
| CID4523 | Myeloid_c1_LAM1_FABP5 | 174 | 0.099201824 | TNBC |
| CID3946 | Myeloid_c1_LAM1_FABP5 | 105 | 0.135658915 | TNBC |
| CID3963 | Myeloid_c1_LAM1_FABP5 | 105 | 0.029770343 | TNBC |
| CID4461 | Myeloid_c1_LAM1_FABP5 | 21 | 0.033280507 | ER+ |
| CID4463 | Myeloid_c1_LAM1_FABP5 | 22 | 0.019332162 | ER+ |
| CID4471 | Myeloid_c1_LAM1_FABP5 | 49 | 0.005691718 | ER+ |
| CID4530N | Myeloid_c1_LAM1_FABP5 | 12 | 0.002721706 | ER+ |
| CID4535 | Myeloid_c1_LAM1_FABP5 | 51 | 0.012875536 | ER+ |
| CID4040 | Myeloid_c1_LAM1_FABP5 | 11 | 0.004346108 | ER+ |
| CID3941 | Myeloid_c1_LAM1_FABP5 | 23 | 0.036450079 | ER+ |
| CID3948 | Myeloid_c1_LAM1_FABP5 | 63 | 0.027073485 | ER+ |
| CID4067 | Myeloid_c1_LAM1_FABP5 | 52 | 0.01381509 | ER+ |
| CID4290A | Myeloid_c1_LAM1_FABP5 | 140 | 0.024183797 | ER+ |
| CID4398 | Myeloid_c1_LAM1_FABP5 | 47 | 0.010559425 | ER+ |
| CID3586 | Myeloid_c10_Macrophage_1_EGR1 | 37 | 0.005988993 | HER2+ |
| CID3921 | Myeloid_c10_Macrophage_1_EGR1 | 79 | 0.026124339 | HER2+ |
| CID45171 | Myeloid_c10_Macrophage_1_EGR1 | 2 | 0.000817327 | HER2+ |
| CID3838 | Myeloid_c10_Macrophage_1_EGR1 | 182 | 0.077348066 | HER2+ |
| CID4066 | Myeloid_c10_Macrophage_1_EGR1 | 62 | 0.011678282 | HER2+ |
| CID44041 | Myeloid_c10_Macrophage_1_EGR1 | 33 | 0.015485687 | TNBC |
| CID4465 | Myeloid_c10_Macrophage_1_EGR1 | 1 | 0.000639386 | TNBC |
| CID4495 | Myeloid_c10_Macrophage_1_EGR1 | 153 | 0.019160927 | TNBC |
| CID44971 | Myeloid_c10_Macrophage_1_EGR1 | 53 | 0.006636614 | TNBC |
| CID44991 | Myeloid_c10_Macrophage_1_EGR1 | 45 | 0.006407518 | TNBC |
| CID4513 | Myeloid_c10_Macrophage_1_EGR1 | 967 | 0.172094679 | TNBC |
| CID4515 | Myeloid_c10_Macrophage_1_EGR1 | 61 | 0.014702338 | TNBC |
| CID4523 | Myeloid_c10_Macrophage_1_EGR1 | 17 | 0.009692132 | TNBC |
| CID3946 | Myeloid_c10_Macrophage_1_EGR1 | 0 | 0 | TNBC |
| CID3963 | Myeloid_c10_Macrophage_1_EGR1 | 101 | 0.028636235 | TNBC |
| CID4461 | Myeloid_c10_Macrophage_1_EGR1 | 5 | 0.00792393 | ER+ |
| CID4463 | Myeloid_c10_Macrophage_1_EGR1 | 26 | 0.0228471 | ER+ |
| CID4471 | Myeloid_c10_Macrophage_1_EGR1 | 92 | 0.010686491 | ER+ |
| CID4530N | Myeloid_c10_Macrophage_1_EGR1 | 19 | 0.004309367 | ER+ |
| CID4535 | Myeloid_c10_Macrophage_1_EGR1 | 29 | 0.007321383 | ER+ |
| CID4040 | Myeloid_c10_Macrophage_1_EGR1 | 10 | 0.003951008 | ER+ |
| CID3941 | Myeloid_c10_Macrophage_1_EGR1 | 3 | 0.004754358 | ER+ |
| CID3948 | Myeloid_c10_Macrophage_1_EGR1 | 9 | 0.003867641 | ER+ |
| CID4067 | Myeloid_c10_Macrophage_1_EGR1 | 75 | 0.019925611 | ER+ |
| CID4290A | Myeloid_c10_Macrophage_1_EGR1 | 69 | 0.011919157 | ER+ |
| CID4398 | Myeloid_c10_Macrophage_1_EGR1 | 20 | 0.004493372 | ER+ |
| CID3586 | Myeloid_c11_cDC2_CD1C | 9 | 0.001456782 | HER2+ |
| CID3921 | Myeloid_c11_cDC2_CD1C | 28 | 0.009259259 | HER2+ |
| CID45171 | Myeloid_c11_cDC2_CD1C | 7 | 0.002860646 | HER2+ |
| CID3838 | Myeloid_c11_cDC2_CD1C | 10 | 0.004249894 | HER2+ |
| CID4066 | Myeloid_c11_cDC2_CD1C | 9 | 0.001695235 | HER2+ |
| CID44041 | Myeloid_c11_cDC2_CD1C | 11 | 0.005161896 | TNBC |
| CID4465 | Myeloid_c11_cDC2_CD1C | 3 | 0.001918159 | TNBC |
| CID4495 | Myeloid_c11_cDC2_CD1C | 45 | 0.005635567 | TNBC |
| CID44971 | Myeloid_c11_cDC2_CD1C | 49 | 0.006135738 | TNBC |
| CID44991 | Myeloid_c11_cDC2_CD1C | 8 | 0.001139114 | TNBC |
| CID4513 | Myeloid_c11_cDC2_CD1C | 20 | 0.003559352 | TNBC |
| CID4515 | Myeloid_c11_cDC2_CD1C | 18 | 0.004338395 | TNBC |
| CID4523 | Myeloid_c11_cDC2_CD1C | 1 | 0.000570125 | TNBC |
| CID3946 | Myeloid_c11_cDC2_CD1C | 0 | 0 | TNBC |
| CID3963 | Myeloid_c11_cDC2_CD1C | 18 | 0.005103487 | TNBC |
| CID4461 | Myeloid_c11_cDC2_CD1C | 2 | 0.003169572 | ER+ |
| CID4463 | Myeloid_c11_cDC2_CD1C | 3 | 0.002636204 | ER+ |
| CID4471 | Myeloid_c11_cDC2_CD1C | 23 | 0.002671623 | ER+ |
| CID4530N | Myeloid_c11_cDC2_CD1C | 5 | 0.001134044 | ER+ |
| CID4535 | Myeloid_c11_cDC2_CD1C | 9 | 0.002272153 | ER+ |
| CID4040 | Myeloid_c11_cDC2_CD1C | 4 | 0.001580403 | ER+ |
| CID3941 | Myeloid_c11_cDC2_CD1C | 0 | 0 | ER+ |
| CID3948 | Myeloid_c11_cDC2_CD1C | 3 | 0.001289214 | ER+ |
| CID4067 | Myeloid_c11_cDC2_CD1C | 21 | 0.005579171 | ER+ |
| CID4290A | Myeloid_c11_cDC2_CD1C | 18 | 0.003109345 | ER+ |
| CID4398 | Myeloid_c11_cDC2_CD1C | 13 | 0.002920692 | ER+ |
| CID3586 | Myeloid_c12_Monocyte_1_IL1B | 30 | 0.00485594 | HER2+ |
| CID3921 | Myeloid_c12_Monocyte_1_IL1B | 49 | 0.016203704 | HER2+ |
| CID45171 | Myeloid_c12_Monocyte_1_IL1B | 69 | 0.028197793 | HER2+ |
| CID3838 | Myeloid_c12_Monocyte_1_IL1B | 47 | 0.019974501 | HER2+ |
| CID4066 | Myeloid_c12_Monocyte_1_IL1B | 34 | 0.006404219 | HER2+ |
| CID44041 | Myeloid_c12_Monocyte_1_IL1B | 9 | 0.004223369 | TNBC |
| CID4465 | Myeloid_c12_Monocyte_1_IL1B | 33 | 0.021099744 | TNBC |
| CID4495 | Myeloid_c12_Monocyte_1_IL1B | 132 | 0.016530996 | TNBC |
| CID44971 | Myeloid_c12_Monocyte_1_IL1B | 95 | 0.011895818 | TNBC |
| CID44991 | Myeloid_c12_Monocyte_1_IL1B | 23 | 0.003274954 | TNBC |
| CID4513 | Myeloid_c12_Monocyte_1_IL1B | 365 | 0.064958178 | TNBC |
| CID4515 | Myeloid_c12_Monocyte_1_IL1B | 67 | 0.01614847 | TNBC |
| CID4523 | Myeloid_c12_Monocyte_1_IL1B | 85 | 0.048460661 | TNBC |
| CID3946 | Myeloid_c12_Monocyte_1_IL1B | 44 | 0.056847545 | TNBC |
| CID3963 | Myeloid_c12_Monocyte_1_IL1B | 29 | 0.008222285 | TNBC |
| CID4461 | Myeloid_c12_Monocyte_1_IL1B | 4 | 0.006339144 | ER+ |
| CID4463 | Myeloid_c12_Monocyte_1_IL1B | 6 | 0.005272408 | ER+ |
| CID4471 | Myeloid_c12_Monocyte_1_IL1B | 33 | 0.003833198 | ER+ |
| CID4530N | Myeloid_c12_Monocyte_1_IL1B | 7 | 0.001587662 | ER+ |
| CID4535 | Myeloid_c12_Monocyte_1_IL1B | 50 | 0.012623075 | ER+ |
| CID4040 | Myeloid_c12_Monocyte_1_IL1B | 6 | 0.002370605 | ER+ |
| CID3941 | Myeloid_c12_Monocyte_1_IL1B | 6 | 0.009508716 | ER+ |
| CID3948 | Myeloid_c12_Monocyte_1_IL1B | 17 | 0.007305544 | ER+ |
| CID4067 | Myeloid_c12_Monocyte_1_IL1B | 27 | 0.00717322 | ER+ |
| CID4290A | Myeloid_c12_Monocyte_1_IL1B | 39 | 0.006736915 | ER+ |
| CID4398 | Myeloid_c12_Monocyte_1_IL1B | 33 | 0.007414064 | ER+ |
| CID3586 | Myeloid_c2_LAM2_APOE | 19 | 0.003075429 | HER2+ |
| CID3921 | Myeloid_c2_LAM2_APOE | 61 | 0.020171958 | HER2+ |
| CID45171 | Myeloid_c2_LAM2_APOE | 1 | 0.000408664 | HER2+ |
| CID3838 | Myeloid_c2_LAM2_APOE | 47 | 0.019974501 | HER2+ |
| CID4066 | Myeloid_c2_LAM2_APOE | 25 | 0.004708985 | HER2+ |
| CID44041 | Myeloid_c2_LAM2_APOE | 9 | 0.004223369 | TNBC |
| CID4465 | Myeloid_c2_LAM2_APOE | 1 | 0.000639386 | TNBC |
| CID4495 | Myeloid_c2_LAM2_APOE | 123 | 0.015403882 | TNBC |
| CID44971 | Myeloid_c2_LAM2_APOE | 103 | 0.012897571 | TNBC |
| CID44991 | Myeloid_c2_LAM2_APOE | 32 | 0.004556457 | TNBC |
| CID4513 | Myeloid_c2_LAM2_APOE | 334 | 0.059441182 | TNBC |
| CID4515 | Myeloid_c2_LAM2_APOE | 57 | 0.01373825 | TNBC |
| CID4523 | Myeloid_c2_LAM2_APOE | 4 | 0.002280502 | TNBC |
| CID3946 | Myeloid_c2_LAM2_APOE | 3 | 0.003875969 | TNBC |
| CID3963 | Myeloid_c2_LAM2_APOE | 110 | 0.031187978 | TNBC |
| CID4461 | Myeloid_c2_LAM2_APOE | 3 | 0.004754358 | ER+ |
| CID4463 | Myeloid_c2_LAM2_APOE | 14 | 0.012302285 | ER+ |
| CID4471 | Myeloid_c2_LAM2_APOE | 42 | 0.004878615 | ER+ |
| CID4530N | Myeloid_c2_LAM2_APOE | 12 | 0.002721706 | ER+ |
| CID4535 | Myeloid_c2_LAM2_APOE | 25 | 0.006311537 | ER+ |
| CID4040 | Myeloid_c2_LAM2_APOE | 9 | 0.003555907 | ER+ |
| CID3941 | Myeloid_c2_LAM2_APOE | 1 | 0.001584786 | ER+ |
| CID3948 | Myeloid_c2_LAM2_APOE | 2 | 0.000859476 | ER+ |
| CID4067 | Myeloid_c2_LAM2_APOE | 52 | 0.01381509 | ER+ |
| CID4290A | Myeloid_c2_LAM2_APOE | 31 | 0.005354984 | ER+ |
| CID4398 | Myeloid_c2_LAM2_APOE | 6 | 0.001348012 | ER+ |
| CID3586 | Myeloid_c3_cDC1_CLEC9A | 6 | 0.000971188 | HER2+ |
| CID3921 | Myeloid_c3_cDC1_CLEC9A | 8 | 0.002645503 | HER2+ |
| CID45171 | Myeloid_c3_cDC1_CLEC9A | 3 | 0.001225991 | HER2+ |
| CID3838 | Myeloid_c3_cDC1_CLEC9A | 10 | 0.004249894 | HER2+ |
| CID4066 | Myeloid_c3_cDC1_CLEC9A | 10 | 0.001883594 | HER2+ |
| CID44041 | Myeloid_c3_cDC1_CLEC9A | 2 | 0.000938527 | TNBC |
| CID4465 | Myeloid_c3_cDC1_CLEC9A | 3 | 0.001918159 | TNBC |
| CID4495 | Myeloid_c3_cDC1_CLEC9A | 10 | 0.001252348 | TNBC |
| CID44971 | Myeloid_c3_cDC1_CLEC9A | 21 | 0.002629602 | TNBC |
| CID44991 | Myeloid_c3_cDC1_CLEC9A | 6 | 0.000854336 | TNBC |
| CID4513 | Myeloid_c3_cDC1_CLEC9A | 27 | 0.004805125 | TNBC |
| CID4515 | Myeloid_c3_cDC1_CLEC9A | 5 | 0.00120511 | TNBC |
| CID4523 | Myeloid_c3_cDC1_CLEC9A | 2 | 0.001140251 | TNBC |
| CID3946 | Myeloid_c3_cDC1_CLEC9A | 0 | 0 | TNBC |
| CID3963 | Myeloid_c3_cDC1_CLEC9A | 2 | 0.000567054 | TNBC |
| CID4461 | Myeloid_c3_cDC1_CLEC9A | 1 | 0.001584786 | ER+ |
| CID4463 | Myeloid_c3_cDC1_CLEC9A | 1 | 0.000878735 | ER+ |
| CID4471 | Myeloid_c3_cDC1_CLEC9A | 2 | 0.000232315 | ER+ |
| CID4530N | Myeloid_c3_cDC1_CLEC9A | 3 | 0.000680426 | ER+ |
| CID4535 | Myeloid_c3_cDC1_CLEC9A | 3 | 0.000757384 | ER+ |
| CID4040 | Myeloid_c3_cDC1_CLEC9A | 0 | 0 | ER+ |
| CID3941 | Myeloid_c3_cDC1_CLEC9A | 1 | 0.001584786 | ER+ |
| CID3948 | Myeloid_c3_cDC1_CLEC9A | 3 | 0.001289214 | ER+ |
| CID4067 | Myeloid_c3_cDC1_CLEC9A | 6 | 0.001594049 | ER+ |
| CID4290A | Myeloid_c3_cDC1_CLEC9A | 5 | 0.000863707 | ER+ |
| CID4398 | Myeloid_c3_cDC1_CLEC9A | 16 | 0.003594698 | ER+ |
| CID3586 | Myeloid_c4_DCs_pDC_IRF7 | 38 | 0.006150858 | HER2+ |
| CID3921 | Myeloid_c4_DCs_pDC_IRF7 | 11 | 0.003637566 | HER2+ |
| CID45171 | Myeloid_c4_DCs_pDC_IRF7 | 8 | 0.003269309 | HER2+ |
| CID3838 | Myeloid_c4_DCs_pDC_IRF7 | 5 | 0.002124947 | HER2+ |
| CID4066 | Myeloid_c4_DCs_pDC_IRF7 | 7 | 0.001318516 | HER2+ |
| CID44041 | Myeloid_c4_DCs_pDC_IRF7 | 2 | 0.000938527 | TNBC |
| CID4465 | Myeloid_c4_DCs_pDC_IRF7 | 4 | 0.002557545 | TNBC |
| CID4495 | Myeloid_c4_DCs_pDC_IRF7 | 39 | 0.004884158 | TNBC |
| CID44971 | Myeloid_c4_DCs_pDC_IRF7 | 72 | 0.009015778 | TNBC |
| CID44991 | Myeloid_c4_DCs_pDC_IRF7 | 5 | 0.000711946 | TNBC |
| CID4513 | Myeloid_c4_DCs_pDC_IRF7 | 7 | 0.001245773 | TNBC |
| CID4515 | Myeloid_c4_DCs_pDC_IRF7 | 33 | 0.007953724 | TNBC |
| CID4523 | Myeloid_c4_DCs_pDC_IRF7 | 2 | 0.001140251 | TNBC |
| CID3946 | Myeloid_c4_DCs_pDC_IRF7 | 2 | 0.002583979 | TNBC |
| CID3963 | Myeloid_c4_DCs_pDC_IRF7 | 4 | 0.001134108 | TNBC |
| CID4461 | Myeloid_c4_DCs_pDC_IRF7 | 1 | 0.001584786 | ER+ |
| CID4463 | Myeloid_c4_DCs_pDC_IRF7 | 0 | 0 | ER+ |
| CID4471 | Myeloid_c4_DCs_pDC_IRF7 | 6 | 0.000696945 | ER+ |
| CID4530N | Myeloid_c4_DCs_pDC_IRF7 | 7 | 0.001587662 | ER+ |
| CID4535 | Myeloid_c4_DCs_pDC_IRF7 | 39 | 0.009845998 | ER+ |
| CID4040 | Myeloid_c4_DCs_pDC_IRF7 | 1 | 0.000395101 | ER+ |
| CID3941 | Myeloid_c4_DCs_pDC_IRF7 | 0 | 0 | ER+ |
| CID3948 | Myeloid_c4_DCs_pDC_IRF7 | 5 | 0.002148689 | ER+ |
| CID4067 | Myeloid_c4_DCs_pDC_IRF7 | 4 | 0.001062699 | ER+ |
| CID4290A | Myeloid_c4_DCs_pDC_IRF7 | 2 | 0.000345483 | ER+ |
| CID4398 | Myeloid_c4_DCs_pDC_IRF7 | 27 | 0.006066053 | ER+ |
| CID3586 | Myeloid_c5_Macrophage_3_SIGLEC1 | 0 | 0 | HER2+ |
| CID3921 | Myeloid_c5_Macrophage_3_SIGLEC1 | 0 | 0 | HER2+ |
| CID45171 | Myeloid_c5_Macrophage_3_SIGLEC1 | 0 | 0 | HER2+ |
| CID3838 | Myeloid_c5_Macrophage_3_SIGLEC1 | 0 | 0 | HER2+ |
| CID4066 | Myeloid_c5_Macrophage_3_SIGLEC1 | 0 | 0 | HER2+ |
| CID44041 | Myeloid_c5_Macrophage_3_SIGLEC1 | 0 | 0 | TNBC |
| CID4465 | Myeloid_c5_Macrophage_3_SIGLEC1 | 0 | 0 | TNBC |
| CID4495 | Myeloid_c5_Macrophage_3_SIGLEC1 | 0 | 0 | TNBC |
| CID44971 | Myeloid_c5_Macrophage_3_SIGLEC1 | 0 | 0 | TNBC |
| CID44991 | Myeloid_c5_Macrophage_3_SIGLEC1 | 0 | 0 | TNBC |
| CID4513 | Myeloid_c5_Macrophage_3_SIGLEC1 | 26 | 0.004627158 | TNBC |
| CID4515 | Myeloid_c5_Macrophage_3_SIGLEC1 | 0 | 0 | TNBC |
| CID4523 | Myeloid_c5_Macrophage_3_SIGLEC1 | 0 | 0 | TNBC |
| CID3946 | Myeloid_c5_Macrophage_3_SIGLEC1 | 0 | 0 | TNBC |
| CID3963 | Myeloid_c5_Macrophage_3_SIGLEC1 | 0 | 0 | TNBC |
| CID4461 | Myeloid_c5_Macrophage_3_SIGLEC1 | 0 | 0 | ER+ |
| CID4463 | Myeloid_c5_Macrophage_3_SIGLEC1 | 0 | 0 | ER+ |
| CID4471 | Myeloid_c5_Macrophage_3_SIGLEC1 | 0 | 0 | ER+ |
| CID4530N | Myeloid_c5_Macrophage_3_SIGLEC1 | 0 | 0 | ER+ |
| CID4535 | Myeloid_c5_Macrophage_3_SIGLEC1 | 0 | 0 | ER+ |
| CID4040 | Myeloid_c5_Macrophage_3_SIGLEC1 | 0 | 0 | ER+ |
| CID3941 | Myeloid_c5_Macrophage_3_SIGLEC1 | 0 | 0 | ER+ |
| CID3948 | Myeloid_c5_Macrophage_3_SIGLEC1 | 0 | 0 | ER+ |
| CID4067 | Myeloid_c5_Macrophage_3_SIGLEC1 | 0 | 0 | ER+ |
| CID4290A | Myeloid_c5_Macrophage_3_SIGLEC1 | 0 | 0 | ER+ |
| CID4398 | Myeloid_c5_Macrophage_3_SIGLEC1 | 0 | 0 | ER+ |
| CID3586 | Myeloid_c7_Monocyte_3_FCGR3A | 1 | 0.000161865 | HER2+ |
| CID3921 | Myeloid_c7_Monocyte_3_FCGR3A | 4 | 0.001322751 | HER2+ |
| CID45171 | Myeloid_c7_Monocyte_3_FCGR3A | 0 | 0 | HER2+ |
| CID3838 | Myeloid_c7_Monocyte_3_FCGR3A | 1 | 0.000424989 | HER2+ |
| CID4066 | Myeloid_c7_Monocyte_3_FCGR3A | 1 | 0.000188359 | HER2+ |
| CID44041 | Myeloid_c7_Monocyte_3_FCGR3A | 1 | 0.000469263 | TNBC |
| CID4465 | Myeloid_c7_Monocyte_3_FCGR3A | 0 | 0 | TNBC |
| CID4495 | Myeloid_c7_Monocyte_3_FCGR3A | 5 | 0.000626174 | TNBC |
| CID44971 | Myeloid_c7_Monocyte_3_FCGR3A | 7 | 0.000876534 | TNBC |
| CID44991 | Myeloid_c7_Monocyte_3_FCGR3A | 0 | 0 | TNBC |
| CID4513 | Myeloid_c7_Monocyte_3_FCGR3A | 3 | 0.000533903 | TNBC |
| CID4515 | Myeloid_c7_Monocyte_3_FCGR3A | 4 | 0.000964088 | TNBC |
| CID4523 | Myeloid_c7_Monocyte_3_FCGR3A | 0 | 0 | TNBC |
| CID3946 | Myeloid_c7_Monocyte_3_FCGR3A | 0 | 0 | TNBC |
| CID3963 | Myeloid_c7_Monocyte_3_FCGR3A | 2 | 0.000567054 | TNBC |
| CID4461 | Myeloid_c7_Monocyte_3_FCGR3A | 1 | 0.001584786 | ER+ |
| CID4463 | Myeloid_c7_Monocyte_3_FCGR3A | 2 | 0.001757469 | ER+ |
| CID4471 | Myeloid_c7_Monocyte_3_FCGR3A | 6 | 0.000696945 | ER+ |
| CID4530N | Myeloid_c7_Monocyte_3_FCGR3A | 4 | 0.000907235 | ER+ |
| CID4535 | Myeloid_c7_Monocyte_3_FCGR3A | 3 | 0.000757384 | ER+ |
| CID4040 | Myeloid_c7_Monocyte_3_FCGR3A | 0 | 0 | ER+ |
| CID3941 | Myeloid_c7_Monocyte_3_FCGR3A | 0 | 0 | ER+ |
| CID3948 | Myeloid_c7_Monocyte_3_FCGR3A | 0 | 0 | ER+ |
| CID4067 | Myeloid_c7_Monocyte_3_FCGR3A | 3 | 0.000797024 | ER+ |
| CID4290A | Myeloid_c7_Monocyte_3_FCGR3A | 2 | 0.000345483 | ER+ |
| CID4398 | Myeloid_c7_Monocyte_3_FCGR3A | 4 | 0.000898674 | ER+ |
| CID3586 | Myeloid_c8_Monocyte_2_S100A9 | 7 | 0.001133053 | HER2+ |
| CID3921 | Myeloid_c8_Monocyte_2_S100A9 | 30 | 0.009920635 | HER2+ |
| CID45171 | Myeloid_c8_Monocyte_2_S100A9 | 63 | 0.025745811 | HER2+ |
| CID3838 | Myeloid_c8_Monocyte_2_S100A9 | 24 | 0.010199745 | HER2+ |
| CID4066 | Myeloid_c8_Monocyte_2_S100A9 | 12 | 0.002260313 | HER2+ |
| CID44041 | Myeloid_c8_Monocyte_2_S100A9 | 8 | 0.003754106 | TNBC |
| CID4465 | Myeloid_c8_Monocyte_2_S100A9 | 3 | 0.001918159 | TNBC |
| CID4495 | Myeloid_c8_Monocyte_2_S100A9 | 72 | 0.009016907 | TNBC |
| CID44971 | Myeloid_c8_Monocyte_2_S100A9 | 53 | 0.006636614 | TNBC |
| CID44991 | Myeloid_c8_Monocyte_2_S100A9 | 12 | 0.001708672 | TNBC |
| CID4513 | Myeloid_c8_Monocyte_2_S100A9 | 324 | 0.057661506 | TNBC |
| CID4515 | Myeloid_c8_Monocyte_2_S100A9 | 123 | 0.029645698 | TNBC |
| CID4523 | Myeloid_c8_Monocyte_2_S100A9 | 45 | 0.025655644 | TNBC |
| CID3946 | Myeloid_c8_Monocyte_2_S100A9 | 0 | 0 | TNBC |
| CID3963 | Myeloid_c8_Monocyte_2_S100A9 | 40 | 0.011341083 | TNBC |
| CID4461 | Myeloid_c8_Monocyte_2_S100A9 | 1 | 0.001584786 | ER+ |
| CID4463 | Myeloid_c8_Monocyte_2_S100A9 | 10 | 0.008787346 | ER+ |
| CID4471 | Myeloid_c8_Monocyte_2_S100A9 | 13 | 0.001510048 | ER+ |
| CID4530N | Myeloid_c8_Monocyte_2_S100A9 | 18 | 0.004082558 | ER+ |
| CID4535 | Myeloid_c8_Monocyte_2_S100A9 | 19 | 0.004796768 | ER+ |
| CID4040 | Myeloid_c8_Monocyte_2_S100A9 | 5 | 0.001975504 | ER+ |
| CID3941 | Myeloid_c8_Monocyte_2_S100A9 | 2 | 0.003169572 | ER+ |
| CID3948 | Myeloid_c8_Monocyte_2_S100A9 | 10 | 0.004297379 | ER+ |
| CID4067 | Myeloid_c8_Monocyte_2_S100A9 | 12 | 0.003188098 | ER+ |
| CID4290A | Myeloid_c8_Monocyte_2_S100A9 | 10 | 0.001727414 | ER+ |
| CID4398 | Myeloid_c8_Monocyte_2_S100A9 | 19 | 0.004268704 | ER+ |
| CID3586 | Myeloid_c9_Macrophage_2_CXCL10 | 3 | 0.000485594 | HER2+ |
| CID3921 | Myeloid_c9_Macrophage_2_CXCL10 | 21 | 0.006944444 | HER2+ |
| CID45171 | Myeloid_c9_Macrophage_2_CXCL10 | 4 | 0.001634655 | HER2+ |
| CID3838 | Myeloid_c9_Macrophage_2_CXCL10 | 20 | 0.008499788 | HER2+ |
| CID4066 | Myeloid_c9_Macrophage_2_CXCL10 | 8 | 0.001506875 | HER2+ |
| CID44041 | Myeloid_c9_Macrophage_2_CXCL10 | 2 | 0.000938527 | TNBC |
| CID4465 | Myeloid_c9_Macrophage_2_CXCL10 | 5 | 0.003196931 | TNBC |
| CID4495 | Myeloid_c9_Macrophage_2_CXCL10 | 113 | 0.014151534 | TNBC |
| CID44971 | Myeloid_c9_Macrophage_2_CXCL10 | 34 | 0.004257451 | TNBC |
| CID44991 | Myeloid_c9_Macrophage_2_CXCL10 | 20 | 0.002847786 | TNBC |
| CID4513 | Myeloid_c9_Macrophage_2_CXCL10 | 133 | 0.023669692 | TNBC |
| CID4515 | Myeloid_c9_Macrophage_2_CXCL10 | 29 | 0.006989636 | TNBC |
| CID4523 | Myeloid_c9_Macrophage_2_CXCL10 | 12 | 0.006841505 | TNBC |
| CID3946 | Myeloid_c9_Macrophage_2_CXCL10 | 0 | 0 | TNBC |
| CID3963 | Myeloid_c9_Macrophage_2_CXCL10 | 40 | 0.011341083 | TNBC |
| CID4461 | Myeloid_c9_Macrophage_2_CXCL10 | 7 | 0.011093502 | ER+ |
| CID4463 | Myeloid_c9_Macrophage_2_CXCL10 | 5 | 0.004393673 | ER+ |
| CID4471 | Myeloid_c9_Macrophage_2_CXCL10 | 3 | 0.000348473 | ER+ |
| CID4530N | Myeloid_c9_Macrophage_2_CXCL10 | 4 | 0.000907235 | ER+ |
| CID4535 | Myeloid_c9_Macrophage_2_CXCL10 | 14 | 0.003534461 | ER+ |
| CID4040 | Myeloid_c9_Macrophage_2_CXCL10 | 1 | 0.000395101 | ER+ |
| CID3941 | Myeloid_c9_Macrophage_2_CXCL10 | 1 | 0.001584786 | ER+ |
| CID3948 | Myeloid_c9_Macrophage_2_CXCL10 | 2 | 0.000859476 | ER+ |
| CID4067 | Myeloid_c9_Macrophage_2_CXCL10 | 10 | 0.002656748 | ER+ |
| CID4290A | Myeloid_c9_Macrophage_2_CXCL10 | 9 | 0.001554673 | ER+ |
| CID4398 | Myeloid_c9_Macrophage_2_CXCL10 | 5 | 0.001123343 | ER+ |
| CID3586 | Myoepithelial | 136 | 0.022013597 | HER2+ |
| CID3921 | Myoepithelial | 0 | 0 | HER2+ |
| CID45171 | Myoepithelial | 0 | 0 | HER2+ |
| CID3838 | Myoepithelial | 0 | 0 | HER2+ |
| CID4066 | Myoepithelial | 103 | 0.019401017 | HER2+ |
| CID44041 | Myoepithelial | 9 | 0.004223369 | TNBC |
| CID4465 | Myoepithelial | 0 | 0 | TNBC |
| CID4495 | Myoepithelial | 0 | 0 | TNBC |
| CID44971 | Myoepithelial | 124 | 0.015527173 | TNBC |
| CID44991 | Myoepithelial | 4 | 0.000569557 | TNBC |
| CID4513 | Myoepithelial | 0 | 0 | TNBC |
| CID4515 | Myoepithelial | 9 | 0.002169197 | TNBC |
| CID4523 | Myoepithelial | 0 | 0 | TNBC |
| CID3946 | Myoepithelial | 0 | 0 | TNBC |
| CID3963 | Myoepithelial | 0 | 0 | TNBC |
| CID4461 | Myoepithelial | 0 | 0 | ER+ |
| CID4463 | Myoepithelial | 4 | 0.003514938 | ER+ |
| CID4471 | Myoepithelial | 657 | 0.076315484 | ER+ |
| CID4530N | Myoepithelial | 46 | 0.010433205 | ER+ |
| CID4535 | Myoepithelial | 2 | 0.000504923 | ER+ |
| CID4040 | Myoepithelial | 0 | 0 | ER+ |
| CID3941 | Myoepithelial | 0 | 0 | ER+ |
| CID3948 | Myoepithelial | 0 | 0 | ER+ |
| CID4067 | Myoepithelial | 0 | 0 | ER+ |
| CID4290A | Myoepithelial | 4 | 0.000690966 | ER+ |
| CID4398 | Myoepithelial | 0 | 0 | ER+ |
| CID3586 | Plasmablasts | 0 | 0 | HER2+ |
| CID3921 | Plasmablasts | 175 | 0.05787037 | HER2+ |
| CID45171 | Plasmablasts | 0 | 0 | HER2+ |
| CID3838 | Plasmablasts | 51 | 0.021674458 | HER2+ |
| CID4066 | Plasmablasts | 0 | 0 | HER2+ |
| CID44041 | Plasmablasts | 0 | 0 | TNBC |
| CID4465 | Plasmablasts | 110 | 0.070332481 | TNBC |
| CID4495 | Plasmablasts | 1020 | 0.127739512 | TNBC |
| CID44971 | Plasmablasts | 48 | 0.006010518 | TNBC |
| CID44991 | Plasmablasts | 1453 | 0.206891642 | TNBC |
| CID4513 | Plasmablasts | 0 | 0 | TNBC |
| CID4515 | Plasmablasts | 36 | 0.00867679 | TNBC |
| CID4523 | Plasmablasts | 0 | 0 | TNBC |
| CID3946 | Plasmablasts | 0 | 0 | TNBC |
| CID3963 | Plasmablasts | 0 | 0 | TNBC |
| CID4461 | Plasmablasts | 32 | 0.050713154 | ER+ |
| CID4463 | Plasmablasts | 0 | 0 | ER+ |
| CID4471 | Plasmablasts | 51 | 0.005924033 | ER+ |
| CID4530N | Plasmablasts | 55 | 0.012474484 | ER+ |
| CID4535 | Plasmablasts | 96 | 0.024236304 | ER+ |
| CID4040 | Plasmablasts | 74 | 0.029237456 | ER+ |
| CID3941 | Plasmablasts | 0 | 0 | ER+ |
| CID3948 | Plasmablasts | 232 | 0.099699183 | ER+ |
| CID4067 | Plasmablasts | 0 | 0 | ER+ |
| CID4290A | Plasmablasts | 0 | 0 | ER+ |
| CID4398 | Plasmablasts | 91 | 0.020444844 | ER+ |
| CID3586 | PVL Differentiated s3 | 10 | 0.001618647 | HER2+ |
| CID3921 | PVL Differentiated s3 | 46 | 0.01521164 | HER2+ |
| CID45171 | PVL Differentiated s3 | 10 | 0.004086637 | HER2+ |
| CID3838 | PVL Differentiated s3 | 82 | 0.034849129 | HER2+ |
| CID4066 | PVL Differentiated s3 | 402 | 0.075720475 | HER2+ |
| CID44041 | PVL Differentiated s3 | 66 | 0.030971375 | TNBC |
| CID4465 | PVL Differentiated s3 | 187 | 0.119565217 | TNBC |
| CID4495 | PVL Differentiated s3 | 112 | 0.014026299 | TNBC |
| CID44971 | PVL Differentiated s3 | 29 | 0.003631355 | TNBC |
| CID44991 | PVL Differentiated s3 | 20 | 0.002847786 | TNBC |
| CID4513 | PVL Differentiated s3 | 49 | 0.008720413 | TNBC |
| CID4515 | PVL Differentiated s3 | 86 | 0.020727886 | TNBC |
| CID4523 | PVL Differentiated s3 | 5 | 0.002850627 | TNBC |
| CID3946 | PVL Differentiated s3 | 175 | 0.226098191 | TNBC |
| CID3963 | PVL Differentiated s3 | 12 | 0.003402325 | TNBC |
| CID4461 | PVL Differentiated s3 | 38 | 0.06022187 | ER+ |
| CID4463 | PVL Differentiated s3 | 26 | 0.0228471 | ER+ |
| CID4471 | PVL Differentiated s3 | 868 | 0.100824718 | ER+ |
| CID4530N | PVL Differentiated s3 | 340 | 0.077114992 | ER+ |
| CID4535 | PVL Differentiated s3 | 430 | 0.108558445 | ER+ |
| CID4040 | PVL Differentiated s3 | 271 | 0.107072303 | ER+ |
| CID3941 | PVL Differentiated s3 | 12 | 0.019017433 | ER+ |
| CID3948 | PVL Differentiated s3 | 28 | 0.01203266 | ER+ |
| CID4067 | PVL Differentiated s3 | 43 | 0.011424017 | ER+ |
| CID4290A | PVL Differentiated s3 | 79 | 0.013646571 | ER+ |
| CID4398 | PVL Differentiated s3 | 61 | 0.013704785 | ER+ |
| CID3586 | PVL Immature s1 | 6 | 0.000971188 | HER2+ |
| CID3921 | PVL Immature s1 | 13 | 0.004298942 | HER2+ |
| CID45171 | PVL Immature s1 | 2 | 0.000817327 | HER2+ |
| CID3838 | PVL Immature s1 | 30 | 0.012749681 | HER2+ |
| CID4066 | PVL Immature s1 | 131 | 0.02467508 | HER2+ |
| CID44041 | PVL Immature s1 | 39 | 0.018301267 | TNBC |
| CID4465 | PVL Immature s1 | 60 | 0.038363171 | TNBC |
| CID4495 | PVL Immature s1 | 42 | 0.005259862 | TNBC |
| CID44971 | PVL Immature s1 | 32 | 0.004007012 | TNBC |
| CID44991 | PVL Immature s1 | 8 | 0.001139114 | TNBC |
| CID4513 | PVL Immature s1 | 16 | 0.002847482 | TNBC |
| CID4515 | PVL Immature s1 | 20 | 0.004820439 | TNBC |
| CID4523 | PVL Immature s1 | 3 | 0.001710376 | TNBC |
| CID3946 | PVL Immature s1 | 63 | 0.081395349 | TNBC |
| CID3963 | PVL Immature s1 | 12 | 0.003402325 | TNBC |
| CID4461 | PVL Immature s1 | 3 | 0.004754358 | ER+ |
| CID4463 | PVL Immature s1 | 3 | 0.002636204 | ER+ |
| CID4471 | PVL Immature s1 | 325 | 0.037751191 | ER+ |
| CID4530N | PVL Immature s1 | 74 | 0.016783851 | ER+ |
| CID4535 | PVL Immature s1 | 73 | 0.018429689 | ER+ |
| CID4040 | PVL Immature s1 | 109 | 0.043065982 | ER+ |
| CID3941 | PVL Immature s1 | 12 | 0.019017433 | ER+ |
| CID3948 | PVL Immature s1 | 27 | 0.011602922 | ER+ |
| CID4067 | PVL Immature s1 | 13 | 0.003453773 | ER+ |
| CID4290A | PVL Immature s1 | 35 | 0.006045949 | ER+ |
| CID4398 | PVL Immature s1 | 18 | 0.004044035 | ER+ |
| CID3586 | PVL_Immature s2 | 5 | 0.000809323 | HER2+ |
| CID3921 | PVL_Immature s2 | 13 | 0.004298942 | HER2+ |
| CID45171 | PVL_Immature s2 | 1 | 0.000408664 | HER2+ |
| CID3838 | PVL_Immature s2 | 42 | 0.017849554 | HER2+ |
| CID4066 | PVL_Immature s2 | 91 | 0.017140704 | HER2+ |
| CID44041 | PVL_Immature s2 | 23 | 0.010793055 | TNBC |
| CID4465 | PVL_Immature s2 | 63 | 0.04028133 | TNBC |
| CID4495 | PVL_Immature s2 | 35 | 0.004383219 | TNBC |
| CID44971 | PVL_Immature s2 | 30 | 0.003756574 | TNBC |
| CID44991 | PVL_Immature s2 | 12 | 0.001708672 | TNBC |
| CID4513 | PVL_Immature s2 | 15 | 0.002669514 | TNBC |
| CID4515 | PVL_Immature s2 | 15 | 0.003615329 | TNBC |
| CID4523 | PVL_Immature s2 | 2 | 0.001140251 | TNBC |
| CID3946 | PVL_Immature s2 | 10 | 0.012919897 | TNBC |
| CID3963 | PVL_Immature s2 | 3 | 0.000850581 | TNBC |
| CID4461 | PVL_Immature s2 | 7 | 0.011093502 | ER+ |
| CID4463 | PVL_Immature s2 | 2 | 0.001757469 | ER+ |
| CID4471 | PVL_Immature s2 | 92 | 0.010686491 | ER+ |
| CID4530N | PVL_Immature s2 | 55 | 0.012474484 | ER+ |
| CID4535 | PVL_Immature s2 | 79 | 0.019944458 | ER+ |
| CID4040 | PVL_Immature s2 | 59 | 0.023310944 | ER+ |
| CID3941 | PVL_Immature s2 | 0 | 0 | ER+ |
| CID3948 | PVL_Immature s2 | 7 | 0.003008165 | ER+ |
| CID4067 | PVL_Immature s2 | 25 | 0.00664187 | ER+ |
| CID4290A | PVL_Immature s2 | 25 | 0.004318535 | ER+ |
| CID4398 | PVL_Immature s2 | 6 | 0.001348012 | ER+ |
| CID3586 | T_cells_c0_CD4+_CCR7 | 941 | 0.152314665 | HER2+ |
| CID3921 | T_cells_c0_CD4+_CCR7 | 211 | 0.069775132 | HER2+ |
| CID45171 | T_cells_c0_CD4+_CCR7 | 197 | 0.080506743 | HER2+ |
| CID3838 | T_cells_c0_CD4+_CCR7 | 239 | 0.101572461 | HER2+ |
| CID4066 | T_cells_c0_CD4+_CCR7 | 167 | 0.031456018 | HER2+ |
| CID44041 | T_cells_c0_CD4+_CCR7 | 88 | 0.041295167 | TNBC |
| CID4465 | T_cells_c0_CD4+_CCR7 | 1 | 0.000639386 | TNBC |
| CID4495 | T_cells_c0_CD4+_CCR7 | 738 | 0.092423294 | TNBC |
| CID44971 | T_cells_c0_CD4+_CCR7 | 1051 | 0.131605309 | TNBC |
| CID44991 | T_cells_c0_CD4+_CCR7 | 68 | 0.009682472 | TNBC |
| CID4513 | T_cells_c0_CD4+_CCR7 | 132 | 0.023491725 | TNBC |
| CID4515 | T_cells_c0_CD4+_CCR7 | 58 | 0.013979272 | TNBC |
| CID4523 | T_cells_c0_CD4+_CCR7 | 13 | 0.007411631 | TNBC |
| CID3946 | T_cells_c0_CD4+_CCR7 | 9 | 0.011627907 | TNBC |
| CID3963 | T_cells_c0_CD4+_CCR7 | 268 | 0.075985257 | TNBC |
| CID4461 | T_cells_c0_CD4+_CCR7 | 0 | 0 | ER+ |
| CID4463 | T_cells_c0_CD4+_CCR7 | 10 | 0.008787346 | ER+ |
| CID4471 | T_cells_c0_CD4+_CCR7 | 112 | 0.013009641 | ER+ |
| CID4530N | T_cells_c0_CD4+_CCR7 | 29 | 0.006577455 | ER+ |
| CID4535 | T_cells_c0_CD4+_CCR7 | 23 | 0.005806614 | ER+ |
| CID4040 | T_cells_c0_CD4+_CCR7 | 234 | 0.092453576 | ER+ |
| CID3941 | T_cells_c0_CD4+_CCR7 | 34 | 0.053882726 | ER+ |
| CID3948 | T_cells_c0_CD4+_CCR7 | 58 | 0.024924796 | ER+ |
| CID4067 | T_cells_c0_CD4+_CCR7 | 33 | 0.008767269 | ER+ |
| CID4290A | T_cells_c0_CD4+_CCR7 | 18 | 0.003109345 | ER+ |
| CID4398 | T_cells_c0_CD4+_CCR7 | 220 | 0.049427095 | ER+ |
| CID3586 | T_cells_c1_CD4+_IL7R | 1329 | 0.215118161 | HER2+ |
| CID3921 | T_cells_c1_CD4+_IL7R | 315 | 0.104166667 | HER2+ |
| CID45171 | T_cells_c1_CD4+_IL7R | 389 | 0.158970168 | HER2+ |
| CID3838 | T_cells_c1_CD4+_IL7R | 226 | 0.096047599 | HER2+ |
| CID4066 | T_cells_c1_CD4+_IL7R | 607 | 0.11433415 | HER2+ |
| CID44041 | T_cells_c1_CD4+_IL7R | 278 | 0.130455185 | TNBC |
| CID4465 | T_cells_c1_CD4+_IL7R | 37 | 0.023657289 | TNBC |
| CID4495 | T_cells_c1_CD4+_IL7R | 186 | 0.023293676 | TNBC |
| CID44971 | T_cells_c1_CD4+_IL7R | 350 | 0.043826697 | TNBC |
| CID44991 | T_cells_c1_CD4+_IL7R | 163 | 0.023209455 | TNBC |
| CID4513 | T_cells_c1_CD4+_IL7R | 264 | 0.046983449 | TNBC |
| CID4515 | T_cells_c1_CD4+_IL7R | 39 | 0.009399855 | TNBC |
| CID4523 | T_cells_c1_CD4+_IL7R | 31 | 0.017673888 | TNBC |
| CID3946 | T_cells_c1_CD4+_IL7R | 22 | 0.028423773 | TNBC |
| CID3963 | T_cells_c1_CD4+_IL7R | 465 | 0.131840091 | TNBC |
| CID4461 | T_cells_c1_CD4+_IL7R | 28 | 0.04437401 | ER+ |
| CID4463 | T_cells_c1_CD4+_IL7R | 86 | 0.075571178 | ER+ |
| CID4471 | T_cells_c1_CD4+_IL7R | 194 | 0.022534557 | ER+ |
| CID4530N | T_cells_c1_CD4+_IL7R | 69 | 0.015649807 | ER+ |
| CID4535 | T_cells_c1_CD4+_IL7R | 166 | 0.041908609 | ER+ |
| CID4040 | T_cells_c1_CD4+_IL7R | 253 | 0.09996049 | ER+ |
| CID3941 | T_cells_c1_CD4+_IL7R | 61 | 0.096671949 | ER+ |
| CID3948 | T_cells_c1_CD4+_IL7R | 449 | 0.192952299 | ER+ |
| CID4067 | T_cells_c1_CD4+_IL7R | 212 | 0.056323061 | ER+ |
| CID4290A | T_cells_c1_CD4+_IL7R | 202 | 0.034893764 | ER+ |
| CID4398 | T_cells_c1_CD4+_IL7R | 1365 | 0.306672658 | ER+ |
| CID3586 | T_cells_c10_NKT_cells_FCGR3A | 95 | 0.015377145 | HER2+ |
| CID3921 | T_cells_c10_NKT_cells_FCGR3A | 17 | 0.005621693 | HER2+ |
| CID45171 | T_cells_c10_NKT_cells_FCGR3A | 206 | 0.084184716 | HER2+ |
| CID3838 | T_cells_c10_NKT_cells_FCGR3A | 28 | 0.011899703 | HER2+ |
| CID4066 | T_cells_c10_NKT_cells_FCGR3A | 39 | 0.007346016 | HER2+ |
| CID44041 | T_cells_c10_NKT_cells_FCGR3A | 6 | 0.00281558 | TNBC |
| CID4465 | T_cells_c10_NKT_cells_FCGR3A | 5 | 0.003196931 | TNBC |
| CID4495 | T_cells_c10_NKT_cells_FCGR3A | 31 | 0.003882279 | TNBC |
| CID44971 | T_cells_c10_NKT_cells_FCGR3A | 43 | 0.005384423 | TNBC |
| CID44991 | T_cells_c10_NKT_cells_FCGR3A | 47 | 0.006692297 | TNBC |
| CID4513 | T_cells_c10_NKT_cells_FCGR3A | 45 | 0.008008542 | TNBC |
| CID4515 | T_cells_c10_NKT_cells_FCGR3A | 73 | 0.017594601 | TNBC |
| CID4523 | T_cells_c10_NKT_cells_FCGR3A | 12 | 0.006841505 | TNBC |
| CID3946 | T_cells_c10_NKT_cells_FCGR3A | 4 | 0.005167959 | TNBC |
| CID3963 | T_cells_c10_NKT_cells_FCGR3A | 94 | 0.026651545 | TNBC |
| CID4461 | T_cells_c10_NKT_cells_FCGR3A | 3 | 0.004754358 | ER+ |
| CID4463 | T_cells_c10_NKT_cells_FCGR3A | 19 | 0.016695958 | ER+ |
| CID4471 | T_cells_c10_NKT_cells_FCGR3A | 32 | 0.00371704 | ER+ |
| CID4530N | T_cells_c10_NKT_cells_FCGR3A | 45 | 0.010206396 | ER+ |
| CID4535 | T_cells_c10_NKT_cells_FCGR3A | 15 | 0.003786922 | ER+ |
| CID4040 | T_cells_c10_NKT_cells_FCGR3A | 22 | 0.008692217 | ER+ |
| CID3941 | T_cells_c10_NKT_cells_FCGR3A | 9 | 0.014263074 | ER+ |
| CID3948 | T_cells_c10_NKT_cells_FCGR3A | 40 | 0.017189514 | ER+ |
| CID4067 | T_cells_c10_NKT_cells_FCGR3A | 39 | 0.010361318 | ER+ |
| CID4290A | T_cells_c10_NKT_cells_FCGR3A | 24 | 0.004145794 | ER+ |
| CID4398 | T_cells_c10_NKT_cells_FCGR3A | 129 | 0.028982251 | ER+ |
| CID3586 | T_cells_c11_MKI67 | 56 | 0.009064422 | HER2+ |
| CID3921 | T_cells_c11_MKI67 | 34 | 0.011243386 | HER2+ |
| CID45171 | T_cells_c11_MKI67 | 18 | 0.007355946 | HER2+ |
| CID3838 | T_cells_c11_MKI67 | 42 | 0.017849554 | HER2+ |
| CID4066 | T_cells_c11_MKI67 | 38 | 0.007157657 | HER2+ |
| CID44041 | T_cells_c11_MKI67 | 5 | 0.002346316 | TNBC |
| CID4465 | T_cells_c11_MKI67 | 10 | 0.006393862 | TNBC |
| CID4495 | T_cells_c11_MKI67 | 430 | 0.053850971 | TNBC |
| CID44971 | T_cells_c11_MKI67 | 271 | 0.033934385 | TNBC |
| CID44991 | T_cells_c11_MKI67 | 149 | 0.021216005 | TNBC |
| CID4513 | T_cells_c11_MKI67 | 181 | 0.032212137 | TNBC |
| CID4515 | T_cells_c11_MKI67 | 20 | 0.004820439 | TNBC |
| CID4523 | T_cells_c11_MKI67 | 14 | 0.007981756 | TNBC |
| CID3946 | T_cells_c11_MKI67 | 1 | 0.00129199 | TNBC |
| CID3963 | T_cells_c11_MKI67 | 73 | 0.020697477 | TNBC |
| CID4461 | T_cells_c11_MKI67 | 8 | 0.012678288 | ER+ |
| CID4463 | T_cells_c11_MKI67 | 5 | 0.004393673 | ER+ |
| CID4471 | T_cells_c11_MKI67 | 8 | 0.00092926 | ER+ |
| CID4530N | T_cells_c11_MKI67 | 1 | 0.000226809 | ER+ |
| CID4535 | T_cells_c11_MKI67 | 19 | 0.004796768 | ER+ |
| CID4040 | T_cells_c11_MKI67 | 25 | 0.009877519 | ER+ |
| CID3941 | T_cells_c11_MKI67 | 5 | 0.00792393 | ER+ |
| CID3948 | T_cells_c11_MKI67 | 24 | 0.010313709 | ER+ |
| CID4067 | T_cells_c11_MKI67 | 6 | 0.001594049 | ER+ |
| CID4290A | T_cells_c11_MKI67 | 8 | 0.001381931 | ER+ |
| CID4398 | T_cells_c11_MKI67 | 77 | 0.017299483 | ER+ |
| CID3586 | T_cells_c2_CD4+_T-regs_FOXP3 | 355 | 0.057461962 | HER2+ |
| CID3921 | T_cells_c2_CD4+_T-regs_FOXP3 | 234 | 0.077380952 | HER2+ |
| CID45171 | T_cells_c2_CD4+_T-regs_FOXP3 | 88 | 0.035962403 | HER2+ |
| CID3838 | T_cells_c2_CD4+_T-regs_FOXP3 | 330 | 0.140246494 | HER2+ |
| CID4066 | T_cells_c2_CD4+_T-regs_FOXP3 | 243 | 0.045771332 | HER2+ |
| CID44041 | T_cells_c2_CD4+_T-regs_FOXP3 | 52 | 0.024401689 | TNBC |
| CID4465 | T_cells_c2_CD4+_T-regs_FOXP3 | 23 | 0.014705882 | TNBC |
| CID4495 | T_cells_c2_CD4+_T-regs_FOXP3 | 428 | 0.053600501 | TNBC |
| CID44971 | T_cells_c2_CD4+_T-regs_FOXP3 | 651 | 0.081517656 | TNBC |
| CID44991 | T_cells_c2_CD4+_T-regs_FOXP3 | 154 | 0.021927951 | TNBC |
| CID4513 | T_cells_c2_CD4+_T-regs_FOXP3 | 151 | 0.026873109 | TNBC |
| CID4515 | T_cells_c2_CD4+_T-regs_FOXP3 | 29 | 0.006989636 | TNBC |
| CID4523 | T_cells_c2_CD4+_T-regs_FOXP3 | 9 | 0.005131129 | TNBC |
| CID3946 | T_cells_c2_CD4+_T-regs_FOXP3 | 17 | 0.021963824 | TNBC |
| CID3963 | T_cells_c2_CD4+_T-regs_FOXP3 | 196 | 0.055571307 | TNBC |
| CID4461 | T_cells_c2_CD4+_T-regs_FOXP3 | 8 | 0.012678288 | ER+ |
| CID4463 | T_cells_c2_CD4+_T-regs_FOXP3 | 14 | 0.012302285 | ER+ |
| CID4471 | T_cells_c2_CD4+_T-regs_FOXP3 | 94 | 0.010918806 | ER+ |
| CID4530N | T_cells_c2_CD4+_T-regs_FOXP3 | 22 | 0.004989794 | ER+ |
| CID4535 | T_cells_c2_CD4+_T-regs_FOXP3 | 31 | 0.007826306 | ER+ |
| CID4040 | T_cells_c2_CD4+_T-regs_FOXP3 | 187 | 0.07388384 | ER+ |
| CID3941 | T_cells_c2_CD4+_T-regs_FOXP3 | 11 | 0.017432647 | ER+ |
| CID3948 | T_cells_c2_CD4+_T-regs_FOXP3 | 248 | 0.106574989 | ER+ |
| CID4067 | T_cells_c2_CD4+_T-regs_FOXP3 | 89 | 0.023645058 | ER+ |
| CID4290A | T_cells_c2_CD4+_T-regs_FOXP3 | 93 | 0.016064951 | ER+ |
| CID4398 | T_cells_c2_CD4+_T-regs_FOXP3 | 489 | 0.109862952 | ER+ |
| CID3586 | T_cells_c3_CD4+_Tfh_CXCL13 | 283 | 0.045807705 | HER2+ |
| CID3921 | T_cells_c3_CD4+_Tfh_CXCL13 | 215 | 0.071097884 | HER2+ |
| CID45171 | T_cells_c3_CD4+_Tfh_CXCL13 | 30 | 0.01225991 | HER2+ |
| CID3838 | T_cells_c3_CD4+_Tfh_CXCL13 | 120 | 0.050998725 | HER2+ |
| CID4066 | T_cells_c3_CD4+_Tfh_CXCL13 | 91 | 0.017140704 | HER2+ |
| CID44041 | T_cells_c3_CD4+_Tfh_CXCL13 | 14 | 0.006569686 | TNBC |
| CID4465 | T_cells_c3_CD4+_Tfh_CXCL13 | 3 | 0.001918159 | TNBC |
| CID4495 | T_cells_c3_CD4+_Tfh_CXCL13 | 389 | 0.048716343 | TNBC |
| CID44971 | T_cells_c3_CD4+_Tfh_CXCL13 | 195 | 0.024417731 | TNBC |
| CID44991 | T_cells_c3_CD4+_Tfh_CXCL13 | 85 | 0.01210309 | TNBC |
| CID4513 | T_cells_c3_CD4+_Tfh_CXCL13 | 50 | 0.00889838 | TNBC |
| CID4515 | T_cells_c3_CD4+_Tfh_CXCL13 | 38 | 0.009158833 | TNBC |
| CID4523 | T_cells_c3_CD4+_Tfh_CXCL13 | 7 | 0.003990878 | TNBC |
| CID3946 | T_cells_c3_CD4+_Tfh_CXCL13 | 2 | 0.002583979 | TNBC |
| CID3963 | T_cells_c3_CD4+_Tfh_CXCL13 | 136 | 0.038559682 | TNBC |
| CID4461 | T_cells_c3_CD4+_Tfh_CXCL13 | 8 | 0.012678288 | ER+ |
| CID4463 | T_cells_c3_CD4+_Tfh_CXCL13 | 5 | 0.004393673 | ER+ |
| CID4471 | T_cells_c3_CD4+_Tfh_CXCL13 | 12 | 0.00139389 | ER+ |
| CID4530N | T_cells_c3_CD4+_Tfh_CXCL13 | 7 | 0.001587662 | ER+ |
| CID4535 | T_cells_c3_CD4+_Tfh_CXCL13 | 19 | 0.004796768 | ER+ |
| CID4040 | T_cells_c3_CD4+_Tfh_CXCL13 | 156 | 0.061635717 | ER+ |
| CID3941 | T_cells_c3_CD4+_Tfh_CXCL13 | 2 | 0.003169572 | ER+ |
| CID3948 | T_cells_c3_CD4+_Tfh_CXCL13 | 90 | 0.038676407 | ER+ |
| CID4067 | T_cells_c3_CD4+_Tfh_CXCL13 | 19 | 0.005047821 | ER+ |
| CID4290A | T_cells_c3_CD4+_Tfh_CXCL13 | 32 | 0.005527725 | ER+ |
| CID4398 | T_cells_c3_CD4+_Tfh_CXCL13 | 239 | 0.053695799 | ER+ |
| CID3586 | T_cells_c4_CD8+_ZFP36 | 953 | 0.154257041 | HER2+ |
| CID3921 | T_cells_c4_CD8+_ZFP36 | 234 | 0.077380952 | HER2+ |
| CID45171 | T_cells_c4_CD8+_ZFP36 | 130 | 0.053126277 | HER2+ |
| CID3838 | T_cells_c4_CD8+_ZFP36 | 118 | 0.050148746 | HER2+ |
| CID4066 | T_cells_c4_CD8+_ZFP36 | 614 | 0.115652665 | HER2+ |
| CID44041 | T_cells_c4_CD8+_ZFP36 | 192 | 0.090098545 | TNBC |
| CID4465 | T_cells_c4_CD8+_ZFP36 | 2 | 0.001278772 | TNBC |
| CID4495 | T_cells_c4_CD8+_ZFP36 | 225 | 0.028177833 | TNBC |
| CID44971 | T_cells_c4_CD8+_ZFP36 | 410 | 0.051339845 | TNBC |
| CID44991 | T_cells_c4_CD8+_ZFP36 | 72 | 0.010252029 | TNBC |
| CID4513 | T_cells_c4_CD8+_ZFP36 | 204 | 0.036305392 | TNBC |
| CID4515 | T_cells_c4_CD8+_ZFP36 | 40 | 0.009640877 | TNBC |
| CID4523 | T_cells_c4_CD8+_ZFP36 | 25 | 0.014253136 | TNBC |
| CID3946 | T_cells_c4_CD8+_ZFP36 | 15 | 0.019379845 | TNBC |
| CID3963 | T_cells_c4_CD8+_ZFP36 | 550 | 0.155939892 | TNBC |
| CID4461 | T_cells_c4_CD8+_ZFP36 | 5 | 0.00792393 | ER+ |
| CID4463 | T_cells_c4_CD8+_ZFP36 | 27 | 0.023725835 | ER+ |
| CID4471 | T_cells_c4_CD8+_ZFP36 | 89 | 0.010338018 | ER+ |
| CID4530N | T_cells_c4_CD8+_ZFP36 | 47 | 0.010660014 | ER+ |
| CID4535 | T_cells_c4_CD8+_ZFP36 | 57 | 0.014390305 | ER+ |
| CID4040 | T_cells_c4_CD8+_ZFP36 | 342 | 0.135124457 | ER+ |
| CID3941 | T_cells_c4_CD8+_ZFP36 | 48 | 0.076069731 | ER+ |
| CID3948 | T_cells_c4_CD8+_ZFP36 | 214 | 0.091963902 | ER+ |
| CID4067 | T_cells_c4_CD8+_ZFP36 | 78 | 0.020722635 | ER+ |
| CID4290A | T_cells_c4_CD8+_ZFP36 | 28 | 0.004836759 | ER+ |
| CID4398 | T_cells_c4_CD8+_ZFP36 | 344 | 0.077286003 | ER+ |
| CID3586 | T_cells_c5_CD8+_GZMK | 2 | 0.000323729 | HER2+ |
| CID3921 | T_cells_c5_CD8+_GZMK | 0 | 0 | HER2+ |
| CID45171 | T_cells_c5_CD8+_GZMK | 0 | 0 | HER2+ |
| CID3838 | T_cells_c5_CD8+_GZMK | 0 | 0 | HER2+ |
| CID4066 | T_cells_c5_CD8+_GZMK | 0 | 0 | HER2+ |
| CID44041 | T_cells_c5_CD8+_GZMK | 0 | 0 | TNBC |
| CID4465 | T_cells_c5_CD8+_GZMK | 0 | 0 | TNBC |
| CID4495 | T_cells_c5_CD8+_GZMK | 270 | 0.0338134 | TNBC |
| CID44971 | T_cells_c5_CD8+_GZMK | 8 | 0.001001753 | TNBC |
| CID44991 | T_cells_c5_CD8+_GZMK | 1 | 0.000142389 | TNBC |
| CID4513 | T_cells_c5_CD8+_GZMK | 1 | 0.000177968 | TNBC |
| CID4515 | T_cells_c5_CD8+_GZMK | 0 | 0 | TNBC |
| CID4523 | T_cells_c5_CD8+_GZMK | 0 | 0 | TNBC |
| CID3946 | T_cells_c5_CD8+_GZMK | 0 | 0 | TNBC |
| CID3963 | T_cells_c5_CD8+_GZMK | 0 | 0 | TNBC |
| CID4461 | T_cells_c5_CD8+_GZMK | 0 | 0 | ER+ |
| CID4463 | T_cells_c5_CD8+_GZMK | 0 | 0 | ER+ |
| CID4471 | T_cells_c5_CD8+_GZMK | 0 | 0 | ER+ |
| CID4530N | T_cells_c5_CD8+_GZMK | 0 | 0 | ER+ |
| CID4535 | T_cells_c5_CD8+_GZMK | 0 | 0 | ER+ |
| CID4040 | T_cells_c5_CD8+_GZMK | 2 | 0.000790202 | ER+ |
| CID3941 | T_cells_c5_CD8+_GZMK | 0 | 0 | ER+ |
| CID3948 | T_cells_c5_CD8+_GZMK | 0 | 0 | ER+ |
| CID4067 | T_cells_c5_CD8+_GZMK | 0 | 0 | ER+ |
| CID4290A | T_cells_c5_CD8+_GZMK | 0 | 0 | ER+ |
| CID4398 | T_cells_c5_CD8+_GZMK | 0 | 0 | ER+ |
| CID3586 | T_cells_c6_IFIT1 | 82 | 0.013272904 | HER2+ |
| CID3921 | T_cells_c6_IFIT1 | 12 | 0.003968254 | HER2+ |
| CID45171 | T_cells_c6_IFIT1 | 29 | 0.011851246 | HER2+ |
| CID3838 | T_cells_c6_IFIT1 | 31 | 0.013174671 | HER2+ |
| CID4066 | T_cells_c6_IFIT1 | 34 | 0.006404219 | HER2+ |
| CID44041 | T_cells_c6_IFIT1 | 16 | 0.007508212 | TNBC |
| CID4465 | T_cells_c6_IFIT1 | 0 | 0 | TNBC |
| CID4495 | T_cells_c6_IFIT1 | 358 | 0.044834064 | TNBC |
| CID44971 | T_cells_c6_IFIT1 | 114 | 0.014274981 | TNBC |
| CID44991 | T_cells_c6_IFIT1 | 20 | 0.002847786 | TNBC |
| CID4513 | T_cells_c6_IFIT1 | 57 | 0.010144154 | TNBC |
| CID4515 | T_cells_c6_IFIT1 | 26 | 0.00626657 | TNBC |
| CID4523 | T_cells_c6_IFIT1 | 4 | 0.002280502 | TNBC |
| CID3946 | T_cells_c6_IFIT1 | 4 | 0.005167959 | TNBC |
| CID3963 | T_cells_c6_IFIT1 | 79 | 0.022398639 | TNBC |
| CID4461 | T_cells_c6_IFIT1 | 1 | 0.001584786 | ER+ |
| CID4463 | T_cells_c6_IFIT1 | 2 | 0.001757469 | ER+ |
| CID4471 | T_cells_c6_IFIT1 | 9 | 0.001045418 | ER+ |
| CID4530N | T_cells_c6_IFIT1 | 7 | 0.001587662 | ER+ |
| CID4535 | T_cells_c6_IFIT1 | 13 | 0.003281999 | ER+ |
| CID4040 | T_cells_c6_IFIT1 | 29 | 0.011457922 | ER+ |
| CID3941 | T_cells_c6_IFIT1 | 2 | 0.003169572 | ER+ |
| CID3948 | T_cells_c6_IFIT1 | 32 | 0.013751612 | ER+ |
| CID4067 | T_cells_c6_IFIT1 | 3 | 0.000797024 | ER+ |
| CID4290A | T_cells_c6_IFIT1 | 4 | 0.000690966 | ER+ |
| CID4398 | T_cells_c6_IFIT1 | 49 | 0.011008762 | ER+ |
| CID3586 | T_cells_c7_CD8+_IFNG | 279 | 0.045160246 | HER2+ |
| CID3921 | T_cells_c7_CD8+_IFNG | 117 | 0.038690476 | HER2+ |
| CID45171 | T_cells_c7_CD8+_IFNG | 149 | 0.060890887 | HER2+ |
| CID3838 | T_cells_c7_CD8+_IFNG | 75 | 0.031874203 | HER2+ |
| CID4066 | T_cells_c7_CD8+_IFNG | 197 | 0.0371068 | HER2+ |
| CID44041 | T_cells_c7_CD8+_IFNG | 65 | 0.030502112 | TNBC |
| CID4465 | T_cells_c7_CD8+_IFNG | 32 | 0.020460358 | TNBC |
| CID4495 | T_cells_c7_CD8+_IFNG | 42 | 0.005259862 | TNBC |
| CID44971 | T_cells_c7_CD8+_IFNG | 377 | 0.047207613 | TNBC |
| CID44991 | T_cells_c7_CD8+_IFNG | 75 | 0.010679197 | TNBC |
| CID4513 | T_cells_c7_CD8+_IFNG | 118 | 0.021000178 | TNBC |
| CID4515 | T_cells_c7_CD8+_IFNG | 15 | 0.003615329 | TNBC |
| CID4523 | T_cells_c7_CD8+_IFNG | 3 | 0.001710376 | TNBC |
| CID3946 | T_cells_c7_CD8+_IFNG | 12 | 0.015503876 | TNBC |
| CID3963 | T_cells_c7_CD8+_IFNG | 286 | 0.081088744 | TNBC |
| CID4461 | T_cells_c7_CD8+_IFNG | 5 | 0.00792393 | ER+ |
| CID4463 | T_cells_c7_CD8+_IFNG | 44 | 0.038664323 | ER+ |
| CID4471 | T_cells_c7_CD8+_IFNG | 61 | 0.007085608 | ER+ |
| CID4530N | T_cells_c7_CD8+_IFNG | 53 | 0.012020866 | ER+ |
| CID4535 | T_cells_c7_CD8+_IFNG | 21 | 0.005301691 | ER+ |
| CID4040 | T_cells_c7_CD8+_IFNG | 83 | 0.032793362 | ER+ |
| CID3941 | T_cells_c7_CD8+_IFNG | 68 | 0.107765452 | ER+ |
| CID3948 | T_cells_c7_CD8+_IFNG | 238 | 0.102277611 | ER+ |
| CID4067 | T_cells_c7_CD8+_IFNG | 158 | 0.041976621 | ER+ |
| CID4290A | T_cells_c7_CD8+_IFNG | 81 | 0.013992054 | ER+ |
| CID4398 | T_cells_c7_CD8+_IFNG | 514 | 0.115479667 | ER+ |
| CID3586 | T_cells_c8_CD8+_LAG3 | 91 | 0.014729686 | HER2+ |
| CID3921 | T_cells_c8_CD8+_LAG3 | 24 | 0.007936508 | HER2+ |
| CID45171 | T_cells_c8_CD8+_LAG3 | 23 | 0.009399264 | HER2+ |
| CID3838 | T_cells_c8_CD8+_LAG3 | 67 | 0.028474288 | HER2+ |
| CID4066 | T_cells_c8_CD8+_LAG3 | 40 | 0.007534376 | HER2+ |
| CID44041 | T_cells_c8_CD8+_LAG3 | 5 | 0.002346316 | TNBC |
| CID4465 | T_cells_c8_CD8+_LAG3 | 1 | 0.000639386 | TNBC |
| CID4495 | T_cells_c8_CD8+_LAG3 | 355 | 0.044458359 | TNBC |
| CID44971 | T_cells_c8_CD8+_LAG3 | 802 | 0.100425745 | TNBC |
| CID44991 | T_cells_c8_CD8+_LAG3 | 48 | 0.006834686 | TNBC |
| CID4513 | T_cells_c8_CD8+_LAG3 | 58 | 0.010322121 | TNBC |
| CID4515 | T_cells_c8_CD8+_LAG3 | 40 | 0.009640877 | TNBC |
| CID4523 | T_cells_c8_CD8+_LAG3 | 15 | 0.008551881 | TNBC |
| CID3946 | T_cells_c8_CD8+_LAG3 | 5 | 0.006459948 | TNBC |
| CID3963 | T_cells_c8_CD8+_LAG3 | 252 | 0.071448823 | TNBC |
| CID4461 | T_cells_c8_CD8+_LAG3 | 0 | 0 | ER+ |
| CID4463 | T_cells_c8_CD8+_LAG3 | 2 | 0.001757469 | ER+ |
| CID4471 | T_cells_c8_CD8+_LAG3 | 0 | 0 | ER+ |
| CID4530N | T_cells_c8_CD8+_LAG3 | 1 | 0.000226809 | ER+ |
| CID4535 | T_cells_c8_CD8+_LAG3 | 7 | 0.00176723 | ER+ |
| CID4040 | T_cells_c8_CD8+_LAG3 | 72 | 0.028447254 | ER+ |
| CID3941 | T_cells_c8_CD8+_LAG3 | 8 | 0.012678288 | ER+ |
| CID3948 | T_cells_c8_CD8+_LAG3 | 14 | 0.00601633 | ER+ |
| CID4067 | T_cells_c8_CD8+_LAG3 | 4 | 0.001062699 | ER+ |
| CID4290A | T_cells_c8_CD8+_LAG3 | 2 | 0.000345483 | ER+ |
| CID4398 | T_cells_c8_CD8+_LAG3 | 19 | 0.004268704 | ER+ |
| CID3586 | T_cells_c9_NK_cells_AREG | 130 | 0.021042409 | HER2+ |
| CID3921 | T_cells_c9_NK_cells_AREG | 60 | 0.01984127 | HER2+ |
| CID45171 | T_cells_c9_NK_cells_AREG | 87 | 0.035553739 | HER2+ |
| CID3838 | T_cells_c9_NK_cells_AREG | 75 | 0.031874203 | HER2+ |
| CID4066 | T_cells_c9_NK_cells_AREG | 101 | 0.019024298 | HER2+ |
| CID44041 | T_cells_c9_NK_cells_AREG | 21 | 0.009854528 | TNBC |
| CID4465 | T_cells_c9_NK_cells_AREG | 2 | 0.001278772 | TNBC |
| CID4495 | T_cells_c9_NK_cells_AREG | 52 | 0.00651221 | TNBC |
| CID44971 | T_cells_c9_NK_cells_AREG | 94 | 0.011770599 | TNBC |
| CID44991 | T_cells_c9_NK_cells_AREG | 20 | 0.002847786 | TNBC |
| CID4513 | T_cells_c9_NK_cells_AREG | 205 | 0.03648336 | TNBC |
| CID4515 | T_cells_c9_NK_cells_AREG | 41 | 0.009881899 | TNBC |
| CID4523 | T_cells_c9_NK_cells_AREG | 44 | 0.025085519 | TNBC |
| CID3946 | T_cells_c9_NK_cells_AREG | 1 | 0.00129199 | TNBC |
| CID3963 | T_cells_c9_NK_cells_AREG | 273 | 0.077402892 | TNBC |
| CID4461 | T_cells_c9_NK_cells_AREG | 2 | 0.003169572 | ER+ |
| CID4463 | T_cells_c9_NK_cells_AREG | 3 | 0.002636204 | ER+ |
| CID4471 | T_cells_c9_NK_cells_AREG | 30 | 0.003484725 | ER+ |
| CID4530N | T_cells_c9_NK_cells_AREG | 11 | 0.002494897 | ER+ |
| CID4535 | T_cells_c9_NK_cells_AREG | 25 | 0.006311537 | ER+ |
| CID4040 | T_cells_c9_NK_cells_AREG | 107 | 0.04227578 | ER+ |
| CID3941 | T_cells_c9_NK_cells_AREG | 18 | 0.028526149 | ER+ |
| CID3948 | T_cells_c9_NK_cells_AREG | 58 | 0.024924796 | ER+ |
| CID4067 | T_cells_c9_NK_cells_AREG | 48 | 0.012752391 | ER+ |
| CID4290A | T_cells_c9_NK_cells_AREG | 50 | 0.00863707 | ER+ |
| CID4398 | T_cells_c9_NK_cells_AREG | 288 | 0.064704561 | ER+ |

### Lymphocytes and Innate Lymphoid Cells

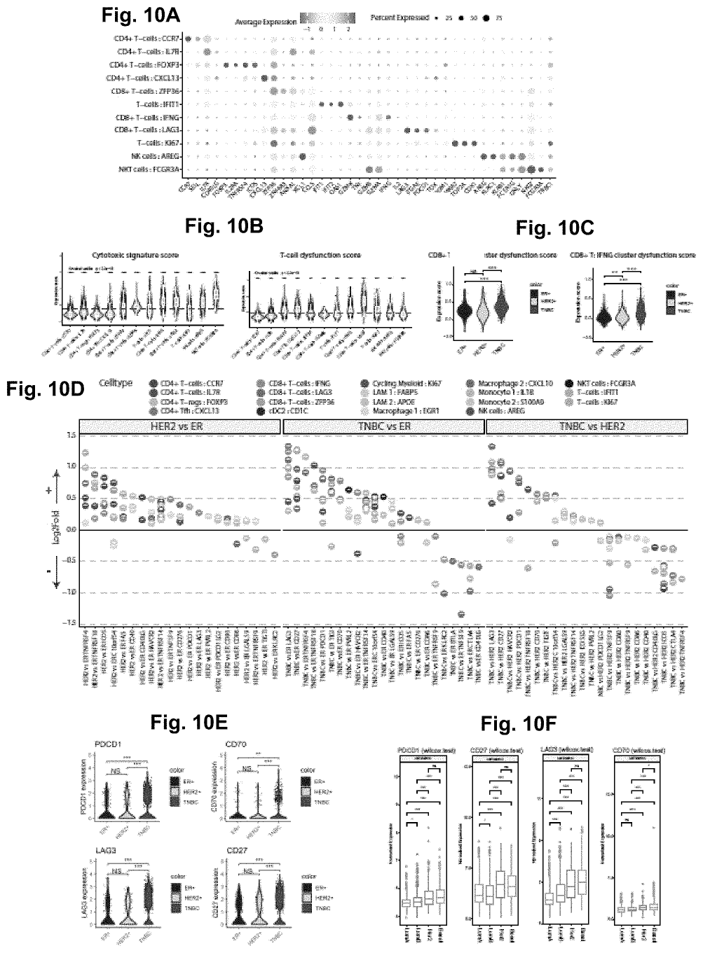

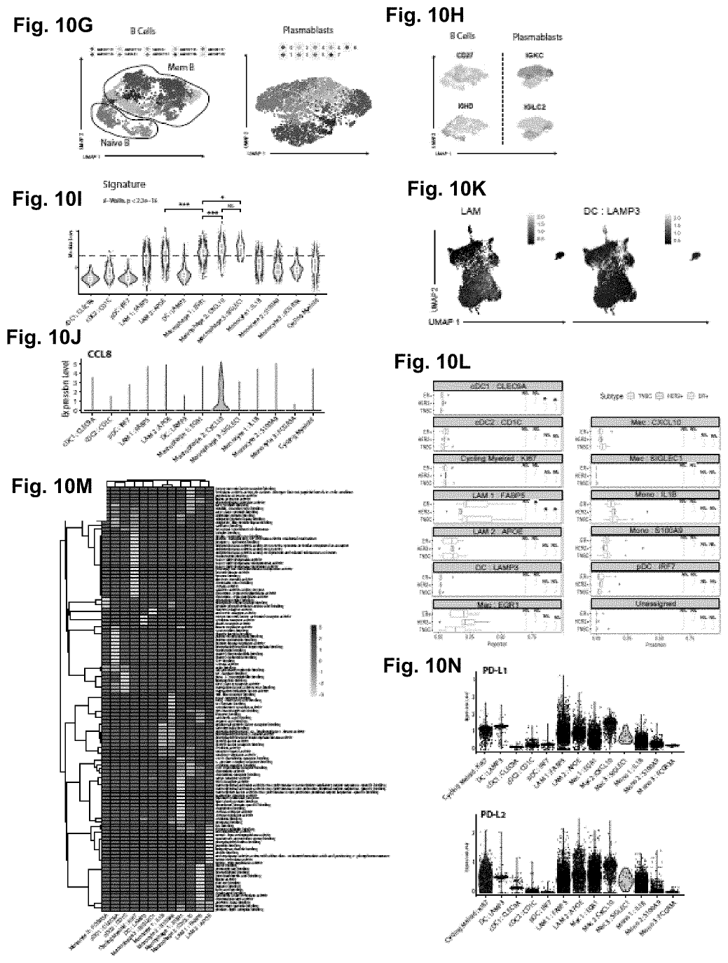

A total of 18 T-cell and innate lymphoid clusters were identified based on RNA expression, which were detected in the majority of cases (FIG. 8A). CD4 clusters (c0, c1, c2 and c3) were comprised of regulatory T cells (T-Regs) marked by FOXP3 mRNA and CD25 protein expression (CD4+ T-cells:FOXP3/c2), T follicular helper (Tfh) cells with high CXCL13, IL21 and PDCD1 expression (CD4+ T-cells:CXCL13/c3), naïve/central memory CD4+(CD4+ T-cells:CCR7/c0), and a Th1 CD4 T effector memory (EM) cluster (CD4+ T-cells:IL7R/c1) (FIG. 8B; FIG. 10A). The significant numbers of Tfh cells observed is consistent with the frequent observation of tertiary lymphoid structures (TLS) in BrCa.

We identified five CD8 T-cell clusters (c4, c5, c7, c8 and c17), two of which were specific to individual tumours (c8, c17). The remaining three were exhausted tissue resident memory (TRM) CD8+ T-cells expressing high levels of inhibitory checkpoint molecules including LAG3, PDCD1 and TIGIT (CD8+ T-cells:LAG3/c8), TRM PDCD1low CD8+ T-cells that expressed relatively high levels of IFNG and TNF (CD8+ T-cells:IFNG/c7), and CD8+ effector memory (EM) chemokine expressing T-cells (CD8+ T-cells:ZFP36/c4) (FIG. 10A). Two additional T-cell clusters were identified. One cluster was driven by a type 1 interferon (IFN) signature including high mRNA levels of IFN-induced genes SG15, IFIT1 and OAS1 (T-cells:IFIT1/c6) and was composed of roughly equal numbers of CD4+ and CD8+ T-cells. A proliferating T-cell cluster (T-cells:MKI67/c11) was also made up of CD4+ and CD8+ T-cells. The remaining four clusters (c12, c13, c15 and c16) were unassigned, with the latter two being tumour specific and the former two not mapped to any known cell type, potentially comprising cell doublets. We also identified an NK cell cluster (NK cells:AREG/c9) and NKT-like cell cluster (NKT cells:FCGR3A/c10) by their expression of αβ T-cell receptor and NK markers (KLRC1, KLRB1, NKG7) (FIG. 8B; FIG. 10A).

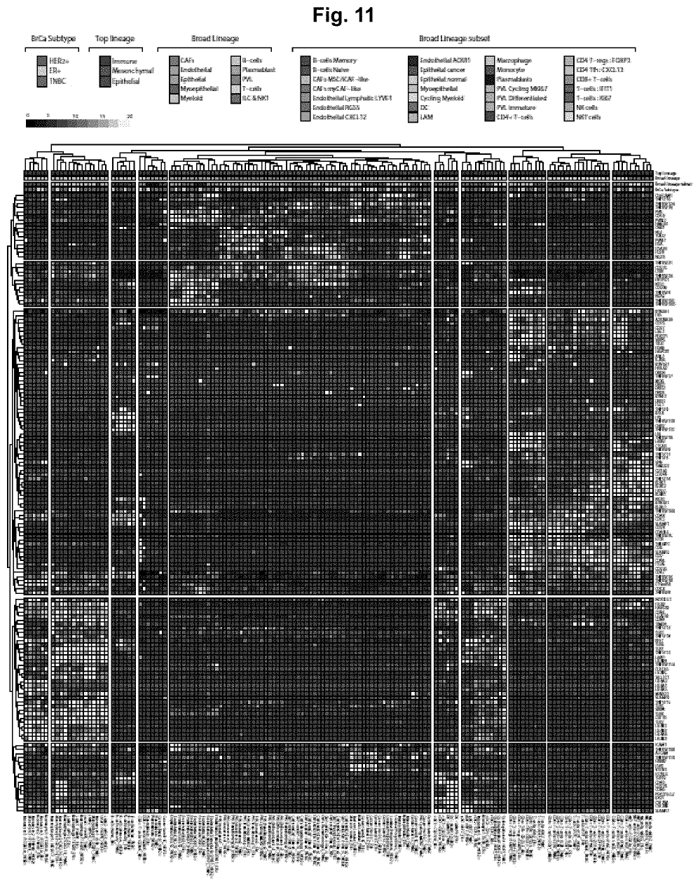

TNBC have more TILs in general and CD8+ T-cells in particular. We also observed that T cell clusters IFIT1/c6, LAG3/c8 and MKI67/c11 made up a higher proportion of T cells in TNBC samples compared to other subsets (FIG. 8C). These clusters had qualitative differences between subtypes of BrCa, with CD8+ T-cells from both the LAG3/c8 and IFNG/c7 clusters possessing substantially higher dysfunction scores (Li, H. et al., (2019) Cell 176, 775-789 e18). in TNBC cases (FIG. 8D; FIGS. 10B-10C). Furthermore, luminal and HER2+ BrCa tended to have checkpoint molecule expression distinct from TNBC (FIG. 8I; FIG. 10D). Notably, The LAG3/c8 exhausted CD8 subset had altered expression of immunoregulatory molecules in TNBC, including significantly higher expression of PD-1 (PDCD1), LAG3 and the ligand-receptor pair of CD27 and CD70, known to enhance T-cell cytotoxicity42 (FIG. 8I; FIG. 10E). We examined the expression of PDCD1, CD27 and CD70 in the METABRIC and TCGA bulk tumour cohorts, which showed consistent enrichment of these markers in basal-like and HER2+ BrCa (FIG. 10F). Furthermore, basal-like and HER2+ BrCa had higher infiltration of PD-1+ T-cells in recent immunofluorescence studies. When we examined a wider list of immune checkpoint molecules across the entire dataset using unsupervised hierarchical clustering (FIG. 11), differences in checkpoint molecule expression among BrCa subtypes were more apparent, including on non-immune cells such as CAFs. These data provide insights into the immunotherapeutic strategies most appropriate for each subtype of disease.

When we reclustered B cells, we observed two major subclusters (naive and memory), with plasmablasts forming a separate cluster (FIGS. 10G-10I). The additional subclusters seemed largely driven by BCR specific gene segments rather than variable biological gene expression programs.

### Myeloid Cells

Myeloid cells formed 13 clusters which could be identified in all tumours at varying frequencies, with the exception of macrophage cluster 5 that was mostly limited to an individual tumour (FIG. 8E). No granulocytes were detected, likely due to their sensitivity to tumour dissociation protocols and their low abundance. Monocytes formed 3 clusters: Mono:IL1B/c12; Mono:S100A9/c8; and Mono:FCGR3A/c7, with the Mono:FCGR3A population forming a small distinct cluster characterized by high CD16 protein expression. We identified conventional dendritic cells (cDC) that expressed either CLEC9A (cDC1:CLEC9A/c3) or CD1C (cDC2:CD1C/c11); plasmacytoid DC (pDC) that expressed IRF7 (pDC:IRF7/c4); and a LAMP3 high DC population46 (DC:LAMP3/c0), which was previously not reported in single cell studies of BrCa.

Macrophages formed 6 clusters, including a cluster (Mac:CXCL10/c9) with features previously associated with an “M1-like” phenotype and two clusters (Mac:EGR1/c10 and Mac:SIGLEC1/c5) resembling the “M2-like” phenotype. All of which bear some resemblance to TAMs previously described in BrCa (FIG. 10J). Notably, we identified two novel macrophage populations (LAM1:FABP5/c1 and LAM2:APOE/c2) outside of the conventional “M1/M2” classification that comprised 30-40% of the total myeloid cells but do not appear to have been reported in BrCa previously (FIG. 8F; FIG. 10K). These cells bear close transcriptomic similarity to a recently described population of lipid-associated macrophages (LAM) that expand in obese mice and humans, including high expression of TREM2 and lipid/fatty acid metabolic genes such as FABP5 and APOE (FIG. 8F; FIG. 10L). LAM1/2 were also unique amongst myeloid cells in expressing CCL18, which encodes a chemokine with roles in immune regulation and direct tumour promotion (Chen et al., (2011) Cancer Cell 19, 541-55). We observed a substantially reduced proportion of LAM 1:FABP5 cells in the HER2+ tumours (FIG. 8C; FIG. 10M), suggesting that unique features of the tumour microenvironment regulate LAM1/2 differentiation or survival. Survival analysis using the METABRIC cohort showed that the LAM 1:FABP5 signature correlates with worse survival in BrCa patients (FIG. 8G). While the RNA encoding PD-L1 (CD274) and PD-L2 (PDCD1LG2) were highly co-expressed by the Mac:CXCL10 and DC:LAMP3 myeloid populations (FIG. 8I), analysis of CITE-Seq data demonstrated a broader distribution of PD-L1 and PD-L2 protein expression across the Mac:CXCL10, LAM1:FABP5, LAM2:APOE and DC:LAMP3 (FIG. 8H; FIG. 10N), highlighting LAM1/2 as important sources of immunoregulatory molecules and demonstrating the value of CITE-Seq data to immune cell profiling.

### Mesenchymal Subclasses in Breast Cancer Resemble Diverse Differentiation States

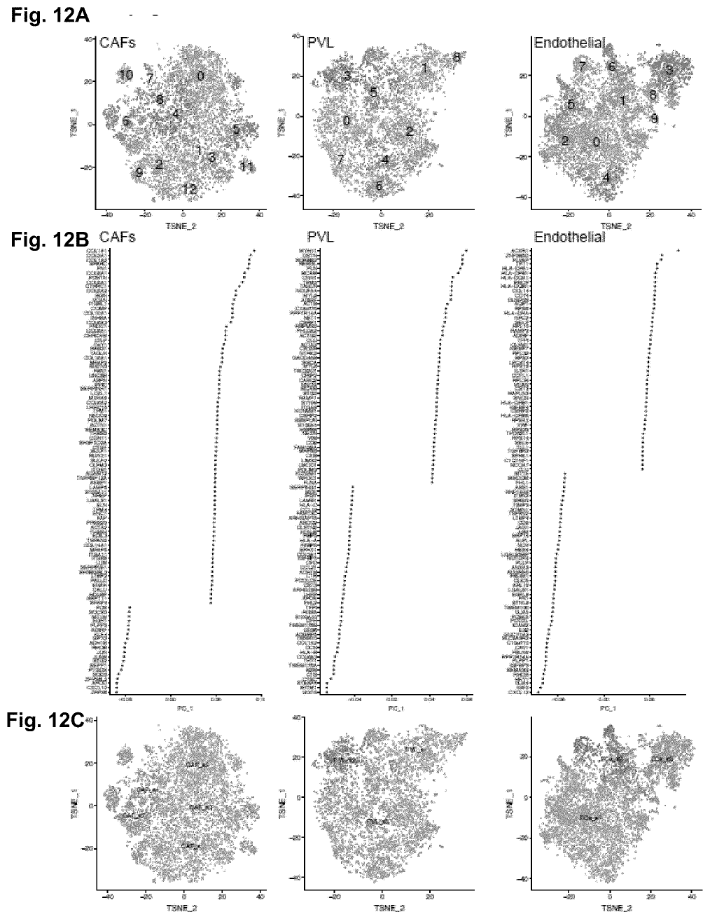

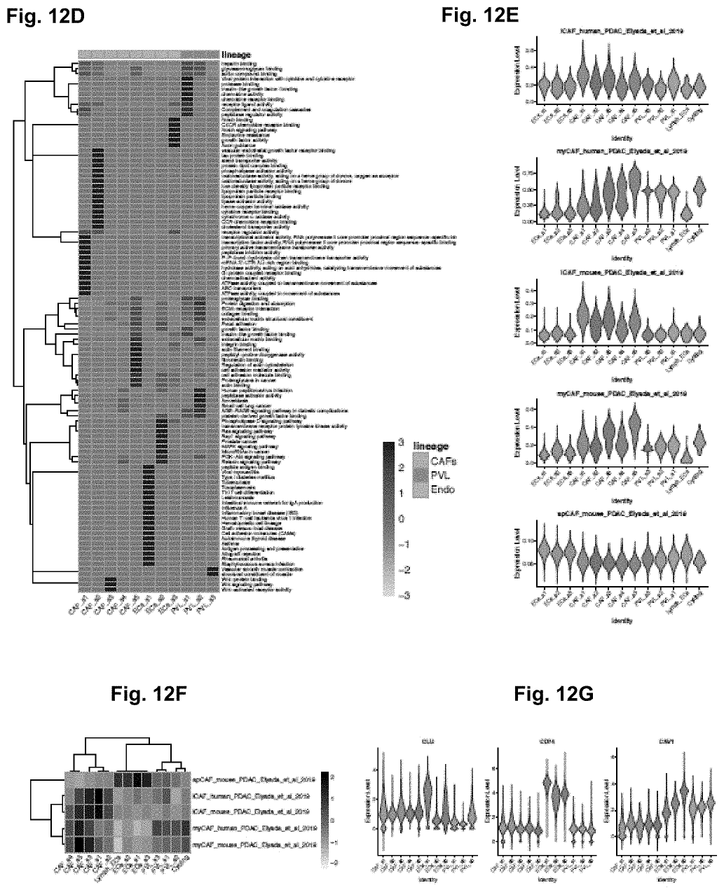

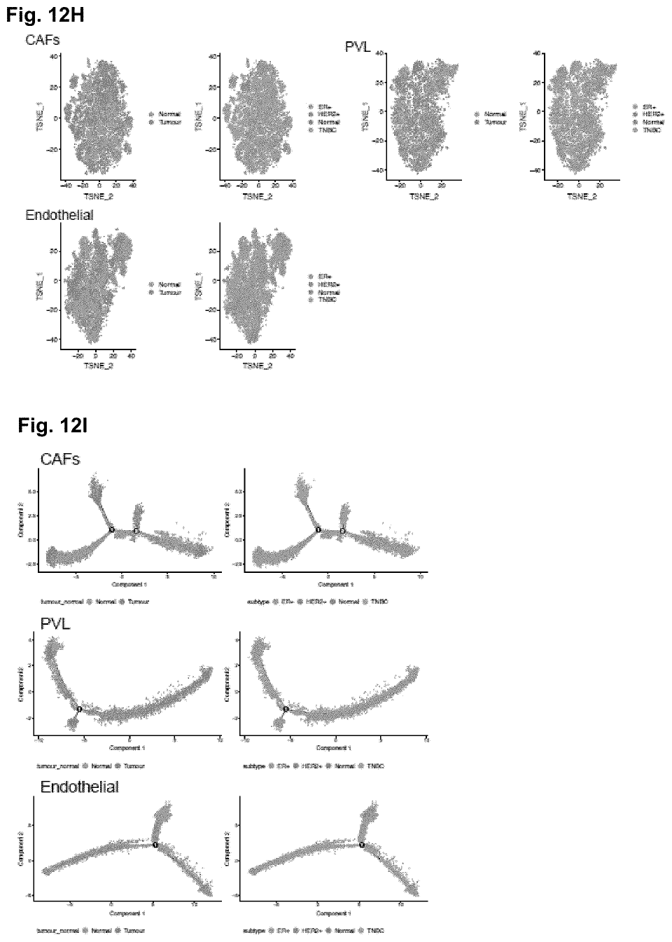

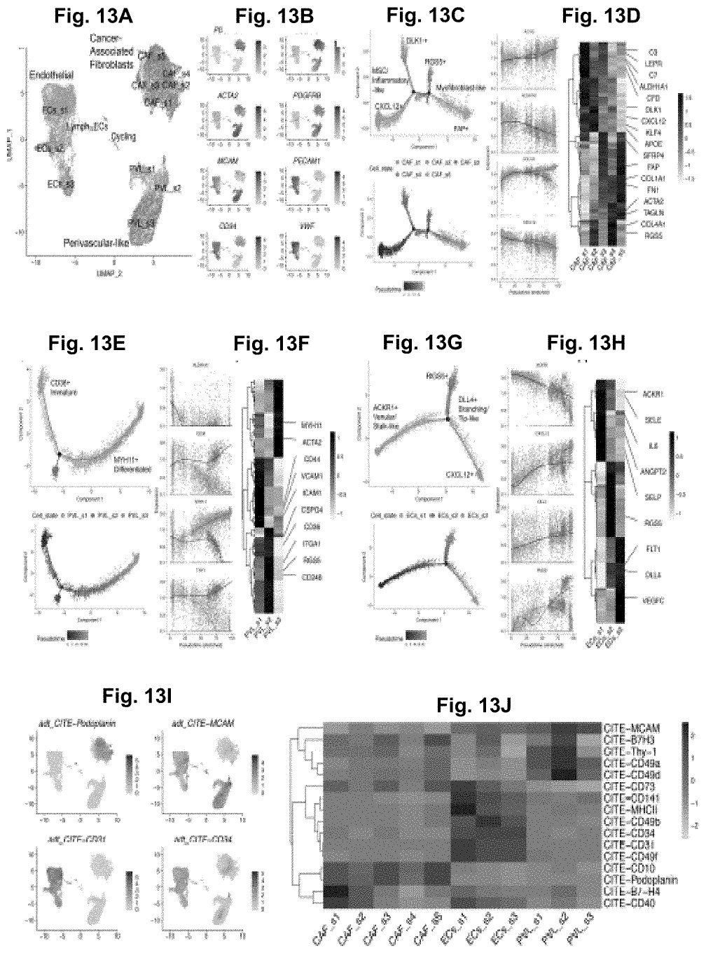

The stromal cell types and subclasses present in human BrCa are yet to be profiled at high resolution and across clinical subtypes. We identified three major mesenchymal cell types including CAFs (PDGFRA and COL1A1), perivascular-like cells (PVL; MCAM/CD146, ACTA2 and PDGFRB), endothelial cells (PECAM1/CD31 and CD34), and two smaller clusters of lymphatic endothelial cells (LYVE1) and cycling PVL cells (MKI67) (FIGS. 13A-13B; FIG. 12A). Reclustering within each cell type revealed an enrichment of cell differentiation markers in the principal component (PC1) explaining most of the variance, including cytoskeletal components (ACTA2, TAGLN and MYH11), fibroblast activation markers (FAP, THY1 and VWF) and ECM synthesis (COL1A1 and FN1) (FIG. 12B). From this we hypothesized that sub-clusters represented a spectrum of cell differentiation states rather than distinct phenotypes. For each of the three major lineages, we applied the Monocle49 method to order cells along a pseudo-temporal trajectory to define cell states and independently estimate genes and proteins expression which change throughout differentiation (FIGS. 13C-13H; FIG. 12C).

### Cancer-Associated Fibroblasts

Trajectory analysis revealed five CAF states with two distinct branch points (FIG. 13C). State 1 (referred to as s1 herein) had features of mesenchymal stem cells and inflammatory-like fibroblasts (iCAFs), with high expression of stem-cell markers (ALDH1A1, KLF4 and LEPR) and chemokines and complement factors (CXCL12 and C3) (FIGS. 13C-13D). The expression of these markers decreased as cells transitioned towards differentiated states s4 and s5, which rather resembled a myofibroblast-like (myCAF) state through the increased expression of ACTA2 (aSMA), TAGLN, FAP and COL1A1 (FIGS. 13C-13D)16. Gene ontology (GO) analysis revealed that pathways related to transcriptional factor activity, chemoattraction and complement/coagulation cascades were enriched in CAF s1 whereas CAF s2 was enriched for lipoprotein and cytokine/chemokine receptor binding pathways (FIG. 12D). Consistent with the predicted phenotypes of myCAFs, CAF state s5 was enriched for ECM synthesis, actin and integrin binding and focal adhesion (FIG. 12D).

Previously reported pancreatic ductal adenocarcinoma (PDAC) CAF signatures20, defined by iCAFs and myCAFs, were predominantly enriched in CAF s1 and s5, respectively (FIGS. 12E-12F). No CAF states were enriched for PDAC antigen presentation (apCAFs) gene signatures (FIGS. 12E-12F), however, selected apCAF markers CD74, CLU and CAV1 were expressed by cells within CAF s1 (FIG. 12G) Immunoregulatory molecules B7-H4 and CD40 were highly expressed by the MSC/inflammatory-like CAF s1 and s2 by CITE-Seq (FIGS. 131-13J), suggesting an immunoregulatory role of these subclasses.

### Perivascular-Like Cells

Trajectory analysis revealed three main PVL states with a single branch point (FIG. 13E). PVL s1 and s2 expressed stem-cell and immature perivascular markers including PDGFRB, ALDH1A1, CD44, CSPG4, RGS5 and CD36 (FIGS. 13E-13F). The branching of s2 was defined by markers including RGS5, CD248 and THY1 (Tables 9 and 10). PVL s1 and s2 also expressed adhesion molecules including ICAM1, VCAM1 and ITGB1 (FIGS. 13E-13F).

The expression of these genes decreased along the pseudotime trajectory as cells transitioned to PVL s3, which was enriched for contractile related genes including MYH11 and ACTA2 (FIGS. 13E-13F). PVL s3 was further defined by pathways related to vascular smooth muscle contraction and muscle system processes, and likely resemble a smooth muscle phenotype (FIG. 12D). In contrast, the immature states PVL s1 and s2 were enriched for receptor binding and PDGF activity (FIG. 12D).

Interestingly, all PVL states were also modestly enriched for PDAC myCAF gene signatures, suggesting that they have shared transcriptional features related to contractility (FIGS. 12E-12F). ACTA2 appears extensively in the literature as a marker of CAFs, suggesting that PVL s3 has historically been misclassified in immunohistochemical assays as CAFs. Consistent with the scRNA-Seq findings, CITE-Seq revealed an enrichment of the cell surface molecules CD90 (THY1) and integrin molecules CD49a and CD49d in early PVL states s1 and s2 (FIGS. 131-13J), while these adhesion molecules decreased in PVL s3. Our findings suggest subclasses of PVL cells resemble early and late differentiation states, showing features of cell adhesion and contractility, respectively.

### Endothelial Cells

Endothelial cells sub-clustered into three pseudotime states with one distinct branch point (FIG. 13G). Endothelial s1 had high expression of ACKR1, SELE and SELP (FIGS. 13G-13H). These markers are highly expressed by stalk-like and venular endothelial cells, which regulate leukocyte migration into tissue sites through integrin mediated adhesion molecules. Consistent with this, endothelial s1 had high expression of adhesion (ICAM1 and VCAM1) and MHC molecules (HLA-DRA) (FIGS. 13G-13H). These markers decreased along the pseudotime trajectory as cells branched into two states, which both had elevated expression of the notch activating ligand gene DLL4, a marker reported for endothelial sprouting, branching, expansion and tip-like cells (FIGS. 13G-13H). Endothelial s2 could be distinguished from s3 through the expression of RGS5 and ESM1 (FIGS. 13G-13H). Key regulators of cell migration and angiogenesis, including CXCL12 and VEGFC54, distinguished endothelial s3 from s2 (FIGS. 13G-13H). Consistent with these predicted phenotypes, endothelial s1 was enriched for pathways related to immune response, antigen processing and presentation, hematopoietic cell lineage and cell adhesion molecules (FIG. 12D). In contrast, endothelial s3 was enriched for Notch signalling, chemokine binding and axon guidance (FIG. 12A). CITE-Seq (FIGS. 131-13J) revealed an enrichment of the cell surface molecules CD49f, CD73, CD141, CD40 and MHC class II in endothelial s1. As angiogenesis is known to be a dynamic process involving the transition between endothelial stalk and tip cells, it is likely that these states are dynamic and interconvertible. In summary, we identified three major endothelial cell states defined by markers ACRK1, RGS5 and CXCL12, for venular stalk-like and two sprouting tip-like subsets, respectively.

To determine whether stromal states were unique to the TME, we performed scRNA-Seq on three normal breast tissue samples and were surprised to find that no clusters or cell states were unique to disease status or subtypes (FIGS. 12H-121). This suggests that the mesenchymal subsets described in this study are likely resident cell types that undergo quantitative remodelling in the TME.

Deconvolution of Breast Cancer Cohorts Reveals Nine Ecotypes Associated with Patient Survival

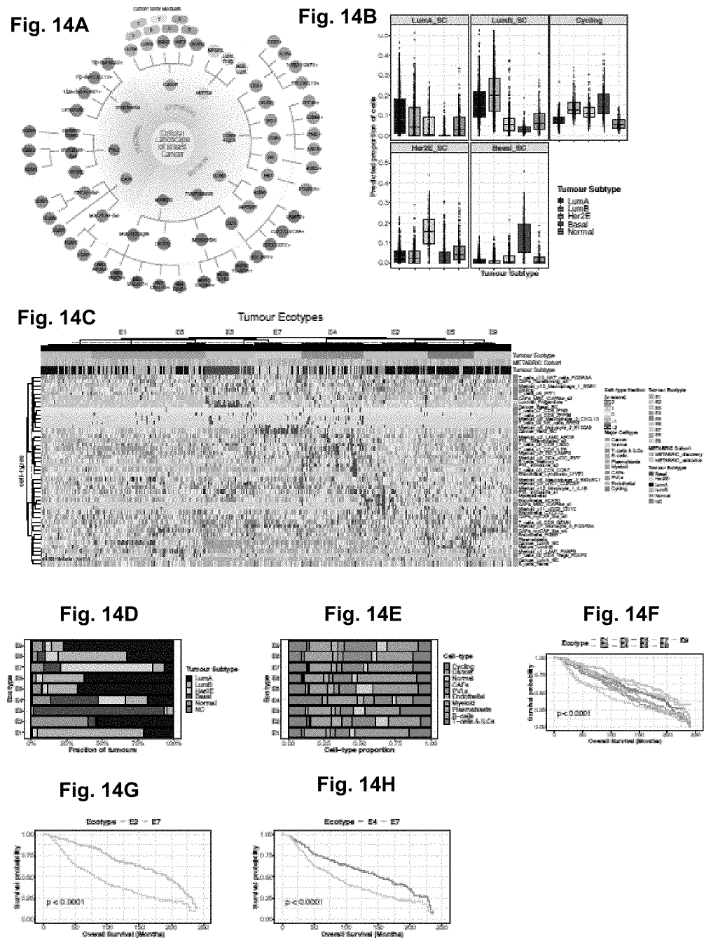

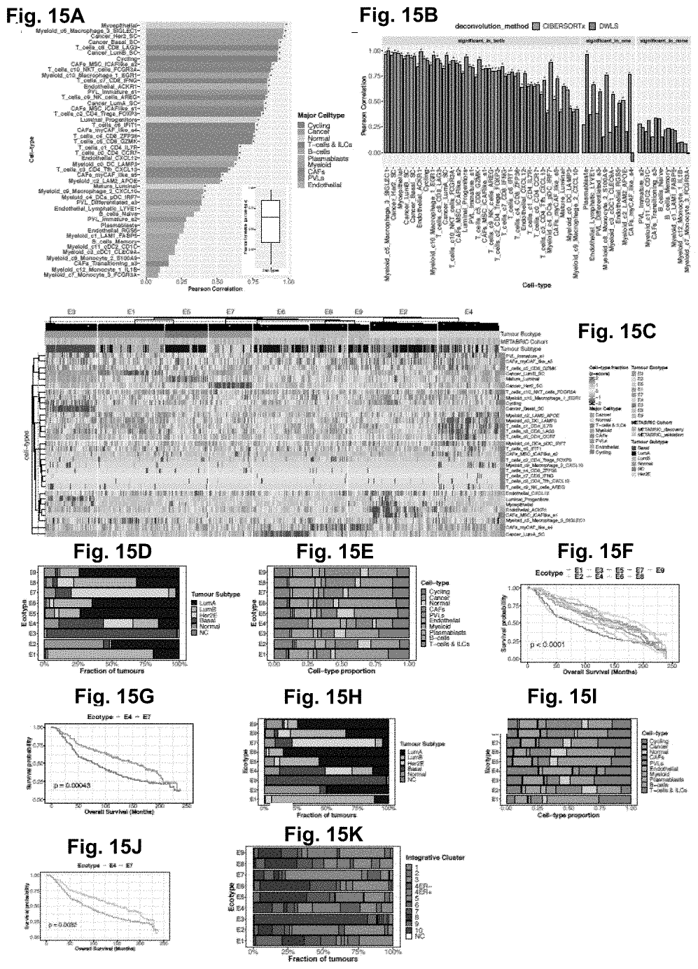

Our single cell data has generated a draft cellular taxonomy of BrCa, with at least three tiers of cell types and states (Major, Minor and Subset; FIG. 14A). We observed marked variation in cellular frequencies across 26 tumours, with some recurring patterns observed. We hypothesized that far from being random, subsets of BrCa may have similarities in their cellular composition, resulting in similarities in tumour biology. To test this hypothesis at a large scale, we estimated cellular proportions in bulk RNA-Seq samples by using our single-cell signatures with the CIBERSORTx method. Estimating cell fractions from pseudo-bulk samples generated from our single-cell datasets showed good overall correlation between the actual captured cell-fractions and the CIBERSORTx predicted proportions (median correlation ˜0.64) (FIG. 15A), with a majority (32) of cell-types showing a significant correlation (FIG. 15A). An alternative deconvolution method, DWLS, showed similar results (FIG. 15B). This suggests that deconvolution methods can effectively predict high-resolution BrCa cellular composition from bulk RNA-Seq data.

We deconvoluted all primary breast tumour datasets in the METABRIC cohort. Supporting the validity of the predictions (and the scSubtype signatures), we observed significant enrichment (Wilcox test, p<2.2e-16) of the four scSubtypes (Basal_SC, HER2E_SC, LumA_SC and LumB_SC) in tumours with matching bulk-PAM50 classifications and significant enrichment (Wilcox test, p<2.2e-16) of cycling cells in Basal, LumB and HER2E tumours (FIG. 15B). Consensus clustering of our “subset” cell classification tier revealed 9 tumour clusters with similar estimated cellular composition (“Ecotypes”) (FIG. 15C). These ecotypes displayed some correlations with tumour subtype and scSubtype cell distributions and a diverse mix of the major cell-types (FIG. 15C). Ecotype-3 (E3) was enriched for tumours containing Basal_SC, Cycling, and Luminal_Progenitor cells (the presumptive cell of origin for basal breast cancers) and a Basal bulk PAM50 subtype (FIGS. 15C-15D). In contrast, E1, E5, E6, E8 and E9 consisted predominantly of luminal cells. Beyond cancer cell phenotypes, ecotypes also possessed unique patterns of stromal and immune cell enrichment. For instance, E4 was highly enriched for immune cells associated with anti-tumour immunity (FIG. 15C), including exhausted CD8 T cells (LAG3/c8), along with Th1-(IL7R/c1) and central memory (CCR7/c0) CD4 T cells. E2 primarily consisted of LumA and Normal-like tumours (FIG. 15D) and was defined by a cluster of mesenchymal cell types including Endothelial CXCL12+ and ACKR1+ cells, s1 MSC iCAFs and a depletion of cycling cells (FIG. 15E).

We next investigated the prognostic differences between all ecotypes (FIG. 15F). Patients with E2 tumours had the best prognosis (FIGS. 15F-15G), while tumours in E3 were associated with poor overall 5-year survival (FIG. 15F), consistent with known poor prognosis of Basal-like and highly proliferative tumours. E7 also had a poor prognosis and was dominated by HER2E tumours and enrichment of HER2E_SC cells. Interestingly, E4 also had a substantial proportion of HER2E tumours as well as basal-like tumours (FIG. 15D), yet patients with tumours in E4 had significantly better prognosis than those in E7 (FIG. 15H), perhaps as a consequence of infiltration with anti-tumour immune cells.

To further assess the robustness of the ecotypes, we repeated the consensus clustering using only the 32 significantly correlated cell-types, as well as the DWLS method. Substantial overlap of tumours (Table 4 and Table 5) ecotype features (FIGS. 15D-15E, 15H-15I) and overall survival was seen (FIGS. 15F-15G, 15J), suggesting that cells with lower deconvolution performance or specific deconvolution methods were not confounding ecotyping.

Finally, we investigated the association between ecotypes and the integrative genomic clusters (int-clusters) identified by METABRIC (FIG. 15K). Ecotype E3 has a high proportion of cancers from int-cluster 10, which also predominantly consists of basal-like tumours with similarly poor 5-year survival. E7 has a high proportion of int-cluster 5 tumours (defined by ERBB2 amplification and enrichment of Her2E tumours). These are the worst prognosis groups in both the METABRIC and ecotype analysis. However, a majority of ecotypes don't clearly associate with a specific int-cluster or PAM50 subtype, suggesting that cellular ecotypes can identify mixed subtype tumour groups not easily resolved by bulk genomic studies, reflected by the role of the stromal and immune cells in defining ecotypes. This lack of unique associations to ecotypes suggests that ecotypes are not a simple surrogate for molecular or genomic subtypes.

We use deconvolution to define nine ecotypes amongst thousands of primary breast cancers. Interestingly, clustering of most ecotypes is driven by cells spanning the major lineages (epithelial, immune and stromal), features not captured by previous studies that stratified disease based on mass cytometry primarily using immune markers. Integration of our data with these datasets is an important future direction for the field. While ecotypes partially associated with intrinsic subtype and genomic classifiers, they are not simply surrogates for previous methods stratification. Future work will investigate the molecular mechanisms organizing tissue architecture and tumour ecotypes, aiming to explain their differences in clinical outcome and examine whether tumour ecotypes can be used to personalise therapy.

1. A method for the identification of an ecotype within cancer samples, the method comprising:
i. performing or having performed single cell RNA sequencing on cancer sample training sets comprising different cell types and/or cell states;
ii. generating gene expression profiles from the cells of the cancer sample training sets based on the single cell RNA sequencing, wherein each gene expression profile correlates with a distinct cell type and/or cell state;
iii. generating cell abundance profiles, each cell abundance profile being based on the gene expression profile of a respective cancer sample training set; and
iv. using consensus-based clustering on the cancer samples with respect to the cell abundance profiles to identify an ecotype.

2. (canceled)

3. The method according to claim 1, wherein the ecotype is selected from the group consisting of E1, E2, E3, E4, E5, E6, E7, E8 or E9.

4. A method for diagnosing or prognosing cancer in a subject, the method comprising:
i. performing or having performed single cell RNA sequencing on cancer sample training sets, each training set comprising different cell types and/or cell states;
ii. generating cell gene expression profiles from the cells of the cancer sample training sets based on the single cell RNA sequencing, each cell gene expression profile correlating with a distinct cell type and/or cell state;
iii. performing or having performed bulk gene expression RNA sequencing on cancer samples to generate a bulk gene expression profile of the cancer samples;
iv. processing the bulk gene expression profile based on the cell gene expression profiles to generate cell abundance profiles of the cancer samples, each cell abundance profile corresponding to a deconvolution of the bulk gene expression profile with respect to a respective cell gene expression profile;
v. using consensus-based clustering on the cancer samples with respect to the cell abundance profiles to identify an ecotype within cancer samples, and
vi. optionally administering a treatment to the subject based on the diagnosis or prognosis of cancer in the subject,
wherein the ecotype is indicative of a diagnosis or prognosis of cancer in the subject.

5. The method according to claim 4, wherein the method comprises identifying a treatment for the subject based on the identification of an ecotype within the cancer samples, preferably wherein the treatment is selected from the group consisting of chemotherapy, hormonal therapy, radiation therapy, biological therapy such as immunotherapy, small molecule therapy or antibody therapy, or a combination thereof.

6. The method according to claim 5, wherein the method comprises a step of administering the identified treatment.

7. The method according to claim 6, wherein the cancer is selected from the group consisting of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intraepithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumours), and Meigs' syndrome, preferably wherein the cancer is breast cancer.

8. The method according to claim 4, wherein the sample comprises bulk tissue, cells, blood or body fluid, preferably wherein the sample comprises bulk tissue.

9. The method according to claim 8, wherein the sample is a formalin-fixed, paraffin-embedded (FFPE) tissue or a frozen tissue.

10. The method according to claim 4, wherein the sample is obtained from a subject who has, or is suspected of having breast cancer and exhibits one or more of the following symptoms: presence of a lump in the breast or underarm; thickening or swelling of part of the breast; irritation or dimpling of breast skin; redness or flaky skin in the nipple area or the breast; pulling in of the nipple or pain in the nipple area; nipple discharge including blood; any change in the size or the shape of the breast; and pain in an area of the breast.

11. The method according to claim 4, wherein the sample is obtained from a subject who has not received treatment for the cancer.

12. The method according to claim 4, wherein the gene expression profile is normalised to a control, preferably one or more housekeeping genes.

13. The method according to claim 4, wherein the gene expression profile is based on expression of one or more of the genes obtained from a cancer sample.

14. The method according to claim 4, wherein the method comprises one or more diagnostic tests selected from the group consisting of ultrasound; diagnostic x-ray; magnetic resonance imaging (MRI); and biopsy.

15.-18. (canceled)

19. A method for treating cancer in a subject having or suspected of having cancer, the method comprising:
i. performing or having performed single cell RNA sequencing on cancer sample training sets, each training set comprising different cell types and/or cell states;
ii. generating cell gene expression profiles from the cells of the cancer sample training sets based on the single cell RNA sequencing, each cell gene expression profile correlating with a distinct cell type and/or cell state;
iii. performing or having performed bulk gene expression RNA sequencing on cancer samples to generate a bulk gene expression profile of the cancer samples;
iv. processing the bulk gene expression profile based on the cell gene expression profiles to generate cell abundance profiles of the cancer samples, each cell abundance profile corresponding to a deconvolution of the bulk gene expression profile with respect to a respective cell gene expression profile;
v. using consensus-based clustering on the cancer samples with respect to the cell abundance profiles to identify an ecotype within cancer samples; and
vi. administering a treatment to the subject based on the ecotype in the cancer samples,
thereby treating cancer in a subject having or suspected of having cancer.

20. The method according to claim 4, wherein the method comprises providing or having provided cancer samples comprising different cell types.

21. The method according to claim 4, wherein the method comprises training a predictor set of cancer samples from subjects with a known ecotype, diagnosis, prognosis, survival outcome or prediction to drug therapy and applying the predictor to the cancer sample to determine ecotype, diagnosis, prognosis, survival outcome or prediction to drug therapy of the subject.

22. The method according to claim 4, wherein deconvolution comprises estimating cell type abundance using a CIBERSORTx or DWLS deconvolution method.

23. The method according to claim 4, wherein the ecotype comprises cell type abundances selected from the group comprising or consisting of immune enriched cells; cycling cells; normal or healthy cells; PVLs; endothelial cells; myeloid cells; plasmablasts; B-cells; T-cells; innate lymphoid cells (ILCs); cancer associated fibroblasts; immune depleted; high cancer heterogenicity; and combinations thereof.

24. (canceled)

25. The method according to claim 4, wherein the step of performing or having performed bulk gene expression RNA sequencing on cancer samples to generate a bulk gene expression matrix of the cancer samples comprises the generation of bulk gene expression profiles from the same samples or the generation an independent dataset of bulk expression profiles, e.g., METABRIC.

26. The method according to claim 4, wherein the step of generating a gene expression profile from the cells of the training set samples comprises annotating cells within the cancer samples as a specific cell type or cell state.

27.-32. (canceled)
